(19) United States
(12) Patent Application Publication
(10) Pub. No.: US 2023/0373939 A1
(43) Pub. Date: Nov. 23, 2023

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Thomas CLEVELAND, San Marcos, CA (US); Timothy A. DWIGHT, Quincy, MA (US); Bryan A. FRIEMAN, La Jolla, CA (US); Peter GROOTENHUIS, Del Mar, CA (US); Sara Sabina HADIDA RUAH, La Jolla, CA (US); Yoshihiro ISHIHARA, San Diego, CA (US); Mark Thomas MILLER, Rancho Santa Fe, CA (US); Alina SILINA, Needham, MA (US); Jinglan ZHOU, San Diego, CA (US)

(21) Appl. No.: 18/030,519

(22) PCT Filed: Oct. 6, 2021
(86) PCT No.: PCT/US2021/053864

§ 371 Date: Apr. 6, 2023

Related U.S. Application Data
(60) Provisional application No. 63/088,876, filed on Oct. 7, 2020.

(51) Int. Cl. C07D 279/06; C07D 241/22; C07D 251/24; C07D 239/69; C07D 403/04; C07D 213/76; C07D 401/14; C07D 401/12; A61K 45/06

(52) U.S. Cl. CPC C07D 279/06; C07D 241/22; C07D 251/24; C07D 239/69; C07D 403/04; C07D 213/76; C07D 401/14; C07D 401/12; A61K 45/06; C07B 2200/05

(57) ABSTRACT

This disclosure provides modulators of Cystic Fibrosis Transmembrane Conductance Regulator (CFTR) having the structure: (I), pharmaceutical compositions containing at least one such modulator, methods of treatment of cystic fibrosis using such modulators and pharmaceutical compositions, combination therapies, and processes and intermediates for making such modulators.

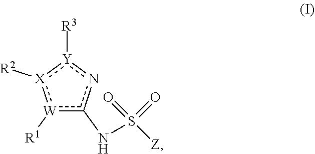

This application claims the benefit of priority of U.S. Provisional Application No. 63/088,876, filed Oct. 7, 2020, the contents of which are incorporated by reference herein in their entirety.

This disclosure relates to modulators of Cystic Fibrosis Transmembrane Conductance Regulator (CFTR), pharmaceutical compositions containing the modulators, methods of treatment of cystic fibrosis using such modulators and pharmaceutical compositions, combination therapies, processes for making such modulators, and intermediates used in making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl— channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, compounds of Formula Ia, compounds of Formula Ib, compounds of Formula Ic, compounds of Formula II, compounds of Formula IIa, compounds of Formula IIb, compounds of Formula IIc, compounds of Formula IId, Compounds of Formula IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing.

Formula I encompasses compounds falling within the following structure:

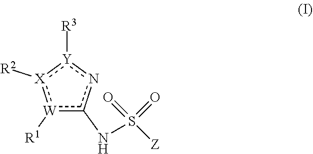

and includes tautomers of those compounds, deuterated derivatives of any of the compounds and tautomers, and pharmaceutically acceptable salts of any of the foregoing, wherein:
W, X, and Y are each independently selected from C, S, O, and N;
   wherein at least two of W, X, and Y are C;
Z is selected from phenyl (optionally substituted with —NH₂) and pyrazole (optionally substituted with —C₁₋₃ alkyl);
R¹ is absent or is selected from hydrogen, phenyl (optionally substituted with C₁₋₃ alkyl, halogen, —C₁₋₄ alkoxy), —C₅₋₆ cycloalkyl, and —C₃₋₄ alkenyl,
R² is selected from hydrogen, phenyl (optionally substituted with halogen, —C₁₋₃ alkyl, —C₁₋₄ alkoxy, —O-phenyl), —C₁₋₆ alkyl (optionally substituted with 1-2 groups selected from ═O and —C₁₋₄ alkoxy), halogen, —C₃₋₆ cycloalkyl (optionally substituted with phenyl which is further optionally substituted with —C₁₋₄ alkoxy), —C₃₋₄ alkenyl, -benzyl, —S(O)₂-phenyl, —C(O)NHC₁₋₆ alkyl, —C(O)NHbenzyl, 5-6 membered heterocyclyl substituted with NH-phenyl (substituted with 1-2 groups selected from —C₁₋₄ alkoxy and halogen), 5-6 membered heteroaryl substituted with NH-phenyl (substituted with 1-2 groups selected from —C₁₋₄ alkoxy and halogen), heterocycle (e.g., 5-6 membered heterocycle) substituted with NH-heterocyclyl (e.g., NH-(5-6 membered heterocycle)), and 5-6 membered heteroaryl substituted with NH-(5-6 membered heteroaryl),
wherein R² is not tert-butyl or —C(O)NH-tert-butyl, and
wherein at least one of R¹ and R² is absent or is hydrogen; and
R³ is selected from phenyl (optionally substituted with 1-3 groups selected from halogen, —C₁₋₄ alkyl, and —C₁₋₄ alkoxy), -10 membered heteroaryl, and —C₁₋₄ alkyl (optionally substituted with 1-2 groups selected from C₁₋₃ alkoxy, ═O, and phenyl).

Formula II encompasses compounds falling within the following structure:

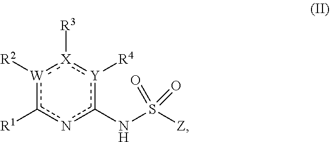

tautomers of those compounds, deuterated derivatives of any of the compounds and tautomers, and pharmaceutically acceptable salts of any of the foregoing, wherein:
W, X, and Y are independently selected from C, N, and S, wherein at least one of W, X, and Y is C, and wherein Y cannot be N unless W is also N;
Z is selected from phenyl (optionally substituted with NH₂) and pyrazole (optionally substituted with C₁₋₃ alkyl);
R¹ is absent or is selected from hydrogen, -phenyl (optionally substituted with 1-3 groups independently selected from —C₁₋₃ alkyl), —O-phenyl, and —C₁₋₄ alkoxy;
R² is absent or is selected from hydrogen, —C₁₋₃ alkyl (optionally substituted with 1-3 halogen), —C₁₋₃ alkenyl, —C₁₋₄ alkoxy, —C(O)C₁₋₄ alkoxy, and -phenyl (optionally substituted with —C₁₋₃ alkyl);
R³ is absent or is selected from hydrogen, halogen, —C₁₋₃ alkyl, -phenyl (optionally substituted with —C₁₋₃ alkyl), —C₁₋₄ alkoxy, —O-phenyl (optionally substituted with 5-6 membered heterocyclyl which is further optionally substituted with C₁₋₃ alkyl), —O-benzyl, and 5-6 membered heterocyclyl (optionally substituted with 1-3 groups independently selected from —C₁₋₃ alkyl); and
R⁴ is absent or is selected from hydrogen, phenyl, NH-benzyl, and ═O.

Another aspect of the disclosure provides pharmaceutical compositions comprising at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, which compositions may further include at least one additional active pharmaceutical ingredient. Thus, another aspect of the disclosure provides methods of treating the CFTR-mediated disease cystic fibrosis comprising administering at least one of compound chosen from the novel compounds disclosed herein, tautomers thereof, deuterated derivatives of those compounds and tautomers, and pharmaceutically acceptable salts of any of the foregoing, and at least one pharmaceutically acceptable carrier, optionally as part of a pharmaceutical composition comprising at least one additional component, to a subject in need thereof.

In certain embodiments, the pharmaceutical compositions disclosed herein comprise at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing. In some embodiments, compositions comprising at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing may optionally further comprise: (a) at least one compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor), and deuterated derivatives and pharmaceutically acceptable salts of tezacaftor and lumacaftor; and/or (b) at least one compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.

Another aspect of the disclosure provides methods of treating the CFTR-mediated disease cystic fibrosis comprising administering to a patient in need thereof at least one compound chosen from the novel compounds disclosed herein, tautomers thereof, deuterated derivatives of those compounds and tautomers, and pharmaceutically acceptable salts of any of the foregoing, and optionally further administering one or more additional CFTR modulating agents selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

The disclosure further provides intermediates and methods of making the compounds and compositions disclosed herein.

### Definitions

“Chosen from” and “selected from” are used interchangeably herein.

Compounds 1-158 in this disclosure is intended to represent a reference to each of Compounds 1 through 158 individually and a reference to groups of compounds, such as, e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158.

“Tezacaftor,” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

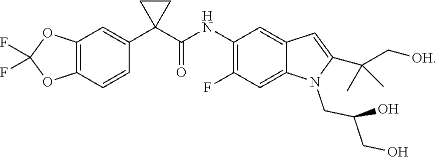 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated herein by reference.

“Ivacaftor,” as used throughout this disclosure, refers to N-(2,4-di-tert-butyl-5-hydroxyphenyl)-1,4-dihydro-4-oxoquinoline-3-carboxamide, which is depicted by the structure:

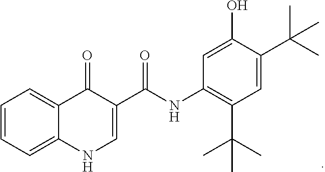 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

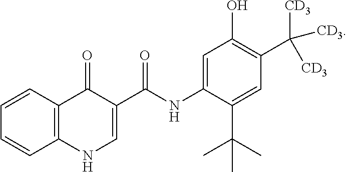 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor,” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

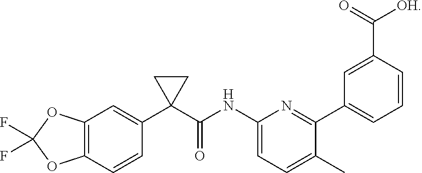 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using lumacaftor are disclosed in WO 2007/056341, WO 2009/073757, and WO 2009/076142, each of which is incorporated herein by reference.

As used herein, the term “alkyl” refers to a saturated or partially saturated, branched or unbranched aliphatic hydrocarbon containing carbon atoms (such as, for example, 1, 2, 3, 4, 5, 6, 7, 8, 9, 10, 11, or 12 carbon atoms), wherein one or more adjacent carbon atoms may be interrupted by double (alkenyl) or triple (alkynyl) bonds. Alkyl groups may be substituted or unsubstituted.

As used herein, the term “haloalkyl group” refers to an alkyl group substituted with one or more halogen atoms, e.g., fluoroalkyl, wherein the alkyl group is substituted with one or more fluorine atoms.

The term “alkoxy,” as used herein, refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example, 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The term “heteroaryl ring,” as used herein, refers to an aromatic ring system comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline. A further non-limiting example of a heteroaryl ring is 2,3-dihydrobenzo[b][1,4]dioxinyl.

As used herein, the term “heterocyclyl ring” refers to a non-aromatic hydrocarbon containing 3 to 12 atoms in a ring (such as, for example, 3-10 atoms) comprising at least one ring atom that is a heteroatom, such as O, N, or S, and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Non-limiting examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom. In chemical structures, deuterium is represented as “D.” In some embodiments, the one or more hydrogens replaced by deuterium are part of an alkyl group. In some embodiments, the one or more hydrogens replaced by deuterium are part of a methyl group. The phrase “deuterated derivatives and pharmaceutically acceptable salts of [a specified compound or compounds]” as used herein refers to deuterated derivatives of the compound or compounds as well as pharmaceutically acceptable salts of the compound or compounds and pharmaceutically acceptable salts of the deuterated derivative of the compound or compounds. The phrase “and deuterated derivatives and pharmaceutically acceptable salts thereof” is used interchangeably with “and deuterated derivatives and pharmaceutically acceptable salts thereof of any of the forgoing” in reference to one or more compounds or formulae of the disclosure. These phrases are intended to encompass pharmaceutically acceptable salts of any one of the referenced compounds, deuterated derivatives of any one of the referenced compounds, as well as pharmaceutically acceptable salts of those deuterated derivatives.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

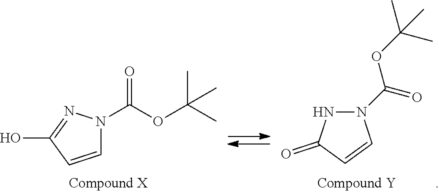

It will be appreciated that certain compounds of this disclosure may exist as separate stereoisomers or enantiomers and/or mixtures of those stereoisomers or enantiomers.

As used herein, the term “pharmaceutically acceptable salt” refers to a salt form of a compound of this disclosure, wherein the salt is nontoxic. Pharmaceutically acceptable salts of the compounds of this disclosure include those derived from suitable inorganic and organic acids and bases. A “free base” form of a compound, for example, does not contain an ionically bonded salt.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

As used herein, “CFTR” means cystic fibrosis transmembrane conductance regulator.

As used herein, the term “CFTR modulator” refers to a compound that increases the activity of CFTR. The increase in activity resulting from a CFTR modulator includes, but is not limited to, compounds that correct, potentiate, stabilize, and/or amplify CFTR.

As used herein, the term “CFTR corrector” refers to a compound that facilitates the processing and trafficking of CFTR to increase the amount of CFTR at the cell surface. The novel compounds disclosed herein are CFTR correctors.

As used herein, the term “CFTR potentiator” refers to a compound that increases the channel activity of CFTR protein located at the cell surface, resulting in enhanced ion transport. Ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol referenced herein are CFTR potentiators. It will be appreciated that, in a combination described herein of a compound selected from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and other specified CFTR modulating agents, the other CFTR modulating agent(s) will typically, but not necessarily, include at least one potentiator. In some embodiments, the potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

As used herein, the term “active pharmaceutical ingredient” or “therapeutic agent” (“API”) refers to a biologically active compound.

The terms “patient” and “subject” are used interchangeably herein and refer to an animal, including a human.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the values is measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/l).

As used herein, the term “room temperature” or “ambient temperature” means 15° C. to 30° C.

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

### Detailed Description of Embodiments

In addition to compounds of Formula I, tautomers thereof, deuterated derivatives of those compounds and tautomers, and pharmaceutically acceptable salts of any of the foregoing, the disclosure provides compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing.

For example, in some embodiments, the compound of Formula I is a compound of Formula Ia:

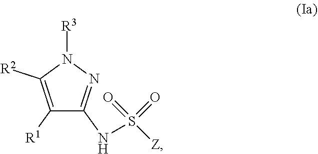

a tautomer thereof, a deuterated derivative of the compound or tautomer, or a pharmaceutically acceptable salt of any of the foregoing, wherein variables R¹, R², R³, and Z are as defined for Formula I.

In some embodiments, the compound of Formula I is a compound of Formula Ib:

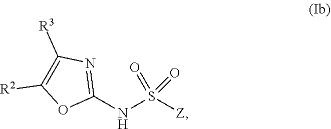

a tautomer thereof, a deuterated derivative of the compound or tautomer, or a pharmaceutically acceptable salt of any of the foregoing, wherein variables R², R³, and Z are as defined for Formula I.

In some embodiments, the compound of Formula I is a compound of Formula Ic:

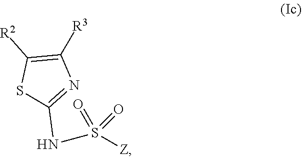

a tautomer thereof, a deuterated derivative of the compound or tautomer, or a pharmaceutically acceptable salt of any of the foregoing, wherein variables R², R³, and Z are as defined for Formula I.

In some embodiments, the compound of Formula II is a compound of one of the following Formulae:

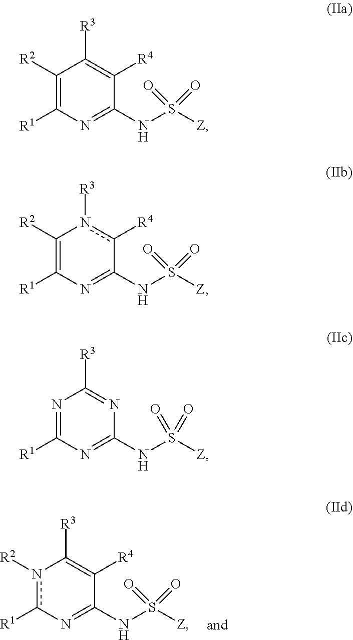

a tautomer thereof, a deuterated derivative of the compound or tautomer, or a pharmaceutically acceptable salt of any of the foregoing, wherein all variables are as defined above for Formula II.

Also disclosed herein are Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing.

### Methods of Treatment

Any of the novel compounds disclosed herein, such as, for example, compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as, for example, compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, alone or in combination with another active ingredient, such as one or more CFTR modulating agents. In some embodiments, the one or more CFTR modulating agents are selected from ivacaftor, deutivacaftor, lumacaftor, tezacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol (e.g., from ivacaftor, deutivacaftor, lumacaftor, and tezacaftor). In some embodiments, the patient has an F508del/minimal function (MF) genotype, F508del/F508del genotype (homozygous for the F508del mutation), F508del/gating genotype, or F508del/residual function (RF) genotype. In some embodiments, the patient is heterozygous and has one F508del mutation. In some embodiments, the patient is homozygous for the N1303K mutation.

In some embodiments, 5 mg to 500 mg of a compound disclosed herein, a tautomer thereof, a deuterated derivative of the compound or tautomer, or a pharmaceutically acceptable salt of any of the foregoing are administered daily.

In some embodiments, the patient has at least one F508del mutation in the CFTR gene. In some embodiments, the patient has a CFTR gene mutation that is responsive to a compound, tautomer, deuterated derivative, or pharmaceutically acceptable salt of the disclosure based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense | Q2X | L218X | Q525X | R792X | E1104X |
| mutations | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical | 185 + 1G→T | 711 + 5G→A | 1717 − 8G→A | 2622 + 1G→A | 3121 − 1G→A |
| splice | 296 + 1G→A | 712 − 1G→T | 1717 − 1G→A | 2790 − 1G→C | 3500 − 2A→G |
| mutations | 296 + 1G→T | 1248 + 1G→A | 1811 + 1G→C | 3040G→C | 3600 + 2insT |
| | 405 + 1G→A | 1249 − 1G→A | 1811 + 1.6kbA→G | (G970R) | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 | 182delT | 1078delT | 1677delTA | 2711delT | 3737delA |
| nucleotide) | 306insA | 1119delA | 1782delA | 2732insA | 3791delC |
| insertion/ | 306delTAGA | 1138insG | 1824delA | 2869insG | 3821delT |
| deletion | 365-366insT | 1154insTC | 1833delT | 2896insAG | 3876delA |
| (ins/del) | 394delTT | 1161delC | 2043delG | 2942insT | 3878delG |
| frameshift | 442delA | 1213delT | 2143delT | 2957delT | 3905insT |
| mutations | 444delA | 1259insA | 2183AA→G ^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |

| | | | |
|---|---|---|---|
| Non-small | CFTRdele1 | CFTRdele16-17b | 1461ins4 |
| (>3 nucleotide) | CFTRdele2 | CFTRdele17a, 17b | 1924del7 |
| insertion/ | CFTRdele2, 3 | CFTRdele17a-18 | 2055del9→A |
| deletion | CFTRdele2-4 | CFTRdele19 | 2105-2117del13insAGAAA |
| (ins/del) | CFTRdele3-10, 14b-16 | CFTRdele19-21 | 2372de18 |
| frameshift | CFTRdele4-7 | CFTRdele21 | 2721del11 |
| mutations | CFTRdele4-11 | CFTRdele22-24 | 2991del32 |
| | CFTR50kbdel | CFTRdele22, 23 | 3667ins4 |
| | CFTRdup6b-10 | 124del23bp | 4010del4 |
| | CFTRdele11 | 602del14 | 4209TGTT→AA |
| | CFTRdele13, 14a | 852del22 | |
| | CFTRdele14b-17b | 991del5 | |

| | | | | |
|---|---|---|---|---|
| Missense mutations that | A46D | V520F | Y569D | N1303K |
| Are not responsive in | G85E | A559T | L1065P | |
| vitro to TEZ, IVA, or | R347P | R560T | R1066C | |
| TEZ/IVA and | L467P | R560S | L1077P | |
| % PI >50% and SwCl⁻ | I507del | A561E | M1101K | |
| >86 mmol/L | | | | |

CFTR: cystic fibrosis transmembrane conductance regulator;
IVA: ivacaftor;
SwCl: sweat chloride;
TEZ: tezacaftor
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic
insufficient;
SwCl: mean sweat chloride of F508del-CFTR heterozygous patients in the CFTR2 patient registry.
^{a} Also known as 2183delAA→G.

In some embodiments, the disclosure is also directed to methods of treatment using isotope-labelled compounds of the aforementioned compounds, or pharmaceutically acceptable salts thereof, wherein the formula and variables of such compounds and salts are each independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salts of the disclosure may be defined by the isotopic enrichment factor. The term “isotopic enrichment factor,” as used herein, means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as, for example, compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, in combination with at least one additional active pharmaceutical ingredient.

In some embodiments, the at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful in combination therapies described herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodilators include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary anti-inflammatory agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, the at least one additional active pharmaceutical ingredient is selected from CFTR modulating agents. In some embodiments, the at least one additional active pharmaceutical ingredient is selected from CFTR potentiators. In some embodiments, the potentiators are selected from ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the additional active pharmaceutical ingredient is chosen from CFTR correctors. In some embodiments, the correctors are selected from lumacaftor, tezacaftor, deuterated derivatives of lumacaftor and tezacaftor, and pharmaceutically acceptable salts of any of the foregoing. In some embodiments, the at least one additional active pharmaceutical ingredient is chosen from (a) tezacaftor, lumacaftor, and deuterated derivatives and pharmaceutically acceptable salts of tezacaftor and lumacaftor; and (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. Thus, in some embodiments, the combination therapies provided herein comprise (a) a compound selected from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing; (b) at least one compound selected from tezacaftor and deuterated derivatives and pharmaceutically acceptable salts thereof, and (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the combination therapies provided herein comprise (a) at least one compound selected from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing; (b) at least one compound selected from lumacaftor and deuterated derivatives and pharmaceutically acceptable salts thereof; and (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

In some embodiments, at least one compound chosen from compounds of compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from ivacaftor and deuterated derivatives and pharmaceutically acceptable salts thereof. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from deutivacaftor and further deuterated derivatives and pharmaceutically acceptable salts thereof. In some embodiments, at least one compound chosen from compounds of Formula I, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound selected from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salt thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof, and at least one compound chosen from ivacaftor and deuterated derivatives and pharmaceutically acceptable salts thereof. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and pharmaceutically acceptable salts thereof. In some embodiments at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in combination with lumacaftor and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered once daily. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered twice daily.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered once daily. In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered twice daily.

In some embodiments, (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and (b) at least one compound chosen from tezacaftor and deuterated derivatives and pharmaceutically acceptable salts thereof, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered once daily. In some embodiments, (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from lumacaftor and deuterated derivatives and pharmaceutically acceptable salts thereof, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered once daily. In some embodiments, (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered twice daily. In some embodiments, (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from lumacaftor and pharmaceutically acceptable salts thereof, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered twice daily.

In some embodiments, (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered twice daily. In some embodiments, (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one compound chosen from lumacaftor and pharmaceutically acceptable salts thereof, are administered once daily, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, is administered twice daily.

Compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, along with at least one compound selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, can be administered in a single pharmaceutical composition or separate pharmaceutical compositions. Such pharmaceutical compositions can be administered once daily or multiple times daily, such as, e.g., twice daily. As used herein, the phrase that a given amount of API (e.g., tezacaftor, (ivacaftor or deutivacaftor) or a pharmaceutically acceptable salt thereof) is administered once or twice daily or per day means that said given amount is administered per dosing once or twice daily.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in a first pharmaceutical composition; at least one compound chosen from tezacaftor and pharmaceutically acceptable salts thereof is administered in a second pharmaceutical composition; and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, is administered in a third pharmaceutical composition.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in a first pharmaceutical composition; at least one compound chosen from lumacaftor and pharmaceutically acceptable salts thereof is administered in a second pharmaceutical composition; at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, is administered in a third pharmaceutical composition.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in a first pharmaceutical composition; at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, is administered in a second pharmaceutical composition.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, is administered in a first pharmaceutical composition; and at least one compound chosen from tezacaftor and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, is administered in a second pharmaceutical composition. In some embodiments, the second pharmaceutical composition comprises a half of a daily dose of said at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and the other half of said at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, is administered in a third pharmaceutical composition.

In some embodiments, at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing; at least one compound chosen from tezacaftor and deuterated derivatives and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and a second composition comprising only ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or deuterated derivatives and pharmaceutically acceptable salts of any of the foregoing, is administered once daily.

Any suitable pharmaceutical compositions can be used for compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, all of which are incorporated herein by reference.

### Pharmaceutical Compositions

Another aspect of the disclosure provides a pharmaceutical composition comprising at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, and a potentiator compound. In some embodiments, the pharmaceutical composition comprises at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, a potentiator compound, and a corrector compound. In some embodiments, the corrector compound is selected from tezacaftor, lumacaftor, and deuterated derivatives and pharmaceutically acceptable salts of any of the foregoing. In some embodiments, the potentiator compound is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

In some embodiments, the disclosure provides a pharmaceutical composition comprising (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from tezacaftor and pharmaceutically acceptable salts thereof, and (c) at least one pharmaceutically acceptable carrier.

In some embodiments, the disclosure provides a pharmaceutical composition comprising (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and (c) at least one pharmaceutically acceptable carrier.

In some embodiments, the disclosure provides a pharmaceutical composition comprising (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from ivacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from deutivacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, Ia, Ib, Ic, II, IIa, IIb, IIc, IId, IIe, Compounds 1-158 (e.g., Compounds 1-115; Compounds 116-158; Compounds 1-3, 5-10, 13-27, 29, 30, 40, 41, 48-52, 54-70, 85-87, 90-92, 94, 96, 101; Compounds 116-120, 124-126, 128, 130-144, 146-158), tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, deuterated derivatives and pharmaceutically acceptable salts of any of the foregoing, (c) at least one compound chosen from lumacaftor and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York disclose various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose, and sucrose), starches (such as corn starch and potato starch), cellulose and its derivatives (such as sodium carboxymethyl cellulose, ethyl cellulose, and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Exemplary Embodiments

Without limitation, some embodiments of the disclosure include:

1. A compound of Formula I:

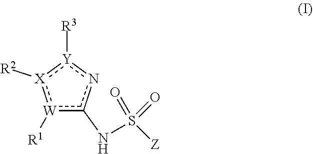

or a tautomer thereof, a deuterated derivative of the compound or tautomer, or a pharmaceutically acceptable salt of any of the foregoing, wherein:
W, X, and Y are each independently selected from C, S, O, and N;
   wherein at least two of W, X, and Y are C;
Z is selected from phenyl (optionally substituted with NH₂) and pyrazole (optionally substituted with C₁₋₃ alkyl);
R¹ is absent or is selected from hydrogen, phenyl (optionally substituted with —C₁₋₃ alkyl, halogen, —C₁₋₄ alkoxy), —C₅₋₆ cycloalkyl, and —C₃₋₄ alkenyl;
R² is selected from hydrogen, phenyl (optionally substituted with halogen, —C₁₋₃ alkyl, —C₁₋₄ alkoxy, —O-phenyl), —C₁₋₆ alkyl (optionally substituted with 1-2 groups selected from ═O and —C₁₋₄ alkoxy), halogen, —C₃₋₆ cycloalkyl (optionally substituted with phenyl which is further optionally substituted with —C₁₋₄ alkoxy), —C₃₋₄ alkenyl, benzyl, —S(O)₂-phenyl, —C(O)NHC₁₋₆ alkyl, C(O)NHbenzyl, 5-6 membered heterocycle substituted with NH-phenyl (substituted with 1-2 groups selected from —C₁₋₄ alkoxy and halogen), 5-6 membered heteroaryl substituted with NH-phenyl (substituted with 1-2 groups selected from —C₁₋₄ alkoxy and halogen), heterocycle substituted with NH-heterocyclyl, and 5-6 membered heteroaryl substituted with NH-(5-6 membered heteroaryl),
   wherein R² is not tert-butyl or C(O)NH-tert butyl, and
   wherein at least one of R¹ and R² is absent or hydrogen; and
R³ is selected from phenyl (optionally substituted with 1-3 groups selected from halogen, C₁₋₄ alkyl, C₁₋₄ alkoxy), -10 membered heteroaryl, and C₁₋₄ alkyl (optionally substituted with 1-2 groups selected from C₁₋₃ alkoxy, ═O, and phenyl).
   2. The compound, tautomer, deuterated derivative, or pharmaceutically acceptable salt according to Embodiment 1, selected from compounds of Formula Ia:

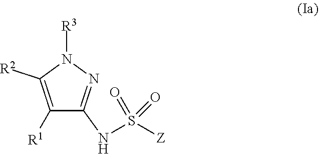

or a tautomer thereof, a deuterated derivative of the compound or tautomer, or a pharmaceutically acceptable salt of any of the foregoing, wherein variables R¹, R², R³, and Z are as defined in Embodiment 1.

3. The compound, tautomer, deuterated derivative, or pharmaceutically acceptable salt according to Embodiment 1, selected from compounds of Formula Ib:

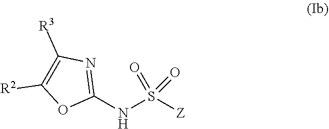

or a tautomer thereof, a deuterated derivative of the compound or tautomer, or a pharmaceutically acceptable salt of any of the foregoing, wherein variables R², R³, and Z are as defined in Embodiment 1.

4. The compound, tautomer, deuterated derivative, or pharmaceutically acceptable salt according to Embodiment 1, selected from compounds of Formula Ic:

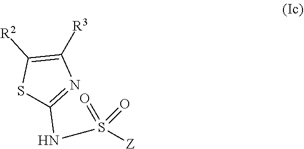

or a tautomer thereof, a deuterated derivative of the compound or tautomer, or a pharmaceutically acceptable salt of any of the foregoing, wherein variables R², R³, and Z are as defined in Embodiment 1.

5. The compound, tautomer, deuterated derivative, or pharmaceutically acceptable salt according to any one of Embodiments 1-4, wherein W is C and R¹ is selected from hydrogen and optionally substituted phenyl.
6. The compound, tautomer, deuterated derivative, or pharmaceutically acceptable salt according to any one of Embodiments 1-5, wherein R² and R³ are optionally substituted phenyl.
7. A compound selected from:
N-[3,4-bis(4-chlorophenyl)isoxazol-5-yl]benzenesulfonamide (Compound 1);
N-(4-phenyl-5-propyl-oxazol-2-yl)benzenesulfonamide (Compound 2);
N-[4,5-bis(p-tolyl)oxazol-2-yl]benzenesulfonamide (Compound 3);
N-[5-[(2-methoxyphenyl)methyl]oxazol-2-yl]benzenesulfonamide (Compound 4);
N-[5-[(2-methoxyphenyl)methyl]oxazol-2-yl]benzenesulfonamide (Compound 5);
N-[4-(2,4-dimethylphenyl)-5-propyl-thiazol-2-yl]benzenesulfonamide (Compound 6);
N-(4,5-diphenylthiazol-2-yl)benzenesulfonamide (Compound 7);
N-[4-(2,5-dimethylphenyl)-5-methyl-thiazol-2-yl]benzenesulfonamide (Compound 8);
3-amino-N-(5-benzyl-4-phenyl-thiazol-2-yl)benzenesulfonamide (Compound 9);
N-[4,5-bis(p-tolyl)thiazol-2-yl]benzenesulfonamide (Compound 10);
N-(5-phenoxy-4-phenyl-thiazol-2-yl)benzenesulfonamide (Compound 11);
N-(4-cyclopropylthiazol-2-yl)benzenesulfonamide (Compound 12);
methyl 2-(benzenesulfonamido)-4-(p-tolyl)thiazole-5-carboxylate (Compound 13);
2-(benzenesulfonamido)-N-benzyl-4-phenyl-thiazole-5-carboxamide (Compound 14);
2-(benzenesulfonamido)-N-(3,3-dimethylbutyl)-4-phenyl-thiazole-5-carboxamide (Compound 15);
N-[5-(benzenesulfonyl)-4-(4-chlorophenyl)thiazol-2-yl]benzenesulfonamide (Compound 16);
N-(5-isopropyl-4-phenyl-thiazol-2-yl)benzenesulfonamide (Compound 17);
N-[4-(2,5-dimethylphenyl)-5-propyl-thiazol-2-yl]benzenesulfonamide (Compound 18);
N-[4-(4-ethoxyphenyl)-5-propyl-thiazol-2-yl]benzenesulfonamide (Compound 19);
N-[4-(4-methoxyphenyl)-5-propyl-thiazol-2-yl]benzenesulfonamide (Compound 20);
N-(5-ethyl-4-phenyl-thiazol-2-yl)benzenesulfonamide (Compound 21);
N-[4-(2,3-dihydro-1,4-benzodioxin-6-yl)-5-methyl-thiazol-2-yl]benzenesulfonamide (Compound 22);
N-(5-methyl-4-phenyl-thiazol-2-yl)benzenesulfonamide (Compound 23);
ethyl 2-(benzenesulfonamido)-4-phenyl-thiazole-5-carboxylate (Compound 24);
N-[5-[1-(2-methoxyphenyl)cyclopropyl]thiazol-2-yl]benzenesulfonamide (Compound 25);
N-(4-methyl-5-phenyl-thiazol-2-yl)benzenesulfonamide (Compound 26);
N-[5-[2-(5-chloro-2-methoxy-anilino)thiazol-4-yl]-4-methyl-thiazol-2-yl]benzenesulfonamide (Compound 27);
3-[[2-[(3-aminophenyl)sulfonylamino]-4-phenyl-thiazol-5-yl]methyl]benzoic acid (Compound 28);
N-[5-[2-(2-methoxyanilino)thiazol-4-yl]-4-methyl-thiazol-2-yl]benzenesulfonamide (Compound 29);
N-[4-methyl-5-[2-(2-pyridylamino)thiazol-4-yl]thiazol-2-yl]benzenesulfonamide (Compound 30);
N-[5-(pyrazin-2-ylmethyl)thiazol-2-yl]benzenesulfonamide (Compound 31);
N-[5-benzyl-thiazol-2-yl]benzenesulfonamide (Compound 32);
N,4-diphenyl-2-(phenylsulfonamido)thiazole-5-carboxamide (Compound 33);
N-[4-phenyl-5-(piperidine-1-carbonyl)thiazol-2-yl]benzenesulfonamide (Compound 34);
2-(benzenesulfonamido)-N-(2-methoxyethyl)-4-phenyl-thiazole-5-carboxamide (Compound 35);
2-(benzenesulfonamido)-N-tert-butyl-4-phenyl-thiazole-5-carboxamide (Compound 36);
2-(benzenesulfonamido)-N-[2-(cyclopropylmethoxy)ethyl]-4-phenyl-thiazole-5-carboxamide (Compound 37);
2-(benzenesulfonamido)-N-methyl-4-phenyl-N-propyl-thiazole-5-carboxamide (Compound 38);
2-(benzenesulfonamido)-4-phenyl-N-propyl-thiazole-5-carboxamide (Compound 39);
methyl 2-[2-(benzenesulfonamido)-5-phenyl-thiazol-4-yl]acetate (Compound 40);
ethyl 2-(benzenesulfonamido)-5-phenyl-thiazole-4-carboxylate (Compound 41);
4-phenyl-2-(phenylsulfonamido)thiazole-5-carboxylic acid (Compound 42);
N-[5-methyl-4-(4-pyridyl)thiazol-2-yl]benzenesulfonamide (Compound 43);
N-[4-methyl-5-[2-(pyrimidin-2-ylamino)thiazol-4-yl]thiazol-2-yl]benzenesulfonamide (Compound 44);
N-(4-tert-butyl-5-cyano-thiazol-2-yl)benzenesulfonamideN-(4-tert-butyl-5-cyano-thiazol-2-yl)benzenesulfonamide (Compound 45);
N-(4,5-dimethylthiazol-2-yl)benzenesulfonamide (Compound 46);
N-(5-methyl-4-propyl-thiazol-2-yl)benzenesulfonamide (Compound 47);
N-[5-(m-tolyl)-1-(2,4,6-trimethylphenyl)pyrazol-3-yl]benzenesulfonamide (Compound 48);
N-[5-(4-phenoxyphenyl)-1-(2,4,6-trimethylphenyl)pyrazol-3-yl]benzenesulfonamide (Compound 49);
N-[5-(3-methoxyphenyl)-1-(2,4,6-trimethylphenyl)pyrazol-3-yl]benzenesulfonamide (Compound 50);
N-[5-(2-methylprop-1-enyl)-1-(2,4,6-trimethylphenyl)pyrazol-3-yl]benzenesulfonamide (Compound 51);
N-[5-isobutyl-1-(2,4,6-trimethylphenyl)pyrazol-3-yl]benzenesulfonamide (Compound 52);
N-[5-tert-butyl-1-(2,4,6-trimethylphenyl)pyrazol-3-yl]benzenesulfonamide (Compound 53);
N-[5-(3-phenoxyphenyl)-1-(2,4,6-trimethylphenyl)pyrazol-3-yl]benzenesulfonamide (Compound 54);
N-[5-(2-phenoxyphenyl)-1-(2,4,6-trimethylphenyl)pyrazol-3-yl]benzenesulfonamide (Compound 55);
N-[5-(4-chlorophenyl)-1-(2,4,6-trimethylphenyl)pyrazol-3-yl]benzenesulfonamide (Compound 56);
N-[5-(3-chlorophenyl)-1-(2,4,6-trimethylphenyl)pyrazol-3-yl]benzenesulfonamide (Compound 57);
N-[5-(2-chlorophenyl)-1-(2,4,6-trimethylphenyl)pyrazol-3-yl]benzenesulfonamide (Compound 58);
N-[5-(4-methoxyphenyl)-1-(2,4,6-trimethylphenyl)pyrazol-3-yl]benzenesulfonamide (Compound 59);
N-[5-(2-methoxyphenyl)-1-(2,4,6-trimethylphenyl)pyrazol-3-yl]benzenesulfonamide (Compound 60);
N-[5-(p-tolyl)-1-(2,4,6-trimethylphenyl)pyrazol-3-yl]benzenesulfonamide (Compound 61);
N-[5-(p-tolyl)-1-(2,4,6-trimethylphenyl)pyrazol-3-yl]benzenesulfonamide (Compound 62);
N-[5-(cyclohexen-1-yl)-1-(2,4,6-trimethylphenyl)pyrazol-3-yl]benzenesulfonamide (Compound 63);
N-[5-cyclohexyl-1-(2,4,6-trimethylphenyl)pyrazol-3-yl]benzenesulfonamide (Compound 64);
N-[5-isopropenyl-1-(2,4,6-trimethylphenyl)pyrazol-3-yl]benzenesulfonamide (Compound 65);
N-[5-isopropyl-1-(2,4,6-trimethylphenyl)pyrazol-3-yl]benzenesulfonamide (Compound 66);
N-[5-bromo-1-(2,4,6-trimethylphenyl)pyrazol-3-yl]benzenesulfonamide (Compound 67);
N-[5-phenyl-1-(2,4,6-trimethylphenyl)pyrazol-3-yl]benzenesulfonamide (Compound 68);
N-[1-(3-chlorophenyl)-4-phenyl-pyrazol-3-yl]benzenesulfonamide (Compound 69);
N-[1,4-bis(3-chlorophenyl)pyrazol-3-yl]benzenesulfonamide (Compound 70);
N-(5-benzyl-1-phenyl-pyrazol-3-yl)benzenesulfonamide (Compound 71);
N-(4-benzyl-1-phenyl-pyrazol-3-yl)-1-methyl-pyrazole-4-sulfonamide (Compound 72);
3-amino-N-(4-benzyl-1-phenyl-pyrazol-3-yl)benzenesulfonamide (Compound 73);
N-(4-benzyl-1-phenyl-pyrazol-3-yl)benzenesulfonamide (Compound 74);
N-[1-phenyl-4-(2-thienyl)pyrazol-3-yl]benzenesulfonamide (Compound 75);
N-(2,5-diphenylpyrazol-3-yl)benzenesulfonamide (Compound 76);
N-(4-phenyl-1H-pyrazol-3-yl)benzenesulfonamide (Compound 77);
N-[2-(cyclopropylmethyl)pyrazol-3-yl]benzenesulfonamide (Compound 78);
N-(2-cyclohexylpyrazol-3-yl)benzenesulfonamide (Compound 79);
N-[5-tert-butyl-1-(p-tolyl)pyrazol-3-yl]benzenesulfonamide (Compound 80);
N-[1-(3-chlorophenyl)-4-isobutyl-pyrazol-3-yl]benzenesulfonamide (Compound 81);
N-(4-isobutyl-1-phenyl-pyrazol-3-yl)benzenesulfonamide (Compound 82);
N-[1-(3-chlorophenyl)-4-cyclohexyl-pyrazol-3-yl]benzenesulfonamide (Compound 83);
N-(4-cyclohexyl-1-phenyl-pyrazol-3-yl)benzenesulfonamide (Compound 84);
N-[1-(3-chlorophenyl)-4-(2-methylprop-1-enyl)pyrazol-3-yl]benzenesulfonamide (Compound 85);
N-[1-(3-chlorophenyl)-4-isopropenyl-pyrazol-3-yl]benzenesulfonamide (Compound 86);
N-[1-(3-chlorophenyl)-4-(cyclohexen-1-yl)pyrazol-3-yl]benzenesulfonamide (Compound 87);
N-[1-(3-chlorophenyl)-4-(4-chlorophenyl)pyrazol-3-yl]benzenesulfonamide (Compound 88);
N-[4-(2-chlorophenyl)-1-(3-chlorophenyl)pyrazol-3-yl]benzenesulfonamide (Compound 89);
N-[1-(3-chlorophenyl)-4-(4-methoxyphenyl)pyrazol-3-yl]benzenesulfonamide (Compound 90);
N-[1-(3-chlorophenyl)-4-(3-methoxyphenyl)pyrazol-3-yl]benzenesulfonamide (Compound 91);
N-[1-(3-chlorophenyl)-4-(2-methoxyphenyl)pyrazol-3-yl]benzenesulfonamide (Compound 92);
N-[1-(3-chlorophenyl)-4-(p-tolyl)pyrazol-3-yl]benzenesulfonamide (Compound 93);
N-[1-(3-chlorophenyl)-4-(m-tolyl)pyrazol-3-yl]benzenesulfonamide (Compound 94);
N-[1-(3-chlorophenyl)-4-(o-tolyl)pyrazol-3-yl]benzenesulfonamide (Compound 95);
N-[4-phenyl-1-(2,4,6-trimethylphenyl)pyrazol-3-yl]benzenesulfonamide (Compound 96);
N-[4-isopropenyl-1-(2,4,6-trimethylphenyl)pyrazol-3-yl]benzenesulfonamide (Compound 97);
N-[4-isopropenyl-1-(2,4,6-trimethylphenyl)pyrazol-3-yl]benzenesulfonamide (Compound 98);
N-[4-bromo-1-(2,4,6-trimethylphenyl)pyrazol-3-yl]benzenesulfonamide (Compound 99);
N-[5-methyl-1-(p-tolyl)pyrazol-3-yl]benzenesulfonamide (Compound 100);
N-[1-benzyl-4-(p-tolyl)pyrazol-3-yl]benzenesulfonamide (Compound 101);
N-(1-benzylpyrazol-3-yl)benzenesulfonamide (Compound 102);
N-(1-benzyl-4-cyclohexyl-pyrazol-3-yl)benzenesulfonamide (Compound 103);
N-[1-benzyl-4-(2-methylprop-1-enyl)pyrazol-3-yl]benzenesulfonamide (Compound 104);
N-(1-benzyl-4-isopropenyl-pyrazol-3-yl)benzenesulfonamide (Compound 105);
N-[1-benzyl-4-(cyclohexen-1-yl)pyrazol-3-yl]benzenesulfonamide (Compound 106);
N-(1-benzyl-4-phenyl-pyrazol-3-yl)benzenesulfonamide (Compound 107);
N-[1-benzyl-4-(4-chlorophenyl)pyrazol-3-yl]benzenesulfonamide (Compound 108);
N-[1-benzyl-4-(3-chlorophenyl)pyrazol-3-yl]benzenesulfonamide (Compound 109);
N-[1-benzyl-4-(2-chlorophenyl)pyrazol-3-yl]benzenesulfonamide (Compound 110);
N-[1-benzyl-4-(4-methoxyphenyl)pyrazol-3-yl]benzenesulfonamide (Compound 111);
N-[1-benzyl-4-(p-tolyl)pyrazol-3-yl]benzenesulfonamide (Compound 112);
N-[1-benzyl-4-(2-methoxyphenyl)pyrazol-3-yl]benzenesulfonamide (Compound 113);
N-[1-benzyl-4-(m-tolyl)pyrazol-3-yl]benzenesulfonamide (Compound 114); and
N-[5-cyclohexyl-1-(2,4,6-trimethylphenyl)pyrazol-3-yl]benzenesulfonamide (Compound 115), or

or a tautomer thereof, a deuterated derivative of the compound or tautomer, or a pharmaceutically acceptable salt of any of the foregoing.

8. A compound selected from:
N-[3,4-bis(4-chlorophenyl)isoxazol-5-yl]benzenesulfonamide (Compound 1);
N-(4-phenyl-5-propyl-oxazol-2-yl)benzenesulfonamide (Compound 2);
N-[4,5-bis(p-tolyl)oxazol-2-yl]benzenesulfonamide (Compound 3);
N-[5-[(2-methoxyphenyl)methyl]oxazol-2-yl]benzenesulfonamide (Compound 5);
N-[4-(2,4-dimethylphenyl)-5-propyl-thiazol-2-yl]benzenesulfonamide (Compound 6);
N-(4,5-diphenylthiazol-2-yl)benzenesulfonamide (Compound 7);
N-[4-(2,5-dimethylphenyl)-5-methyl-thiazol-2-yl]benzenesulfonamide (Compound 8);
3-amino-N-(5-benzyl-4-phenyl-thiazol-2-yl)benzenesulfonamide (Compound 9);
N-[4,5-bis(p-tolyl)thiazol-2-yl]benzenesulfonamide (Compound 10);
methyl 2-(benzenesulfonamido)-4-(p-tolyl)thiazole-5-carboxylate (Compound 13);
2-(benzenesulfonamido)-N-benzyl-4-phenyl-thiazole-5-carboxamide (Compound 14);
2-(benzenesulfonamido)-N-(3,3-dimethylbutyl)-4-phenyl-thiazole-5-carboxamide (Compound 15);
N-[5-(benzenesulfonyl)-4-(4-chlorophenyl)thiazol-2-yl]benzenesulfonamide (Compound 16);
N-(5-isopropyl-4-phenyl-thiazol-2-yl)benzenesulfonamide (Compound 17);
N-[4-(2,5-dimethylphenyl)-5-propyl-thiazol-2-yl]benzenesulfonamide (Compound 18);
N-[4-(4-ethoxyphenyl)-5-propyl-thiazol-2-yl]benzenesulfonamide (Compound 19);
N-[4-(4-methoxyphenyl)-5-propyl-thiazol-2-yl]benzenesulfonamide (Compound 20);
N-(5-ethyl-4-phenyl-thiazol-2-yl)benzenesulfonamide (Compound 21);
N-[4-(2,3-dihydro-1,4-benzodioxin-6-yl)-5-methyl-thiazol-2-yl]benzenesulfonamide (Compound 22);
N-(5-methyl-4-phenyl-thiazol-2-yl)benzenesulfonamide (Compound 23);
ethyl 2-(benzenesulfonamido)-4-phenyl-thiazole-5-carboxylate (Compound 24);
N-[5-[1-(2-methoxyphenyl)cyclopropyl]thiazol-2-yl]benzenesulfonamide (Compound 25);
N-(4-methyl-5-phenyl-thiazol-2-yl)benzenesulfonamide (Compound 26);
N-[5-[2-(5-chloro-2-methoxy-anilino)thiazol-4-yl]-4-methyl-thiazol-2-yl]benzenesulfonamide (Compound 27);
N-[5-[2-(2-methoxyanilino)thiazol-4-yl]-4-methyl-thiazol-2-yl]benzenesulfonamide (Compound 29);
N-[4-methyl-5-[2-(2-pyridylamino)thiazol-4-yl]thiazol-2-yl]benzenesulfonamide (Compound 30);
methyl 2-[2-(benzenesulfonamido)-5-phenyl-thiazol-4-yl]acetate (Compound 40);
ethyl 2-(benzenesulfonamido)-5-phenyl-thiazole-4-carboxylate (Compound 41);
N-[5-(m-tolyl)-1-(2,4,6-trimethylphenyl)pyrazol-3-yl]benzenesulfonamide (Compound 48);
N-[5-(4-phenoxyphenyl)-1-(2,4,6-trimethylphenyl)pyrazol-3-yl]benzenesulfonamide (Compound 49);
N-[5-(3-methoxyphenyl)-1-(2,4,6-trimethylphenyl)pyrazol-3-yl]benzenesulfonamide (Compound 50);
N-[5-(2-methylprop-1-enyl)-1-(2,4,6-trimethylphenyl)pyrazol-3-yl]benzenesulfonamide (Compound 51);
N-[5-isobutyl-1-(2,4,6-trimethylphenyl)pyrazol-3-yl]benzenesulfonamide (Compound 52);
N-[5-(3-phenoxyphenyl)-1-(2,4,6-trimethylphenyl)pyrazol-3-yl]benzenesulfonamide (Compound 54);
N-[5-(2-phenoxyphenyl)-1-(2,4,6-trimethylphenyl)pyrazol-3-yl]benzenesulfonamide (Compound 55);
N-[5-(4-chlorophenyl)-1-(2,4,6-trimethylphenyl)pyrazol-3-yl]benzenesulfonamide (Compound 56);
N-[5-(3-chlorophenyl)-1-(2,4,6-trimethylphenyl)pyrazol-3-yl]benzenesulfonamide (Compound 57);
N-[5-(2-chlorophenyl)-1-(2,4,6-trimethylphenyl)pyrazol-3-yl]benzenesulfonamide (Compound 58);
N-[5-(4-methoxyphenyl)-1-(2,4,6-trimethylphenyl)pyrazol-3-yl]benzenesulfonamide (Compound 59);
N-[5-(2-methoxyphenyl)-1-(2,4,6-trimethylphenyl)pyrazol-3-yl]benzenesulfonamide (Compound 60);
N-[5-(p-tolyl)-1-(2,4,6-trimethylphenyl)pyrazol-3-yl]benzenesulfonamide (Compound 61);
N-[5-(p-tolyl)-1-(2,4,6-trimethylphenyl)pyrazol-3-yl]benzenesulfonamide (Compound 62);
N-[5-(cyclohexen-1-yl)-1-(2,4,6-trimethylphenyl)pyrazol-3-yl]benzenesulfonamide (Compound 63);
N-[5-cyclohexyl-1-(2,4,6-trimethylphenyl)pyrazol-3-yl]benzenesulfonamide (Compound 64);
N-[5-isopropenyl-1-(2,4,6-trimethylphenyl)pyrazol-3-yl]benzenesulfonamide (Compound 65);
N-[5-isopropyl-1-(2,4,6-trimethylphenyl)pyrazol-3-yl]benzenesulfonamide (Compound 66);
N-[5-bromo-1-(2,4,6-trimethylphenyl)pyrazol-3-yl]benzenesulfonamide (Compound 67);
N-[5-phenyl-1-(2,4,6-trimethylphenyl)pyrazol-3-yl]benzenesulfonamide (Compound 68);
N-[1-(3-chlorophenyl)-4-phenyl-pyrazol-3-yl]benzenesulfonamide (Compound 69);
N-[1,4-bis(3-chlorophenyl)pyrazol-3-yl]benzenesulfonamide (Compound 70);
N-[1-(3-chlorophenyl)-4-(2-methylprop-1-enyl)pyrazol-3-yl]benzenesulfonamide (Compound 85);
N-[1-(3-chlorophenyl)-4-isopropenyl-pyrazol-3-yl]benzenesulfonamide (Compound 86);
N-[1-(3-chlorophenyl)-4-(cyclohexen-1-yl)pyrazol-3-yl]benzenesulfonamide (Compound 87);
N-[1-(3-chlorophenyl)-4-(4-methoxyphenyl)pyrazol-3-yl]benzenesulfonamide (Compound 90);
N-[1-(3-chlorophenyl)-4-(3-methoxyphenyl)pyrazol-3-yl]benzenesulfonamide (Compound 91);
N-[1-(3-chlorophenyl)-4-(2-methoxyphenyl)pyrazol-3-yl]benzenesulfonamide (Compound 92);
N-[1-(3-chlorophenyl)-4-(m-tolyl)pyrazol-3-yl]benzenesulfonamide (Compound 94);
N-[4-phenyl-1-(2,4,6-trimethylphenyl)pyrazol-3-yl]benzenesulfonamide (Compound 96); and
N-[1-benzyl-4-(p-tolyl)pyrazol-3-yl]benzenesulfonamide (Compound 101),

or a tautomer thereof, a deuterated derivative of the compound or tautomer, or a pharmaceutically acceptable salt of any of the foregoing.

9. A compound of Formula II:

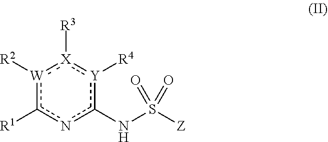

or a tautomer thereof, a deuterated derivative of the compound or tautomer, or a pharmaceutically acceptable salt of any of the foregoing, wherein:
W, X, and Y are independently selected from C, N, and S, wherein at least one of W, X, and Y is C, and wherein Y cannot be N unless W is also N;
Z is selected from phenyl (optionally substituted with NH₂) and pyrazole (optionally substituted with —C₁₋₃ alkyl);
R¹ is absent or is selected from hydrogen, -phenyl (optionally substituted with 1-3 groups independently selected from —C₁₋₃ alkyl), —O-phenyl, and —C₁₋₄ alkoxy;
R² is absent or is selected from hydrogen, —C₁₋₃ alkyl (optionally substituted with 1-3 halogen), —C₁₋₃ alkenyl, —C₁₋₄ alkoxy, —C(O)C₁₋₄ alkoxy, and -phenyl (optionally substituted with —C₁₋₃ alkyl);
R³ is absent or is selected from hydrogen, halogen, C₁₋₃ alkyl, -phenyl (optionally substituted with —C₁₋₃ alkyl), —C₁₋₄ alkoxy, —O-phenyl (optionally substituted with 5-6 membered heterocycle which is further optionally substituted with —C₁₋₃ alkyl), —O-benzyl, and 5-6 membered heterocycle (optionally substituted with 1-3 groups independently selected from —C₁₋₃ alkyl); and
R⁴ is absent or is selected from hydrogen, phenyl, NH-benzyl, and ═O.
   10. The compound, tautomer, deuterated derivative, or pharmaceutically acceptable salt according to Embodiment 9, selected from compounds of Formula IIa:

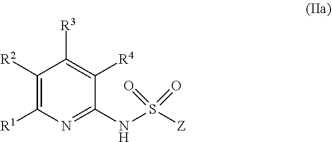

or a tautomer thereof, a deuterated derivative of the compound or tautomer, or a pharmaceutically acceptable salt of any of the foregoing, wherein variables R¹, R², R³, R⁴, and Z are as defined in Embodiment 9.

11. The compound, tautomer, deuterated derivative, or pharmaceutically acceptable salt according to Embodiment 9, selected from compounds of Formula IIb:

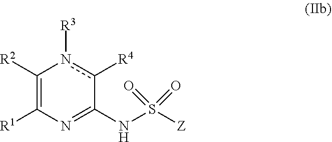

or a tautomer thereof, a deuterated derivative of the compound or tautomer, or a pharmaceutically acceptable salt of any of the foregoing, wherein variables R¹, R², R³, R⁴, and Z are as defined in Embodiment 9.

12. The compound, tautomer, deuterated derivative, or pharmaceutically acceptable salt according to Embodiment 9, selected from compounds of Formula IIc:

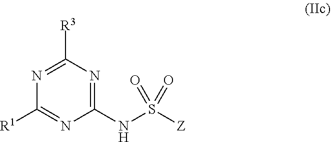

or a tautomer thereof, a deuterated derivative of the compound or tautomer, or a pharmaceutically acceptable salt of any of the foregoing, wherein variables R¹, R², and Z are as defined in Embodiment 9.

13. The compound, tautomer, deuterated derivative, or pharmaceutically acceptable salt according to Embodiment 9, selected from compounds of Formula IId:

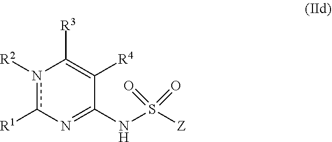

or a tautomer thereof, a deuterated derivative of the compound or tautomer, or a pharmaceutically acceptable salt of any of the foregoing, wherein variables R¹, R², R³, R⁴, and Z are as defined in Embodiment 9.

14. The compound, tautomer, deuterated derivative, or pharmaceutically acceptable salt according to Embodiment 9, selected from compounds of Formula IIe:

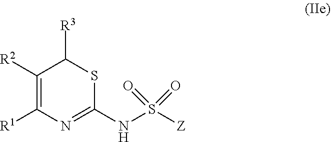

or a tautomer thereof, a deuterated derivative of the compound or tautomer, or a pharmaceutically acceptable salt of any of the foregoing, wherein variables R¹, R², R³, and Z are as defined in Embodiment 9.

15. The compound, tautomer, deuterated derivative, or pharmaceutically acceptable salt according to any one of Embodiments 9-14, wherein R¹ is selected from optionally substituted phenyl and O-phenyl.
16. The compound, tautomer, deuterated derivative, or pharmaceutically acceptable salt according to any one of Embodiments 9-15, wherein R³ is selected from optionally substituted phenyl and optionally substituted O-phenyl.
17. A compound selected from:

| Cmpd No. | Structure |
|---|---|
| 116 | 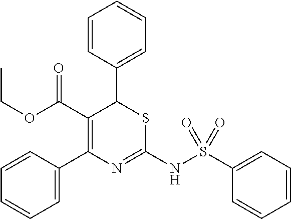 [SMILES: CCOC(=O)C1=C(c2ccccc2)N=C(NS(=O)(=O)c2ccccc2)SC1c1ccccc1] |
| 117 | 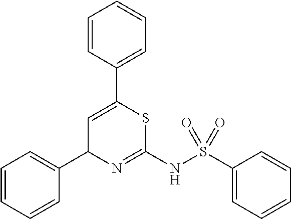 [SMILES: O=S(=O)(NC1=NC(c2ccccc2)C=C(c2ccccc2)S1)c1ccccc1] |
| 118 | 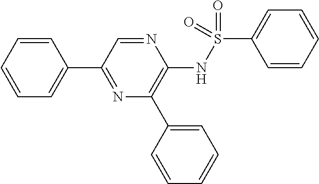 [SMILES: O=S(=O)(Nc1ncc(-c2ccccc2)nc1-c1ccccc1)c1ccccc1] |
| 119 | 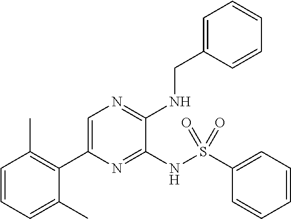 [SMILES: Cc1cccc(C)c1-c1cnc(NCc2ccccc2)c(NS(=O)(=O)c2ccccc2)n1] |
| 120 | 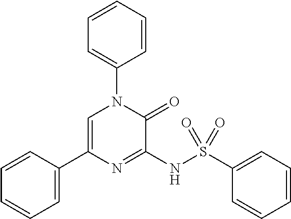 [SMILES: O=c1c(NS(=O)(=O)c2ccccc2)nc(-c2ccccc2)cn1-c1ccccc1] |
| 121 | 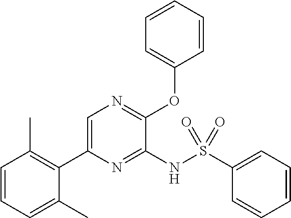 [SMILES: Cc1cccc(C)c1-c1cnc(Oc2ccccc2)c(NS(=O)(=O)c2ccccc2)n1] |
| 122 | 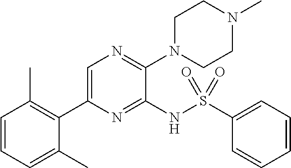 [SMILES: Cc1cccc(C)c1-c1cnc(N2CCN(C)CC2)c(NS(=O)(=O)c2ccccc2)n1] |
| 123 | 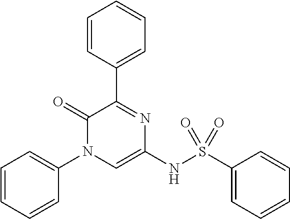 [SMILES: O=c1c(-c2ccccc2)nc(NS(=O)(=O)c2ccccc2)cn1-c1ccccc1] |
| 124 | 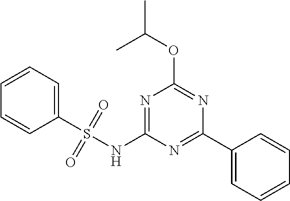 [SMILES: CC(C)Oc1nc(NS(=O)(=O)c2ccccc2)nc(-c2ccccc2)n1] |
| 125 | 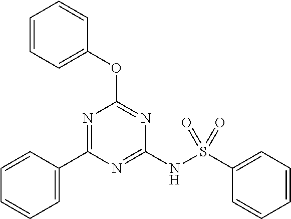 [SMILES: O=S(=O)(Nc1nc(Oc2ccccc2)nc(-c2ccccc2)n1)c1ccccc1] |
| 126 | 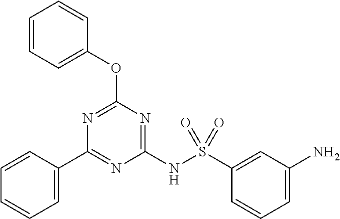 [SMILES: Nc1cccc(S(=O)(=O)Nc2nc(Oc3ccccc3)nc(-c3ccccc3)n2)c1] |
| 127 | 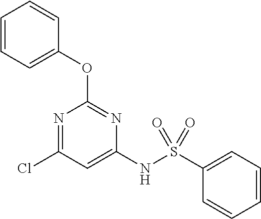 [SMILES: O=S(=O)(Nc1cc(Cl)nc(Oc2ccccc2)n1)c1ccccc1] |
| 128 | 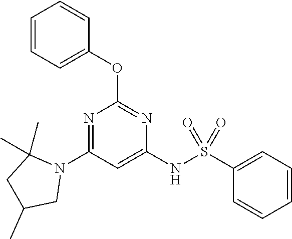 [SMILES: CC1CN(c2cc(NS(=O)(=O)c3ccccc3)nc(Oc3ccccc3)n2)C(C)(C)C1] |
| 129 | 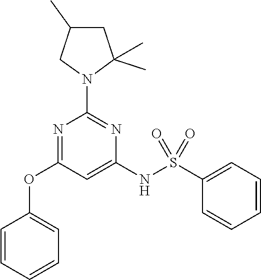 [SMILES: CC1CN(c2nc(NS(=O)(=O)c3ccccc3)cc(Oc3ccccc3)n2)C(C)(C)C1] |
| 130 | 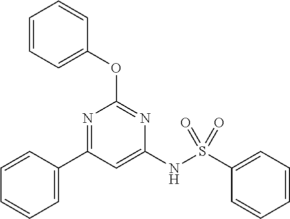 [SMILES: O=S(=O)(Nc1cc(-c2ccccc2)nc(Oc2ccccc2)n1)c1ccccc1] |
| 131 | 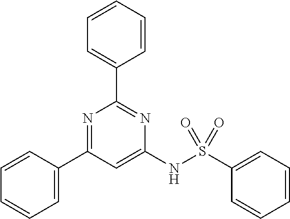 [SMILES: O=S(=O)(Nc1cc(-c2ccccc2)nc(-c2ccccc2)n1)c1ccccc1] |
| 132 | 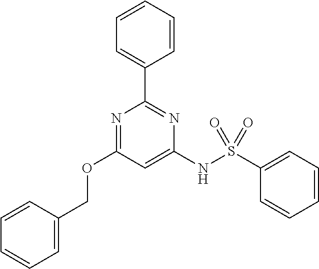 [SMILES: O=S(=O)(Nc1cc(OCc2ccccc2)nc(-c2ccccc2)n1)c1ccccc1] |
| 133 | 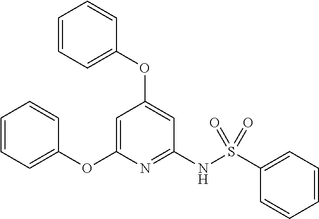 [SMILES: O=S(=O)(Nc1cc(Oc2ccccc2)cc(Oc2ccccc2)n1)c1ccccc1] |
| 134 | 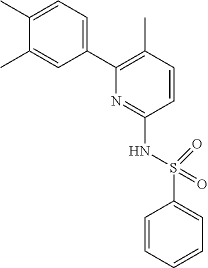 [SMILES: Cc1ccc(-c2nc(NS(=O)(=O)c3ccccc3)ccc2C)cc1C] |
| 135 | 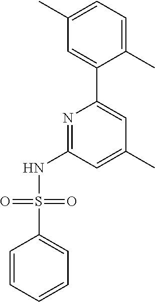 [SMILES: Cc1cc(NS(=O)(=O)c2ccccc2)nc(-c2cc(C)ccc2C)c1] |
| 136 | 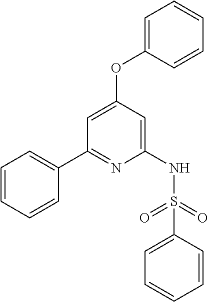 [SMILES: O=S(=O)(Nc1cc(Oc2ccccc2)cc(-c2ccccc2)n1)c1ccccc1] |
| 137 | 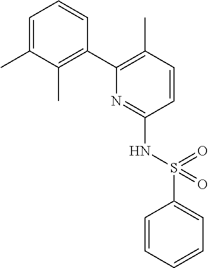 [SMILES: Cc1ccc(NS(=O)(=O)c2ccccc2)nc1-c1cccc(C)c1C] |
| 138 | 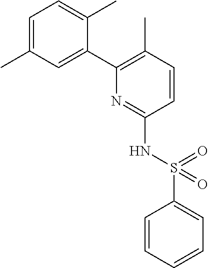 [SMILES: Cc1ccc(C)c(-c2nc(NS(=O)(=O)c3ccccc3)ccc2C)c1] |
| 139 | 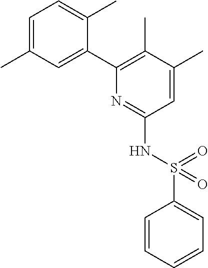 |
| 140 | 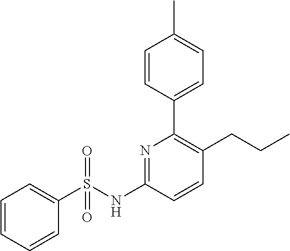 [SMILES: CCCc1ccc(NS(=O)(=O)c2ccccc2)nc1-c1ccc(C)cc1] |
| 141 | 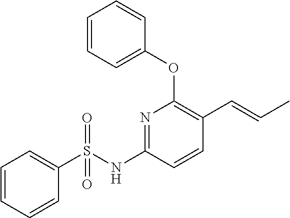 [SMILES: C/C=C/c1ccc(NS(=O)(=O)c2ccccc2)nc1Oc1ccccc1] |
| 142 | 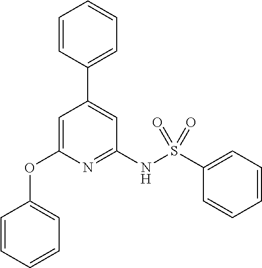 [SMILES: O=S(=O)(Nc1cc(-c2ccccc2)cc(Oc2ccccc2)n1)c1ccccc1] |
| 143 | 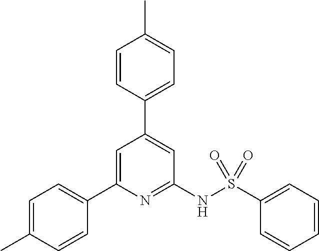 [SMILES: Cc1ccc(-c2cc(NS(=O)(=O)c3ccccc3)nc(-c3ccc(C)cc3)c2)cc1] |
| 144 | 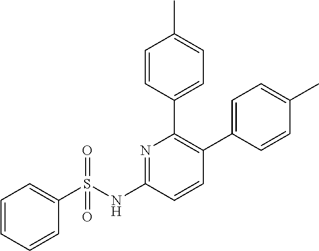 [SMILES: Cc1ccc(-c2ccc(NS(=O)(=O)c3ccccc3)nc2-c2ccc(C)cc2)cc1] |
| 145 | 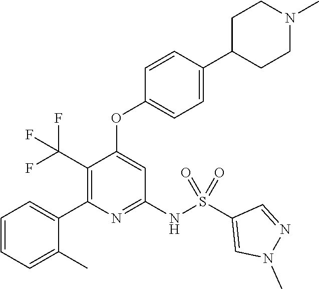 [SMILES: Cc1ccccc1-c1nc(NS(=O)(=O)c2cnn(C)c2)cc(Oc2ccc(C3CCN(C)CC3)cc2)c1C(F)(F)F] |
| 146 | 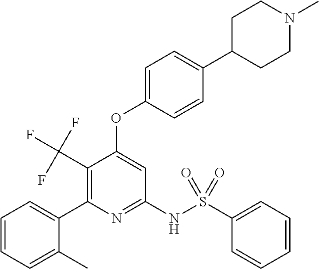 [SMILES: Cc1ccccc1-c1nc(NS(=O)(=O)c2ccccc2)cc(Oc2ccc(C3CCN(C)CC3)cc2)c1C(F)(F)F] |
| 147 | 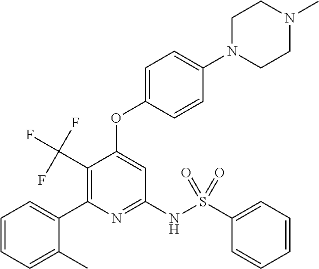 [SMILES: Cc1ccccc1-c1nc(NS(=O)(=O)c2ccccc2)cc(Oc2ccc(N3CCN(C)CC3)cc2)c1C(F)(F)F] |
| 148 | 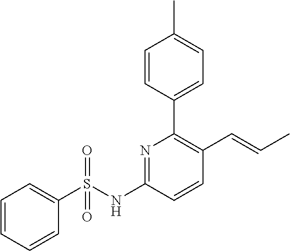 [SMILES: C/C=C/c1ccc(NS(=O)(=O)c2ccccc2)nc1-c1ccc(C)cc1] |
| 149 | 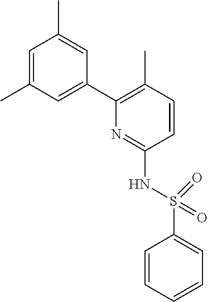 [SMILES: Cc1cc(C)cc(-c2nc(NS(=O)(=O)c3ccccc3)ccc2C)c1] |
| 150 | 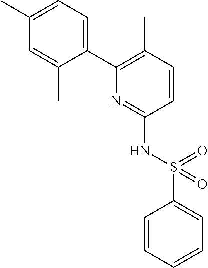 [SMILES: Cc1ccc(-c2nc(NS(=O)(=O)c3ccccc3)ccc2C)c(C)c1] |
| 151 | 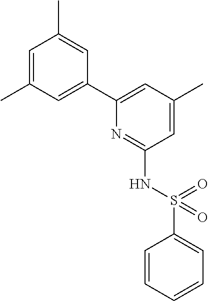 |
| 152 | 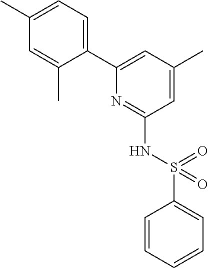 |
| 153 | 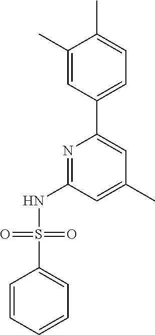 [SMILES: Cc1cc(NS(=O)(=O)c2ccccc2)nc(-c2ccc(C)c(C)c2)c1] |
| 154 | 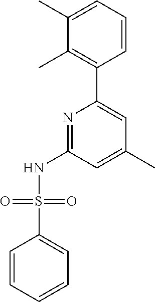 [SMILES: Cc1cc(NS(=O)(=O)c2ccccc2)nc(-c2cccc(C)c2C)c1] |
| 155 | 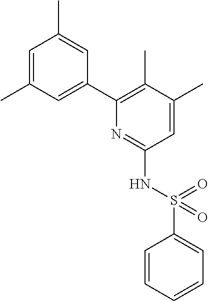 |
| 156 | 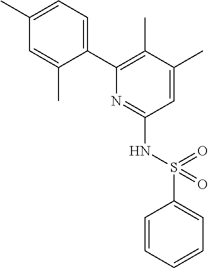 |
| 157 | 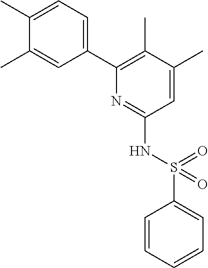 |
| 158 | 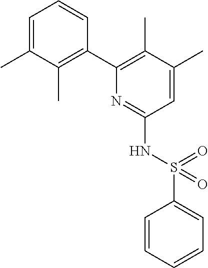 |

or a tautomer thereof, a deuterated derivative of the compound or tautomer, or a pharmaceutically acceptable salt of any of the foregoing.

18. A compound selected from:

| Cmpd No. | Structure |
|---|---|
| 116 | 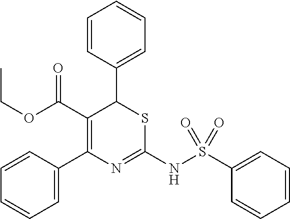 [SMILES: CCOC(=O)C1=C(c2ccccc2)N=C(NS(=O)(=O)c2ccccc2)SC1c1ccccc1] |
| 117 | 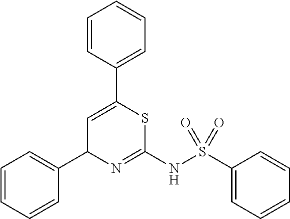 [SMILES: O=S(=O)(NC1=NC(c2ccccc2)C=C(c2ccccc2)S1)c1ccccc1] |
| 118 | 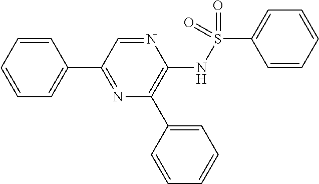 [SMILES: O=S(=O)(Nc1ncc(-c2ccccc2)nc1-c1ccccc1)c1ccccc1] |
| 119 | 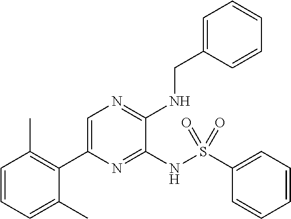 [SMILES: Cc1cccc(C)c1-c1cnc(NCc2ccccc2)c(NS(=O)(=O)c2ccccc2)n1] |
| 120 | 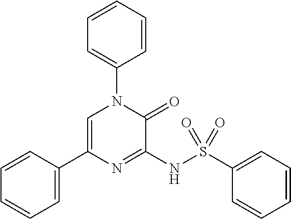 [SMILES: O=c1c(NS(=O)(=O)c2ccccc2)nc(-c2ccccc2)cn1-c1ccccc1] |
| 124 | 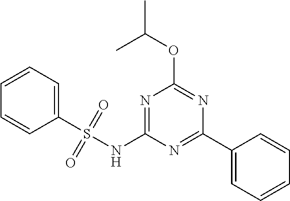 [SMILES: CC(C)Oc1nc(NS(=O)(=O)c2ccccc2)nc(-c2ccccc2)n1] |
| 125 | 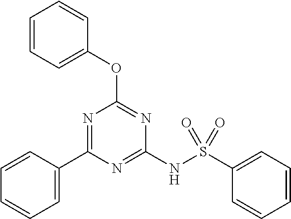 [SMILES: O=S(=O)(Nc1nc(Oc2ccccc2)nc(-c2ccccc2)n1)c1ccccc1] |
| 126 | 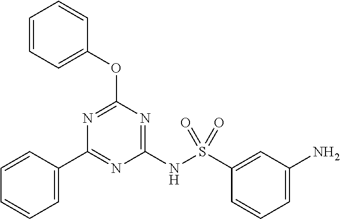 [SMILES: Nc1cccc(S(=O)(=O)Nc2nc(Oc3ccccc3)nc(-c3ccccc3)n2)c1] |
| 128 | 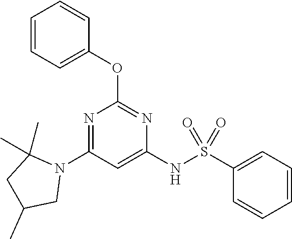 [SMILES: CC1CN(c2cc(NS(=O)(=O)c3ccccc3)nc(Oc3ccccc3)n2)C(C)(C)C1] |
| 130 | 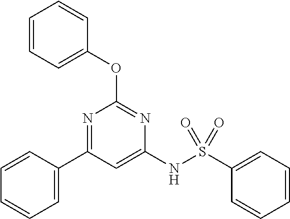 [SMILES: O=S(=O)(Nc1cc(-c2ccccc2)nc(Oc2ccccc2)n1)c1ccccc1] |
| 131 | 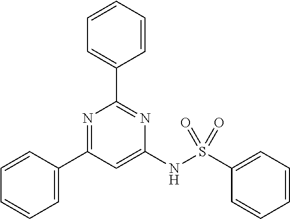 [SMILES: O=S(=O)(Nc1cc(-c2ccccc2)nc(-c2ccccc2)n1)c1ccccc1] |
| 132 | 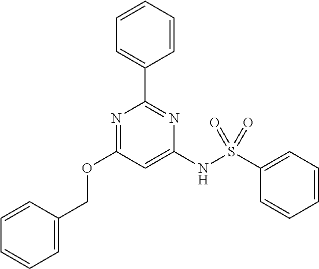 [SMILES: O=S(=O)(Nc1cc(OCc2ccccc2)nc(-c2ccccc2)n1)c1ccccc1] |
| 133 | 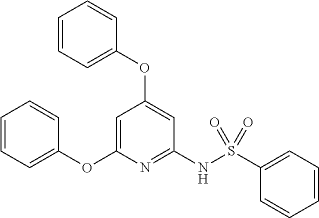 [SMILES: O=S(=O)(Nc1cc(Oc2ccccc2)cc(Oc2ccccc2)n1)c1ccccc1] |
| 134 | 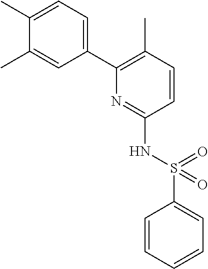 [SMILES: Cc1ccc(-c2nc(NS(=O)(=O)c3ccccc3)ccc2C)cc1C] |
| 135 | 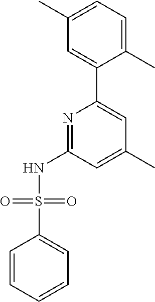 [SMILES: Cc1cc(NS(=O)(=O)c2ccccc2)nc(-c2cc(C)ccc2C)c1] |
| 136 | 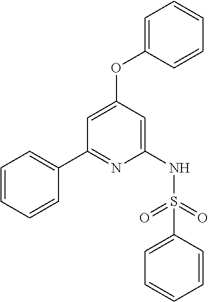 [SMILES: O=S(=O)(Nc1cc(Oc2ccccc2)cc(-c2ccccc2)n1)c1ccccc1] |
| 137 | 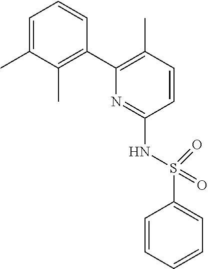 [SMILES: Cc1ccc(NS(=O)(=O)c2ccccc2)nc1-c1cccc(C)c1C] |
| 138 | 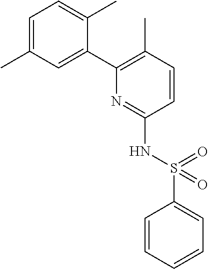 [SMILES: Cc1ccc(C)c(-c2nc(NS(=O)(=O)c3ccccc3)ccc2C)c1] |
| 139 | 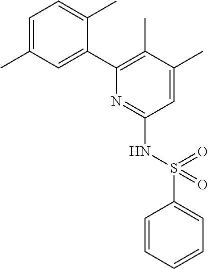 |
| 140 | 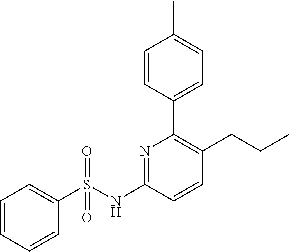 [SMILES: CCCc1ccc(NS(=O)(=O)c2ccccc2)nc1-c1ccc(C)cc1] |
| 141 | 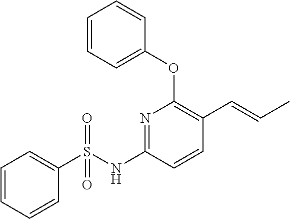 [SMILES: C/C=C/c1ccc(NS(=O)(=O)c2ccccc2)nc1Oc1ccccc1] |
| 142 | 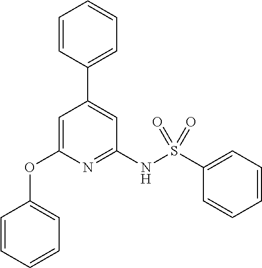 [SMILES: O=S(=O)(Nc1cc(-c2ccccc2)cc(Oc2ccccc2)n1)c1ccccc1] |
| 143 | 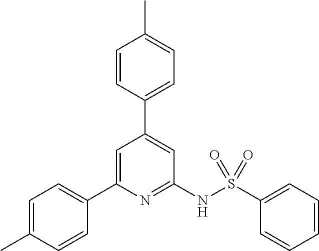 [SMILES: Cc1ccc(-c2cc(NS(=O)(=O)c3ccccc3)nc(-c3ccc(C)cc3)c2)cc1] |
| 144 | 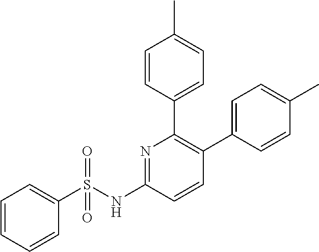 [SMILES: Cc1ccc(-c2ccc(NS(=O)(=O)c3ccccc3)nc2-c2ccc(C)cc2)cc1] |
| 146 | 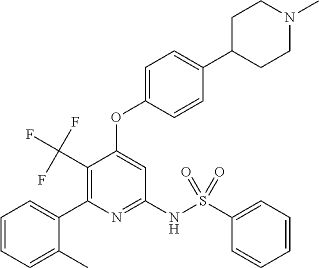 [SMILES: Cc1ccccc1-c1nc(NS(=O)(=O)c2ccccc2)cc(Oc2ccc(C3CCN(C)CC3)cc2)c1C(F)(F)F] |
| 147 | 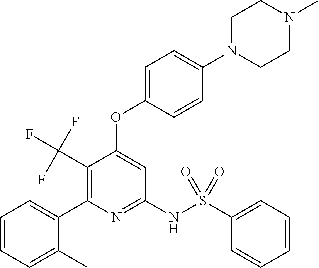 [SMILES: Cc1ccccc1-c1nc(NS(=O)(=O)c2ccccc2)cc(Oc2ccc(N3CCN(C)CC3)cc2)c1C(F)(F)F] |
| 148 | 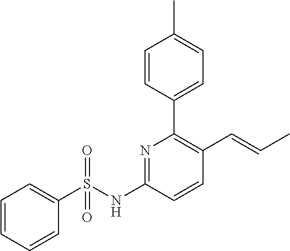 [SMILES: C/C=C/c1ccc(NS(=O)(=O)c2ccccc2)nc1-c1ccc(C)cc1] |
| 149 | 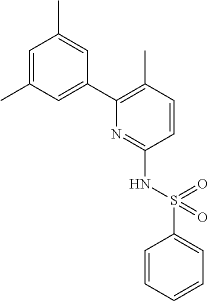 [SMILES: Cc1cc(C)cc(-c2nc(NS(=O)(=O)c3ccccc3)ccc2C)c1] |
| 150 | 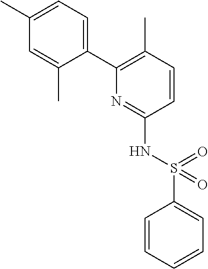 [SMILES: Cc1ccc(-c2nc(NS(=O)(=O)c3ccccc3)ccc2C)c(C)c1] |
| 151 | 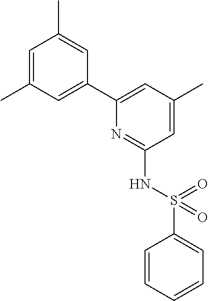 [SMILES: Cc1cc(C)cc(-c2cc(C)cc(NS(=O)(=O)c3ccccc3)n2)c1] |
| 152 | 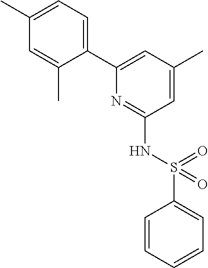 |
| 153 | 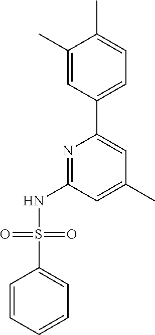 [SMILES: Cc1cc(NS(=O)(=O)c2ccccc2)nc(-c2ccc(C)c(C)c2)c1] |
| 154 | 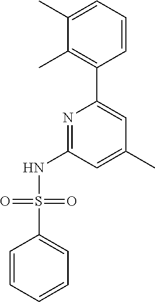 [SMILES: Cc1cc(NS(=O)(=O)c2ccccc2)nc(-c2cccc(C)c2C)c1] |
| 155 | 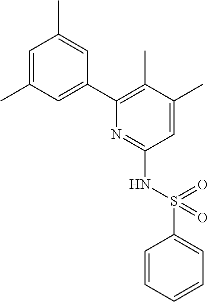 [SMILES: Cc1cc(C)cc(-c2nc(NS(=O)(=O)c3ccccc3)cc(C)c2C)c1] |
| 156 | 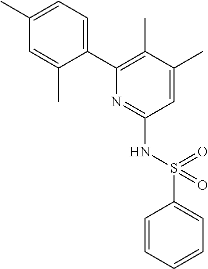 [SMILES: Cc1ccc(-c2nc(NS(=O)(=O)c3ccccc3)cc(C)c2C)c(C)c1] |
| 157 | 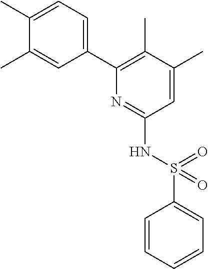 [SMILES: Cc1ccc(-c2nc(NS(=O)(=O)c3ccccc3)cc(C)c2C)cc1C] |
| 158 | 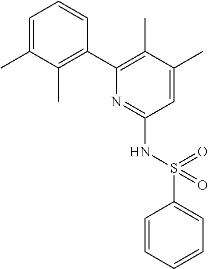 [SMILES: Cc1cccc(-c2nc(NS(=O)(=O)c3ccccc3)cc(C)c2C)c1C] |

or a tautomer thereof, a deuterated derivative of the compound or tautomer, or a pharmaceutically acceptable salt of any of the foregoing.

19. A pharmaceutical composition comprising a compound, tautomer, deuterated derivative, or pharmaceutically acceptable salt of any one of Embodiments 1-18 and a pharmaceutically acceptable carrier.
20. The pharmaceutical composition of Embodiment 19, further comprising one or more additional therapeutic agent(s).
21. The pharmaceutical composition of Embodiment 20, where the one or more additional therapeutic agent(s) is selected from CFTR modulators.
22. The pharmaceutical composition of Embodiment 21, where CFTR modulator(s) is a potentiator.
23. The pharmaceutical composition of Embodiment 21, where CFTR modulator(s) is a corrector.
24. The pharmaceutical composition of Embodiment 21, wherein one or more additional therapeutic agents are a potentiator and a corrector.
25. A pharmaceutical composition comprising (a) a compound, tautomer, deuterated derivative, or pharmaceutically acceptable salt of any one of Embodiments 1-18, (b) a pharmaceutically acceptable carrier, and (c) one or more CFTR modulator(s) selected from lumacaftor, tezacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
26. A pharmaceutical composition comprising:
(a) at least one compound, tautomer, deuterated derivative, or pharmaceutically acceptable salt according to any one of Embodiments 1-18;
(b) at least one pharmaceutically acceptable carrier; and optionally one or more of:
(c) (i) a compound chosen from
tezacaftor:

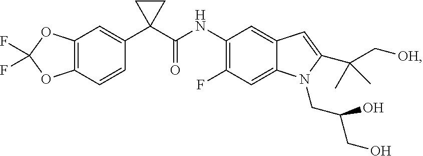 [SMILES: CC(C)(CO)c1cc2cc(NC(=O)C3(c4ccc5c(c4)OC(F)(F)O5)CC3)c(F)cc2n1C[C@@H](O)CO]

lumacaftor:

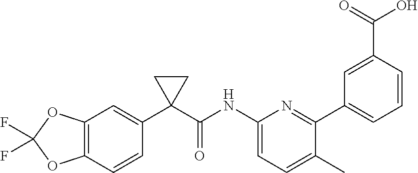 [SMILES: Cc1ccc(NC(=O)C2(c3ccc4c(c3)OC(F)(F)O4)CC2)nc1-c1cccc(C(=O)O)c1]

and deuterated derivatives and pharmaceutically acceptable salts of tezacaftor and lumacaftor; and
   (ii) a compound chosen from
ivacaftor:

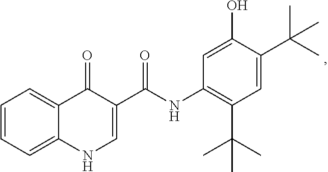 [SMILES: CC(C)(C)c1cc(C(C)(C)C)c(NC(=O)c2c[nH]c3ccccc3c2=O)cc1O]

deutivacaftor:

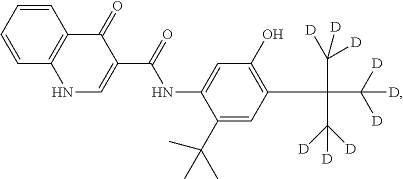

(6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol:

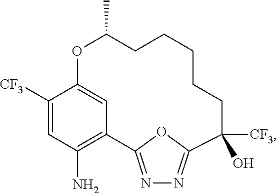 [SMILES: C[C@@H]1CCCCC[C@](O)(C(F)(F)F)c2nnc(o2)-c2cc(c(C(F)(F)F)cc2N)O1]

and deuterated derivatives and pharmaceutically acceptable salts of any of the foregoing.
27. The pharmaceutical composition of Embodiment 26, comprising:
(a) at least one compound, tautomer, deuterated derivative, or pharmaceutically acceptable salt according to any one of Embodiments 1-18;
(b) at least one pharmaceutically acceptable carrier; and
(c) a compound chosen from

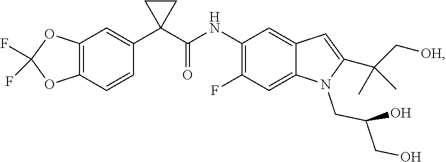 [SMILES: CC(C)(CO)c1cc2cc(NC(=O)C3(c4ccc5c(c4)OC(F)(F)O5)CC3)c(F)cc2n1C[C@@H](O)CO]

tezacaftor:
   and deuterated derivatives and pharmaceutically acceptable salts of tezacaftor.
   28. The pharmaceutical composition of Embodiment 26, comprising:
(a) at least one compound, tautomer, deuterated derivative, or pharmaceutically acceptable salt according to any one of Embodiments 1-18;
(b) at least one pharmaceutically acceptable carrier; and
(c) (i) a compound chosen from

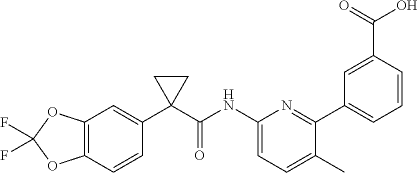 [SMILES: Cc1ccc(NC(=O)C2(c3ccc4c(c3)OC(F)(F)O4)CC2)nc1-c1cccc(C(=O)O)c1]

lumacaftor:
   and deuterated derivatives and pharmaceutically acceptable salts of lumacaftor.
   29. The pharmaceutical composition of Embodiment 26, comprising:
(a) at least one compound, tautomer, deuterated derivative, or pharmaceutically acceptable salt according to any one of Embodiments 1-18;
(b) at least one pharmaceutically acceptable carrier; and
(c) a compound chosen from

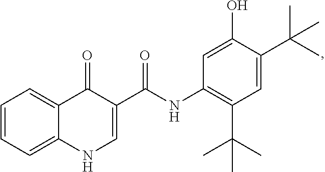 [SMILES: CC(C)(C)c1cc(C(C)(C)C)c(NC(=O)c2c[nH]c3ccccc3c2=O)cc1O]

ivacaftor:
   and deuterated derivatives and pharmaceutically acceptable salts thereof.
   30. The pharmaceutical composition of Embodiment 26, comprising:
(a) at least one compound, tautomer, deuterated derivative, or pharmaceutically acceptable salt according to any one of Embodiments 1-18;
(b) at least one pharmaceutically acceptable carrier; and optionally one or more of:
(c) a compound chosen from
deutivacaftor:

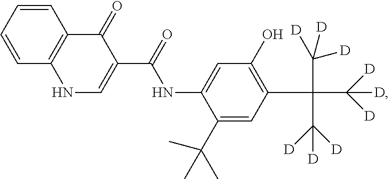

and deuterated derivatives and pharmaceutically acceptable salts thereof.
31. The pharmaceutical composition of Embodiment 26, comprising:
(a) at least one compound, tautomer, deuterated derivative, or pharmaceutically acceptable salt according to any one of Embodiments 1-18;
(b) at least one pharmaceutically acceptable carrier; and
(c) a compound chosen from
(6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol:

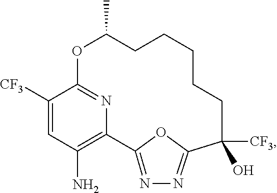 [SMILES: C[C@@H]1CCCCC[C@](O)(C(F)(F)F)c2nnc(o2)-c2nc(c(C(F)(F)F)cc2N)O1]

and deuterated derivatives and pharmaceutically acceptable salts of any of the foregoing.
32. The pharmaceutical composition of Embodiment 26, wherein the pharmaceutical composition comprises:
(a) at least one compound, tautomer, deuterated derivative, or pharmaceutically acceptable salt according to any one of Embodiments 1-18;
(b) at least one pharmaceutically acceptable carrier;
(c) tezacaftor; and
(d) ivacaftor.
   33. The pharmaceutical composition of Embodiment 26, wherein the pharmaceutical composition comprises:
(a) at least one compound, tautomer, deuterated derivative, or pharmaceutically acceptable salt according to any one of Embodiments 1-18;
(b) at least one pharmaceutically acceptable carrier;
(c) tezacaftor; and
(d) deutivacaftor.
   34. The pharmaceutical composition of Embodiment 26, wherein the pharmaceutical composition comprises:
(a) at least one compound, tautomer, deuterated derivative, or pharmaceutically acceptable salt according to any one of Embodiments 1-18;
(b) at least one pharmaceutically acceptable carrier;
(c) tezacaftor, and
(d) (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
   35. The pharmaceutical composition of Embodiment 26, wherein the pharmaceutical composition comprises:
(a) at least one compound, tautomer, deuterated derivative, or pharmaceutically acceptable salt according to any one of Embodiments 1-18;
(b) at least one pharmaceutically acceptable carrier;
(c) lumacaftor; and
(d) ivacaftor.
   36. The pharmaceutical composition of Embodiment 26, wherein the pharmaceutical composition comprises:
(a) at least one compound, tautomer, deuterated derivative, or pharmaceutically acceptable salt according to any one of Embodiments 1-18;
(b) at least one pharmaceutically acceptable carrier;
(c) lumacaftor; and
(d) deutivacaftor.
   37. The pharmaceutical composition of Embodiment 26, wherein the pharmaceutical composition comprises:
(a) at least one compound, tautomer, deuterated derivative, or pharmaceutically acceptable salt according to any one of Embodiments 1-18;
(b) at least one pharmaceutically acceptable carrier;
(c) lumacaftor; and
(d) (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
   38. A method of treating cystic fibrosis comprising administering to a patient in need thereof a compound, tautomer, deuterated derivative, or pharmaceutically acceptable salt of any one of Embodiments 1-18 or a pharmaceutical composition according to any one of Embodiments 19-37.
   39. The method of Embodiment 38, comprising administering to a patient in need thereof a compound, tautomer, deuterated derivative, or pharmaceutically acceptable salt of any one of Embodiments 1-18 or a pharmaceutical composition according to any one of Embodiments 19-37, and further comprising administering to the patient one or more additional therapeutic agent(s) prior to, concurrent with, or subsequent to the compound or the pharmaceutical composition.
   40. The method of Embodiment 38, wherein the one or more additional therapeutic agent(s) comprise(s) a CFTR modulating agent.
   41. The method of Embodiment 38, wherein the CFTR modulating agent is a potentiator.
   42. The method of Embodiment 38, wherein the CFTR modulating agent is a corrector.
   43. The method of Embodiment 38, wherein the one or more additional therapeutic agent(s) is selected from tezacaftor, ivacaftor, deutivacaftor, lumacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
   44. The method of Embodiment 38, wherein the one or more additional therapeutic agent(s) comprise(s) tezacaftor and ivacaftor.
   45. The method of Embodiment 38, wherein the one or more additional therapeutic agent(s) comprise(s) tezacaftor and deutivacaftor.
   46. The method of Embodiment 38, wherein the one or more additional therapeutic agent(s) comprise(s) tezacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
   47. The method of Embodiment 38, wherein the one or more additional therapeutic agent(s) comprise(s) lumacaftor and ivacaftor.
   48. The method of Embodiment 38, wherein the one or more additional therapeutic agent(s) comprise(s) lumacaftor and deutivacaftor.
   49. The method of Embodiment 38, wherein the one or more additional therapeutic agent(s) comprise(s) lumacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
   50. The compound, tautomer, deuterated derivative, or pharmaceutically acceptable salt of any one of Embodiments 1-18 or the pharmaceutical composition according to any one of Embodiments 19-37 for use in the treatment of cystic fibrosis.
   51. The compound, tautomer, deuterated derivative, or pharmaceutically acceptable salt of any one of Embodiments 1-18 or the pharmaceutical composition according to any one of Embodiments 19-37 for use in the manufacture of a medicament for the treatment of cystic fibrosis.

### EXAMPLES

### I. Abbreviation List

API: Active pharmaceutical ingredient
ACN: Acetonitrile
Boc anhydride, ((Boc)₂O): Di-tert-butyl dicarbonate
CDCl₃: Chloroform-d
CDI: Carbonyl diimidazole
CH₂Cl₂: Dichloromethane
CH₃CN: Acetonitrile
COMU: (1-Cyano-2-ethoxy-2-oxoethylidenaminooxy)dimethylamino-morpholino-carbenium hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA; N,N-diisopropylethylamine)
DMA: N,N-Dimethylacetamide
DMAP: 4-Dimethylaminopyridine
DMF: N,N-Dimethylformamide
DMSO: Dimethyl sulfoxide
EA: Ethyl acetate
EDC: 1-Ethyl-3-(3-dimethylaminopropyl)carbodiimide
ELSD: Evaporative light scattering detector
ESI-MS: Electrospray ionization mass spectrometry
Et₂O: Diethyl ether
EtOAc: Ethyl acetate
EtOH: Ethanol
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High performance liquid chromatography
Hoveyda-Grubbs 2^{nd} Generation catalyst: (1,3-Bis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MeTHF or 2-MeTHF: 2-Methyltetrahydrofuran
MgSO₄: Magnesium sulfate
MTBE: Methyl tert-butyl ether
NaHCO₃: Sodium bicarbonate
NaOH: Sodium hydroxide
NMP: N-Methyl-2-pyrrolidone
NMM: N-Methylmorpholine
Pd₂(dba)₃: Tris(dibenzylideneacetone)dipalladium(O)
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
PTSA: para-toluenesulfonic acid
rt: Room temperature
RuPhos: 2-Dicyclohexylphosphino-2′,6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TMS: Trimethylsilyl
TMSCl: Trimethylsilyl chloride
UPLC: Ultra Performance Liquid Chromatography
XANTPHOS: 4,5-Bis(diphenylphosphino)-9,9-dimethylxanthene
XPhos Pd G1: (2-Dicyclohexylphosphino-2′,4′,6′-triisopropyl-1,1′-biphenyl)[2-(2-aminoethyl)phenyl)]palladium(II) chloride

### II. General Methods

Reagents and starting materials were obtained from commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz, respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution, respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz, and 28860 points of acquisition. FID were zero-filled to 32 k points and a line broadening of 0.3 Hz was applied before Fourier transform. ¹⁹F NMR spectra were recorded at 282 MHz using a 30 degree pulse angle; a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz, and 128 k points of acquisition. FID were zero-filled to 256 k points and a line broadening of 0.3 Hz was applied before Fourier transform. ¹⁹F NMR spectra were recorded at 377 MHz using a 30 degree pulse angle; a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### III. General UPLC/HPLC Analytical Methods

LC method A: Analytical reverse phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 L, and column temperature=60° C.

LC method D: Acquity UPLC BEH C₁₈ column (30×2.1 mm, 1.7 μm particle) made by Waters (pn: 186002349), and a dual gradient run from 1-99% mobile phase B over 1.0 minute. Mobile phase A=H₂O (0.05% CF₃CO₂H). Mobile phase B═CH₃CN (0.035% CF₃CO₂H). Flow rate=1.5 mL/min, injection volume=1.5 μL, and column temperature=60° C.

LC method I: Acquity UPLC BEH C₁₈ column (50×2.1 mm, 1.7 μm particle) made by Waters (pn:186002350), and a dual gradient run from 1-99% mobile phase B over 5.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### IV. Synthesis of New Compounds

### Example 1: Preparation of Compound 1

### Step 1: N-[3,4-bis(4-Chlorophenyl)isoxazol-5-yl]benzenesulfonamide

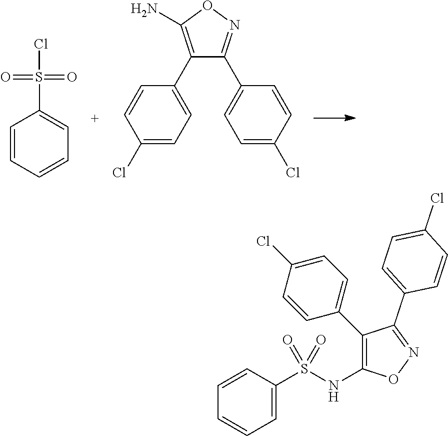 [SMILES: O=S(=O)(Cl)c1ccccc1 ; Nc1onc(-c2ccc(Cl)cc2)c1-c1ccc(Cl)cc1 ; O=S(=O)(Nc1onc(-c2ccc(Cl)cc2)c1-c1ccc(Cl)cc1)c1ccccc1]

To a vial was added 3,4-bis(4-chlorophenyl)isoxazol-5-amine (approximately 43.18 mg, 0.1415 mmol), anhydrous DCM and NaH (approximately 22.64 mg of 60% w/w, 0.5660 mmol). The reaction solution was allowed to stir at 23° C. for 15 minutes prior to the addition of a solution of benzenesulfonyl chloride (25 mg, 0.1415 mmol) in DCM. The reaction solution was allowed to stir at room temperature overnight. The reaction mixtures was filtered and purified by reverse-phase preparative HPLC to afford N-[3,4-bis(4-chlorophenyl)isoxazol-5-yl]benzenesulfonamide (15.4 mg, 24%). ESI-MS m/z calc. 444.01022, found 445.18 (M+1)⁺; Retention time: 2.21 minutes; LC method A. ¹H NMR (400 MHz, DMSO-d₆) δ 7.78 (dd, J=7.2, 1.7 Hz, 2H), 7.59 (dd, J=17.3, 8.2 Hz, 1H), 7.54-7.44 (m, 4H), 7.38-7.25 (m, 4H), 7.13 (dd, J=8.8, 2.1 Hz, 2H).

### Example 2: Preparation of Compound 2

### Step 1: 2-Iodo-1-phenyl-pentan-1-one

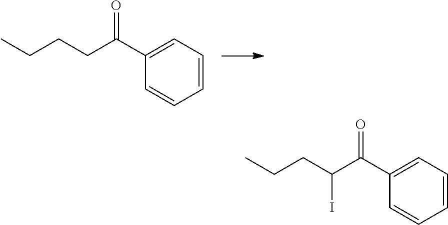

A solution of 1-phenylpentan-1-one (0.5 mL, 3.020 mmol) and Iodine (approximately 3.066 g, 621.9 μL, 12.08 mmol) in DME (4.900 mL) was stirred at 90° C. for 3 hours. The reaction mixture was poured into 0.5 M sodium thiosulfate and extracted with EtOAc (2×). Organics were combined, washed with 1 M sodium thiosulfate, water, brine, dried over Na₂SO₄, and evaporated to dryness. Purification by column chromatography (40 g Silica; 0-30% EtOAc in hexanes) gave 2-iodo-1-phenyl-pentan-1-one (500 mg, 57%) as a yellow oil. ESI-MS m/z calc. 288.0011, found 289.3 (M+1)⁺; Retention time: 0.71 minutes; LC method D.

### Step 2: 4-Phenyl-5-propyl-oxazol-2-amine

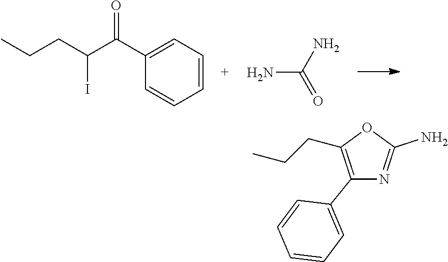

A solution of 2-iodo-1-phenyl-pentan-1-one (246 mg, 0.8538 mmol) and Urea (approximately 102.6 mg, 1.708 mmol) was stirred at 100° C. for 24 hours. The reaction mixture was poured into water, the pH brought to 12 with sat. aq. sodium carbonate and extracted with EtOAc (3×). Organics were combined, washed with water, brine, dried over sodium sulfate, and evaporated to dryness. Purification by column chromatography (12 g silica; 0-50% EtOAc in hexanes) gave 4-phenyl-5-propyl-oxazol-2-amine (15 mg, 9%) as a red-orange oil, which was used without further purification. ESI-MS m/z calc. 202.11061, found 203.2 (M+1)⁺; Retention time: 0.41 minutes; LC method D.

### Step 3: N-(4-Phenyl-5-propyl-oxazol-2-yl)benzenesulfonamide

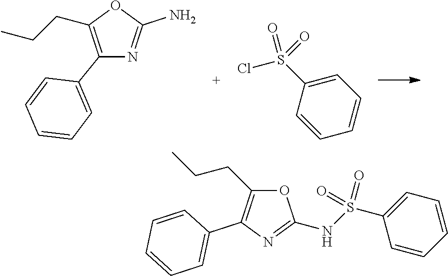

To a solution of 4-phenyl-5-propyl-oxazol-2-amine (15 mg, 0.07416 mmol) and DABCO (approximately 41.59 mg, 0.3708 mmol) in CH₃CN (0.4 mL) was added benzenesulfonyl chloride (approximately 26.19 mg, 18.92 μL, 0.1483 mmol) (exothermic) and the reaction mixture was stirred at room temperature for 1 hour. The reaction mixture was diluted with MeOH and filtered. Purification by HPLC (1-99% ACN in water (HCl modifier)) gave N-(4-phenyl-5-propyl-oxazol-2-yl)benzenesulfonamide (1.5 mg, 6%) as a white solid. ESI-MS m/z calc. 342.10382, found 343.3 (M+1)⁺; Retention time: 1.56 minutes; LC method A.

### Example 3: Preparation of Compound 3

### Step 1: N-[4,5-bis(p-Tolyl)oxazol-2-yl]benzenesulfonamide

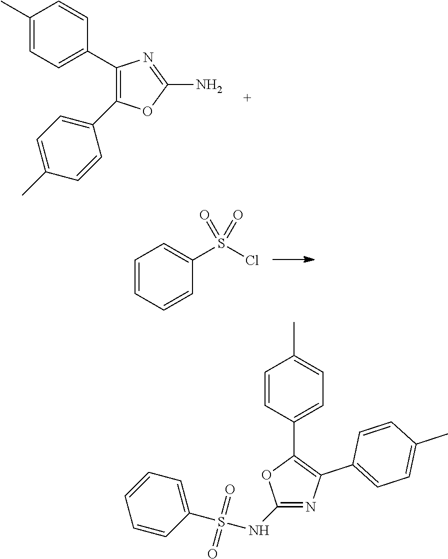 [SMILES: Cc1ccc(-c2nc(N)oc2-c2ccc(C)cc2)cc1 ; O=S(=O)(Cl)c1ccccc1 ; Cc1ccc(-c2nc(NS(=O)(=O)c3ccccc3)oc2-c2ccc(C)cc2)cc1]

Benzenesulfonyl chloride (approximately 50.00 mg, 36.13 μL, 0.2831 mmol) was added to 4,5-bis(p-tolyl)oxazol-2-amine (approximately 18.71 mg, 0.07078 mmol) in pyridine (0.2 mL). The mixture was stirred at 105° C. The crude was filtered and purified on reverse phase HPLC (HCl modifier, 30-99% ACN-H₂O) to give N-[4,5-bis(p-tolyl)oxazol-2-yl]benzenesulfonamide (4.7 mg, 16%). ESI-MS m/z calc. 404.11948, found 405.0 (M+1)⁺; Retention time: 1.89 minutes; LC method A.

### Example 4: Characterization of Compound 4

The compound in the following table was prepared in a manner analogous to that described above using commercially available reagents and intermediates described herein

| | | LCMS | | | | |
|---|---|---|---|---|---|---|
| | | Rt | Calc. | | LCMS | |
| Cmpd # | Structure | (min) | Mass | M + 1 | Met. | NMR |
| 4 | 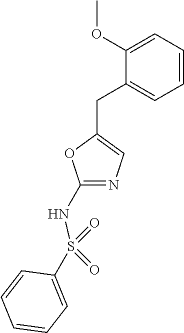 [SMILES: COc1ccccc1Cc1cnc(NS(=O)(=O)c2ccccc2)o1] | 1.34 | 344.083 | 345 | A | ¹H NMR (400 MHz, DMSO) δ 11.86 (s, 1H), 7.79 (d, J = 8.0 Hz, 2H), 7.52 (dt, J = 15.1, 7.8 Hz, 3H), 7.26 (t, J = 7.8 Hz, 1H), 7.09 (d, J = 7.5 Hz, 1H), 7.00 (d, J = 8.2 Hz, 1H), 6.88 (dd, J = 16.9, 9.5 Hz, 2H), 3.77 (s, 5H). |

### Example 5: Preparation of Compound 5

### Step 1: N-[5-Propyl-4-(p-tolyl)thiazol-2-yl]benzenesulfonamide

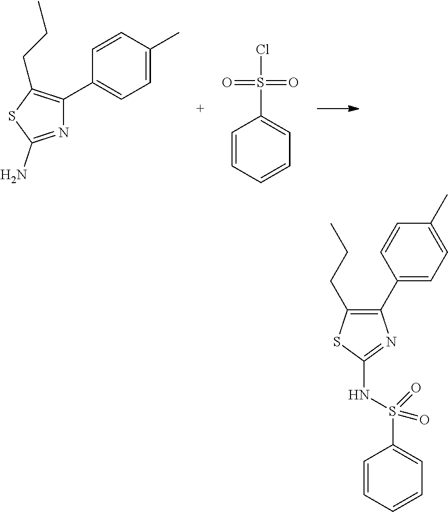 [SMILES: CCCc1sc(N)nc1-c1ccc(C)cc1 ; O=S(=O)(Cl)c1ccccc1 ; CCCc1sc(NS(=O)(=O)c2ccccc2)nc1-c1ccc(C)cc1]

Benzenesulfonyl chloride (28 μL, 0.2194 mmol) was added to 5-propyl-4-(p-tolyl)thiazol-2-amine (25 mg, 0.1076 mmol) and 1,4-diazabicyclo[2.2.2]octane (approximately 241.4 mg, 2.152 mmol) in acetonitrile (1 mL). The mixture was left to stir at room temperature over the weekend. The reaction mixture was filtered and purified on reverse phase HPLC (HCl modifier, 30-99% ACN-H₂O) to give N-[5-propyl-4-(p-tolyl)thiazol-2-yl]benzenesulfonamide. ¹H NMR (400 MHz, DMSO) δ 12.78 (s, 1H), 7.83 (d, J=7.5 Hz, 2H), 7.65-7.52 (m, 3H), 7.29 (q, J=8.2 Hz, 4H), 2.59-2.54 (m, 2H), 2.34 (s, 3H), 1.63-1.47 (m, 2H), 0.85 (t, J=7.3 Hz, 3H). ESI-MS m/z calc. 372.09662, found 373.0 (M+1)⁺; Retention time: 1.84 minutes; LC method A.

### Example 6: Preparation of Compound 6

### Step 1: N-[4-(2,4-Dimethylphenyl)-5-propyl-thiazol-2-yl]benzenesulfonamide

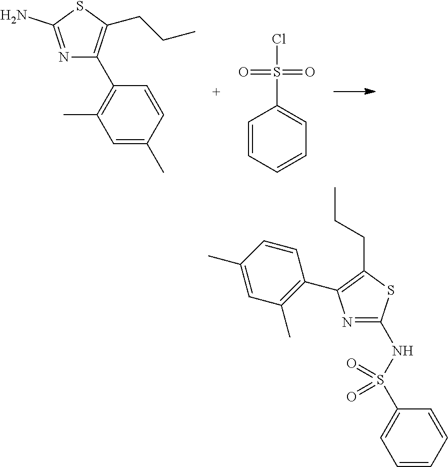

To a solution of 4-(2,4-dimethylphenyl)-5-propyl-thiazol-2-amine (approximately 34.89 mg, 0.1416 mmol) in pyridine (0.5 mL) was added benzenesulfonyl chloride (50 mg, 0.2831 mmol) and the reaction was stirred at 115° C. for 1 hour. The reaction mixture was filtered and purified on reverse phase HPLC (HCl modifier, 25-75% ACN-H₂O) to give N-[4-(2,4-dimethylphenyl)-5-propyl-thiazol-2-yl]benzenesulfonamide (23.2 mg). ESI-MS m/z calc. 386.11227, found 387.0 (M+1)⁺; Retention time: 1.89 minutes; LC method A. ¹H NMR (400 MHz, DMSO) δ 12.57 (s, 1H), 7.83 (d, J=6.7 Hz, 2H), 7.64-7.52 (m, 3H), 7.17-7.04 (m, 3H), 2.30 (s, 5H), 2.10 (s, 3H), 1.44 (dd, J=14.7, 7.4 Hz, 2H), 0.78 (t, J=7.3 Hz, 3H).

### Example 7: Preparation of Compound 7

### Step 1: N-(4,5-Diphenylthiazol-2-yl)benzenesulfonamide

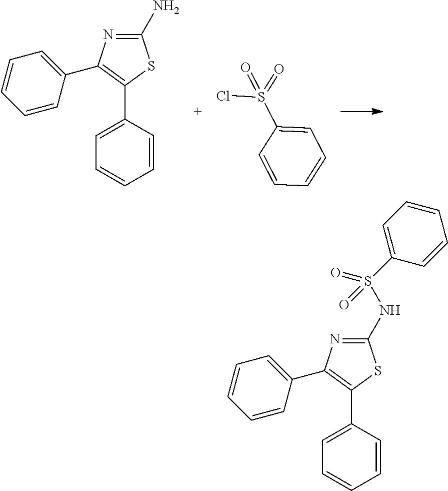 [SMILES: Nc1nc(-c2ccccc2)c(-c2ccccc2)s1 ; O=S(=O)(Cl)c1ccccc1 ; O=S(=O)(Nc1nc(-c2ccccc2)c(-c2ccccc2)s1)c1ccccc1]

Benzenesulfonyl chloride (approximately 39.85 mg, 28.79 μL, 0.2256 mmol) was added to 4,5-diphenylthiazol-2-amine (28 mg, 0.1110 mmol) and 1,4-diazabicyclo[2.2.2]octane (253 mg, 2.255 mmol) in acetonitrile (1 mL). The mixture was left to stir at room temperature overnight. The reaction mixture was filtered and purified on reverse phase HPLC (HCl modifier, 25-75% ACN-H₂O) to give N-(4,5-diphenylthiazol-2-yl)benzenesulfonamide (25.2 mg). ¹H NMR (400 MHz, DMSO) δ 13.15 (s, 1H), 7.88 (d, J=7.7 Hz, 2H), 7.65-7.55 (m, 3H), 7.35 (dd, J=17.5, 7.5 Hz, 8H), 7.27-7.20 (m, 2H). ESI-MS m/z calc. 392.0653, found 393.0 (M+1)⁺; Retention time: 1.77 minutes; LC method A.

### Example 8: Preparation of Compound 8

### Step 1: N-[4-(2,5-Dimethylphenyl)-5-methyl-thiazol-2-yl]benzenesulfonamide

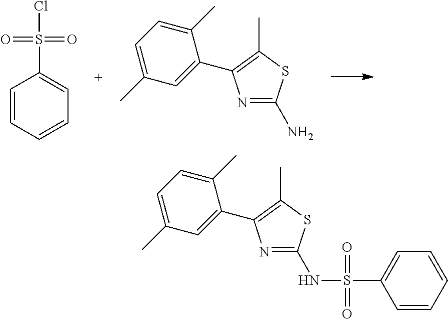

To a solution of 4-(2,5-dimethylphenyl)-5-methyl-thiazol-2-amine (approximately 30.91 mg, 0.1416 mmol) in pyridine (0.5 mL) was added benzenesulfonyl chloride (50 mg, 0.2831 mmol) and the reaction was stirred at 115° C. for 1 hour. The reaction mixture was filtered and purified on reverse phase HPLC (HCl modifier, 25-75% ACN-H₂O) to give N-[4-(2,5-dimethylphenyl)-5-methyl-thiazol-2-yl]benzenesulfonamide (19.5 mg). ESI-MS m/z calc. 358.08096, found 359.0 (M+1)⁺; Retention time: 1.66 minutes; LC method A. ¹H NMR (400 MHz, DMSO) δ 12.57 (s, 1H), 7.83 (d, J=9.6 Hz, 2H), 7.64-7.52 (m, 3H), 7.24-7.15 (m, 2H), 7.07 (s, 1H), 2.27 (s, 3H), 2.10 (s, 3H), 1.99 (s, 3H).

### Example 9: Preparation of Compound 9

### Step 1: N-(5-Bromo-4-phenyl-thiazol-2-yl)-3-nitro-benzenesulfonamide

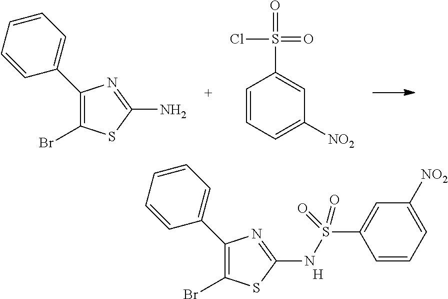

A solution of 5-bromo-4-phenyl-thiazol-2-amine (150.0 mg, 0.5879 mmol) and 3-nitrobenzenesulfonyl chloride (156 mg, 0.7039 mmol) in pyridine (600 μL) was heated in a sealed vial to 75° C. for 1 hour. The reaction was cooled to 23° C. and further stirred for 16 hours. The reaction mixture was diluted with ethyl acetate and a small quantity of methanol. The crude solution was submitted to flash column chromatography on silica gel (ethyl acetate in hexanes) to afford N-(5-bromo-4-phenyl-thiazol-2-yl)-3-nitro-benzenesulfonamide (36 mg, 14%) as an off-white solid. ESI-MS m/z calc. 438.9296, found 442.2 (M+3)⁺; Retention time: 0.65 minutes; LC method D.

### Step 2: 3-Amino-N-(5-benzyl-4-phenyl-thiazol-2-yl)benzenesulfonamide

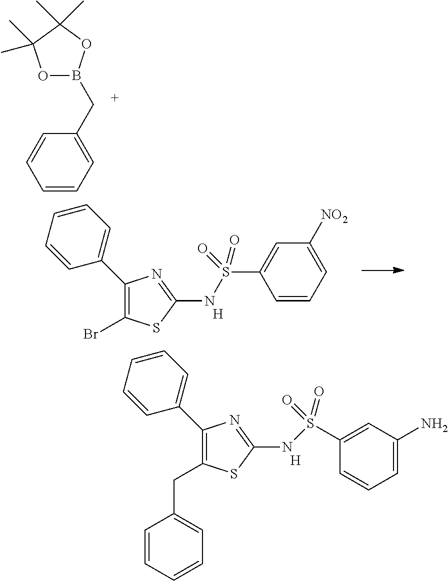

A biphasic mixture consisting of 2-benzyl-4,4,5,5-tetramethyl-1,3,2-dioxaborolane (55 mg, 0.2522 mmol), Pd(dppf)Cl₂ (10 mg, 0.012 mmol), sodium carbonate (180 μL of 2 M, 0.36 mmol), and N-(5-bromo-4-phenyl-thiazol-2-yl)-3-nitro-benzenesulfonamide (36 mg, 0.082 mmol) in dioxane (410 μL) was microwaved in a sealed vial at 80° C. for 20 minutes. The reaction mixture was diluted with diethyl ether and acidified using acetic acid (72 mg, 1.2 mmol). The organic layer was separated, and the aqueous layer was further extracted with diethyl ether (2×). The combined organics were dried using magnesium sulfate, filtered, and concentrated in vacuo. To the crude residue in ethanol (410 μL) was added iron (23 mg, 0.41 mmol) followed by hydrochloric acid (20 μL of 37% w/v, 0.20 mmol). The reaction was stirred at 23° C. for 16 hours before diluting with diethyl ether and subsequently filtering through a pad of Celite. The filtrate was concentrated in vacuo. The crude residue was separated by HPLC (C₁₈, eluent: acetonitrile in water with 0.1% hydrochloric acid) which furnished 3-amino-N-(5-benzyl-4-phenyl-thiazol-2-yl)benzenesulfonamide (2.1 mg, 6%) as a white solid. ESI-MS m/z calc. 421.09186, found 422.1 (M+1)⁺; Retention time: 1.53 minutes; LC method A.

### Example 10: Preparation of Compound 10

### Step 1: N-[4,5-bis(p-Tolyl)thiazol-2-yl]benzenesulfonamide

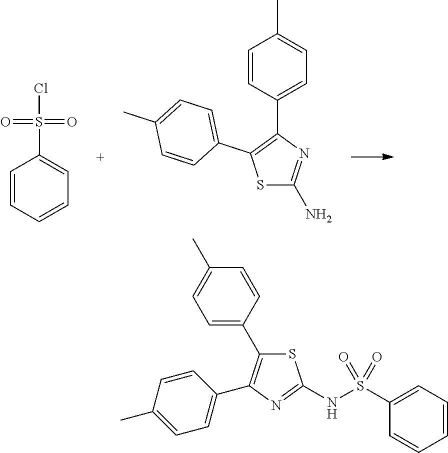 [SMILES: O=S(=O)(Cl)c1ccccc1 ; Cc1ccc(-c2nc(N)sc2-c2ccc(C)cc2)cc1 ; Cc1ccc(-c2nc(NS(=O)(=O)c3ccccc3)sc2-c2ccc(C)cc2)cc1]

To a solution of 4,5-bis(p-tolyl)thiazol-2-amine (approximately 28.04 mg, 0.1000 mmol) and DABCO (approximately 56.09 mg, 0.5000 mmol) in CH₃CN (0.5 ml) was added PhSO₂Cl (approximately 35.32 mg, 25.52 μL, 0.2000 mmol) and the reaction mixture was stirred at room temperature for 16 hours. The reaction mixture was diluted with MeOH and filtered. Purification by HPLC (1-99% ACN in water (HCl modifier)) gave N-[4,5-bis(p-tolyl)thiazol-2-yl]benzenesulfonamide (0.6 mg, 1%). ESI-MS m/z calc. 420.09662, found 421.5 (M+1)⁺; Retention time: 1.99 minutes (LC method A).

### Example 11: Characterization of Compounds 11-24

The compounds in the following tables were prepared in a manner analogous to that described above using commercially available reagents and intermediates described herein.

| Cmpd | | LCMS | Calc. | | LCMS | |
|---|---|---|---|---|---|---|
| # | Structure | Rt (min) | mass | M + 1 | Met. | NMR |
| 11 | 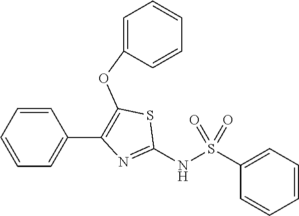 [SMILES: O=S(=O)(Nc1nc(-c2ccccc2)c(Oc2ccccc2)s1)c1ccccc1] | 1.82 | 408.06 | 409.1 | A | ¹H NMR (400 MHz DMSO-d₆) δ 13.03 (s, 1H), 7.89-7.81 (m, 2H), 7.67-7.55 (m, 5H), 7.44- 7.33 (m, 5H), 7.25- 7.16 (m, 3H) |
| 12 | 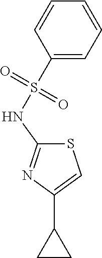 [SMILES: O=S(=O)(Nc1nc(C2CC2)cs1)c1ccccc1] | 1.1 | 280.034 | 281 | A | ¹H NMR (400 MHz, DMSO) δ 12.68 (s, 1H), 7.79 (d, J = 7.0 Hz, 2H), 7.56 (dq, J = 16.0, 7.8 Hz, 3H), 6.36 (s, 1H), 1.76 (t, J = 8.3 Hz, 1H), 0.83 (d, J = 8.4 Hz, 2H), 0.70 (d, J = 11.2 Hz, 2H). |
| 13 | 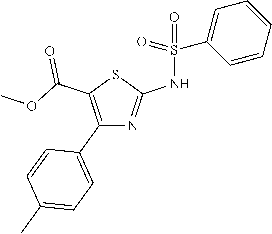 [SMILES: COC(=O)c1sc(NS(=O)(=O)c2ccccc2)nc1-c1ccc(C)cc1] | 1.54 | 388.055 | 389.4 | A | |
| 14 | 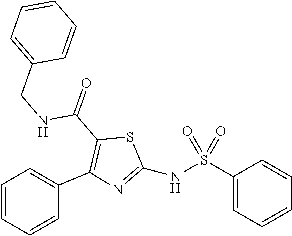 [SMILES: O=C(NCc1ccccc1)c1sc(NS(=O)(=O)c2ccccc2)nc1-c1ccccc1] | 1.51 | 449.087 | 450.5 | A | |
| 15 | 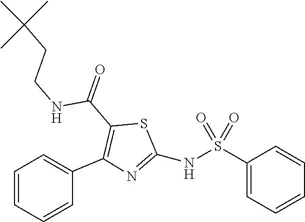 [SMILES: CC(C)(C)CCNC(=O)c1sc(NS(=O)(=O)c2ccccc2)nc1-c1ccccc1] | 1.72 | 443.134 | 444.5 | A | |
| 16 | 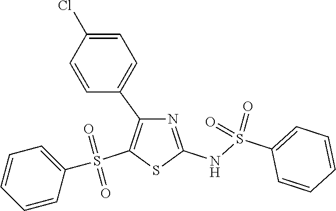 [SMILES: O=S(=O)(Nc1nc(-c2ccc(Cl)cc2)c(S(=O)(=O)c2ccccc2)s1)c1ccccc1] | 1.73 | 489.988 | 491.4 | A | |
| 17 | 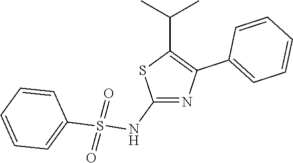 [SMILES: CC(C)c1sc(NS(=O)(=O)c2ccccc2)nc1-c1ccccc1] | 1.68 | 358.081 | 359 | A | |
| 18 | 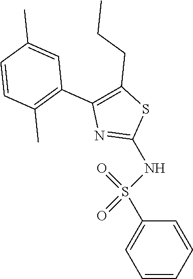 [SMILES: CCCc1sc(NS(=O)(=O)c2ccccc2)nc1-c1cc(C)ccc1C] | 1.89 | 386.112 | 387 | A | ¹H NMR (400 MHz, DMSO) δ 12.58 (s, 1H), 7.82 (d, J = 8.4 Hz, 2H), 7.64-7.52 (m, 3H), 7.24- 7.02 (m, 3H), 2.36- 2.25 (m, 5H), 2.09 (s, 3H), 1.45 (dd, J = 14.7, 7.4 Hz, 2H), 0.79 (t, J = 7.3 Hz, 3H). |
| 19 | 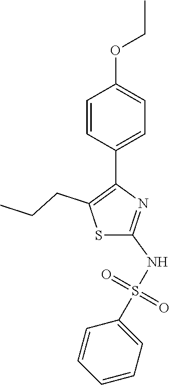 [SMILES: CCCc1sc(NS(=O)(=O)c2ccccc2)nc1-c1ccc(OCC)cc1] | 1.84 | 402.107 | 402 | A | ¹H NMR (400 MHz, DMSO) δ 12.73 (s, 1H), 7.83 (d, J = 9.3 Hz, 2H), 7.58 (dt, J = 18.2, 7.1 Hz, 3H), 7.33 (d, J = 8.7 Hz, 2H), 7.00 (d, J = 8.8 Hz, 2H), 4.06 (q, J = 6.9 Hz, 2H), 2.58-2.53 (m, 2H), 1.53 (dd, J = 18.5, 11.1 Hz, 2H), 1.33 (t, J = 7.0 Hz, 3H), 0.85 (t, J = 7.3 Hz, 3H). |
| 20 | 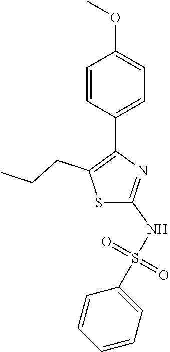 [SMILES: CCCc1sc(NS(=O)(=O)c2ccccc2)nc1-c1ccc(OC)cc1] | 1.72 | 388.092 | 389 | A | ¹H NMR (400 MHz, DMSO) δ 12.74 (s, 1H), 7.83 (d, J = 9.4 Hz, 2H), 7.65-7.52 (m, 3H), 7.35 (d, J = 8.7 Hz, 2H), 7.02 (d, J = 8.7 Hz, 2H), 3.79 (s, 3H), 2.56 (d, J = 7.4 Hz, 2H), 1.53 (dd, J = 14.7, 7.4 Hz, 2H), 0.86 (t, J = 7.3 Hz, 3H). |
| 21 | 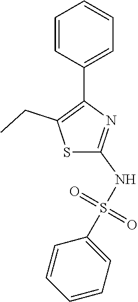 [SMILES: CCc1sc(NS(=O)(=O)c2ccccc2)nc1-c1ccccc1] | 1.58 | 344.065 | 345 | A | ¹H NMR (400 MHz, DMSO) δ 12.82 (s, 1H), 7.84 (d, J = 6.7 Hz, 2H), 7.63-7.54 (m, 3H), 7.45 (q, J = 6.9 Hz, 5H), 2.63 (q, J = 7.5 Hz, 2H), 1.15 (t, J = 7.5 Hz, 3H). |
| 22 | 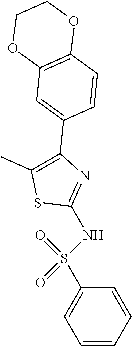 [SMILES: Cc1sc(NS(=O)(=O)c2ccccc2)nc1-c1ccc2c(c1)OCCO2] | 1.47 | 388.055 | 389 | A | ¹H NMR (400 MHz, DMSO) δ 12.71 (s, 1H), 7.83 (d, J = 7.9 Hz, 2H), 7.65-7.52 (m, 3H), 6.94 (d, J = 15.1 Hz, 3H), 4.27 (s, 4H), 2.20 (s, 3H). |
| 23 | 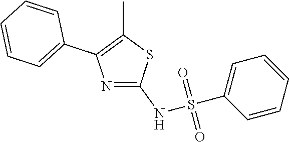 [SMILES: Cc1sc(NS(=O)(=O)c2ccccc2)nc1-c1ccccc1] | 1.46 | 330.05 | 331 | A | ¹H NMR (400 MHz, DMSO) δ 12.83 (s, 1H), 7.84 (d, J = 6.8 Hz, 2H), 7.64-7.53 (m, 3H), 7.50- 7.39 (m, 5H), 2.24 (s, 3H). |
| 24 | 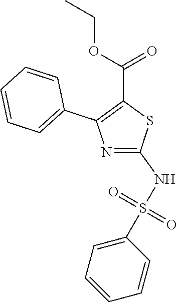 [SMILES: CCOC(=O)c1sc(NS(=O)(=O)c2ccccc2)nc1-c1ccccc1] | 1.55 | 388.055 | 389 | A | ¹H NMR (400 MHz, DMSO) δ 13.43 (s, 1H), 7.94-7.84 (m, 2H), 7.68- 7.54 (m, 5H), 7.53- 7.41 (m, 3H), 4.13 (q, J = 7.1 Hz, 2H), 1.15 (s, 3H). |

### Example 12: Preparation of Compound 25

### Step 1: N-[5-[1-(2-Methoxyphenyl)cyclopropyl]thiazol-2-yl]benzenesulfonamide

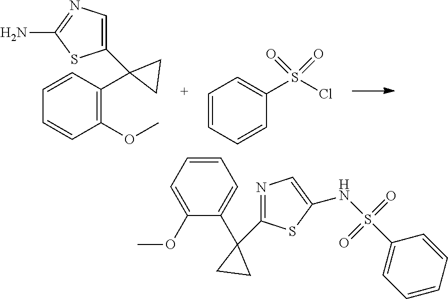

Benzenesulfonyl chloride (25 mg) was added to 5-[1-(2-methoxyphenyl)cyclopropyl]thiazol-2-amine (hydrochloride salt) (approximately 40.01 mg, 0.1415 mmol) in pyridine (0.5 mL). The mixture was stirred at 115° C. for 1 hour. The reaction mixture was filtered and purified by reverse phase HPLC using a gradient of acetonitrile and 5 mM HCl in water to give N-[5-[1-(2-methoxyphenyl)cyclopropyl]thiazol-2-yl]benzenesulfonamide (17.8 mg, 32%). ESI-MS m/z calc. 386.0759, found 387.0 (M+1)⁺; Retention time: 1.58 minutes; LC method A. ¹H NMR (400 MHz, DMSO) δ 12.35 (s, 1H), 7.75 (d, J=7.8 Hz, 2H), 7.54 (dt, J=14.7, 7.7 Hz, 3H), 7.28 (dd, J=15.6, 7.8 Hz, 2H), 7.07-6.87 (m, 3H), 3.81 (s, 3H), 1.26 (s, 2H), 1.11 (s, 2H).

### Example 13: Preparation of Compound 26

### Step 1: N-(4-Methyl-5-phenyl-thiazol-2-yl)benzenesulfonamide

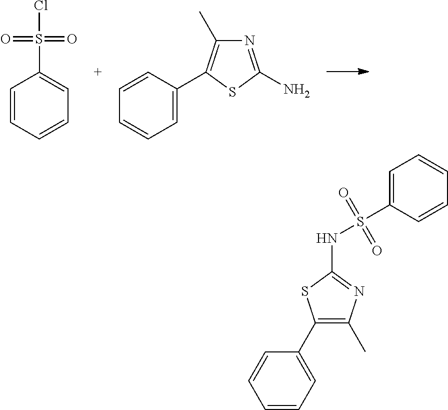 [SMILES: O=S(=O)(Cl)c1ccccc1 ; Cc1nc(N)sc1-c1ccccc1 ; Cc1nc(NS(=O)(=O)c2ccccc2)sc1-c1ccccc1]

To a solution of 4-methyl-5-phenyl-thiazol-2-amine (approximately 26.94 mg, 0.1416 mmol) in pyridine (0.5 mL) was added benzenesulfonyl chloride (50 mg, 0.2831 mmol) and the reaction was stirred at 115° C. for 1 hour. The reaction mixture was filtered and purified on reverse phase HPLC (HCl modifier, 25-75% ACN-H₂O) to give N-(4-methyl-5-phenyl-thiazol-2-yl)benzenesulfonamide (22.3 mg). ESI-MS m/z calc. 330.04968, found 331.0 (M+1)⁺; Retention time: 1.46 minutes; LC method A.

### Example 14: Preparation of Compound 27

### Step 1: N-[5-[2-(5-Chloro-2-methoxy-anilino)thiazol-4-yl]-4-methyl-thiazol-2-yl]benzenesulfonamide

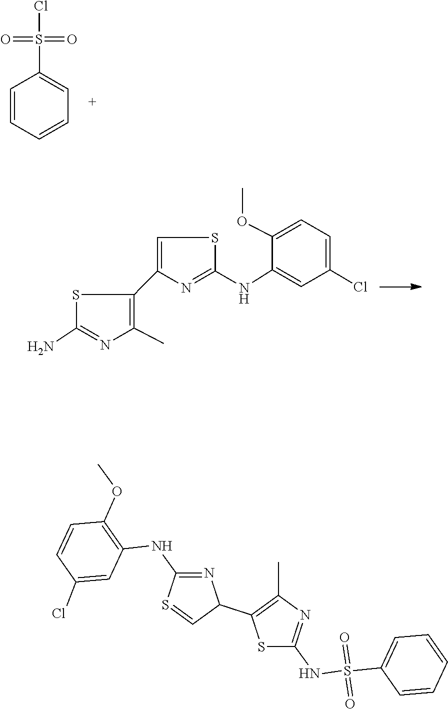

To a solution of 5-[2-(5-chloro-2-methoxy-anilino)thiazol-4-yl]-4-methyl-thiazol-2-amine (approximately 49.97 mg, 0.1416 mmol) in pyridine (0.5 mL) was added benzenesulfonyl chloride (50 mg, 0.2831 mmol) and the reaction was stirred at 115° C. for 1 hour. The reaction mixture was filtered and purified on reverse phase HPLC (HCl modifier, 25-75% ACN-H₂O) to give N-[5-[2-(5-chloro-2-methoxy-anilino)thiazol-4-yl]-4-methyl-thiazol-2-yl]benzenesulfonamide (2.9 mg). ESI-MS m/z calc. 492.01514, found 493.0 (M+1)⁺; Retention time: 1.74 minutes; LC method A.

### Example 15: Characterization of Compounds 28-47

The compounds in the following tables were prepared in a manner analogous to that described above using commercially available reagents and intermediates described herein.

| Compound | | LCMS | Calc. | | LCMS |
|---|---|---|---|---|---|
| number | Structure | Rt (min) | mass | M + 1 | Method |
| 28 | 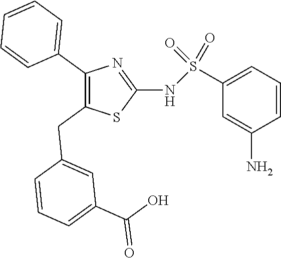 [SMILES: Nc1cccc(S(=O)(=O)Nc2nc(-c3ccccc3)c(Cc3cccc(C(=O)O)c3)s2)c1] | 1.3 | 465.082 | 466.4 | A |
| 29 | 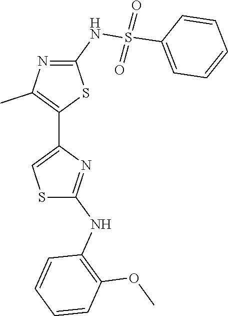 [SMILES: COc1ccccc1Nc1nc(-c2sc(NS(=O)(=O)c3ccccc3)nc2C)cs1] | 1.65 | 458.054 | 459 | A |
| 30 | 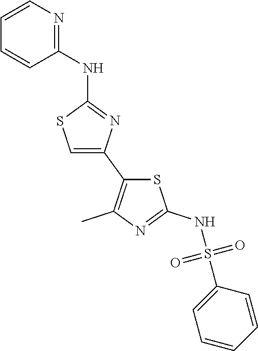 [SMILES: Cc1nc(NS(=O)(=O)c2ccccc2)sc1-c1csc(Nc2ccccn2)n1] | 1.29 | 429.039 | 430 | A |
| 31 | 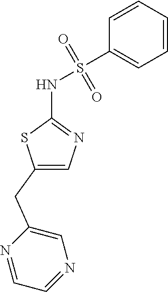 [SMILES: O=S(=O)(Nc1ncc(Cc2cnccn2)s1)c1ccccc1] | 0.92 | 332.04 | 333 | A |
| 32 | 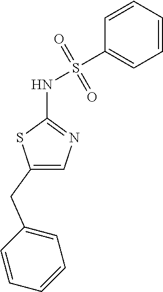 [SMILES: O=S(=O)(Nc1ncc(Cc2ccccc2)s1)c1ccccc1] | 1.41 | 330.05 | 331 | A |
| 33 | 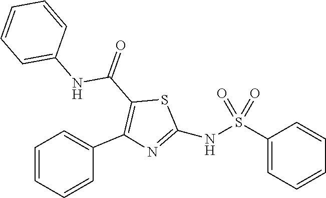 [SMILES: O=C(Nc1ccccc1)c1sc(NS(=O)(=O)c2ccccc2)nc1-c1ccccc1] | 1.53 | 435.071 | 436.2 | A |
| 34 | 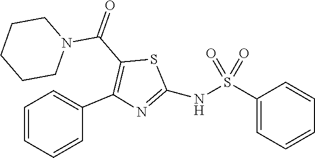 [SMILES: O=C(c1sc(NS(=O)(=O)c2ccccc2)nc1-c1ccccc1)N1CCCCC1] | 1.44 | 427.102 | 428.5 | A |
| 35 | 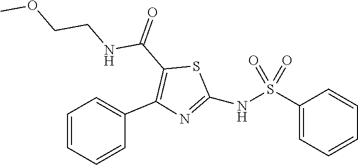 [SMILES: COCCNC(=O)c1sc(NS(=O)(=O)c2ccccc2)nc1-c1ccccc1] | 1.18 | 417.082 | 418.4 | A |
| 36 | 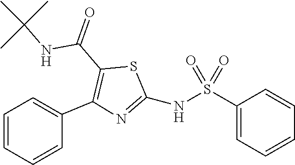 [SMILES: CC(C)(C)NC(=O)c1sc(NS(=O)(=O)c2ccccc2)nc1-c1ccccc1] | 1.5 | 415.102 | 416.3 | A |
| 37 | 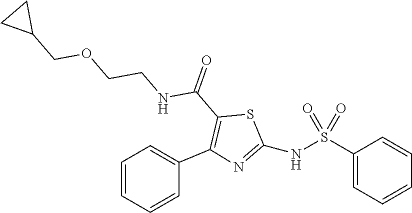 [SMILES: O=C(NCCOCC1CC1)c1sc(NS(=O)(=O)c2ccccc2)nc1-c1ccccc1] | 1.43 | 457.113 | 458.5 | A |
| 38 | 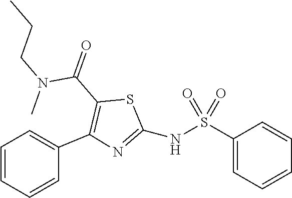 [SMILES: CCCN(C)C(=O)c1sc(NS(=O)(=O)c2ccccc2)nc1-c1ccccc1] | 1.39 | 415.102 | 416.3 | A |
| 39 | 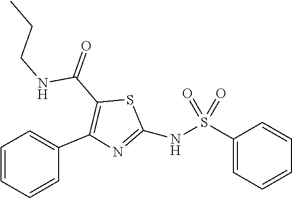 [SMILES: CCCNC(=O)c1sc(NS(=O)(=O)c2ccccc2)nc1-c1ccccc1] | 1.36 | 401.087 | 402.4 | A |
| 40 | 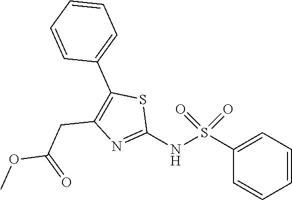 [SMILES: COC(=O)Cc1nc(NS(=O)(=O)c2ccccc2)sc1-c1ccccc1] | 1.37 | 388.055 | 389.4 | A |
| 41 | 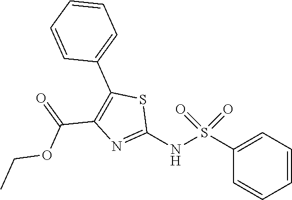 [SMILES: CCOC(=O)c1nc(NS(=O)(=O)c2ccccc2)sc1-c1ccccc1] | 1.62 | 388.055 | 389.4 | A |
| 42 | 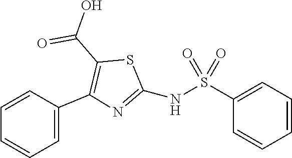 [SMILES: O=C(O)c1sc(NS(=O)(=O)c2ccccc2)nc1-c1ccccc1] | 1.09 | 360.024 | 361.3 | A |
| 43 | 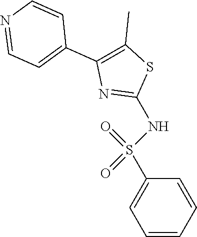 [SMILES: Cc1sc(NS(=O)(=O)c2ccccc2)nc1-c1ccncc1] | 0.81 | 331.045 | 332 | A |
| 44 | 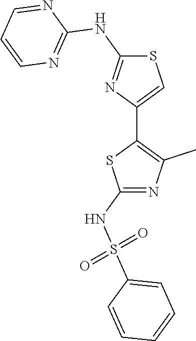 [SMILES: Cc1nc(NS(=O)(=O)c2ccccc2)sc1-c1csc(Nc2ncccn2)n1] | 1.35 | 430.034 | 431 | A |
| 45 | 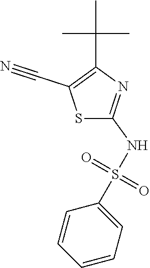 [SMILES: CC(C)(C)c1nc(NS(=O)(=O)c2ccccc2)sc1C#N] | 1.43 | 321.061 | 322 | A |
| 46 | 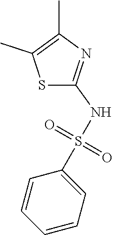 [SMILES: Cc1nc(NS(=O)(=O)c2ccccc2)sc1C] | 1.09 | 268.034 | 269 | A |
| 47 | 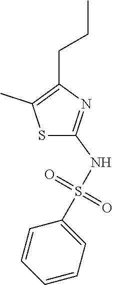 [SMILES: CCCc1nc(NS(=O)(=O)c2ccccc2)sc1C] | 1.36 | 296.065 | 297 | A |

| Cmpd | |
|---|---|
| No. | NMR |
| 29 | ¹H NMR (400 MHz, DMSO) δ 12.70 (s, 1H), 9.66 (s, 1H), |
| | 8.23 (d, J = 9.2 Hz, 1H), 7.83 (d, J = 9.4 Hz, 2H), 7.66-7.50 |
| | (m, 3H), 7.10-6.93 (m, 3H), 6.89 (s, 1H), 3.86 (s, 3H), 2.35 |
| | (s, 1H). |
| 30 | ¹H NMR (400 MHz, DMSO) δ 12.75 (s, 1H), 11.62 (d, J = |
| | 87.7 Hz, 1H), 8.31 (d, J = 5.0 Hz, 1H), 7.83 (d, J = 7.8 Hz, |
| | 2H), 7.73 (t, J = 8.5 Hz, 1H), 7.66-7.51 (m, 3H), 7.14-7.03 |
| | (m, 2H), 7.02-6.92 (m, 1H), 2.35 (s, 3H), 2.08 (s, 1H). |
| 31 | ¹H NMR (400 MHz, DMSO) δ 12.54 (s, 1H), 8.70-8.55 |
| | (m, 3H), 7.77 (d, J = 7.9 Hz, 2H), 7.55 (dt, J = 15.0, 5.8 |
| | Hz, 3H), 7.15 (s, 1H), 4.15 (s, 2H). |
| 39 | ¹H NMR (400 MHz, DMSO-d₆) δ 13.23 (s, 1H), 7.89- |
| | 7.84 (m, 2H), 7.84-7.79 (m, 1H), 7.67-7.55 (m, 3H), 7.53- |
| | 7.48 (m, 2H), 7.48-7.41 (m, 3H), 3.03 (q, J = 6.7 Hz, 2H), |
| | 1.35 (h, J = 7.2 Hz, 2H), 0.74 (t, J = 7.4 Hz, 3H). |
| 43 | ¹H NMR (400 MHz, DMSO) δ 8.87 (d, J = 6.7 Hz, 2H), |
| | 7.93 (d, J = 6.7 Hz, 2H), 7.86 (d, J = 8.5 Hz, 2H), 7.61 |
| | (dt, J = 20.9, 6.5 Hz, 3H), 2.44 (s, 3H). |
| 45 | ¹H NMR (400 MHz, DMSO) δ 7.86 (d, J = 7.6 Hz, 2H), |
| | 7.79-7.51 (m, 3H), 1.35 (s, 9H). |
| 46 | ¹H NMR (400 MHz, DMSO) δ 12.36 (s, 1H), 7.78 (d, |
| | J = 6.8 Hz, 2H), 7.55 (dq, J = 14.3, 7.1 Hz, 3H), 2.08 |
| | (s, 3H), 1.99 (s, 3H). |
| 47 | ¹H NMR (400 MHz, DMSO) δ 12.39 (s, 1H), 7.79 (d, |
| | J = 7.0 Hz, 2H), 7.55 (dd, J = 21.0, 7.0 Hz, 3H), |
| | 2.42-2.31 (m, 2H), 2.10 (s, 3H), 1.48 (dd, J = 16.5, |
| | 9.2 Hz, 2H), 0.82 (t, J = 7.3 Hz, 3H). |

### Example 16: Preparation of Compound 48

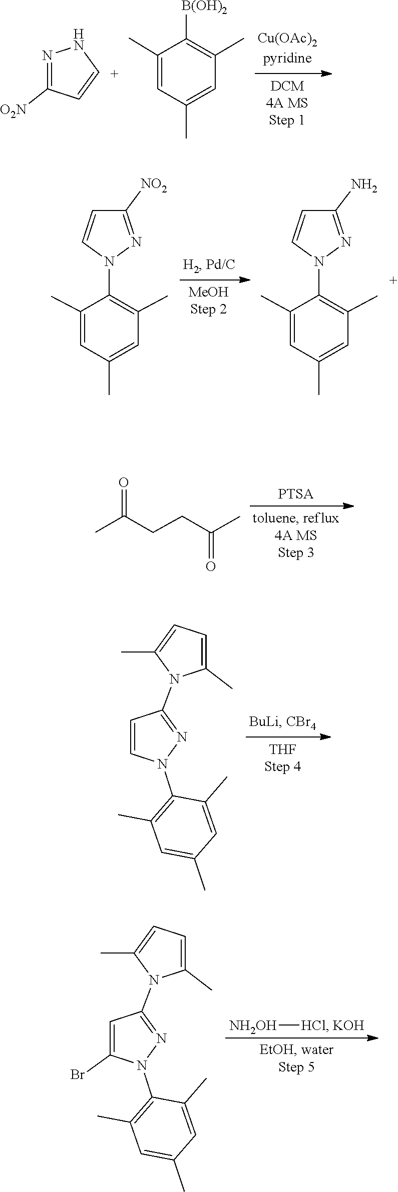

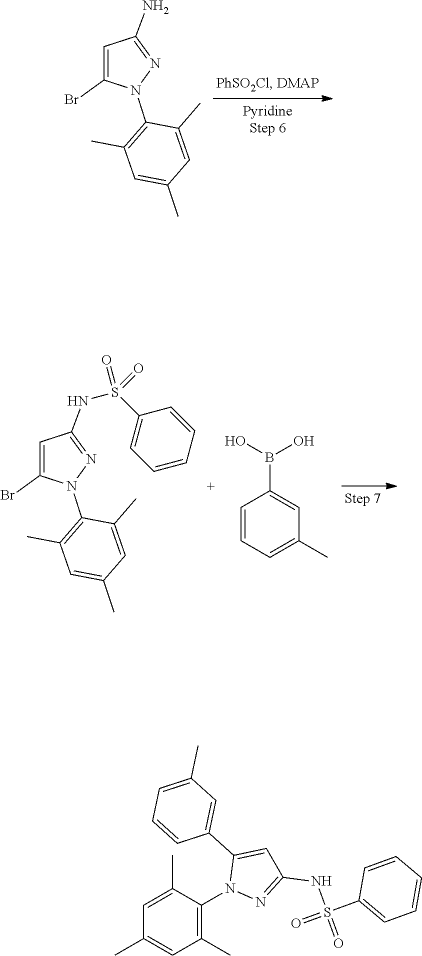

### Step 1: 3-Nitro-1-(2,4,6-trimethylphenyl)-1H-pyrazole

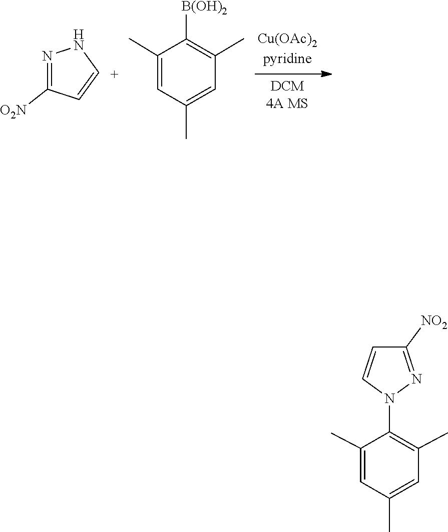

Pyridine (3.93 mL, 48.6 mmol) was added to a solution of 3-nitro-1H-pyrazole (2.75 g, 24.3 mmol), (2,4,6-trimethylphenyl)boronic acid (4.4 g, 26.8 mmol), cupric acetate (6.6 g, 36.5 mmol) and 4 Å molecular sieves (5.5 g) in dichloromethane (120 mL) at room temperature and the mixture was stirred for 2 days in a flask equipped with a reflux condenser over air (sealed with a septum and placed two needles on top). The crude mixture was filtered over Celite. The filtrate was then washed with water (100 mL) and brine (100 mL), dried over anhydrous sodium sulfate, filtered, and concentrated under reduced pressure. The residue was purified by silica gel column chromatography, eluting from 0% to 30% ethyl acetate in heptanes, to provide 3-nitro-1-(2,4,6-trimethylphenyl)-1H-pyrazole (1.3 g, 23% yield) as a white solid. ¹H NMR (300 MHz, CDCl₃) δ ppm 2.00 (s, 6H), 2.34 (s, 3H), 6.94 (s, 2H), 7.08 (d, J=2.3 Hz, 1H), 7.50 (d, J=2.5 Hz, 1H). [M+H]⁺=232.1.

### Step 2: 1-(2,4,6-Trimethylphenyl)-1H-pyrazol-3-amine

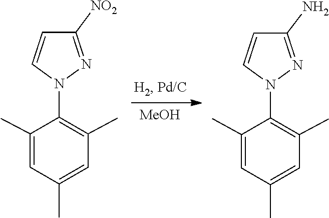

Palladium on carbon [800 mg of 10 wt. % loading (dry basis), wet support] was added to a solution of 3-nitro-1-(2,4,6-trimethylphenyl)-1H-pyrazole (1.3 g, 5.62 mmol) in methanol (25 mL) at room temperature, and the mixture was stirred under one atmosphere of hydrogen overnight. The mixture was then filtered through Celite, and the filtrate was concentrated under reduced pressure to afford crude 1-(2,4,6-trimethylphenyl)-1H-pyrazol-3-amine (1.1 g, 97% yield) as a white solid. ¹H NMR (300 MHz, CDCl₃) δ ppm 2.03 (s, 6H), 2.30 (s, 3H), 3.35 (br. s., 2H), 5.79 (d, J=2.4 Hz, 1H), 6.91 (s, 2H), 7.16 (d, J=2.4 Hz, 1H). [M+H]⁺=202.2.

### Step 3: 3-(2,5-Dimethyl-1H-pyrrol-1-yl)-1-(2,4,6-trimethylphenyl)-1H-pyrazole

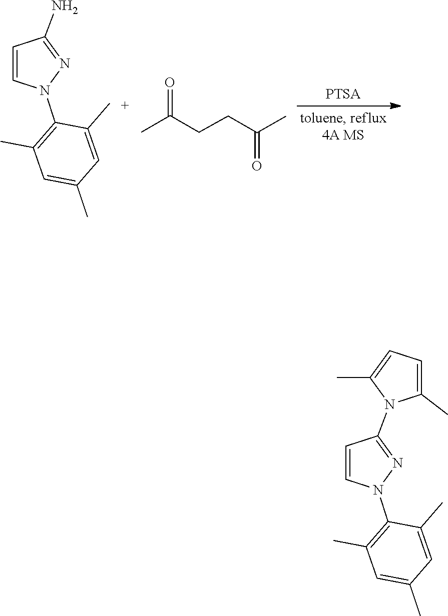

To a solution of 1-methyl-1H-pyrazol-3-ylamine (2.69 g, 13.4 mmol) and hexane-2,5-dione (1.88 mL, 16 mmol) in toluene (62 mL) was added p-toluenesulfonic acid monohydrate (254 mg, 1.34 mmol), and the mixture was refluxed for 3 hours. The toluene was removed under pressure, and water (20 mL) was added. The aqueous layer was then extracted with ethyl acetate (50 mL), and the organic layer was washed with brine (20 mL), dried over sodium sulfate, filtered, and concentrated under pressure. The residue was purified by silica gel chromatography on a 40-g column, eluting from 0% to 10% ethyl acetate in heptanes to afford 3-(2,5-dimethyl-1H-pyrrol-1-yl)-1-(2,4,6-trimethylphenyl)-1H-pyrazole (2.72 g, 73% yield) as an off-white solid. ¹H NMR (300 MHz, CDCl₃) δ ppm 2.04 (s, 6H), 2.17 (s, 6H), 2.34 (s, 3H), 5.88 (s, 2H), 6.36 (d, J=2.3 Hz, 1H), 6.96 (s, 2H), 7.47 (d, J=2.3 Hz, 1H). [M+H]⁺=280.2.

### Step 4: 5-Bromo-3-(2,5-dimethyl-1H-pyrrol-1-yl)-1-(2,4,6-trimethylphenyl)-1H-pyrazole

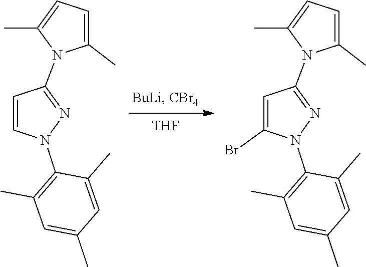

To a solution of 3-(2,5-dimethyl-1H-pyrrol-1-yl)-1-(2,4,6-trimethylphenyl)-1H-pyrazole (3.60 g, 12.9 mmol) in dry THE (36 mL) cooled to −78° C. was added butyllithium (5.68 mL of a 2.5 M solution in hexanes, 14.2 mmol). The reaction mixture was stirred for 2 hours at −78° C. before a solution of carbon tetrabromide (4.71 g, 14.2 mmol) in THE (25 mL) was added dropwise. The mixture was then allowed to warm to room temperature and stirred overnight. Ice-water (5 mL) was added, the volatiles were removed under reduced pressure, and the aqueous layer was extracted with ethyl acetate (80 mL). The organic layer was then washed with brine (50 mL), dried over sodium sulfate, filtered and concentrated under reduced pressure. The residue was purified by silica gel chromatography on a 40-g column, eluting from 0% to 10% ethyl acetate in heptanes, to afford 5-bromo-3-(2,5-dimethyl-1H-pyrrol-1-yl)-1-(2,4,6-trimethylphenyl)-1H-pyrazole (2.0 g, 43% yield) as a white solid. ¹H NMR (300 MHz, CDCl₃) δ ppm 2.01 (s, 6H), 2.17 (s, 6H), 2.35 (s, 3H), 5.87 (s, 2H), 6.41 (s, 1H), 7.00 (s, 2H). [M+H]⁺=358.0.

### Step 5: 5-Bromo-1-(2,4,6-trimethylphenyl)-1H-pyrazol-3-amine

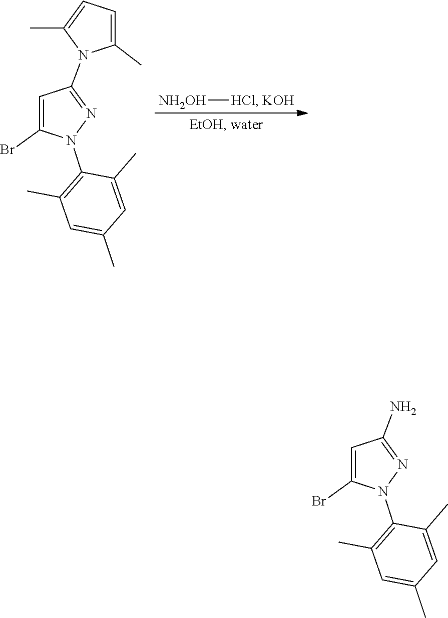

To a solution of hydroxylamine hydrochloride (1.37 g, 19.9 mmol) in ethanol (55 mL) was added potassium hydroxide (686 mg, 12.2 mmol) in water (11 mL) and ethanol (22 mL), followed by 5-bromo-3-(2,5-dimethyl-1H-pyrrol-1-yl)-1-(2,4,6-trimethylphenyl)-1H-pyrazole (2.74 g, 7.65 mmol). The mixture was then heated at 90° C. over 7 days or until LCMS indicated completion. The mixture was then concentrated under reduced pressure and partitioned between ethyl acetate (50 mL) and water (20 mL). The layers were separated, and the organic layer was then washed with brine (10 mL), dried over sodium sulfate, filtered, and concentrated under pressure to afford crude 5-bromo-1-(2,4,6-trimethylphenyl)-1H-pyrazol-3-amine (2.69 g, 126% yield) that was used in the following step without further purification. [M+H]⁺=280.0.

### Step 6: N-(5-Bromo-1-mesityl-1H-pyrazol-3-yl)benzenesulfonamide

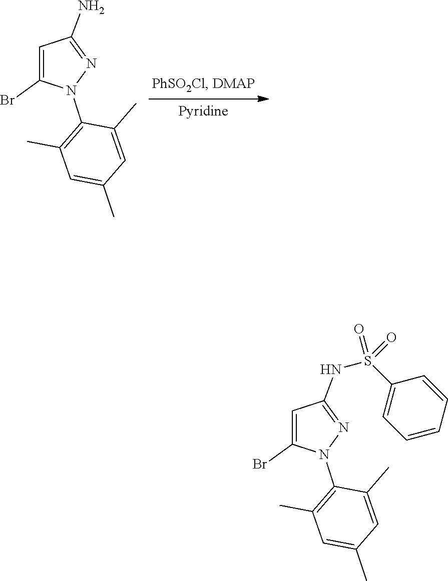 [SMILES: Cc1cc(C)c(-n2nc(N)cc2Br)c(C)c1 ; Cc1cc(C)c(-n2nc(NS(=O)(=O)c3ccccc3)cc2Br)c(C)c1]

A mixture of 5-bromo-1-(2,4,6-trimethylphenyl)-1H-pyrazol-3-amine (2.57 g, 9.17 mmol) and 4-dimethylaminopyridine (224 mg, 1.83 mmol) in pyridine (200 mL) was treated with benzenesulfonyl chloride (3.51 mL, 27.5 mmol) and stirred overnight at room temperature. The solvent was then removed under reduced pressure, and the residue was taken up with dichloromethane (50 mL). The organic layer was then washed with water (10 mL), brine (10 mL), dried over sodium sulfate, filtered, and concentrated under pressure. The residue was purified by silica gel chromatography on a 40-g column, eluting from 0% to 20% ethyl acetate in heptanes to afford N-(5-bromo-1-mesityl-1H-pyrazol-3-yl)benzenesulfonamide (1.55 g, 40% yield) as a pale yellow solid. ¹H NMR (300 MHz, CDCl₃) δ ppm 1.75 (s, 6H), 2.29 (s, 3H), 6.51 (s, 1H), 6.87 (s, 2H), 7.39-7.46 (m, 3H), 7.50-7.57 (m, 1H), 7.72-7.77 (m, 2H). [M+H]⁺=420.0.

### Step 7: N-[5-(m-Tolyl)-1-(2,4,6-trimethylphenyl)pyrazol-3-yl]benzenesulfonamide

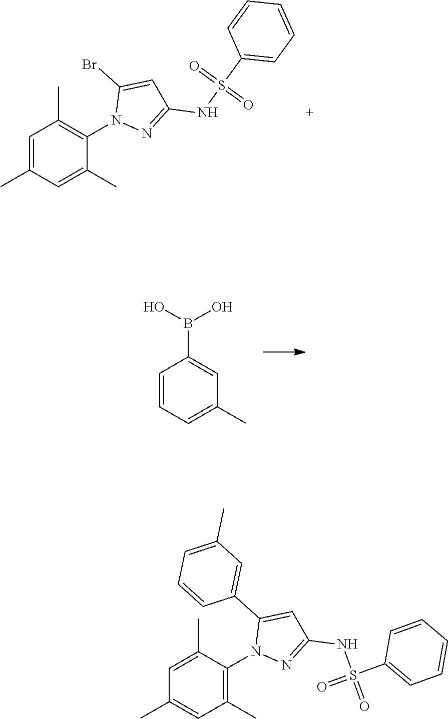

N-[5-bromo-1-(2,4,6-trimethylphenyl)pyrazol-3-yl]benzenesulfonamide (25 mg, 0.05948 mmol), Pd(dppf)Cl₂ (approximately 2.176 mg, 0.002974 mmol), sodium carbonate (approximately 148.7 μL of 2 M, 0.2974 mmol), and m-tolylboronic acid (approximately 12.13 mg, 0.08922 mmol) in dioxane (0.5 mL) were added to a microwave vial. The vial was purged with nitrogen, capped and heated at 140° C. for 45 minutes in a microwave. The crude was filtered and purified by HPLC utilizing a gradient of 25-75% acetonitrile in 5 mM aqueous HCl to give N-[5-(m-tolyl)-1-(2,4,6-trimethylphenyl)pyrazol-3-yl]benzenesulfonamide (10.8 mg, 68%). ESI-MS m/z calc. 431.16675, found 432.0 (M+1)⁺; Retention time: 2.04 minutes; LC method A. ¹H NMR (400 MHz, DMSO) δ 10.59 (s, 1H), 7.81 (d, J=7.4 Hz, 2H), 7.62 (t, J=7.4 Hz, 1H), 7.55 (t, J=7.5 Hz, 2H), 7.10 (d, J=7.1 Hz, 2H), 7.03 (s, 1H), 6.92 (s, 2H), 6.75 (d, J=6.8 Hz, 1H), 6.47 (s, 1H), 2.25 (s, 3H), 2.20 (s, 3H), 1.64 (s, 6H).

### Example 17: Preparation of Compound 49

### Step 1: N-[5-(4-Phenoxyphenyl)-1-(2,4,6-trimethylphenyl)pyrazol-3-yl]benzenesulfonamide

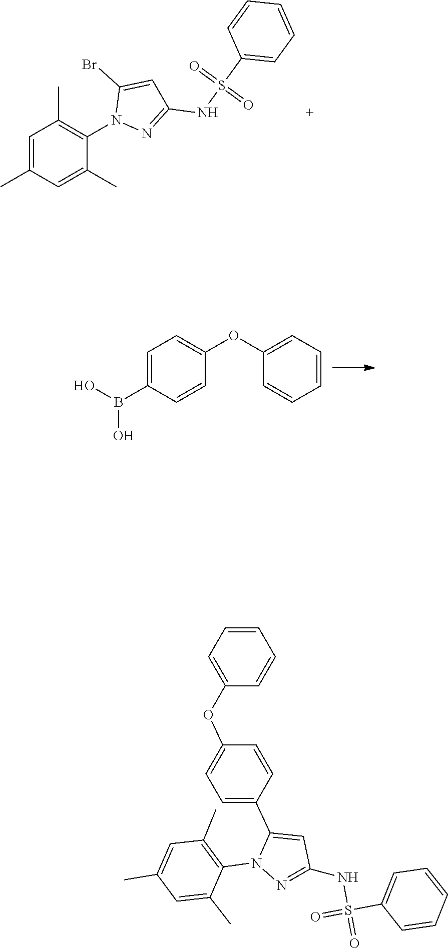 [SMILES: Cc1cc(C)c(-n2nc(NS(=O)(=O)c3ccccc3)cc2Br)c(C)c1 ; OB(O)c1ccc(Oc2ccccc2)cc1 ; Cc1cc(C)c(-n2nc(NS(=O)(=O)c3ccccc3)cc2-c2ccc(Oc3ccccc3)cc2)c(C)c1]

The compound was prepared in a manner analogous to that described above using commercially available (4-phenoxyphenyl)boronic acid. N-[5-(4-phenoxyphenyl)-1-(2,4,6-trimethylphenyl)pyrazol-3-yl]benzenesulfonamide (4.5 mg, 63%). ESI-MS m/z calc. 509.1773, found 510.0 (M+1)⁺; Retention time: 2.17 minutes; LC method A. ¹H NMR (400 MHz, DMSO) δ 10.61 (s, 1H), 7.80 (d, J=7.1 Hz, 2H), 7.70-7.60 (m, 3H), 7.54 (t, J=6.7 Hz, 2H), 7.49-7.32 (m, 5H), 7.18 (t, J=7.4 Hz, 2H), 7.08 (t, J=6.4 Hz, 4H), 7.01 (d, J=7.6 Hz, 2H), 6.93 (s, 2H), 6.86 (d, J=8.8 Hz, 2H), 6.46 (s, 1H), 2.25 (s, 3H), 1.65 (s, 6H).

### Example 18: Preparation of Compound 50

### Step 1: N-[5-(3-Methoxyphenyl)-1-(2,4,6-trimethylphenyl)pyrazol-3-yl]benzenesulfonamide

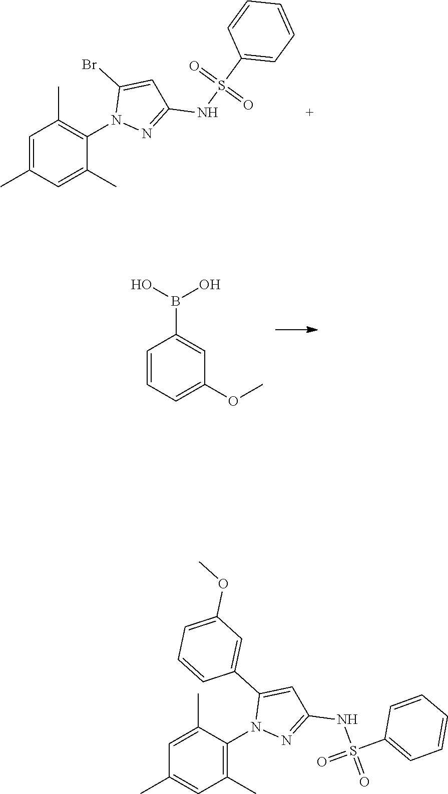 [SMILES: Cc1cc(C)c(-n2nc(NS(=O)(=O)c3ccccc3)cc2Br)c(C)c1 ; COc1cccc(B(O)O)c1 ; COc1cccc(-c2cc(NS(=O)(=O)c3ccccc3)nn2-c2c(C)cc(C)cc2C)c1]

The compound was prepared in a manner analogous to that described above using commercially available (3-methoxyphenyl)boronic acid. N-[5-(3-methoxyphenyl)-1-(2,4,6-trimethylphenyl)pyrazol-3-yl]benzenesulfonamide (7.9 mg, 43%). ESI-MS m/z calc. 447.16165, found 448.0 (M+1)⁺; Retention time: 1.92 minutes; LC method A. ¹H NMR (400 MHz, DMSO) δ 10.59 (s, 1H), 7.80 (d, J=7.9 Hz, 2H), 7.58 (dt, J=15.1, 7.2 Hz, 3H), 7.20 (t, J=8.0 Hz, 1H), 6.93 (s, 2H), 6.84 (d, J=10.7 Hz, 1H), 6.74 (d, J=7.3 Hz, 1H), 6.51 (s, 2H), 3.56 (s, 3H), 2.25 (s, 3H), 1.64 (s, 6H).

### Example 19: Preparation of Compound 51 and Compound 52

### Step 1: N-[5-(2-Methylprop-1-enyl)-1-(2,4,6-trimethylphenyl)pyrazol-3-yl]benzenesulfonamide, Compound 51, and N-[5-isobutyl-1-(2,4,6-trimethylphenyl)pyrazol-3-yl]benzenesulfonamide Compound 52

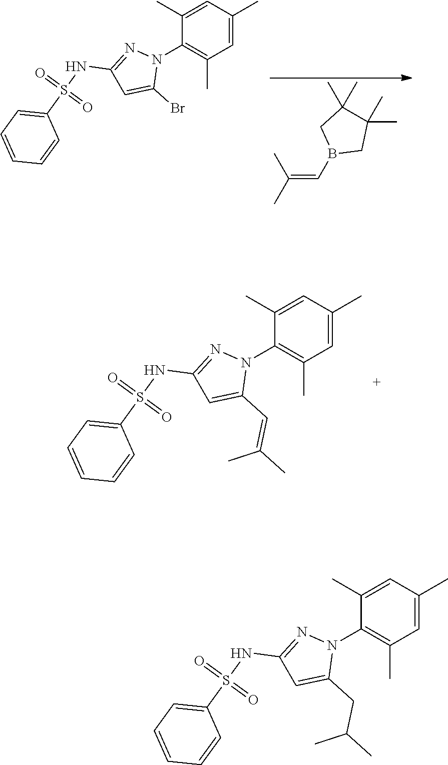

N-[5-bromo-1-(2,4,6-trimethylphenyl)pyrazol-3-yl]benzenesulfonamide (32 mg, 0.07613 mmol), Pd(dppf)Cl₂ (4.4 mg, 0.006013 mmol), sodium carbonate (200 μL of 2 M, 0.4000 mmol), and 4,4,5,5-tetramethyl-2-(2-methylprop-1-enyl)-1,3,2-dioxaborolane (20.8 mg, 0.1142 mmol) in dioxane (1 mL) were added to a microwave vial. The vial was purged with nitrogen, capped, and heated at 140° C. for 45 minutes in a microwave. More 4,4,5,5-tetramethyl-2-(2-methylprop-1-enyl)-1,3,2-dioxaborolane (20.8 mg, 0.1142 mmol) and Pd(dppf)Cl₂ (4.4 mg, 0.006013 mmol) were added, and the reaction was heated in microwave for another 30 minutes at 140° C. The crude was purified by HPLC utilizing a gradient of 25-75% acetonitrile in 5 mM aqueous HCl to give N-[5-(2-methylprop-1-enyl)-1-(2,4,6-trimethylphenyl)pyrazol-3-yl]benzenesulfonamide (14.6 mg, 48%) ESI-MS m/z calc. 395.16675, found 396.0 (M+1)⁺; Retention time: 1.98 minutes (LC method A).

Pd on C, wet, Degussa type (approximately 16.20 mg of 5% w/w, 0.007613 mmol) was added to the above product dissolved in methanol (12.80 mL). The flask was purged with nitrogen, and the mixture was stirred at room temperature under a balloon of hydrogen. The reaction mixture was filtered and purified on reverse phase HPLC (HCl, 30-99% ACN-H₂O) to give N-[5-isobutyl-1-(2,4,6-trimethylphenyl)pyrazol-3-yl]benzenesulfonamide ESI-MS m/z calc. 397.1824, found 398.0 (M+1)⁺; Retention time: 2.05 minutes (LC method A).

### Example 20: Characterization of Compounds 53-68

The compounds in the following tables were prepared in a manner analogous to that described above using commercially available reagents and intermediates described herein.

| | | LCMS | Calc. | | LCMS |
|---|---|---|---|---|---|
| Cmpd No. | Structure | Rt (min) | mass | M + 1 | Method |
| 53 | 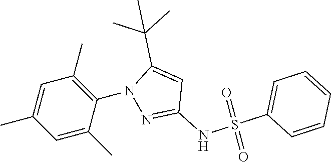 [SMILES: Cc1cc(C)c(-n2nc(NS(=O)(=O)c3ccccc3)cc2C(C)(C)C)c(C)c1] | 3.49 | 397.182 | 398.1 | I |
| 54 | 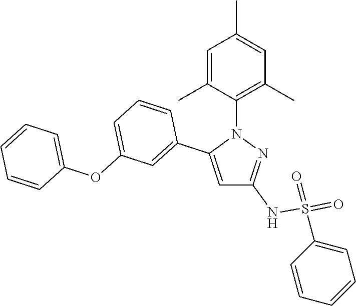 [SMILES: Cc1cc(C)c(-n2nc(NS(=O)(=O)c3ccccc3)cc2-c2cccc(Oc3ccccc3)c2)c(C)c1] | 2.21 | 509.177 | 510 | A |
| 55 | 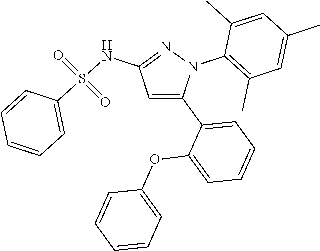 [SMILES: Cc1cc(C)c(-n2nc(NS(=O)(=O)c3ccccc3)cc2-c2ccccc2Oc2ccccc2)c(C)c1] | 2.1 | 509.177 | 510 | A |
| 56 | 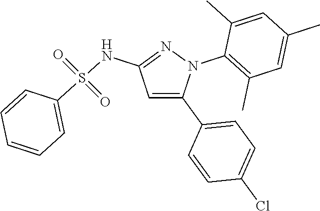 [SMILES: Cc1cc(C)c(-n2nc(NS(=O)(=O)c3ccccc3)cc2-c2ccc(Cl)cc2)c(C)c1] | 2.12 | 451.112 | 452 | A |
| 57 | 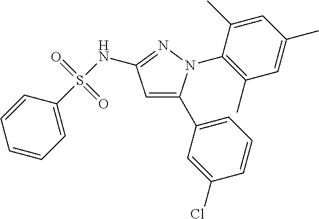 [SMILES: Cc1cc(C)c(-n2nc(NS(=O)(=O)c3ccccc3)cc2-c2cccc(Cl)c2)c(C)c1] | 2.08 | 451.112 | 452 | A |
| 58 | 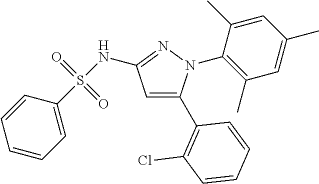 [SMILES: Cc1cc(C)c(-n2nc(NS(=O)(=O)c3ccccc3)cc2-c2ccccc2Cl)c(C)c1] | 1.98 | 451.112 | 452 | A |
| 59 | 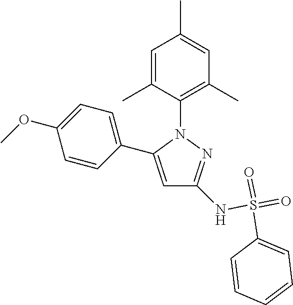 [SMILES: COc1ccc(-c2cc(NS(=O)(=O)c3ccccc3)nn2-c2c(C)cc(C)cc2C)cc1] | 1.91 | 447.162 | 448 | A |
| 60 | 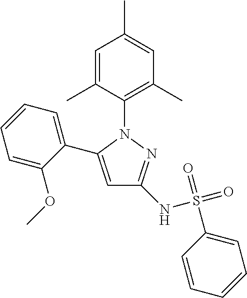 [SMILES: COc1ccccc1-c1cc(NS(=O)(=O)c2ccccc2)nn1-c1c(C)cc(C)cc1C] | 1.91 | 447.162 | 448 | A |
| 61 | 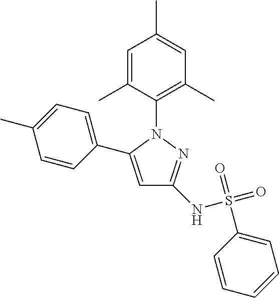 [SMILES: Cc1ccc(-c2cc(NS(=O)(=O)c3ccccc3)nn2-c2c(C)cc(C)cc2C)cc1] | 2.04 | 431.167 | 432 | A |
| 62 | 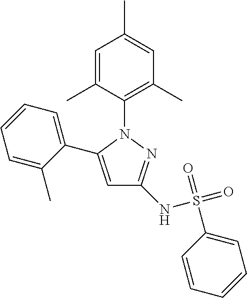 [SMILES: Cc1cc(C)c(-n2nc(NS(=O)(=O)c3ccccc3)cc2-c2ccccc2C)c(C)c1] | 1.98 | 431.167 | 432 | A |
| 63 | 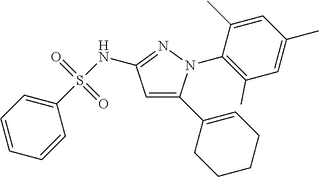 [SMILES: Cc1cc(C)c(-n2nc(NS(=O)(=O)c3ccccc3)cc2C2=CCCCC2)c(C)c1] | 2.13 | 421.182 | 422 | A |
| 64 | 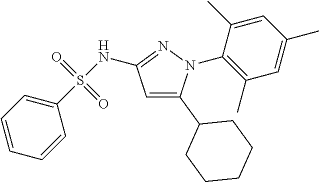 [SMILES: Cc1cc(C)c(-n2nc(NS(=O)(=O)c3ccccc3)cc2C2CCCCC2)c(C)c1] | 2.16 | 423.198 | 424 | A |
| 65 | 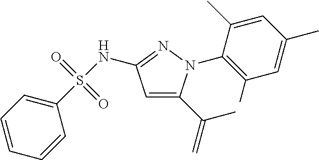 [SMILES: C=C(C)c1cc(NS(=O)(=O)c2ccccc2)nn1-c1c(C)cc(C)cc1C] | 1.92 | 381.151 | 382 | A |
| 66 | 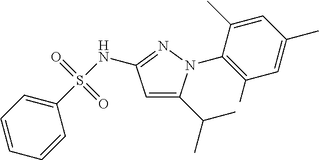 [SMILES: Cc1cc(C)c(-n2nc(NS(=O)(=O)c3ccccc3)cc2C(C)C)c(C)c1] | 1.94 | 383.167 | 384 | A |
| 67 | 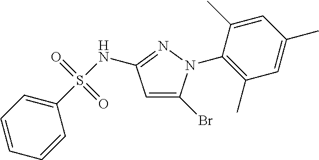 [SMILES: Cc1cc(C)c(-n2nc(NS(=O)(=O)c3ccccc3)cc2Br)c(C)c1] | 1.86 | 419.03 | 421 | A |
| 68 | 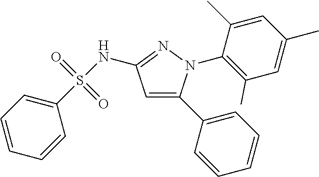 [SMILES: Cc1cc(C)c(-n2nc(NS(=O)(=O)c3ccccc3)cc2-c2ccccc2)c(C)c1] | 1.95 | 417.151 | 418 | A |

| Cmpd | |
|---|---|
| No. | NMR |
| 53 | ¹H NMR (300 MHz, CDCl₃) δ 1.29 (s, 9H), 1.58 (s, |
| | 6H), 2.29 (s, 3H), 6.16 (br. s., 1H), 6.21 (s, 1H), 6.85 |
| | (s, 2H), 7.41-7.51 (m, 2H), 7.55-7.64 (m, 1H), 7.75- |
| | 7.82 (m, 2H). |
| 54 | ¹H NMR (400 MHz, DMSO-d₆) δ 10.62 (s, 1H), |
| | 7.79 (d, J = 7.1 Hz, 2H), 7.61 (t, J = 6.7 Hz, 1H), |
| | 7.53 (t, J = 7.5 Hz, 2H), 7.35 (q, J = 7.6 Hz, 3H), |
| | 7.18 (t, J = 6.9 Hz, 1H), 7.04 (d, J = 8.3 Hz, 1H), |
| | 6.97 (dd, J = 8.2, 3.3 Hz, 1H), 6.80 (d, J = 8.3 Hz, 4H), |
| | 6.50 (s, 1H), 6.43 (s, 1H), 2.22 (s, 3H), 1.54 (s, 6H). |
| 55 | ¹H NMR (400 MHz, DMSO) δ 10.56 (s, 1H), 7.70 |
| | (d, J = 8.5 Hz, 2H), 7.57 (t, J = 7.5 Hz, 1H), 7.44 |
| | (t, J = 7.8 Hz, 2H), 7.39-7.34 (m, 2H), 7.31-7.26 (m, |
| | 1H), 7.15 (t, J = 7.4 Hz, 1H), 7.01 (d, J = 3.9 Hz, 2H), |
| | 6.77 (dd, J = 15.8, 6.4 Hz, 5H), 6.40 (s, 1H), 2.20 (s, |
| | 3H), 1.65 (s, 6H). |
| 56 | ¹H NMR (400 MHz, DMSO) δ 10.63 (s, 1H), 7.81 |
| | (d, J = 8.0 Hz, 2H), 7.58 (dd, J = 21.5, 7.2 Hz, 3H), |
| | 7.34 (d, J = 8.5 Hz, 2H), 7.09 (d, J = 8.5 Hz, 2H), |
| | 6.93 (s, 2H), 6.52 (s, 1H), 2.25 (s, 3H), 1.63 (s, 6H). |
| 57 | ¹H NMR (400 MHz, DMSO) δ 10.64 (s, 1H), 7.81 |
| | (d, J = 7.9 Hz, 2H), 7.61 (dd, J = 9.5, 5.1 Hz, 1H), |
| | 7.54 (t, J = 7.6 Hz, 2H), 7.35 (d, J = 9.1 Hz, 1H), 7.29 |
| | (t, J = 7.9 Hz, 1H), 7.16 (s, 1H), 7.01 (d, J = 7.7 Hz, |
| | 1H), 6.94 (s, 2H), 6.61 (s, 1H), 2.26 (s, 3H), 1.64 |
| | (s, 6H). |
| 60 | ¹H NMR (400 MHz, DMSO) δ 10.53 (s, 1H), 7.79 (t, |
| | J = 9.7 Hz, 2H), 7.59 (dt, J = 15.0, 7.3 Hz, 3H), 7.27 |
| | (t, J = 8.6 Hz, 1H), 6.88 (dt, J = 29.1, 9.3 Hz, 5H), |
| | 6.29 (s, 1H), 3.59 (s, 3H), 2.18 (s, 3H), 1.66 (s, 6H). |
| 61 | ¹H NMR (400 MHz, DMSO) δ 10.57 (s, 1H), 7.81 |
| | (d, J = 7.1 Hz, 2H), 7.62 (t, J = 6.7 Hz, 1H), 7.54 (t, |
| | J = 8.0 Hz, 2H), 7.07 (d, J = 8.0 Hz, 2H), 6.97 (d, J = |
| | 8.2 Hz, 2H), 6.91 (s, 2H), 6.43 (s, 1H), 2.24 (d, J = |
| | 6.2 Hz, 6H), 1.64 (s, 6H). |
| 62 | ¹H NMR (400 MHz, DMSO) δ 10.58 (s, 1H), 7.81 |
| | (d, J = 7.8 Hz, 2H), 7.66-7.52 (m, 3H), 7.26 (d, J = |
| | 7.6 Hz, 1H), 7.17 (t, J = 7.5 Hz, 1H), 6.96 (t, J = 7.5 |
| | Hz, 1H), 6.82 (s, 2H), 6.67 (d, J = 7.7 Hz, 1H), 6.32 |
| | (s, 1H), 2.27 (s, 3H), 2.18 (s, 3H), 1.65 (s, 6H). |
| 65 | ¹H NMR (400 MHz, DMSO) δ 10.50 (s, 1H), 7.76 |
| | (d, J = 7.9 Hz, 2H), 7.55 (t, J = 18.9 Hz, 3H), 6.94 |
| | (s, 2H), 6.26 (s, 1H), 4.91 (s, 1H), 4.47 (s, 1H), 2.26 |
| | (s, 3H), 1.84 (s, 3H), 1.65 (s, 6H). |
| 66 | ¹H NMR (400 MHz, DMSO) δ 10.36 (s, 1H), 7.74 |
| | (d, J = 8.8 Hz, 2H), 7.60 (t, J = 7.4 Hz, 1H), 7.52 |
| | (t, J = 7.6 Hz, 2H), 6.96 (s, 2H), 6.04 (s, 1H), 2.27 |
| | (s, 3H), 1.67 (s, 6H), 1.02 (d, J = 6.9 Hz, 6H). |
| 67 | ¹H NMR (400 MHz, DMSO) δ 10.73 (s, 1H), 7.77 |
| | (d, J = 8.1 Hz, 2H), 7.59 (dt, J = 15.3, 7.1 Hz, 3H), |
| | 6.98 (s, 2H), 6.39 (s, 1H), 2.27 (s, 3H), 1.68 (s, 6H). |
| 68 | ¹H NMR (400 MHz, DMSO) δ 10.60 (s, 1H), 7.81 |
| | (d, J = 8.0 Hz, 2H), 7.65-7.52 (m, 3H), 7.27 (d, J = |
| | 5.7 Hz, 3H), 7.09 (d, J = 5.0 Hz, 2H), 6.91 (s, 2H), |
| | 6.48 (s, 1H), 2.24 (s, 3H), 1.64 (s, 6H). |

### Example 21: Preparation of Compound 69

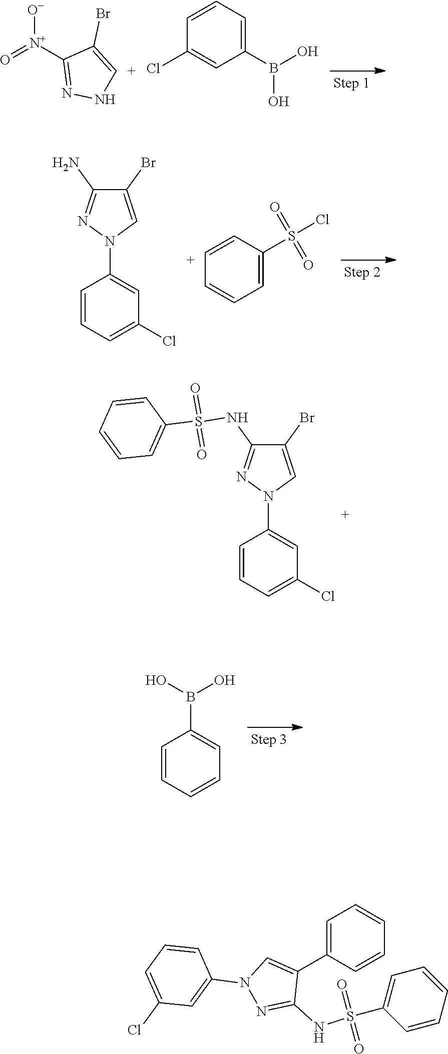

### Step 1: 4-Bromo-1-(3-chlorophenyl)pyrazol-3-amine

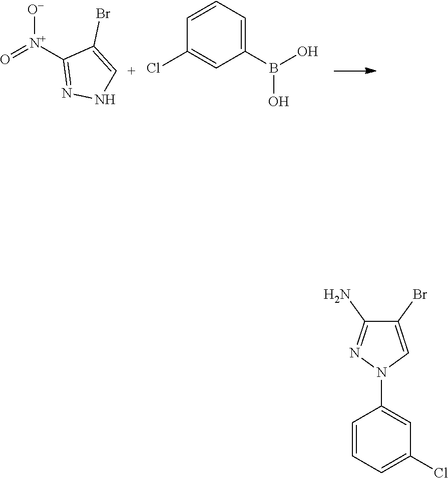

Pyridine (approximately 857.4 mg, 876.7 μL, 10.84 mmol) and (3-chlorophenyl)boronic acid (924.9 mg, 5.915 mmol) were added to 4-bromo-3-nitro-1H-pyrazole (1.03 g, 5.365 mmol) in THE (10.5 mL), followed by diacetoxycopper (approximately 1.462 g, 8.048 mmol). The mixture was stirred at room temperature for 3 days. The reaction mixture was filtered through Celite and concentrated in vacuo. The crude was partitioned between EtOAc (50 mL) and water (15 mL). The aqueous layer was extracted with EtOAc (3×10 mL), and the combined organic layers were washed with brine (15 mL) and dried over Na₂SO₄, concentrated, and purified on silica using a gradient of ethyl acetate/hexanes to give intermediate 4-bromo-1-(3-chlorophenyl)-3-nitro-pyrazole (0.792 g, 49%). The product was combined with iron (1.495 g, 26.77 mmol) in THE (20 mL) and Ethanol (10 mL). The mixture was stirred at 90° C. for 1 hour. The reaction mixture was filtered, concentrated under reduced pressure, and used as is for the next step without any further purification.

### Step 2: N-[4-Bromo-1-(3-chlorophenyl)pyrazol-3-yl]benzenesulfonamide

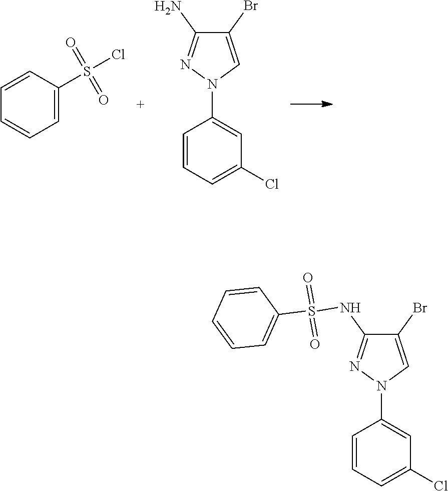 [SMILES: O=S(=O)(Cl)c1ccccc1 ; Nc1nn(-c2cccc(Cl)c2)cc1Br ; O=S(=O)(Nc1nn(-c2cccc(Cl)c2)cc1Br)c1ccccc1]

To a solution of 4-bromo-1-(3-chlorophenyl)pyrazol-3-amine (713 mg, 2.616 mmol) in pyridine (10 mL) was added benzenesulfonyl chloride (approximately 924.1 mg, 667.7 μL, 5.232 mmol), and the mixture was stirred at 90° C. for 1 hour. The reaction mixture was filtered and purified on reverse phase HPLC (HCl modifier, 25-75% ACN-H₂O) to give N-[4-bromo-1-(3-chlorophenyl)pyrazol-3-yl]benzenesulfonamide (430 mg). ¹H NMR (400 MHz, DMSO) δ 10.52 (s, 1H), 8.78 (s, 1H), 7.89 (d, J=7.5 Hz, 2H), 7.75-7.56 (m, 5H), 7.51 (t, J=8.1 Hz, 1H), 7.37 (d, J=7.5 Hz, 1H). ESI-MS m/z calc. 410.9444, found 412.0 (M+1)⁺; Retention time: 1.69 (LC method A).

### Step 3: N-[1-(3-Chlorophenyl)-4-phenyl-pyrazol-3-yl]benzenesulfonamide

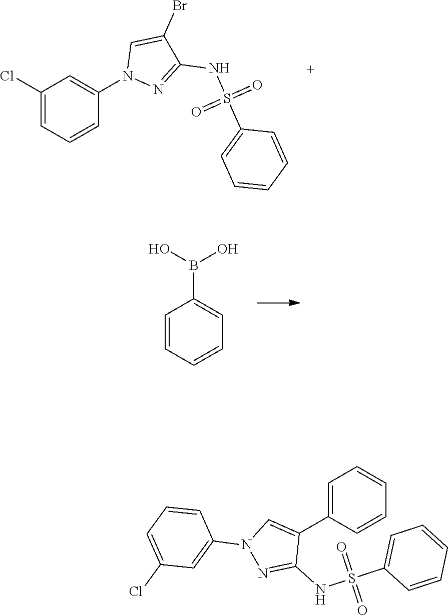 [SMILES: O=S(=O)(Nc1nn(-c2cccc(Cl)c2)cc1Br)c1ccccc1 ; OB(O)c1ccccc1 ; O=S(=O)(Nc1nn(-c2cccc(Cl)c2)cc1-c1ccccc1)c1ccccc1]

N-[4-bromo-1-(3-chlorophenyl)pyrazol-3-yl]benzenesulfonamide (25 mg, 0.06058 mmol), Pd(dppf)Cl₂ (44.3 mg, 0.06 mmol), Na₂CO₃ (3 mL, 2 M aqueous solution, 6.06 mmol), and phenylboronic acid (11.1 mg, 0.091 mmol) in dioxane (0.5 mL) were added to a microwave vial. The vial was purged with nitrogen, capped, and heated at 140-150° C. for 45 minutes in a microwave. The reaction mixture was filtered and purified by HPLC utilizing a gradient of 25-75% acetonitrile in 5 mM aqueous HCl to give N-[1-(3-chlorophenyl)-4-phenyl-pyrazol-3-yl]benzenesulfonamide (10.3 mg, 49%). ESI-MS m/z calc. 409.0652, found 410.0 (M+1)⁺; Retention time: 1.88 minutes; LC method A. ¹H NMR (400 MHz, DMSO) δ 10.34 (s, 1H), 8.90 (s, 1H), 7.93-7.87 (m, 2H), 7.74 (d, J=8.3 Hz, 2H), 7.70 (t, J=2.0 Hz, 1H), 7.67 (d, J=6.4 Hz, 1H), 7.64-7.58 (m, 3H), 7.51 (t, J=8.1 Hz, 1H), 7.44 (t, J=7.7 Hz, 2H), 7.38-7.28 (m, 2H).

### Example 22: Preparation of Compound 70

### Step 1: N-[1,4-bis(3-Chlorophenyl)pyrazol-3-yl]benzenesulfonamide

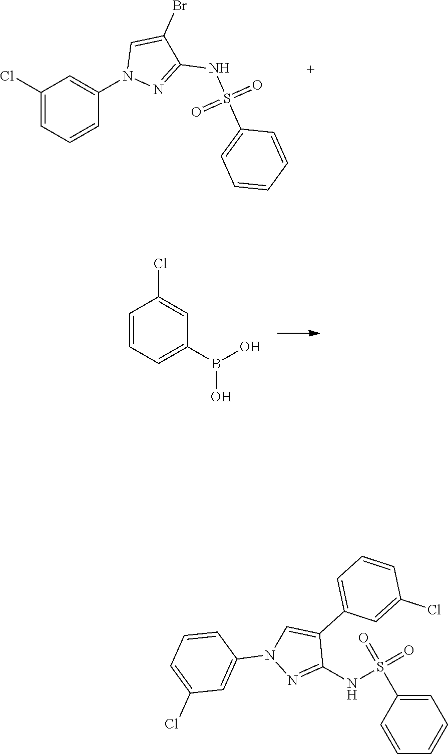 [SMILES: O=S(=O)(Nc1nn(-c2cccc(Cl)c2)cc1Br)c1ccccc1 ; OB(O)c1cccc(Cl)c1 ; O=S(=O)(Nc1nn(-c2cccc(Cl)c2)cc1-c1cccc(Cl)c1)c1ccccc1]

The compound was prepared in a manner analogous to that described above using commercially available (3-chlorophenyl)boronic acid (approximately 14.21 mg, 0.09087 mmol) to give N-[1,4-bis(3-chlorophenyl)pyrazol-3-yl]benzenesulfonamide (12.8 mg, 51%). ESI-MS m/z calc. 443.0262, found 444.0 (M+1)⁺; Retention time: 1.99 minutes; LC method A. ¹H NMR (400 MHz, DMSO) δ 10.42 (s, 1H), 8.98 (s, 1H), 7.86 (t, J=10.8 Hz, 3H), 7.72 (s, 2H), 7.60 (dd, J=20.3, 8.8 Hz, 4H), 7.54-7.43 (m, 2H), 7.36 (s, 2H).

### Example 23: Characterization of Compounds 71-100

The compounds in the following tables were prepared in a manner analogous to that described above using commercially available reagents and intermediates described herein.

| Compound | | LCMS | Calc. | | LCMS |
|---|---|---|---|---|---|
| number | Structure | Rt (min) | mass | M + 1 | Method |
| 71 | 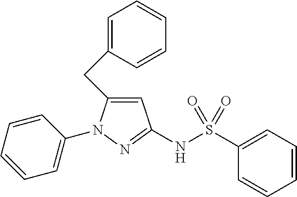 [SMILES: O=S(=O)(Nc1cc(Cc2ccccc2)n(-c2ccccc2)n1)c1ccccc1] | 1.71 | 389.12 | 390.2 | A |
| 72 | 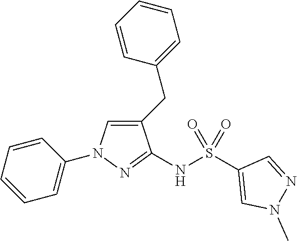 [SMILES: Cn1cc(S(=O)(=O)Nc2nn(-c3ccccc3)cc2Cc2ccccc2)cn1] | 1.53 | 393.126 | 394.1 | A |
| 73 | 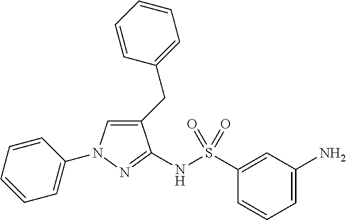 [SMILES: Nc1cccc(S(=O)(=O)Nc2nn(-c3ccccc3)cc2Cc2ccccc2)c1] | 1.57 | 404.131 | 405.1 | A |
| 74 | 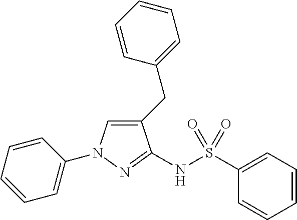 [SMILES: O=S(=O)(Nc1nn(-c2ccccc2)cc1Cc1ccccc1)c1ccccc1] | 1.81 | 389.12 | 390.1 | A |
| 75 | 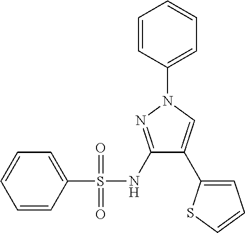 [SMILES: O=S(=O)(Nc1nn(-c2ccccc2)cc1-c1cccs1)c1ccccc1] | 1.81 | 381.061 | 382 | A |
| 76 | 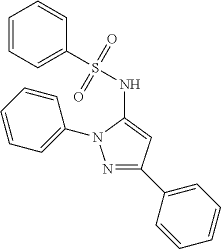 [SMILES: O=S(=O)(Nc1cc(-c2ccccc2)nn1-c1ccccc1)c1ccccc1] | 1.74 | 375.104 | 376 | A |
| 77 | 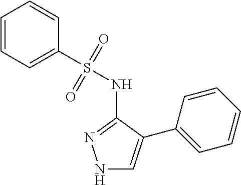 [SMILES: O=S(=O)(Nc1n[nH]cc1-c1ccccc1)c1ccccc1] | 1.16 | 299.073 | 300 | A |
| 78 | 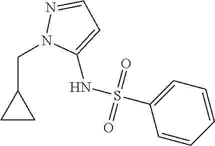 [SMILES: O=S(=O)(Nc1ccnn1CC1CC1)c1ccccc1] | 1.14 | 277.088 | 278 | A |
| 79 | 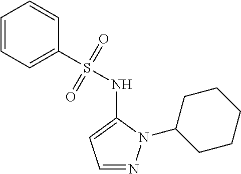 [SMILES: O=S(=O)(Nc1ccnn1C1CCCCC1)c1ccccc1] | 1.41 | 305.12 | 306 | A |
| 80 | 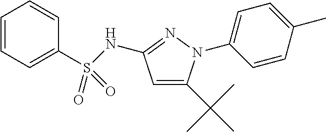 [SMILES: Cc1ccc(-n2nc(NS(=O)(=O)c3ccccc3)cc2C(C)(C)C)cc1] | 1.83 | 369.151 | 370 | A |
| 81 | 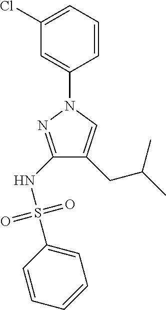 [SMILES: CC(C)Cc1cn(-c2cccc(Cl)c2)nc1NS(=O)(=O)c1ccccc1] | 1.95 | 389.096 | 390 | A |
| 82 | 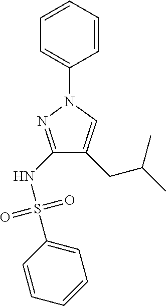 [SMILES: CC(C)Cc1cn(-c2ccccc2)nc1NS(=O)(=O)c1ccccc1] | 1.8 | 355.135 | 356 | A |
| 83 | 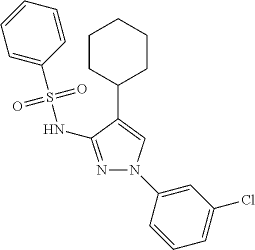 [SMILES: O=S(=O)(Nc1nn(-c2cccc(Cl)c2)cc1C1CCCCC1)c1ccccc1] | 2.06 | 415.112 | 416 | A |
| 84 | 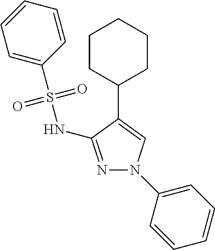 [SMILES: O=S(=O)(Nc1nn(-c2ccccc2)cc1C1CCCCC1)c1ccccc1] | 1.92 | 381.151 | 382 | A |
| 85 | 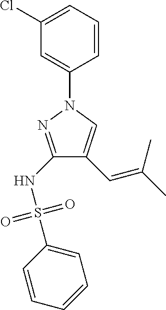 [SMILES: CC(C)=Cc1cn(-c2cccc(Cl)c2)nc1NS(=O)(=O)c1ccccc1] | 1.88 | 387.081 | 388 | A |
| 86 | 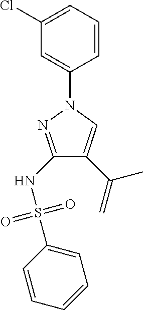 [SMILES: C=C(C)c1cn(-c2cccc(Cl)c2)nc1NS(=O)(=O)c1ccccc1] | 1.79 | 373.065 | 374 | A |
| 87 | 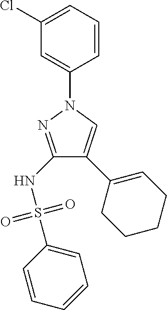 [SMILES: O=S(=O)(Nc1nn(-c2cccc(Cl)c2)cc1C1=CCCCC1)c1ccccc1] | 2.01 | 413.096 | 414 | A |
| 88 | 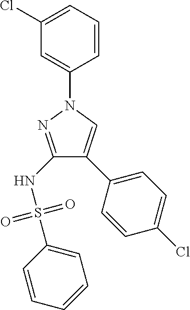 [SMILES: O=S(=O)(Nc1nn(-c2cccc(Cl)c2)cc1-c1ccc(Cl)cc1)c1ccccc1] | 1.99 | 443.026 | 444 | A |
| 89 | 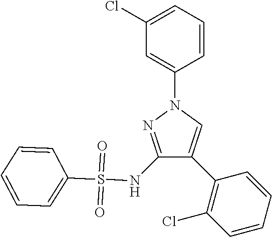 [SMILES: O=S(=O)(Nc1nn(-c2cccc(Cl)c2)cc1-c1ccccc1Cl)c1ccccc1] | 1.95 | 443.026 | 444 | A |
| 90 | 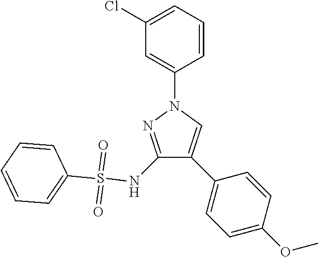 [SMILES: COc1ccc(-c2cn(-c3cccc(Cl)c3)nc2NS(=O)(=O)c2ccccc2)cc1] | 1.87 | 439.076 | 440 | A |
| 91 | 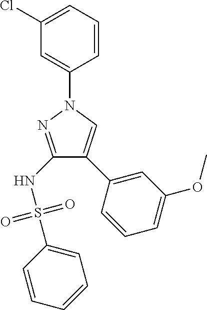 [SMILES: COc1cccc(-c2cn(-c3cccc(Cl)c3)nc2NS(=O)(=O)c2ccccc2)c1] | 1.88 | 439.076 | 440 | A |
| 92 | 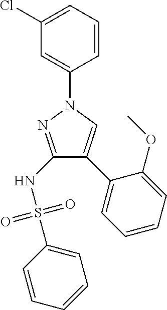 [SMILES: COc1ccccc1-c1cn(-c2cccc(Cl)c2)nc1NS(=O)(=O)c1ccccc1] | 1.92 | 439.076 | 440 | A |
| 93 | 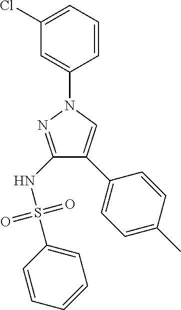 [SMILES: Cc1ccc(-c2cn(-c3cccc(Cl)c3)nc2NS(=O)(=O)c2ccccc2)cc1] | 1.97 | 423.081 | 424 | A |
| 94 | 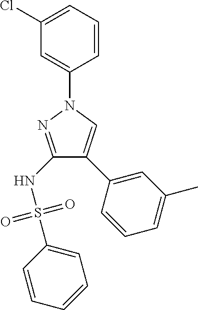 [SMILES: Cc1cccc(-c2cn(-c3cccc(Cl)c3)nc2NS(=O)(=O)c2ccccc2)c1] | 1.97 | 423.081 | 424 | A |
| 95 | 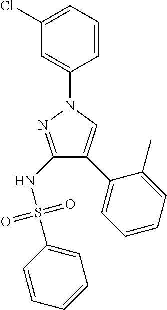 [SMILES: Cc1ccccc1-c1cn(-c2cccc(Cl)c2)nc1NS(=O)(=O)c1ccccc1] | 1.95 | 423.081 | 424 | A |
| 96 | 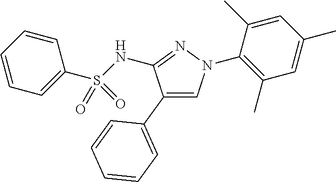 [SMILES: Cc1cc(C)c(-n2cc(-c3ccccc3)c(NS(=O)(=O)c3ccccc3)n2)c(C)c1] | 1.94 | 417.151 | 418 | A |
| 97 | 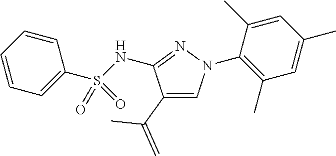 [SMILES: C=C(C)c1cn(-c2c(C)cc(C)cc2C)nc1NS(=O)(=O)c1ccccc1] | 1.86 | 381.151 | 382 | A |
| 98 | 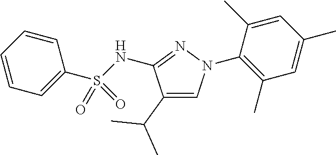 [SMILES: Cc1cc(C)c(-n2cc(C(C)C)c(NS(=O)(=O)c3ccccc3)n2)c(C)c1] | 1.96 | 383.167 | 384 | A |
| 99 | 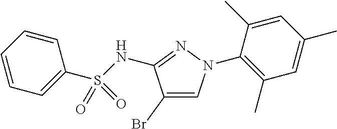 [SMILES: Cc1cc(C)c(-n2cc(Br)c(NS(=O)(=O)c3ccccc3)n2)c(C)c1] | 1.76 | 419.03 | 420 | A |
| 100 | 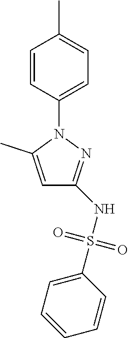 [SMILES: Cc1ccc(-n2nc(NS(=O)(=O)c3ccccc3)cc2C)cc1] | 1.57 | 327.104 | 328 | A |

| Com- | |
|---|---|
| pound | |
| number | NMR |
| 71 | ¹H NMR (400 MHz, dimethylsulfoxide-d₆) δ |
| | 10.41 (s, 1H, D₂O exchangeable), 7.68-7.62 |
| | (m, 3H), 7.56-7.50 (m, 2H), 7.48-7.43 (m, 4H), |
| | 7.40-7.34 (m, 1H), 7.31-7.25 (m, 2H), 7.23- |
| | 7.16 (m, 3H), 5.56 (s, 1H), 3.84 (s, 2H) |
| 75 | ¹H NMR (400 MHz, DMSO) δ 10.64 (s, 1H), |
| | 7.99 (s, 1H), 7.42-7.19 (m, 13H), 6.96 (dd, |
| | J = 5.1, 3.6 Hz, 1H). |
| 77 | ¹H NMR (400 MHz, DMSO) δ 12.73 (s, 1H), |
| | 9.89 (s, 1H), 7.99 (s, 1H), 7.80 (d, J = 7.6 Hz, |
| | 2H), 7.61(dd, J = 21.9, 7.1 Hz, 3H), 7.52 (t, J = |
| | 7.5 Hz, 2H), 7.34 (t, J = 7.6 Hz, 2H), 7.21 (t, |
| | J = 7.1 Hz, 1H). |
| 78 | ¹H NMR (400 MHz, DMSO) δ 10.30 (d, J = |
| | 2.0 Hz, 1H), 7.83-7.59 (m, 5H), 7.29 (d, J = |
| | 1.3 Hz, 1H), 5.55 (d, J = 1.4 Hz, 1H), 3.81 (d, |
| | J = 7.0 Hz, 2H), 1.20-1.03 (m, 1H), 0.47-0.26 |
| | (m, 4H). |
| 80 | ¹H NMR (400 MHz, DMSO) δ 10.28 (s, 1H), |
| | 7.73-7.65 (m, 3H), 7.62-7.54 (m, 2H), 7.32 (d, |
| | J = 8.4 Hz, 2H), 7.24 (d, J = 8.2 Hz, 2H), 5.60 |
| | (s, 1H), 2.35 (s, 3H), 1.17 (s, 9H). |
| 86 | ¹H NMR (400 MHz, DMSO) δ 10.16 (s, 1H), |
| | 8.59 (s, 1H), 7.90 (d, J = 8.2 Hz, 2H), 7.71- |
| | 7.56 (m, 5H), 7.47 (t, J = 7.8 Hz, 1H), 7.32 |
| | (d, J = 7.9 Hz, 1H), 5.57 (s, 1H), 5.03 (s, |
| | 1H), 2.06 (d, J = 17.8 Hz, 3H) |
| 88 | ¹H NMR (400 MHz, DMSO) δ 10.34 (s, 1H), |
| | 8.93 (s, 1H), 7.90-7.85 (m, 2H), 7.75 (d, J = |
| | 8.6 Hz, 2H), 7.70 (t, J = 2.0 Hz, 1H), 7.66 (d, |
| | J = 7.4 Hz, 1H), 7.60 (q, J = 8.1 Hz, 3H), 7.51 |
| | (t, J = 8.9 Hz, 3H), 7.36 (d, J = 7.2 Hz, 1H). |
| 92 | ¹H NMR (400 MHz, DMSO) δ 10.03 (s, 1H), |
| | 8.60 (s, 1H), 7.88 (d, J = 8.7 Hz, 2H), 7.70 |
| | (t, J = 2.0 Hz, 1H), 7.65 (t, J = 8.0 Hz, 2H), |
| | 7.56 (dd, J = 15.7, 8.5 Hz, 3H), 7.49 (t, J = |
| | 8.1 Hz, 1H), 7.32 (dd, J = 12.4, 5.0 Hz, 2H), |
| | 7.07 (d, J = 8.0 Hz, 1H), 7.00 (t, J = 7.5 Hz, |
| | 1H), 3.81 (s, 3H). |
| 93 | ¹H NMR (400 MHz, DMSO) δ 10.26 (s, 1H), |
| | 8.83 (s, 1H), 7.89 (d, J = 7.6 Hz, 2H), 7.72- |
| | 7.57 (m, 7H), 7.50 (t, J = 8.1 Hz, 1H), 7.34 |
| | (d, J = 7.8 Hz, 1H), 7.23 (d, J = 8.1 Hz, 2H), |
| | 2.34 (s, 3H). |
| 94 | ¹H NMR (400 MHz, DMSO) δ 10.28 (s, 1H), |
| | 8.85 (s, 1H), 7.89 (d, J = 7.7 Hz, 2H), 7.71 |
| | (s, 1H), 7.67-7.47 (m, 7H), 7.31 (dd, J = 19.8, |
| | 7.8 Hz, 2H), 7.11 (d, J = 7.5 Hz, 1H), 2.34 (s, |
| | 3H). |
| 96 | ¹H NMR (400 MHz, DMSO) δ 10.07 (s, 1H), |
| | 8.15 (s, 1H), 7.75 (t, J = 8.3 Hz, 4H), 7.59-7.47 |
| | (m, 3H), 7.37 (t, J = 7.6 Hz, 2H), 7.26 (d, J = |
| | 7.2 Hz, 1H), 6.98 (s, 2H), 2.27 (s, 3H), 1.86 |
| | (s, 6H). |
| 98 | ¹H NMR (400 MHz, DMSO) δ 9.82 (s, 1H), |
| | 7.73 (d, J = 8.8 Hz, 2H), 7.55 (dt, J = 14.9, 7.2 |
| | Hz, 4H), 6.93 (s, 2H), 2.92 (d, J = 9.7 Hz, 1H), |
| | 2.25 (s, 3H), 1.76 (s, 6H), 1.12 (d, J = 6.9 Hz, |
| | 6H). |
| 99 | ¹H NMR (400 MHz, DMSO) δ 10.17 (s, 1H), |
| | 8.05 (s, 1H), 7.76 (d, J = 7.5 Hz, 2H), 7.57 (dt, |
| | J = 15.0, 7.2 Hz, 3H), 6.97 (s, 2H), 2.26 (s, 3H), |
| | 1.80 (s, 6H). |

### Example 24: Preparation of Compound 101

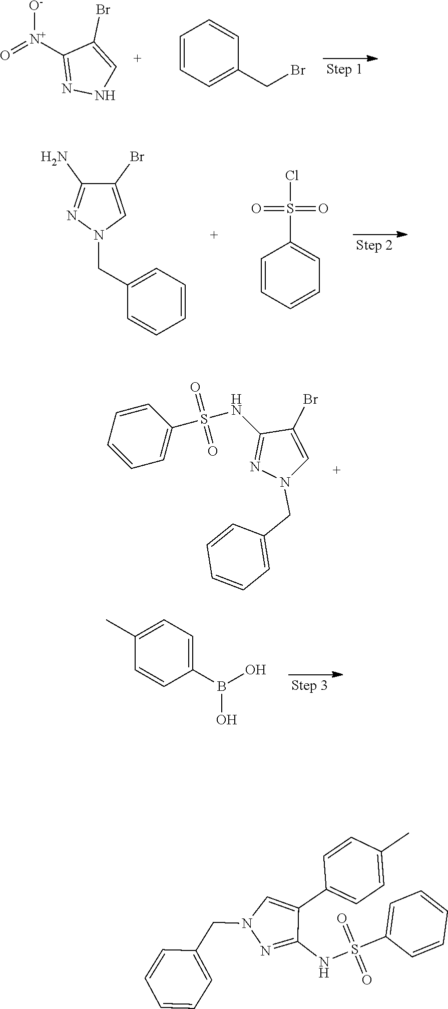

### Step 1: 1-Benzyl-4-bromo-pyrazol-3-amine

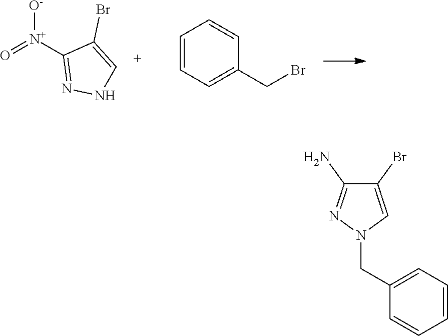 [SMILES: O=[N+]([O-])c1n[nH]cc1Br ; BrCc1ccccc1 ; Nc1nn(Cc2ccccc2)cc1Br]

To a glass vial was added 4-bromo-3-nitro-1H-pyrazole (1 g, 5.209 mmol), followed by DMF (10.00 mL), bromomethylbenzene (approximately 1.336 g, 929.1 μL, 7.814 mmol), and K₂CO₃ (approximately 1.440 g, 10.42 mmol). The reaction mixture was stirred at 85° C. for 3 days. The reaction was worked up by adding water (20 mL) and then extracted with ethyl acetate (2×20 mL). The organic layers were dried over Na₂SO₄, concentrated, and purified on silica using a gradient of ethyl acetate/hexanes to give the nitro intermediate. Iron (1.48 g, 26.50 mmol) was added to 1-benzyl-4-bromo-3-nitro-pyrazole (1.079 g, 73%) in THF (20.00 mL) and Ethanol (10.00 mL), followed by HCl (4.4 mL of 6 M, 26.40 mmol). The mixture was stirred at 90° C. for 1 hour. The reaction mixture was filtered, concentrated under reduced pressure, and used as is without further purification. 1-Benzyl-4-bromo-pyrazol-3-amine (964 mg). ¹H NMR (400 MHz, DMSO) δ 7.90 (s, 1H), 7.38-7.26 (m, 3H), 7.26-7.20 (m, 2H), 5.11 (s, 2H). ESI-MS m/z calc. 251.00581, found 253.0 (M+1)⁺; Retention time: 1.12 minutes (LC method A).

### Step 2: N-(1-Benzyl-4-bromo-pyrazol-3-yl)benzenesulfonamide

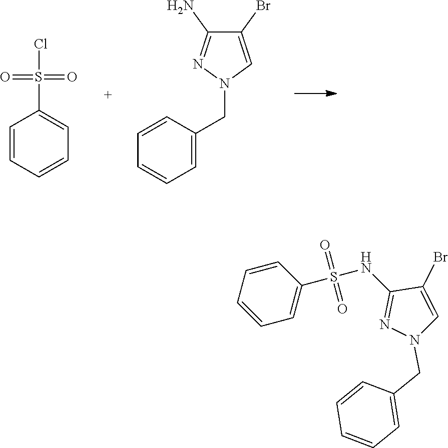 [SMILES: O=S(=O)(Cl)c1ccccc1 ; Nc1nn(Cc2ccccc2)cc1Br ; O=S(=O)(Nc1nn(Cc2ccccc2)cc1Br)c1ccccc1]

To a solution of 1-benzyl-4-bromo-pyrazol-3-amine (964 mg, 3.824 mmol) in pyridine (18.95 mL) was added benzenesulfonyl chloride (approximately 1.351 g, 976.2 μL, 7.648 mmol), and the reaction was stirred at 95° C. for 1 hour. The reaction mixture was filtered and purified on reverse phase HPLC (HCl modifier, 25-75% ACN-H₂O) to give N-(1-benzyl-4-bromo-pyrazol-3-yl)benzenesulfonamide (672.2 mg). ¹H NMR (400 MHz, DMSO) δ 10.04 (s, 1H), 8.02 (s, 1H), 7.73 (d, J=7.4 Hz, 2H), 7.63 (t, J=7.4 Hz, 1H), 7.52 (t, J=7.7 Hz, 2H), 7.37-7.27 (m, 3H), 7.12 (d, J=7.7 Hz, 2H), 5.16 (s, 2H). ESI-MS m/z calc. 390.99902, found 392.0 (M+1)⁺; Retention time: 1.45 minutes (LC method A).

### Step 3: N-[1-Benzyl-4-(p-tolyl)pyrazol-3-yl]benzenesulfonamide

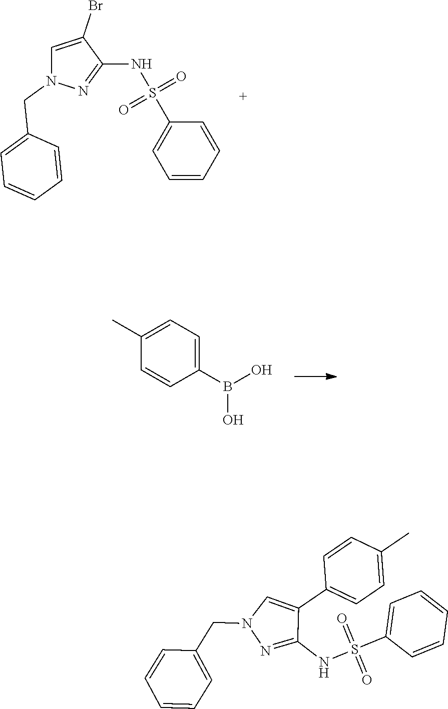

N-(1-benzyl-4-bromo-pyrazol-3-yl)benzenesulfonamide (25 mg, 0.06373 mmol), Pd(dppf)Cl₂, Na₂CO₃, and para-tolyl boronic acid (13 mg, 0.095 mmol) in dioxane (1 mL) were added into a microwave vial. The vial was purged with nitrogen, capped, and heated at 140° C. for 45 minutes in a microwave. The crude mixture was filtered and purified by HPLC utilizing a gradient of 25-75% acetonitrile in 5 mM aqueous HCl to give N-[1-benzyl-4-(p-tolyl)pyrazol-3-yl]benzenesulfonamide (0.7 mg, 30%). ESI-MS m/z calc. 403.13544, found 404.0 (M+1)⁺; Retention time: 1.76 minutes; LC method A. ¹H NMR (400 MHz, DMSO) δ 9.84 (s, 1H), 8.07 (s, 1H), 7.72 (d, J=8.6 Hz, 2H), 7.59 (t, J=7.4 Hz, 1H), 7.49 (dd, J=15.9, 7.9 Hz, 4H), 7.33 (dd, J=16.6, 7.5 Hz, 3H), 7.15 (t, J=6.9 Hz, 4H), 5.15 (s, 2H), 2.30 (s, 3H).

### Example 25: Preparation of Compound 102

### Step 1: 1-Benzylpyrazol-3-amine

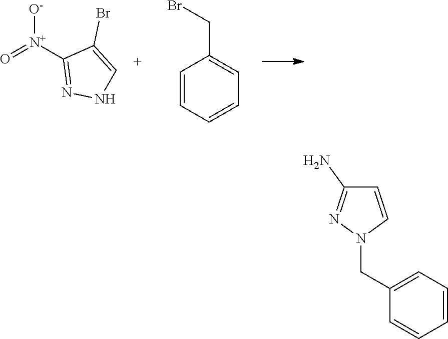 [SMILES: O=[N+]([O-])c1n[nH]cc1Br ; BrCc1ccccc1 ; Nc1ccn(Cc2ccccc2)n1]

To a glass vial was added 4-bromo-3-nitro-1H-pyrazole (1 g, 5.209 mmol) followed by DMF (10.00 mL), bromomethylbenzene (approximately 1.158 g, 805.3 μL, 6.772 mmol), and K₂CO₃ (1.487 g, 10.76 mmol). The reaction mixture was stirred at 85° C. overnight. The reaction was worked up by adding water (20 mL) and then extracted with ethyl acetate (2×20 mL). The organic layers were dried over Na₂SO₄, concentrated, and purified on silica using a gradient of ethyl acetate/hexane to give 1-benzyl-4-bromo-3-nitro-pyrazole (1.0235 g, 69%) ¹H NMR (400 MHz, DMSO) δ 8.48 (s, 1H), 7.43-7.33 (m, 5H), 5.46 (s, 2H). ESI-MS m/z calc. 280.97998, found 282.0 (M+1)⁺; Retention time: 1.49 minutes (LC method A).

Pd on C, wet, Degussa (551.2 mg of 5% w/w, 0.2590 mmol) was added to 1-benzyl-4-bromo-3-nitro-pyrazole (1.0235 g, 69%) in Methanol (50 mL). The flask was purged with nitrogen and stirred at room temperature overnight under a balloon of Hydrogen. After workup, 1-benzylpyrazol-3-amine (853 mg) was isolated. ¹H NMR (400 MHz, DMSO) δ 10.00 (s, 1H), 8.00 (d, J=2.3 Hz, 1H), 7.43-7.16 (m, 5H), 6.21 (d, J=2.3 Hz, 1H), 5.31 (s, 2H).

### Step 2: N-(1-Benzylpyrazol-3-yl)benzenesulfonamide

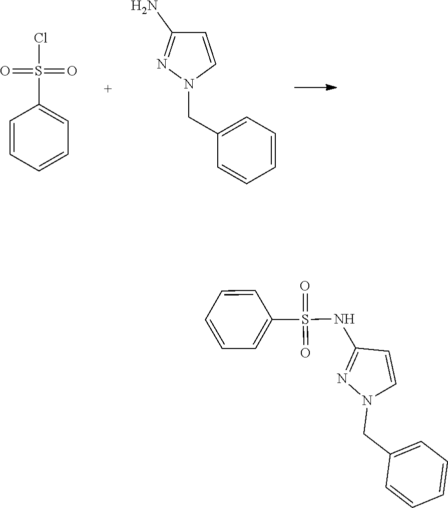 [SMILES: O=S(=O)(Cl)c1ccccc1 ; Nc1ccn(Cc2ccccc2)n1 ; O=S(=O)(Nc1ccn(Cc2ccccc2)n1)c1ccccc1]

To a solution of 1-benzylpyrazol-3-amine (853 mg, 4.92 mmol) in pyridine (17.40 mL) was added benzenesulfonyl chloride (approximately 1.740 g, 1.257 mL, 9.850 mmol), and the reaction was stirred at 115° C. for 1 hour. The reaction mixture was filtered and purified on reverse phase HPLC (HCl modifier, 25-75% ACN-H₂O) to give N-(1-benzylpyrazol-3-yl)benzenesulfonamide (109.7 mg). ESI-MS m/z calc. 313.0885, found 414.0 (M+1)⁺; Retention time: 1.35 minutes; LC method A.

### Example 26: Characterization of Compounds 103-115

The compounds in the following tables were prepared in a manner analogous to that described above using commercially available reagents and intermediates described herein.

| | | LCMS | | | | |
|---|---|---|---|---|---|---|
| Cmpd | | Rt | Calc. | | LCMS | |
| number | Structure | (min) | mass | M + 1 | Met. | NMR |
| 103 | 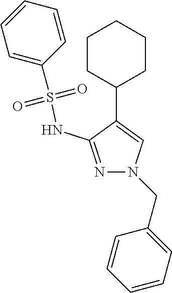 [SMILES: O=S(=O)(Nc1nn(Cc2ccccc2)cc1C1CCCCC1)c1ccccc1] | 1.86 | 395.167 | 396 | A | |
| 104 | 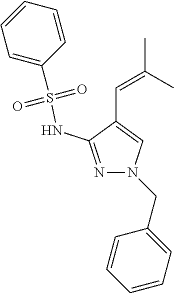 [SMILES: CC(C)=Cc1cn(Cc2ccccc2)nc1NS(=O)(=O)c1ccccc1] | 1.65 | 367.135 | 368 | A | |
| 105 | 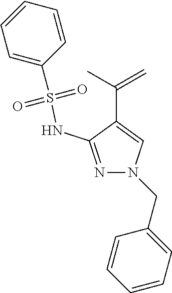 [SMILES: C=C(C)c1cn(Cc2ccccc2)nc1NS(=O)(=O)c1ccccc1] | 1.56 | 353.12 | 354 | A | |
| 106 | 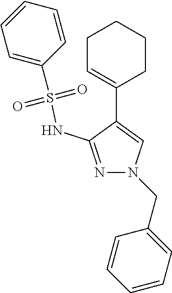 [SMILES: O=S(=O)(Nc1nn(Cc2ccccc2)cc1C1=CCCCC1)c1ccccc1] | 1.79 | 393.151 | 394 | A | |
| 107 | 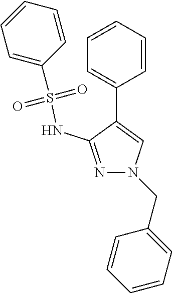 [SMILES: O=S(=O)(Nc1nn(Cc2ccccc2)cc1-c1ccccc1)c1ccccc1] | 1.66 | 389.12 | 390 | A | ¹H NMR (400 MHz, DMSO) δ 9.92 (s, 1H), 8.14 (s, 1H), 7.72 (d, J = 8.5 Hz, 2H), 7.61 (dd, J = 16.1, 7.3 Hz, 3H), 7.48 (t, J = 7.7 Hz, 2H), 7.38-7.30 (m, 5H), 7.22 (t, J = 7.4 Hz, 1H), 7.16 (d, J = 6.4 Hz, 2H), 5.16 (s, 2H). |
| 108 | 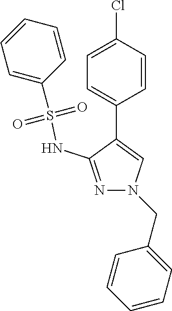 [SMILES: O=S(=O)(Nc1nn(Cc2ccccc2)cc1-c1ccc(Cl)cc1)c1ccccc1] | 1.79 | 423.081 | 424 | A | ¹H NMR (400 MHz, DMSO) δ 9.98 (s, 1H), 8.18 (s, 1H), 7.72-7.67 (m, 2H), 7.63 (d, J = 8.6 Hz, 2H), 7.59 (d, J = 7.5 Hz, 1H), 7.48 (t, J = 7.7 Hz, 2H), 7.40 (d, J = 8.6 Hz, 2H), 7.37-7.29 (m, 3H), 7.15 (d, J = 9.4 Hz, 2H), 5.16 (s, 2H). |
| 109 | 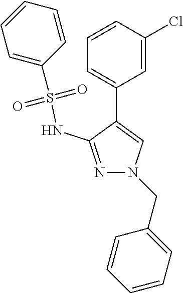 [SMILES: O=S(=O)(Nc1nn(Cc2ccccc2)cc1-c1cccc(Cl)c1)c1ccccc1] | 1.78 | 423.081 | 424 | A | ¹H NMR (400 MHz, DMSO) δ 10.02 (s, 1H), 8.25 (s, 1H), 7.73-7.67 (m, 3H), 7.59 (dd, J = 13.7, 6.4 Hz, 2H), 7.48 (t, J = 7.7 Hz, 2H), 7.39-7.26 (m, 5H), 7.16 (d, J = 9.3 Hz, 2H), 5.17 (s, 2H). |
| 110 | 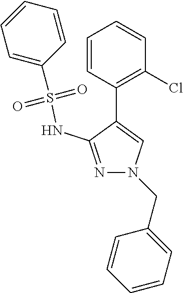 [SMILES: O=S(=O)(Nc1nn(Cc2ccccc2)cc1-c1ccccc1Cl)c1ccccc1] | 1.74 | 423.081 | 424 | A | ¹H NMR (400 MHz, DMSO) δ 9.89 (s, 1H), 8.03 (s, 1H), 7.57 (t, J = 7.4 Hz, 1H), 7.49-7.39 (m, 4H), 7.31 (dd, J = 14.6, 9.1 Hz, 5H), 7.16 (d, J = 94 Hz, 2H), 5.21 (s, 2H). |
| 111 | 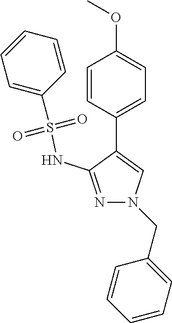 [SMILES: COc1ccc(-c2cn(Cc3ccccc3)nc2NS(=O)(=O)c2ccccc2)cc1] | 1.64 | 419.13 | 420 | A | ¹H NMR (400 MHz, DMSO) δ 9.82 (s, 1H), 8.03 (s, 1H), 7.72 (d, J = 8.6 Hz, 2H), 7.59 (t, J = 8.0 Hz, 1H), 7.54 (d, J = 8.8 Hz, 2H), 7.48 (t, J = 7.0 Hz, 2H), 7.33 (dd, J = 14.3, 10.0 Hz, 3H), 7.16 (d, J = 7.5 Hz, 2H), 6.91 (d, J = 8.9 Hz, 2H), 5.14 (s, 2H), 3.77 (s, 3H). |
| 112 | 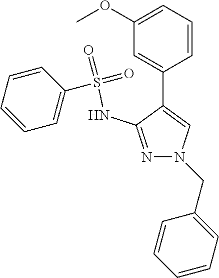 [SMILES: COc1cccc(-c2cn(Cc3ccccc3)nc2NS(=O)(=O)c2ccccc2)c1] | 1.66 | 419.13 | 420 | A | ¹H NMR (400 MHz, DMSO) δ 9.90 (s, 1H), 8.15 (s, 1H), 7.73 (d, J = 7.7 Hz, 2H), 7.59 (t, J = 7.2 Hz, 1H), 7.48 (t, J = 7.7 Hz, 2H), 7.37-7.15 (m, 8H), 6.80 (d, J = 8.1 Hz, 1H), 5.16 (s, 2H), 3.78 (s, 3H). |
| 113 | 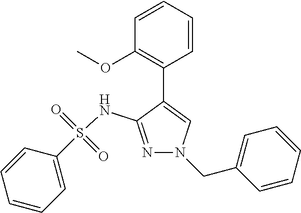 [SMILES: COc1ccccc1-c1cn(Cc2ccccc2)nc1NS(=O)(=O)c1ccccc1] | 1.69 | 419.13 | 420 | A | |
| 114 | 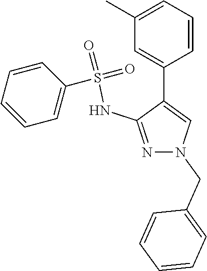 [SMILES: Cc1cccc(-c2cn(Cc3ccccc3)nc2NS(=O)(=O)c2ccccc2)c1] | 1.75 | 403.135 | 404 | A | ¹H NMR (400 MHz, DMSO) δ 9.87 (s, 1H), 8.09 (s, 1H), 7.72 (d, J = 8.5 Hz, 2H), 7.59 (t, J = 7.4 Hz, 1H), 7.47 (t, J = 7.7 Hz, 2H), 7.44-7.38 (m, 2H), 7.38-7.29 (m, 3H), 7.22 (t, J = 7.6 Hz, 1H), 7.17 (d, J = 6.5 Hz, 2H), 7.03 (d, J = 7.5 Hz, 1H), 5.16 (s, 2H), 2.30 (s, 3H). |
| 115 | 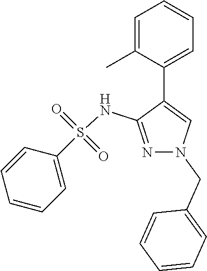 [SMILES: Cc1ccccc1-c1cn(Cc2ccccc2)nc1NS(=O)(=O)c1ccccc1] | 1.73 | 403.135 | 404 | A | ¹H NMR (400 MHz, DMSO) δ 9.76 (s, 1H), 7.86 (s, 1H), 7.65 (d, J = 7.4 Hz, 2H), 7.56 (t, J = 7.4 Hz, 1H), 7.43 (t, J = 7.7 Hz, 2H), 7.38-7.28 (m, 3H), 7.24-7.11 (m, 6H), 5.20 (s, 2H), 2.16 (s, 3H). |

### Example 27: Preparation of Compound 116

### Step 1: Ethyl 2-(benzenesulfonamido)-4,6-diphenyl-6H-1,3-thiazine-5-carboxylate

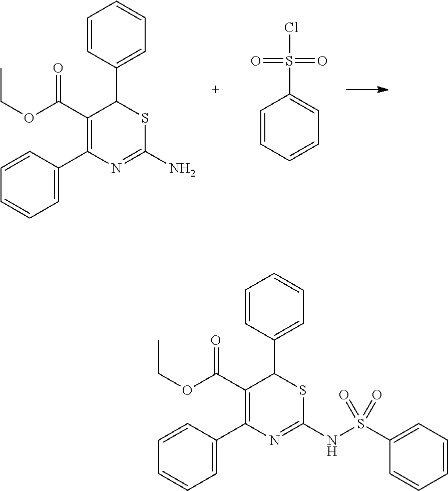 [SMILES: CCOC(=O)C1=C(c2ccccc2)N=C(N)SC1c1ccccc1 ; O=S(=O)(Cl)c1ccccc1 ; CCOC(=O)C1=C(c2ccccc2)N=C(NS(=O)(=O)c2ccccc2)SC1c1ccccc1]

To a mixture of ethyl 2-amino-4,6-diphenyl-6H-1,3-thiazine-5-carboxylate (20 mg, 0.05910 mmol) and DABCO (20 mg) was added PhSO₂Cl (11 μL, 0.08865 mmol), and the reaction mixture stirred at 40° C. for 4 hours. The reaction mixture was diluted with MeOH and filtered. Purification by HPLC (1-99% ACN in water (HCl modifier)) gave ethyl 2-(benzenesulfonamido)-4,6-diphenyl-6H-1,3-thiazine-5-carboxylate (hydrochloride salt) (18 mg, 59%). ESI-MS m/z calc. 478.1021, found 479.2 (M+1)⁺; Retention time: 1.77 minutes; L C method A.

### Example 28: Preparation of Compound 117

### Step 1: N-(4,6-Diphenyl-4H-1,3-thiazin-2-yl)benzenesulfonamide

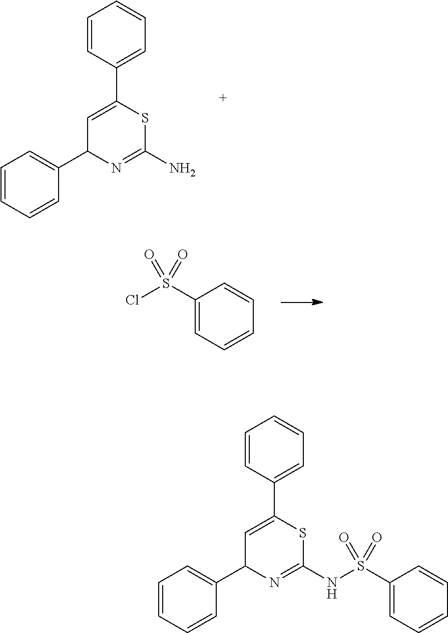 [SMILES: NC1=NC(c2ccccc2)C=C(c2ccccc2)S1 ; O=S(=O)(Cl)c1ccccc1 ; O=S(=O)(NC1=NC(c2ccccc2)C=C(c2ccccc2)S1)c1ccccc1]

To a 10 mL vial equipped with a magnetic stir bar, 4,6-diphenyl-4H-1,3-thiazin-2-amine (16.9 mg, 0.06345 mmol), acetonitrile (500 μL), DABCO (20.0 mg, 0.1783 mmol), and benzenesulfonyl chloride (20 μL, 0.1567 mmol) were added, in this order. The vial was capped and stirred at room temperature for 20 minutes, upon which the reaction mixture was diluted with 1:1 methanol:dimethylsulfoxide (500 NL), filtered, and purified by reverse phase HPLC (1-99% acetonitrile in water using HCl as a modifier) to give N-(4,6-diphenyl-4H-1,3-thiazin-2-yl)benzenesulfonamide (2.1 mg, 7%). ESI-MS m/z calc. 406.08096, found 407.1 (M+1); Retention time: 1.77 minutes; LC method A.

### Example 29: Preparation of Compound 118

### Step 1: N-(3-chloro-5-phenyl-pyrazin-2-yl)benzenesulfonamide

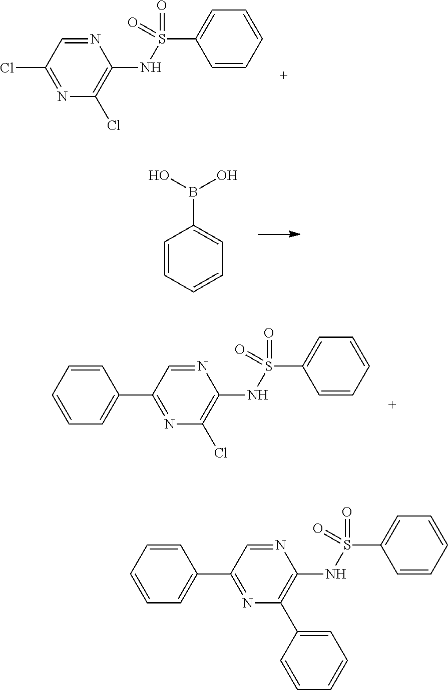 [SMILES: O=S(=O)(Nc1ncc(Cl)nc1Cl)c1ccccc1 ; OB(O)c1ccccc1 ; O=S(=O)(Nc1ncc(-c2ccccc2)nc1Cl)c1ccccc1 ; O=S(=O)(Nc1ncc(-c2ccccc2)nc1-c1ccccc1)c1ccccc1]

N-(3,5-dichloropyrazin-2-yl)benzenesulfonamide (200 mg, 0.6576 mmol), phenylboronic acid (90 mg, 0.7381 mmol), Pd(PPh₃)₄ (40 mg, 0.03462 mmol), and K₂CO₃ (790 μL of 2.5 M, 1.975 mmol) were mixed in n-propanol (6 mL). The mixture was purged with N₂; the vial was sealed and the mixture was heated to 120° C. for 16 hours. The reaction mixture was filtered and subjected to HPLC purification using 25-75% ACN in water (0.05% HCl modifier) over 15 minutes. Fractions were dried to give the product as a white solid, N-(3-chloro-5-phenyl-pyrazin-2-yl)benzenesulfonamide (85.5 mg, 36%), ESI-MS m/z calc. 345.03387, found 346.0 (M+1)⁺; Retention time: 1.61 minutes (LC method A) and N-(3,5-diphenylpyrazin-2-yl)benzenesulfonamide (10.4 mg). ¹H NMR (400 MHz, DMSO) δ 10.95 (s, 1H), 8.80 (s, 1H), 8.09 (d, J=6.9 Hz, 2H), 8.00-7.96 (m, 2H), 7.92 (d, J=7.1 Hz, 2H), 7.67-7.42 (m, 9H). ESI-MS m/z calc. 387.10416, found 388.0 (M+1)⁺; Retention time: 1.87 minutes, (LC method A).

### Example 30: Preparation of Compound 119

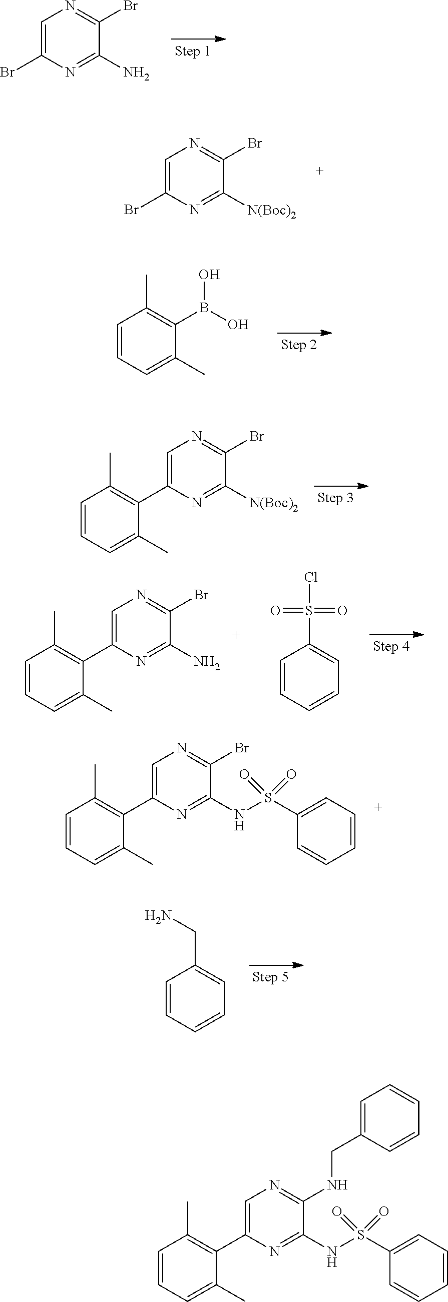

### Step 1: tert-Butyl N-tert-butoxycarbonyl-N-(3,6-dibromopyrazin-2-yl)carbamate

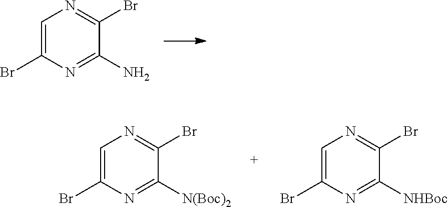

3,6-Dibromopyrazin-2-amine (3 g, 11.86 mmol) was dissolved in dichloromethane (25 mL) at room temperature. Di-tert-butyl carbamate (5.7 g, 26.1 mmol) was added, followed by NEt₃ ((3.5 mL, 23.7 mmol) and 4-dimethylamino pyridine (10 mg, 0.082 mmol). The mixture was stirred under nitrogen for 15 hours. It was then diluted with 20 mL DCM, washed with water, brine and concentrated. The residue was purified by silica gel chromatography using 0-25% EtOAc/hexanes to afford tert-butyl N-tert-butoxycarbonyl-N-(3,6-dibromopyrazin-2-yl)carbamate (4.2 g, ˜70% yield). This material was used in the Suzuki coupling (contaminated with some Mono Boc product). Mono-Boc: ESI-MS m/z calc. 350.9, found 354.0 (M+3, Br)⁺; Retention time 3.28 min, Double-Boc: ESI-MS m/z calc. 450.97, found 454.3 (M+3, Br)⁺; Retention time 4.04 minutes. (LC method H).

### Step 2: tert-Butyl N-[3-bromo-6-(2,6-dimethylphenyl)pyrazin-2-yl]-N-tert-butoxycarbonyl-carbamate

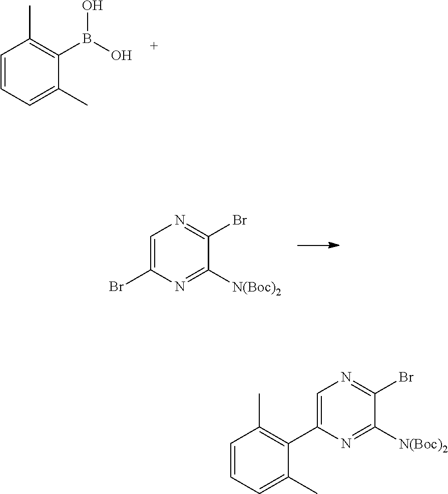

A mixture of Mono- and Double-Boc-3,6-Dibromopyrazin-2-amine from the previous step (2 g, ˜5 mmol assumed) was dissolved in DME (20 mL). 2, 6-Dimethylphenyl boronic acid (700 mg, 4.7 mmol) was added, followed by Na₂CO₃ (5 mL, 2M aq., 10 mmol) and Pd(PPh₃)₂Cl₂ (160 mg, 0.2 4 mmol). The mixture was briefly degassed and then heated in a sealed flask in an 80° C. oil bath for 15 hours. It was then cooled to room temperature and diluted with EtOAc/water (40 mL each). Layers were separated after 10 minutes. The organic layer was washed with brine and concentrated. The residue was purified by silica gel chromatography using 5-30% EtOAc in hexanes to give tert-Butyl N-[3-bromo-6-(2,6-dimethylphenyl)pyrazin-2-yl]-N-tert-butoxycarbonyl-carbamate (˜1.5 g) that was used in the next deprotection step. ESI-MS m/z calc. 477.13, found 477.9 (M+1)⁺; Retention time 4.44 minutes. (LC method H).

### Step 3: 3-Bromo-6-(2,6-dimethyl-phenyl)-pyrazin-2-ylamine

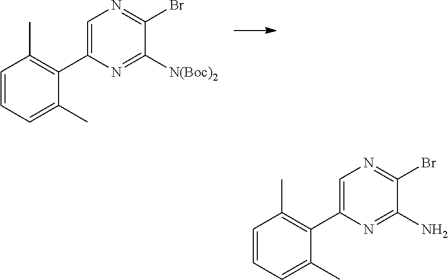

tert-Butyl N-[3-bromo-6-(2,6-dimethylphenyl)pyrazin-2-yl]-N-tert-butoxycarbonyl-carbamate from step 2 was dissolved in DCM (20 mL) and treated with TFA (20 mL). The mixture was stirred at room temperature for 6 hours. It was then concentrated thoroughly. The residue was purified by preparative HPLC to afford 3-bromo-6-(2,6-dimethyl-phenyl)-pyrazin-2-ylamine as a white solid (330 mg). ESI-MS m/z calc. 277.02, found 278.2 (M+1)⁺; Retention time 2.29 minutes. (LC method H). ¹H NMR (250 MHz, DMSO-d₆) 0 (ppm) 7.495 (s, 1H), 7.198 (t, J=3.75 Hz, 1H), 7.10 (d, J=3.75 Hz, 2H),6.770 (bs, 2H), 2.021 (s, 6H).

### Step 4: N-[3-Bromo-6-(2,6-dimethylphenyl)pyrazin-2-yl]benzenesulfonamide

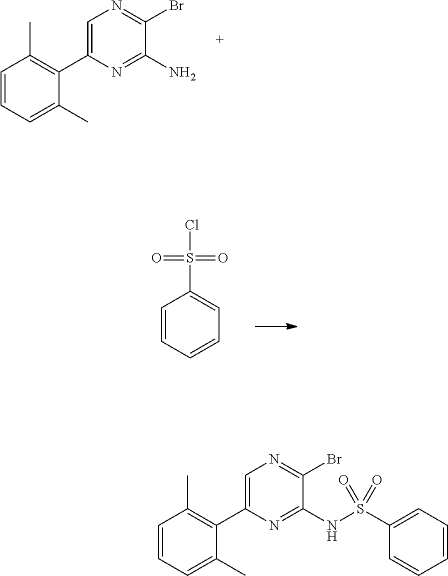 [SMILES: Cc1cccc(C)c1-c1cnc(Br)c(N)n1 ; O=S(=O)(Cl)c1ccccc1 ; Cc1cccc(C)c1-c1cnc(Br)c(NS(=O)(=O)c2ccccc2)n1]

In a dry glass vial was NaH (117.2 mg, 2.930 mmol) and THE (0.5 mL), and the white suspension was cooled to 0° C. with an ice-water bath and purged under nitrogen. 3-Bromo-6-(2,6-dimethylphenyl)pyrazin-2-amine (201.4 mg, 0.7110 mmol) was dissolved in THE (0.6 mL) and was added dropwise to the reaction mixture via syringe. The reaction mixture was stirred for 20 minutes. Benzenesulfonyl chloride (180 μL, 1.410 mmol) was added dropwise, and the reaction mixture was then warmed to room temperature for 30 minutes. It was cooled in an ice-water bath and HCl (3 mL of 1 M, 3.00 mmol) was added dropwise followed by ethyl acetate (10 mL). The organic later was dried over anhydrous sodium sulfate. Filtration and concentration in vacuo gave N-[3-bromo-6-(2,6-dimethylphenyl)pyrazin-2-yl]benzenesulfonamide (273.2 mg, 92%). ESI-MS m/z calc. 417.01465, found 418.32 (M+1)⁺; Retention time: 1.88 minutes; LC method A.

### Step 5: N-[3-(Benzylamino)-6-(2,6-dimethylphenyl)pyrazin-2-yl]benzenesulfonamide

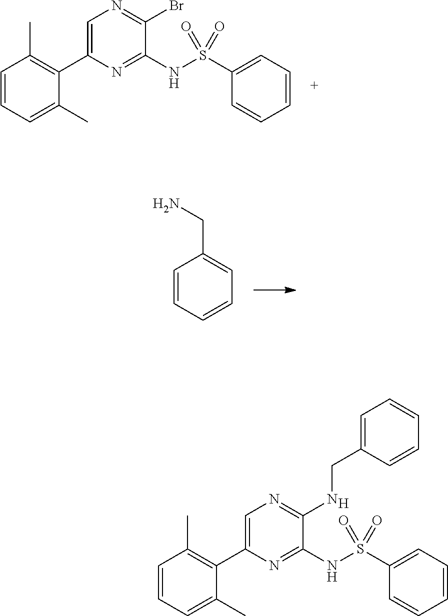

A dioxane (0.8 mL) mixture of N-[3-bromo-6-(2,6-dimethylphenyl)pyrazin-2-yl]benzenesulfonamide (10.2 mg, 0.02438 mmol), phenylmethanamine (8 μL, 0.07 mmol), [2-(2-aminoethyl)phenyl]-chloro-palladium; ditert-butyl-[2-(2,4,6-triisopropylphenyl)phenyl]phosphane (XPhos Pd G1)(6.8 mg, 0.0099 mmol), and sodium tert-butoxide (9.57 mg, 0.0996 mmol) was sparged with nitrogen under sonication for 1 minute and then stirred at room temperature for 10 minutes. The solution was filtered, and the resulting residue was dissolved in 0.8 mL DMSO and purified by reverse phase chromatography using a 15 minute gradient of 1% MeCN in water to 99% MeCN with 0.05 N HCl modifier to give N-[3-(benzylamino)-6-(2,6-dimethylphenyl)pyrazin-2-yl]benzenesulfonamide (4.5 mg, 42%). ¹H NMR (400 MHz, DMSO-d₆) δ 10.58 (s, 1H), 7.80 (d, J=7.8 Hz, 2H), 7.59 (d, J=10.0 Hz, 2H), 7.50-7.22 (m, 7H), 7.22-7.10 (m, 1H), 7.04 (d, J=7.6 Hz, 3H), 4.56 (d, J=5.2 Hz, 2H), 1.77 (s, 6H). ESI-MS m/z calc. 444.162, found 445.49 (M+1)⁺; Retention time: 2.12 minutes (LC method A).

### Example 31: Preparation of Compound 120

### Step 1: N-(3-Oxo-4,6-diphenyl-pyrazin-2-yl)benzenesulfonamide

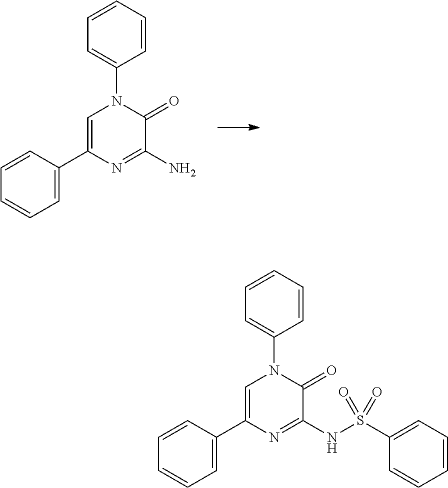 [SMILES: Nc1nc(-c2ccccc2)cn(-c2ccccc2)c1=O ; O=c1c(NS(=O)(=O)c2ccccc2)nc(-c2ccccc2)cn1-c1ccccc1]

To a solution of 3-amino-1,5-diphenyl-pyrazin-2-one (19 mg, 0.07216 mmol) in DMF (400 μL) was added NaH (approximately 7.215 mg of 60% w/w, 0.1804 mmol), and the reaction mixture was stirred at room temperature for 10 minutes. To this solution was added PhSO₂Cl (approximately 14.02 mg, 10.13 μL, 0.07938 mmol) and the reaction mixture was stirred at room temperature for 60 minutes. The reaction mixture was diluted with DMSO and purified by HPLC (1-99% ACN in water (HCl modifier)) to give N-(3-oxo-4,6-diphenyl-pyrazin-2-yl)benzenesulfonamide (8 mg, 27%) as a white solid. ESI-MS m/z calc. 403.09906, found 404.2 (M+1)⁺; Retention time: 1.69 minutes; LC method A.

### Example 32: Preparation of Compound 121

### Step 1: N-[6-(2,6-Dimethylphenyl)-3-phenoxy-pyrazin-2-yl]benzenesulfonamide

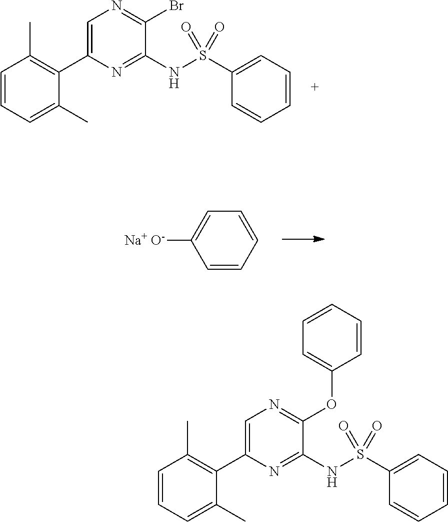

A NMP (1 mL) mixture of Cs₂CO₃ (107.2 mg, 0.3290 mmol), N-[3-bromo-6-(2,6-dimethylphenyl)pyrazin-2-yl]benzenesulfonamide (41.1 mg, 0.0983 mmol), and sodium phenoxide (36.3 mg, 0.313 mmol) was heated to 110° C. for 16 hours and then cooled to room temperature. The solution was filtered, and the resulting residue was dissolved in 0.8 mL MeOH and purified by reverse phase chromatography using a 15 minute gradient of 1% MeCN in water to 99% MeCN (HCl modifier) to give N-[6-(2,6-dimethylphenyl)-3-phenoxy-pyrazin-2-yl]benzenesulfonamide (8.9 mg, 21%). ¹H NMR (400 MHz, DMSO-d₆) δ 11.42 (s, 1H), 7.92-7.87 (m, 2H), 7.66 (s, 1H), 7.65-7.59 (m, 1H), 7.54-7.41 (m, 4H), 7.32-7.24 (m, 3H), 7.21 (dd, J=8.1, 6.9 Hz, 1H), 7.09 (d, J=7.5 Hz, 2H), 1.80 (s, 6H). ESI-MS m/z calc. 431.13037, found 432.1 (M+1)⁺; Retention time: 2.01 minutes (LC method A).

### Example 33: Preparation of Compound 122

### Step 1: N-[6-(2,6-Dimethylphenyl)-3-(4-methylpiperazin-1-yl)pyrazin-2-yl]benzenesulfonamide

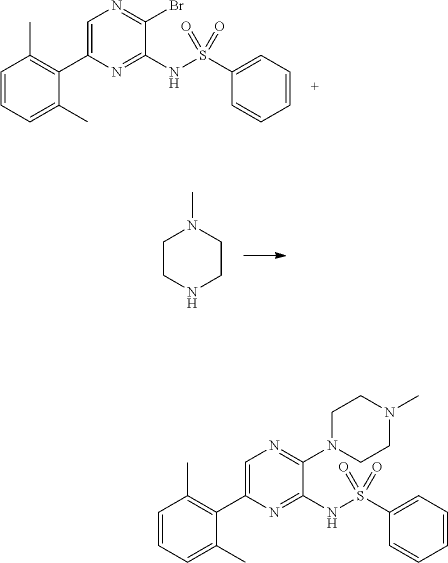

A NMP (0.5 mL) mixture of N-[3-bromo-6-(2,6-dimethylphenyl)pyrazin-2-yl]benzenesulfonamide (8.2 mg, 0.020 mmol), 1-methylpiperazine (6.3 mg, 0.06290 mmol) and Cs₂CO₃ (50.5 mg, 0.155 mmol) was stirred at 110° C. for 16 hours and then cooled to room temperature. The solution was filtered and the resulting residue dissolved in 0.8 mL DMSO, and purified by reverse phase chromatography using a 15 minutes gradient of 1% MeCN in water to 99% MeCN (HCl modifier) to give N-[6-(2,6-dimethylphenyl)-3-(4-methylpiperazin-1-yl)pyrazin-2-yl]benzenesulfonamide (hydrochloride salt) (2.3 mg, 24%). 1H NMR (400 MHz, DMSO-d6) δ 10.80 (s, 1H), 10.24 (s, 1H), 7.89 (s, 1H), 7.85-7.74 (m, 2H), 7.65-7.53 (m, 1H), 7.52-7.34 (m, 2H), 7.26-7.14 (m, 1H), 7.07 (d, J=7.6 Hz, 2H), 3.99-3.80 (m, 2H), 3.65-3.48 (m, 2H), 3.29-3.17 (m, 4H), 2.88 (d, J=4.4, 2.0 Hz, 3H), 1.72 (s, 6H). ESI-MS m/z calc. 437.18854, found 438.49 (M+1)+; Retention time: 1.38 minutes; LC method A.

### Example 34: Preparation of Compound 124

### Step 1: N-(4-isopropoxy-6-phenyl-1,3,5-triazin-2-yl)benzenesulfonamide

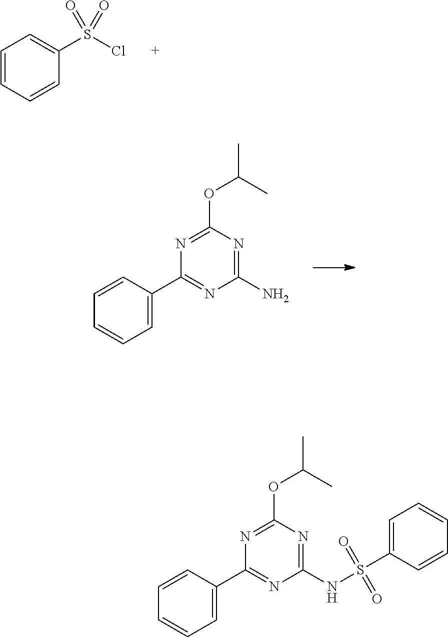 [SMILES: O=S(=O)(Cl)c1ccccc1 ; CC(C)Oc1nc(N)nc(-c2ccccc2)n1 ; CC(C)Oc1nc(NS(=O)(=O)c2ccccc2)nc(-c2ccccc2)n1]

NaH (4.778 mg, 0.1991 mmol) was added to 4-isopropoxy-6-phenyl-1,3,5-triazin-2-amine (approximately 22.93 mg, 0.09956 mmol) in DMF (1 mL). The mixture was stirred at room temperature for 15 minutes. Benzenesulfonyl chloride (35.17 mg, 25.41 μL, 0.1991 mmol) was added, and the reaction mixture was stirred at 150° C. for 1 hour. The reaction mixture was filtered and purified by reverse phase HPLC (HCl modifier, 25-75% ACN-H₂O) to give N-(4-isopropoxy-6-phenyl-1,3,5-triazin-2-yl)benzenesulfonamide (12.3 mg). ¹H NMR (400 MHz, DMSO) δ 12.47 (s, 1H), 8.24 (d, J=8.1 Hz, 2H), 8.03 (d, J=7.9 Hz, 2H), 7.71-7.60 (m, 4H), 7.59-7.50 (m, 2H), 5.18 (hept, J=6.0 Hz, 1H), 1.29 (d, J=6.2 Hz, 6H). ESI-MS m/z calc. 370.10995, found 371.0 (M+1)⁺; Retention time: 1.76 minutes; LC method A.

### Example 35: Preparation of Compound 125

### Step 1: 2-Chloro-4-phenoxy-6-phenyl-1,3,5-triazine

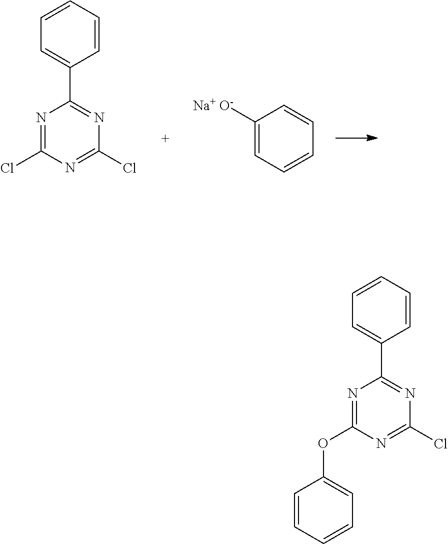

2,4-Dichloro-6-phenyl-1,3,5-triazine (300 mg, 1.327 mmol) was mixed with sodium phenoxide (approximately 184.8 mg, 1.592 mmol) in THE (3 mL) under N₂, and the reaction was allowed to stir for 16 hours at room temperature. The mixture was diluted with 100 mL of water and extracted with EtOAc (3×50 mL), all organics were combined and washed with brine, dried over Na₂SO₄, and concentrated under reduced pressure. The residue was dissolved in a 1:5 mixture of EtOH:EtOAc (6 mL total) and purified by chromatography using 0-30% of EtOAc in hexanes over 30 minutes. The compound was further purified using SFC: Column: Princeton 2-EP (250×21.2 mm), 5 μm, Mobile phase: 10% MeOH (No Modifier), 90% CO₂, 70.0 mL/min to give the desired product as white solid: 2-chloro-4-phenoxy-6-phenyl-1,3,5-triazine (83.6 mg, 22%).

### Step 2: N-(4-phenoxy-6-phenyl-1,3,5-triazin-2-yl)benzenesulfonamide

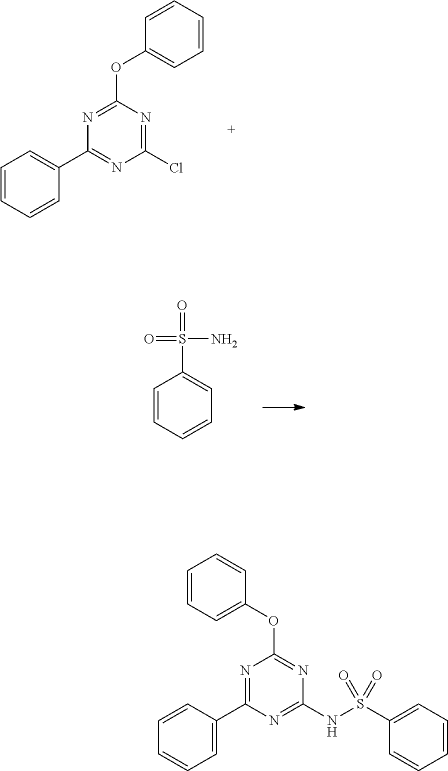 [SMILES: Clc1nc(Oc2ccccc2)nc(-c2ccccc2)n1 ; NS(=O)(=O)c1ccccc1 ; O=S(=O)(Nc1nc(Oc2ccccc2)nc(-c2ccccc2)n1)c1ccccc1]

Nitrogen was bubbled through a mixture of 2-chloro-4-phenoxy-6-phenyl-1,3,5-triazine (20 mg, 0.07049 mmol), benzenesulfonamide (approximately 33.25 mg, 0.2115 mmol), (5-diphenylphosphanyl-9,9-dimethyl-xanthen-4-yl)-diphenyl-phosphane (approximately 6.116 mg, 0.01057 mmol), diacetoxypalladium (approximately 1.187 mg, 0.005287 mmol) and cesium carbonate (approximately 45.94 mg, 0.1410 mmol) in dioxane (500.0 μL) for 25 minutes at room temperature. The reaction mixture was capped and stirred at 100° C. for 1 hour. The reaction mixture was filtered and subjected to HPLC using 20-80% ACN in water (0.05% HCl modifier) over 15 minutes. The desired fractions were collected and concentrated to give the desired product as a white solid. N-(4-phenoxy-6-phenyl-1,3,5-triazin-2-yl)benzenesulfonamide (3.1 mg). ¹H NMR (400 MHz, DMSO) δ 12.58 (bs, 1H), 8.19-8.13 (m, 2H), 7.81-7.75 (m, 2H), 7.66-7.60 (m, 2H), 7.50-7.56 (m, 6H), 7.40-7.34 (m, 1H), 7.31-7.26 (m, 2H). ESI-MS m/z calc. 404.0943, found 405.4 (M+1)⁺; Retention time: 2.58 minutes. (LC method I).

### Example 36: Preparation of Compound 126

### Step 1: 3-Nitro-N-(4-phenoxy-6-phenyl-1,3,5-triazin-2-yl)benzenesulfonamide

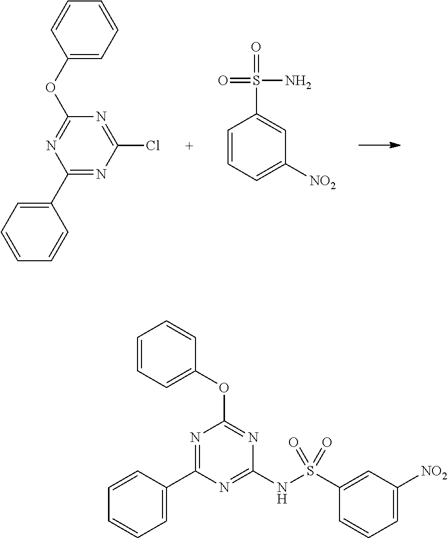 [SMILES: Clc1nc(Oc2ccccc2)nc(-c2ccccc2)n1 ; NS(=O)(=O)c1cccc([N+](=O)[O-])c1 ; O=[N+]([O-])c1cccc(S(=O)(=O)Nc2nc(Oc3ccccc3)nc(-c3ccccc3)n2)c1]

Nitrogen was bubbled through a mixture of 2-chloro-4-phenoxy-6-phenyl-1,3,5-triazine (42 mg, 0.1480 mmol), 3-nitrobenzenesulfonamide (approximately 89.77 mg, 0.4440 mmol), (5-diphenylphosphanyl-9,9-dimethyl-xanthen-4-yl)-diphenyl-phosphane (approximately 12.85 mg, 0.02220 mmol), diacetoxypalladium (approximately 2.492 mg, 0.01110 mmol) and cesium carbonate (approximately 96.44 mg, 0.2960 mmol) in dioxane (1.050 mL) for 25 minutes at room temperature. The reaction mixture was capped and stirred at 100° C. for 1 hour. The mixture was filtered and evaporated, and the residue was dissolved in MeOH and subjected to HPLC using 1-99% ACN in water (0.05% HCl modifier) over 15 minutes. The desired fractions were evaporated, and the product was used for the next step without further purification.

### Step 2: 3-amino-N-(4-phenoxy-6-phenyl-1,3,5-triazin-2-yl)benzenesulfonamide

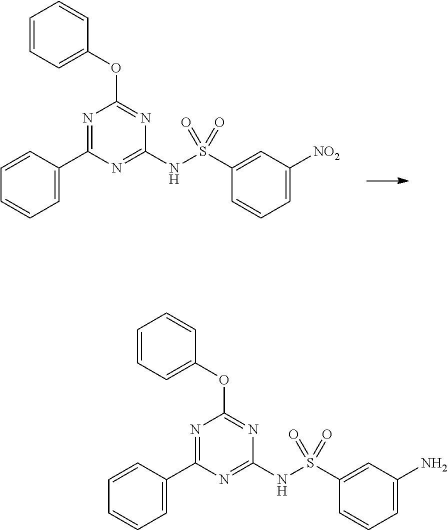 [SMILES: O=[N+]([O-])c1cccc(S(=O)(=O)Nc2nc(Oc3ccccc3)nc(-c3ccccc3)n2)c1 ; Nc1cccc(S(=O)(=O)Nc2nc(Oc3ccccc3)nc(-c3ccccc3)n2)c1]

Iron powder (approximately 2.485 mg, 0.04450 mmol) and HCl (approximately 7.417 μL of 6 M, 0.04450 mmol) were added to 3-nitro-N-(4-phenoxy-6-phenyl-1,3,5-triazin-2-yl)benzenesulfonamide (20 mg, 0.04450 mmol) in THE (249.70 μL) and EtOH (124.2 μL). The mixture was stirred at 95° C. for 30 minutes. The mixture was filtered and purified by HPLC using 1-99% ACN in water (0.05% HCl modifier) over 15 minutes. The desired fractions were evaporated to produce the desired product as white solid. 3-amino-N-(4-phenoxy-6-phenyl-1,3,5-triazin-2-yl)benzenesulfonamide (hydrochloride salt) (6.5 mg, 32%) ESI-MS m/z calc. 419.10522, found 421.3 (M+2)⁺; Retention time: 2.03 minutes. (LC method I). ¹H NMR (400 MHz, DMSO) δ 12.40 (s, 1H), 8.30-7.99 (m, 2H), 7.66-7.58 (m, 1H), 7.57-7.44 (m, 4H), 7.36-7.26 (m, 3H), 7.21 (t, J=2.0 Hz, 1H), 7.15 (t, J=7.9 Hz, 1H), 6.96 (d, J=7.4 Hz, 1H), 6.77 (dd, J=8.1, 2.1 Hz, 1H).

### Example 37: Preparation of Compound 127, Compound 128, and Compound 129

### Step 1: N-(6-chloro-2-phenoxy-pyrimidin-4-yl)benzenesulfonamide (Compound 127)

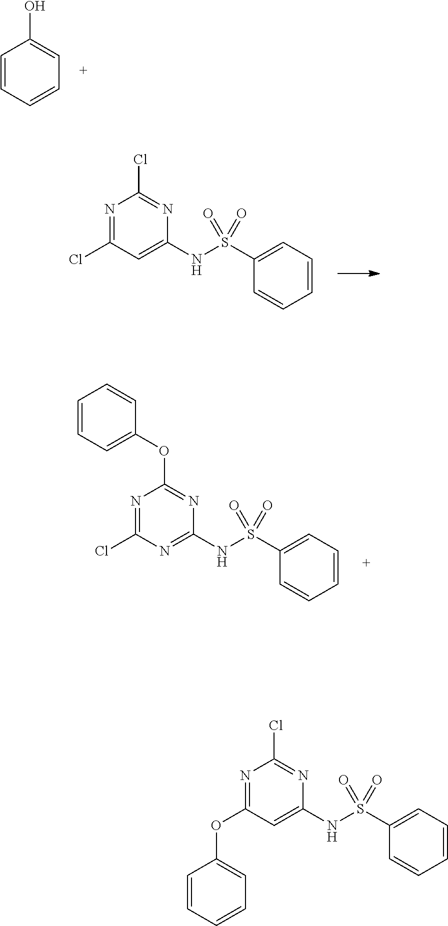 [SMILES: Oc1ccccc1 ; O=S(=O)(Nc1cc(Cl)nc(Cl)n1)c1ccccc1 ; O=S(=O)(Nc1nc(Cl)nc(Oc2ccccc2)n1)c1ccccc1 ; O=S(=O)(Nc1cc(Oc2ccccc2)nc(Cl)n1)c1ccccc1]

A mixture of phenol (300 μL, 3.379 mmol), carbonate (203 mg, 3.383 mmol), N-(2,6-dichloropyrimidin-4-yl)benzenesulfonamide (1.03 g, 3.386 mmol) in DMSO (5 mL) was heated at 100° C. overnight. The reaction mixture was left to stir overnight at 110° C. The reaction mixture was cooled down to room temperature. The reaction mixture was filtered and purified by reverse phase HPLC (HCl modifier, 10-60% ACN-H₂O) to give 2 products with the same mass. The major peak which eluted earlier on HPLC was N-(6-chloro-2-phenoxy-pyrimidin-4-yl)benzenesulfonamide (470 mg, 38%). ¹H NMR (400 MHz, DMSO) δ 12.16 (s, 1H), 7.64 (t, J=7.4 Hz, 1H), 7.56 (d, J=7.7 Hz, 2H), 7.54-7.44 (m, 4H), 7.35 (t, J=7.4 Hz, 1H), 7.18 (d, J=7.6 Hz, 2H), 6.67 (s, 1H). ESI-MS m/z calc. 361.02878, found 362.0 (M+1)⁺; Retention time: 1.59 minutes, (LC method A). The later eluting isomer was N-(2-chloro-6-phenoxy-pyrimidin-4-yl)benzenesulfonamide (50 mg, 3%). ¹H NMR (400 MHz, DMSO) δ 12.22 (s, 1H), 7.91 (d, J=7.6 Hz, 2H), 7.73 (t, J=7.2 Hz, 1H), 7.64 (t, J=7.7 Hz, 2H), 7.49 (t, J=7.9 Hz, 3H), 7.34 (t, J=7.3 Hz, 1H), 7.20 (d, J=7.8 Hz, 2H), 6.67 (s, 1H), 6.32 (s, 1H). ESI-MS m/z calc. 361.02878, found 362.0 (M+1)⁺; Retention time: 1.64 minutes, (LC method A).

### Step 2: N-[2-phenoxy-6-(2,2,4-trimethylpyrrolidin-1-yl)pyrimidin-4-yl]benzenesulfonamide (Compound 128) and N-[6-phenoxy-2-(2,2,4-trimethylpyrrolidin-1-yl)pyrimidin-4-yl]benzenesulfonamide (Compound 129)

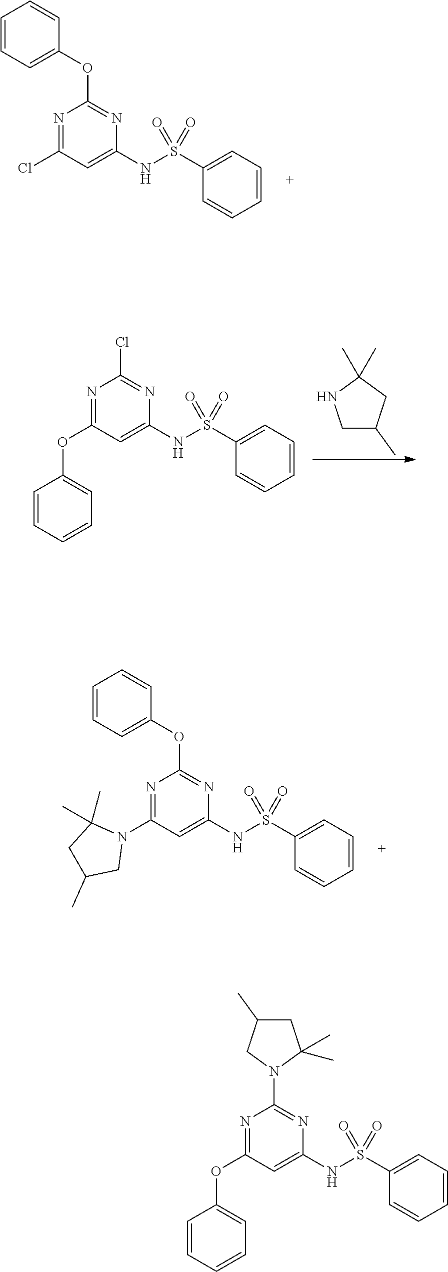

A mixture of N-(6-chloro-2-phenoxy-pyrimidin-4-yl)benzenesulfonamide (approximately 71.46 mg, 0.1975 mmol), 2,2,4-trimethylpyrrolidine (40 mg, 0.3534 mmol), K₂CO₃ (100 mg, 0.7236 mmol), and CsF (60 mg, 0.3950 mmol) in DMSO (500 μL) was stirred at 130° C. overnight. The reaction was stirred overnight at 150° C. The reaction was further stirred overnight at 160° C. The reaction was filtered and purified on reverse phase HPLC (HCl modifier, 30-99% ACN-H₂O) to give N-[2-phenoxy-6-(2,2,4-trimethylpyrrolidin-1-yl)pyrimidin-4-yl]benzenesulfonamide (10 mg). ¹H NMR (400 MHz, DMSO) δ 11.16 (s, 1H), 7.84 (d, J=7.4 Hz, 1H), 7.67-7.49 (m, 4H), 7.43-7.38 (m, 2H), 7.28-7.19 (m, 1H), 7.16-7.06 (m, 2H), 5.73-5.50 (m, 1H), 2.79 (t, J=10.2 Hz, 1H), 2.24 (s, 1H), 1.89-1.57 (m, 1H), 1.55-1.14 (m, 3H), 1.08-0.96 (m, 8H). ESI-MS m/z calc. 438.17255, found 439.0 (M+1)⁺; Retention time: 1.97 minutes (LC method A).

In a separate vial, a mixture of N-(2-chloro-6-phenoxy-pyrimidin-4-yl)benzenesulfonamide (approximately 71.46 mg, 0.1975 mmol), 2,2,4-trimethylpyrrolidine (40 mg, 0.3534 mmol), K₂CO₃ (100 mg, 0.7236 mmol), and CsF (60 mg, 0.3950 mmol) in DMSO (500 μL) was reacted overnight at 150° C. The reaction was filtered and purified on reverse phase HPLC (HCl modifier, 30-99% ACN-H₂O) to give N-[6-phenoxy-2-(2,2,4-trimethylpyrrolidin-1-yl)pyrimidin-4-yl]benzenesulfonamide (20.6 mg, 24%). ¹H NMR (400 MHz, DMSO) δ 11.20 (s, 1H), 7.95-7.79 (m, 2H), 7.66 (d, J=7.3 Hz, 1H), 7.60 (dd, J=8.3, 6.5 Hz, 2H), 7.40 (t, J=7.7 Hz, 2H), 7.29-7.18 (m, 1H), 7.15-7.04 (m, 2H), 5.61 (d, J=45.2 Hz, 1H), 3.49 (t, J=9.2 Hz, 1H), 2.70-2.56 (m, 1H), 2.08 (s, 1H), 1.77 (dd, J=12.1, 6.1 Hz, 1H), 1.34 (t, J=11.9 Hz, 1H), 1.22 (s, 1H), 1.08-0.86 (m, 8H). ESI-MS m/z calc. 438.17255, found 439.0 (M+1)⁺; Retention time: 2.08 minutes (LC method A).

### Example 38: Preparation of N-(2-phenoxy-6-phenylpyrimidin-4-yl)benzenesulfonamide (Compound 130)

### Step 1: N-(2,6-Dichloropyrimidin-4-yl)benzenesulfonamide

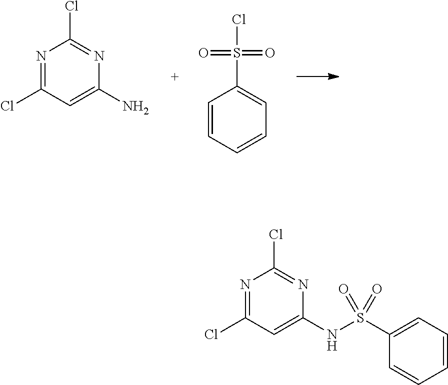 [SMILES: Nc1cc(Cl)nc(Cl)n1 ; O=S(=O)(Cl)c1ccccc1 ; O=S(=O)(Nc1cc(Cl)nc(Cl)n1)c1ccccc1]

To a solution of 2,6-dichloropyrimidin-4-amine (approximately 5.000 g, 30.49 mmol) in DMF (64.63 mL) was added sodium hydride (approximately 1.585 g of 60% w/w, 39.64 mmol) at 0° C., and the reaction was stirred for 10 minutes at 0° C. To this mixture was added dropwise benzenesulfonyl chloride (approximately 6.463 g, 4.670 mL, 36.59 mmol) and the reaction was stirred at 0° C. for 10 minutes. The reaction mixture was slowly poured into ice water. It was acidified with 1 N HCl and extracted with ethyl acetate (2×30 mL). The organic layer was separated, dried over Na₂SO₄, and concentrated, and the residue was purified by silica gel chromatography using a gradient of ethyl acetate/hexane. The product eluted around ˜25% ethyl acetate to give N-(2,6-dichloropyrimidin-4-yl)benzenesulfonamide (3.8 g, 34%) as a white solid. ¹H NMR (400 MHz, DMSO) δ 8.00 (d, J=7.6 Hz, 2H), 7.73 (t, J=7.3 Hz, 1H), 7.65 (t, J=7.6 Hz, 2H), 6.99 (s, 1H). ESI-MS m/z calc. 302.9636, found 304.0 (M+1)⁺; Retention time: 1.38 minutes (LC method A).

### Step 2: N-(6-chloro-2-phenyl-pyrimidin-4-yl)benzenesulfonamide and N-(2-chloro-6-phenyl-pyrimidin-4-yl)benzenesulfonamide

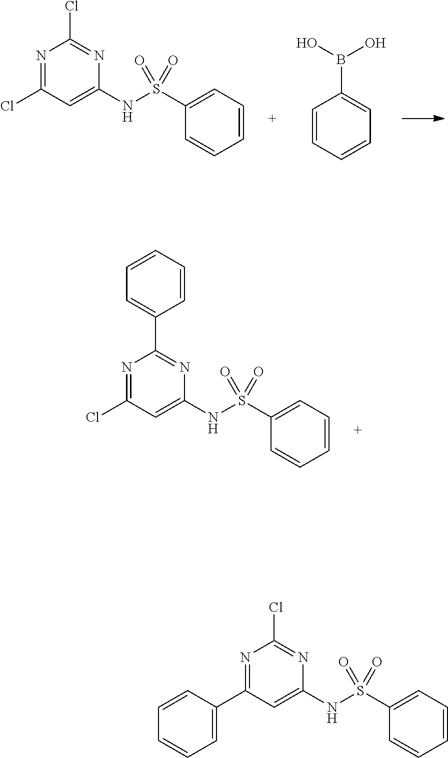 [SMILES: O=S(=O)(Nc1cc(Cl)nc(Cl)n1)c1ccccc1 ; OB(O)c1ccccc1 ; O=S(=O)(Nc1cc(Cl)nc(-c2ccccc2)n1)c1ccccc1 ; O=S(=O)(Nc1cc(-c2ccccc2)nc(Cl)n1)c1ccccc1]

To a mixture of N-(2,6-dichloropyrimidin-4-yl)benzenesulfonamide (500 mg, 1.627 mmol), phenylboronic acid (approximately 238.0 mg, 1.952 mmol) in DMF (7 mL) was added sodium carbonate (approximately 3.254 mL of 2 M, 6.508 mmol), Pd(dppf)Cl₂ (approximately 119.0 mg, 0.1627 mmol). The mixture was thoroughly flushed with nitrogen and heated at 90° C. for 1 hour. The reaction mixture was diluted with ethyl acetate and extracted with 1 N HCl. The organic layer was dried over Na₂SO₄ and concentrated under reduced pressure. The crude was purified on reverse phase HPLC (HCl modifier, 35-70% ACN-H₂O) to give:

Peak 1: N-(6-chloro-2-phenyl-pyrimidin-4-yl)benzenesulfonamide (24.8 mg, 4%). ¹H NMR (400 MHz, DMSO-d₆) δ 12.41 (s, 1H), 8.09-8.02 (m, 2H), 8.00-7.93 (m, 2H), 7.76-7.69 (m, 1H), 7.69-7.63 (m, 2H), 7.60-7.52 (m, 3H), 7.38 (s, 1H). ESI-MS m/z calc. 345.03387, found 346.1 (M+1)⁺; Retention time: 1.68 minutes (LC method A).

Peak 2: N-(2-chloro-6-phenyl-pyrimidin-4-yl)benzenesulfonamide (96.3 mg, 16%). ¹H NMR (400 MHz, DMSO-d₆) δ 12.27 (s, 1H), 8.13 (d, J=6.6 Hz, 2H), 8.06 (d, J=7.9 Hz, 2H), 7.71-7.59 (m, 3H), 7.59-7.48 (m, 3H), 6.91 (s, 1H). ESI-MS m/z calc. 345.03387, found 346.1 (M+1)⁺; Retention time: 1.74 minutes (LC method A).

### Step 3: N-(2-phenoxy-6-phenylpyrimidin-4-yl)benzenesulfonamide (Compound 130) and N-(6-phenoxy-2-phenyl-pyrimidin-4-yl)benzenesulfonamide

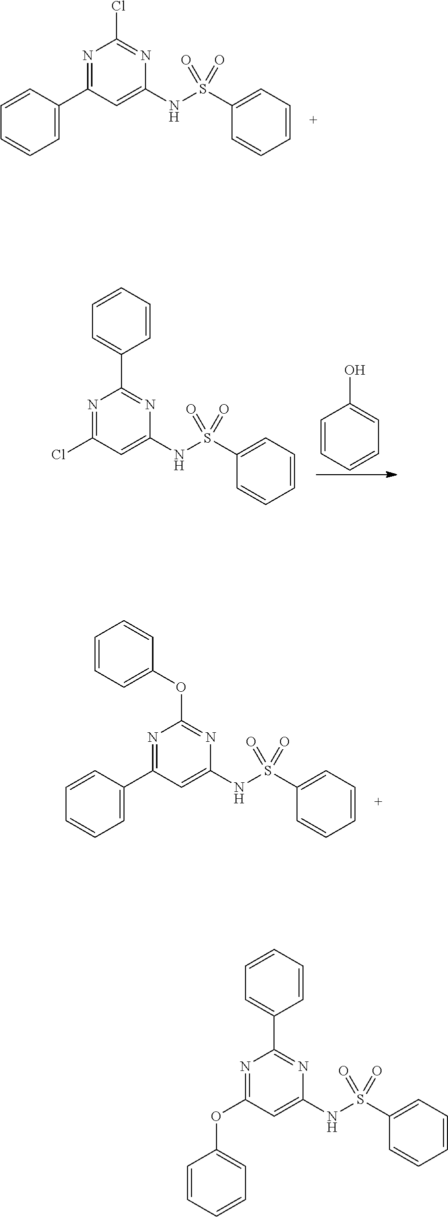

A mixture of phenol (approximately 8.165 mg, 7.703 μL, 0.08676 mmol), sodium carbonate (approximately 10.41 mg, 0.1735 mmol), and either N-(2-chloro-6-phenyl-pyrimidin-4-yl)benzenesulfonamide (approximately 20.00 mg, 0.05784 mmol) or, in a separated vial, N-(6-chloro-2-phenyl-pyrimidin-4-yl)benzenesulfonamide (approximately 20.00 mg, 0.05784 mmol) in DMSO (500 μL) was heated at 105° C. for 15 hours and then at 120° C. for 20 hours. After cooling down, each reaction mixture was filtered and purified by reverse phase HPLC (HCl modifier, 25-75% ACN-H₂O) to give two products:

N-(2-phenoxy-6-phenylpyrimidin-4-yl)benzenesulfonamide (8.6 mg). ¹H NMR (400 MHz, DMSO) δ 11.96 (s, 1H), 7.92-7.86 (m, 2H), 7.65 (d, J=7.4 Hz, 3H), 7.57-7.47 (m, 7H), 7.34 (t, J=7.1 Hz, 1H), 7.22 (d, J=8.5 Hz, 2H), 7.11 (s, 1H). ESI-MS m/z calc. 403.09906, found 404.0 (M+1)⁺; Retention time: 1.86 minutes (LC method A).

N-(6-phenoxy-2-phenyl-pyrimidin-4-yl)benzenesulfonamide (4.4 mg, 18%). ¹H NMR (400 MHz, DMSO) δ 11.86 (s, 1H), 8.04-7.97 (m, 4H), 7.69-7.60 (m, 3H), 7.47 (dt, J=23.4, 7.2 Hz, 5H), 7.33 (t, J=7.4 Hz, 1H), 7.24 (d, J=7.7 Hz, 2H), 6.25 (s, 1H). ESI-MS m/z calc. 403.09906, found 404.0 (M+1)⁺; Retention time: 1.93 minutes (LC method A).

### Example 39: Preparation of Compound 131

### Step 1: N-(2,6-Diphenylpyrimidin-4-yl)benzenesulfonamide

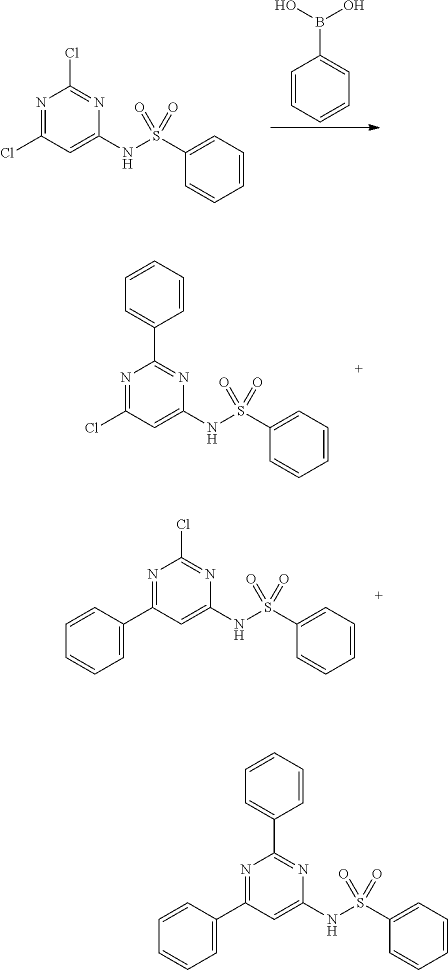 [SMILES: O=S(=O)(Nc1cc(Cl)nc(Cl)n1)c1ccccc1 ; OB(O)c1ccccc1 ; O=S(=O)(Nc1cc(Cl)nc(-c2ccccc2)n1)c1ccccc1 ; O=S(=O)(Nc1cc(-c2ccccc2)nc(Cl)n1)c1ccccc1 ; O=S(=O)(Nc1cc(-c2ccccc2)nc(-c2ccccc2)n1)c1ccccc1]

To a mixture of N-(2,6-dichloropyrimidin-4-yl)benzenesulfonamide (200 mg, 0.6510 mmol), phenylboronic acid (120 mg, 0.9842 mmol) in DMF (3 mL) was added sodium carbonate (1.5 mL of 2 M, 3.000 mmol) and Pd(dppf)Cl₂ (55 mg, 0.07517 mmol). The mixture was thoroughly flushed with nitrogen and heated at 90° C. for 3 hours. The reaction temperature was increased to 110° C. for 90 minutes. The reaction mixture was filtered and purified by reverse phase HPLC using 25-75% acetonitrile in water using HCl as modifier to give 3 products:

Peak 1: —N-(6-chloro-2-phenyl-pyrimidin-4-yl)benzenesulfonamide (20 mg). ¹H NMR (400 MHz, DMSO) δ 12.00 (s, 1H), 8.34-8.27 (m, 2H), 8.16-8.07 (m, 4H), 7.69-7.49 (m, 9H), 7.31 (s, 1H). ESI-MS m/z calc. 345.03387, found 346.0 (M+1)⁺; Retention time: 1.75 minutes (LC method A).

Peak 2: N-(2-chloro-6-phenyl-pyrimidin-4-yl)benzenesulfonamide (60 mg). ¹H NMR (400 MHz, DMSO) δ 12.22 (s, 1H), 8.10 (dd, J=27.1, 7.6 Hz, 4H), 7.74-7.49 (m, 6H), 6.90 (s, 1H). ESI-MS m/z calc. 345.03387, found 346.0 (M+1)⁺; Retention time: 1.78 minutes (LC method A).

Peak 3: N-(2,6-diphenylpyrimidin-4-yl)benzenesulfonamide (Compound 131). (55.7 mg). ¹H NMR (400 MHz, DMSO) δ 12.00 (s, 1H), 8.34-8.27 (m, 2H), 8.16-8.07 (m, 4H), 7.69-7.49 (m, 9H), 7.31 (s, 1H). ESI-MS m/z calc. 387.10416, found 388.0 (M+1)⁺; Retention time: 1.99 minutes (LC method A).

### Example 40: Preparation of Compound 132

### Step 1: N-(6-benzyloxy-2-phenyl-pyrimidin-4-yl)benzenesulfonamide

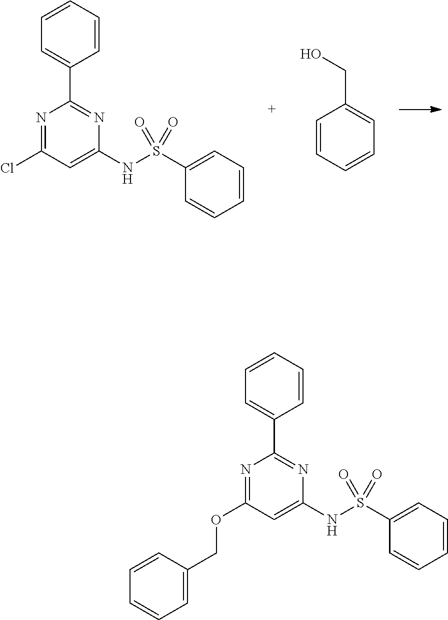 [SMILES: O=S(=O)(Nc1cc(Cl)nc(-c2ccccc2)n1)c1ccccc1 ; OCc1ccccc1 ; O=S(=O)(Nc1cc(OCc2ccccc2)nc(-c2ccccc2)n1)c1ccccc1]

To a solution of N-(6-chloro-2-phenyl-pyrimidin-4-yl)benzenesulfonamide (19 mg, 0.05494 mmol) and phenylmethanol (10 μL, 0.09664 mmol) in CH₃CN (500 μL) was added K₂CO₃ (26 mg, 0.1881 mmol), and the reaction mixture was stirred at 95° C. for 6 hours. The reaction mixture was filtered and purified on reverse phase HPLC (HCl modifier, 30-99% ACN-H₂O) to give N-(6-benzyloxy-2-phenyl-pyrimidin-4-yl)benzenesulfonamide (6.3 mg, 27%) ¹H NMR (400 MHz, DMSO-d₆) δ 12.01 (s, 1H), 8.01 (d, J=7.2 Hz, 2H), 7.98-7.93 (m, 2H), 7.67 (t, J=7.3 Hz, 1H), 7.60 (t, J=7.4 Hz, 2H), 7.53 (d, J=7.1 Hz, 3H), 7.43 (d, J=7.8 Hz, 2H), 7.41-7.30 (m, 3H), 7.08 (s, 1H), 5.31 (s, 2H). ESI-MS m/z calc. 417.11472, found 418.0 (M+1)⁺; Retention time: 1.9 minutes; LC method A.

### Example 41: Preparation of Compound 133

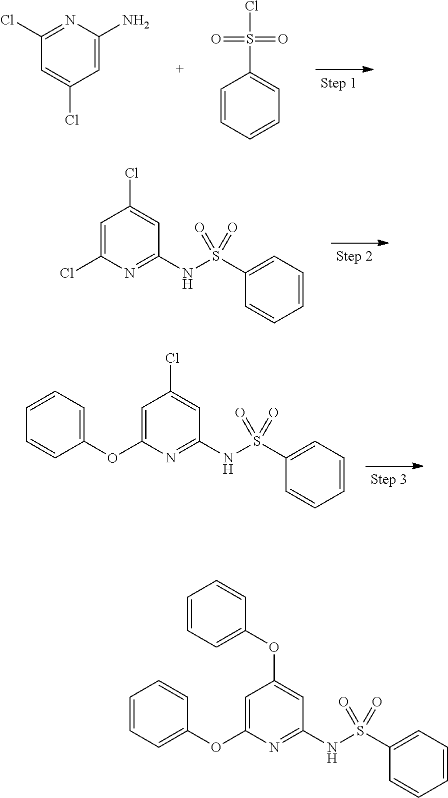 [SMILES: Nc1cc(Cl)cc(Cl)n1 ; O=S(=O)(Cl)c1ccccc1 ; O=S(=O)(Nc1cc(Cl)cc(Cl)n1)c1ccccc1 ; O=S(=O)(Nc1cc(Cl)cc(Oc2ccccc2)n1)c1ccccc1 ; O=S(=O)(Nc1cc(Oc2ccccc2)cc(Oc2ccccc2)n1)c1ccccc1]

### Step 1: N-(4,6-Dichloro-2-pyridyl)benzenesulfonamide

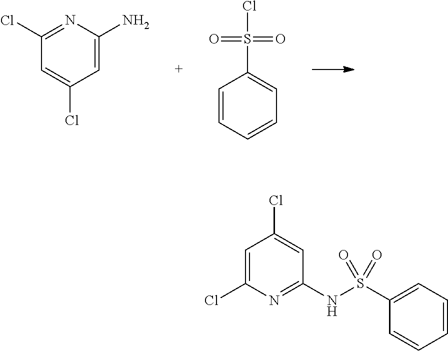 [SMILES: Nc1cc(Cl)cc(Cl)n1 ; O=S(=O)(Cl)c1ccccc1 ; O=S(=O)(Nc1cc(Cl)cc(Cl)n1)c1ccccc1]

To a solution of 4,6-dichloropyridin-2-amine (2 g, 12.27 mmol) in pyridine (10 mL) was added benzenesulfonyl chloride (approximately 4.334 g, 3.132 mL, 24.54 mmol) at 0° C. and the reaction was stirred at room temperature for 4 hours. All the solvents were evaporated and the residue was dissolved in ethyl acetate and washed with water. The organic layer was separated, dried over Na₂SO₄, concentrated and the residue was purified by silica gel chromatography using 0-40% ethyl acetate in hexanes to afford N-(4,6-dichloro-2-pyridyl)benzenesulfonamide (2.3 g, 62%). ¹H NMR (400 MHz, Chloroform-d) δ 7.99-7.90 (m, 2H), 7.65-7.58 (m, 1H), 7.53 (tt, J=7.8, 0.9 Hz, 2H), 7.39 (s, 1H), 7.32 (d, J=1.5 Hz, 1H), 7.02 (d, J=1.4 Hz, 1H). ESI-MS m/z calc. 301.96835, found 303.08 (M+1)⁺; Retention time: 0.61 minutes (LC method D).

### Step 2: N-(4-chloro-6-phenoxy-2-pyridyl)benzenesulfonamide

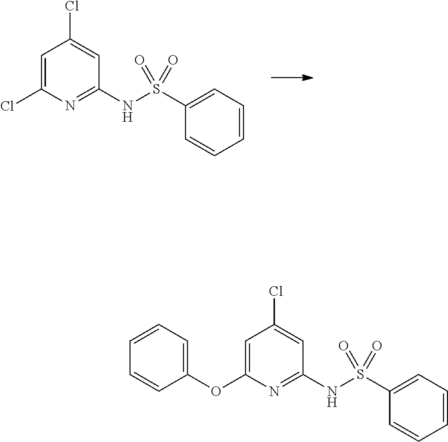 [SMILES: O=S(=O)(Nc1cc(Cl)cc(Cl)n1)c1ccccc1 ; O=S(=O)(Nc1cc(Cl)cc(Oc2ccccc2)n1)c1ccccc1]

To N-(4,6-dichloro-2-pyridyl)benzenesulfonamide (300 mg, 0.9896 mmol), sodium phenoxide (115 mg, 0.9906 mmol) and N,N-dimethyl formamide (5.4 mL) were added and the reaction was stirred at 110° C. for 14 h in a pressure vessel. 230 mg of sodium phenoxide was added to the reaction and heated at 200° C. for 2 hours. Water and EtOAc were added to the reaction and the two layers were separated. The aqueous layer was extracted with EtOAc (×3). The combined organic layer was dried over Na₂SO₄, filtered and the solvent was evaporated under reduced pressure. The crude product was purified on 80 g of silica gel utilizing a gradient of 0-50% ethyl acetate in hexane to yield N-(4-chloro-6-phenoxy-2-pyridyl)benzenesulfonamide (150 mg, 42%) as a viscous solid which on standing became a white solid. The product was not pure. A small amount of the product was dissolved in DMSO, filtered and purified using a reverse phase HPLC C₁₈ column and a dual gradient run from 1-99% mobile phase B over 30 minutes [(Mobile phase A=H₂O (5 mM HCl). Mobile phase B═CH₃CN)] to yield N-(4-chloro-6-phenoxy-2-pyridyl)benzenesulfonamide (150 mg, 42%). ESI-MS m/z calc. 360.03354, found 361.0 (M+1)⁺; Retention time: 0.72 minutes, LC method D.

### Step 3: N-(4,6-Diphenoxy-2-pyridyl)benzenesulfonamide

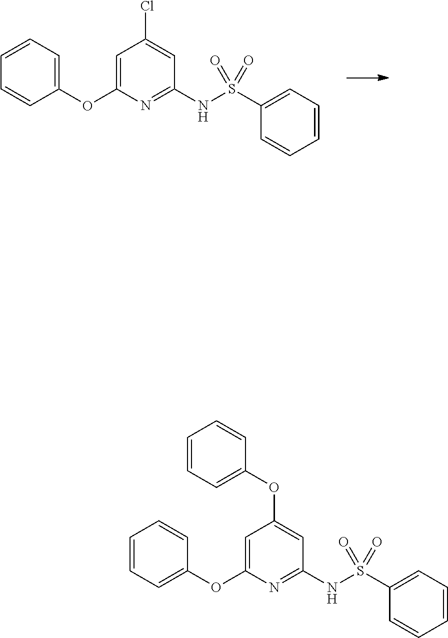 [SMILES: O=S(=O)(Nc1cc(Cl)cc(Oc2ccccc2)n1)c1ccccc1 ; O=S(=O)(Nc1cc(Oc2ccccc2)cc(Oc2ccccc2)n1)c1ccccc1]

To N-(6-chloro-4-phenoxy-2-pyridyl)benzenesulfonamide (50 mg, 0.1386 mmol), sodium phenoxide (49 mg, 0.4221 mmol) and N,N-dimethyl formamide (900.0 μL) were added and the reaction was stirred at 200° C. for 16 hours. More sodium phenoxide (49 mg, 0.4221 mmol) was added to the reaction and stirred at 200° C. for 5 hours. The crude product was filtered and purified using a reverse phase HPLC C₁₈ column and a dual gradient run from 1-99% mobile phase B over 30 minutes [(Mobile phase A=H₂O (5 mM HCl). Mobile phase B═CH₃CN)] to yield N-(4,6-diphenoxy-2-pyridyl)benzenesulfonamide as a viscous solid. ¹H NMR (400 MHz, DMSO-d₆) δ 10.99 (s, 1H), 7.62-7.54 (m, 1H), 7.52-7.38 (m, 8H), 7.37-7.29 (m, 1H), 7.28-7.22 (m, 1H), 7.18-7.13 (m, 2H), 7.11-7.03 (m, 2H), 6.15 (d, J=1.9 Hz, 1H), 6.04 (d, J=1.8 Hz, 1H). ESI-MS m/z calc. 418.09872, found 419.1 (M+1)⁺; Retention time: 1.99 minutes, LC method A.

### Example 42: Preparation of Compound 134

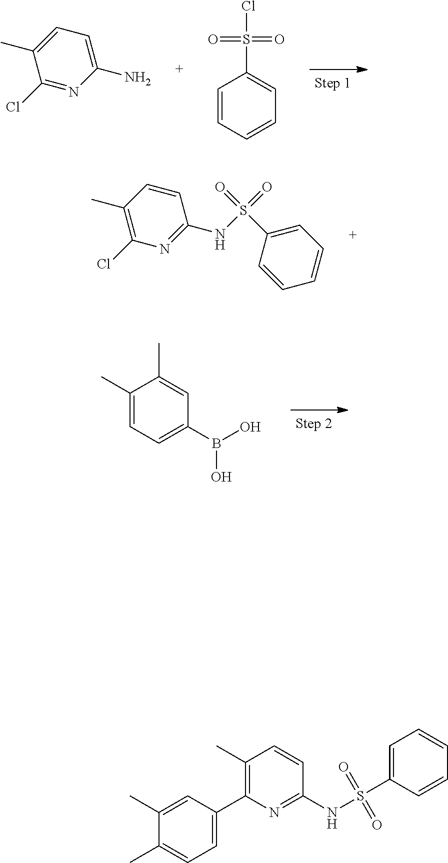

### Step 1: N-(6-chloro-5-methyl-2-pyridyl)benzenesulfonamide

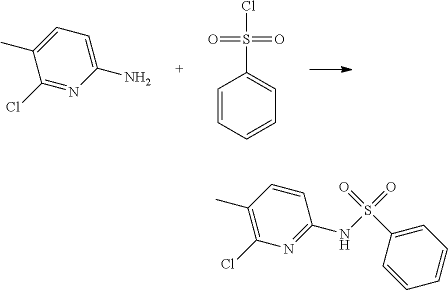

To a solution of 6-chloro-5-methyl-pyridin-2-amine (1.5 g, 10.52 mmol) in pyridine (10.00 mL) was added benzenesulfonyl chloride (approximately 2.044 g, 1.477 mL, 11.57 mmol) and the reaction was stirred at rt overnight. The reaction mixture was diluted with ethyl acetate and extracted with 1 N HCl. The organic layer was extracted with brine, dried over Na₂SO₄, concentrated under reduced pressure. The crude was purified by silica using a gradient of hexane/ethyl acetate. The product (beige solid) came out at ˜30% ethyl acetate. N-(6-chloro-5-methyl-2-pyridyl)benzenesulfonamide (3.04 g). ¹H NMR (400 MHz, DMSO) δ 11.27 (s, 1H), 8.59 (s, 1H), 7.93 (d, J=9.5 Hz, 2H), 7.82-7.76 (m, 1H), 7.66 (dd, J=11.5, 8.0 Hz, 2H), 7.59 (t, J=7.3 Hz, 2H), 7.39 (dd, J=10.5, 2.9 Hz, 1H), 7.00 (d, J=8.1 Hz, 1H), 2.18 (s, 3H), 2.09 (s, 1H). ESI-MS m/z calc. 282.02298, found 283.0 (M+1)⁺; Retention time: 1.43 minutes, LC method A.

### Step 2: N-[6-(3,4-Dimethylphenyl)-5-methyl-2-pyridyl]benzenesulfonamide

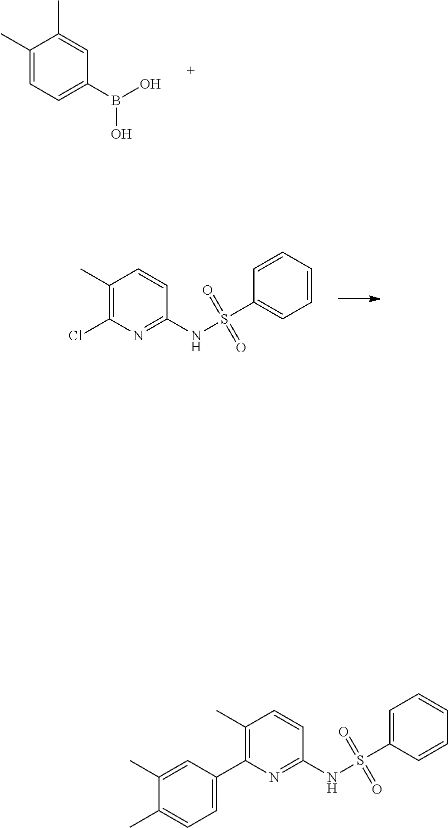 [SMILES: Cc1ccc(B(O)O)cc1C ; Cc1ccc(NS(=O)(=O)c2ccccc2)nc1Cl ; Cc1ccc(-c2nc(NS(=O)(=O)c3ccccc3)ccc2C)cc1C]

N-(6-chloro-5-methyl-2-pyridyl)benzenesulfonamide (25 mg, 0.08753 mmol), Pd(dppf)Cl₂, Na₂CO₃, and (3,4-dimethylphenyl)boronic acid (approximately 19.69 mg, 0.1313 mmol) in dioxane (1 mL) were added to a microwave vial. The vial was purged with nitrogen, capped and heated at 170-190° C. for 45 minutes. in a microwave oven. The crude was filtered and purified by HPLC utilizing a gradient of 25-75% acetonitrile in 5 mM aqueous HCl to give product N-[6-(3,4-dimethylphenyl)-5-methyl-2-pyridyl]benzenesulfonamide (13.9 mg, 54%). ESI-MS m/z calc. 352.12454, found 353.0 (M+1)⁺; Retention time: 1.75 minutes; LC method A. H NMR (400 MHz, DMSO) δ 10.97 (s, 1H), 7.89 (d, J=7.8 Hz, 2H), 7.73-7.49 (m, 4H), 7.18 (d, J=8.1 Hz, 1H), 7.08 (d, J=6.4 Hz, 2H), 6.95 (s, 1H), 2.26 (d, J=5.5 Hz, 6H), 2.17 (s, 3H).

### Example 43: Preparation of Compound 135

### Step 1: N-(6-chloro-4-methyl-2-pyridyl)benzenesulfonamide

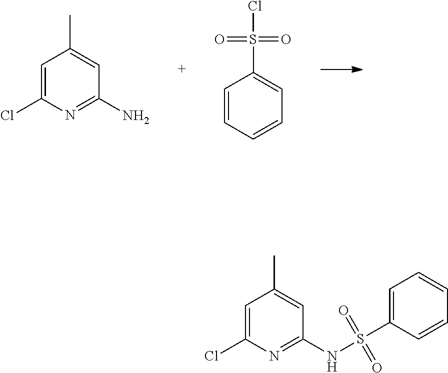

To a solution of 6-chloro-4-methyl-pyridin-2-amine (1.5 g, 10.52 mmol) in pyridine (15.00 mL) was added benzenesulfonyl chloride (approximately 2.044 g, 1.477 mL, 11.57 mmol) and the reaction was stirred at rt overnight. The reaction mixture was diluted with ethyl acetate, extracted with 1 N HCl. The organic layer was washed with brine, dried over Na₂SO₄, and concentrated under reduced pressure. The crude was purified by silica using a gradient of hexane/ethyl acetate. Product (off-white solid) came out ˜30% ethyl acetate. N-(6-chloro-4-methyl-2-pyridyl)benzenesulfonamide (3.04 g) ¹H NMR (400 MHz, DMSO) δ 11.34 (s, 1H), 8.58 (d, J=2.6 Hz, 1H), 7.95 (d, J=10.4 Hz, 2H), 7.79 (t, J=9.5 Hz, 1H), 7.71-7.55 (m, 3H), 7.44-7.34 (m, 1H), 6.96 (s, 1H), 6.84 (s, 1H), 2.24 (s, 3H), 2.12 (s, 1H), 2.09 (s, 1H). ESI-MS m/z calc. 282.02298, found 283.0 (M+1)⁺; Retention time: 1.43 minutes, LC method A.

### Step 2: N-[6-(2,5-Dimethylphenyl)-4-methyl-2-pyridyl]benzenesulfonamide

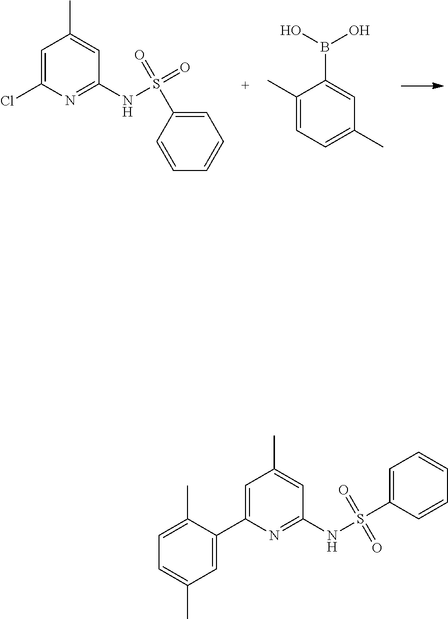

The compound was prepared in a manner analogous to that described above using commercially available (2,5-dimethylphenyl) boronic acid to give N-[6-(2,5-dimethylphenyl)-4-methyl-2-pyridyl]benzenesulfonamide (12.8 mg, 41%). ESI-MS m/z calc. 352.12454, found 353.0 (M+1)⁺; Retention time: 1.73 minutes; LC method A. ¹H NMR (400 MHz, DMSO) δ 11.01 (s, 1H), 7.88 (d, J=7.3 Hz, 2H), 7.65-7.57 (m, 1H), 7.53 (t, J=7.4 Hz, 2H), 7.11 (s, 2H), 6.90 (d, J=23.8 Hz, 3H), 2.28 (d, J=9.0 Hz, 6H), 2.09 (d, J=4.6 Hz, 3H).

### Example 44: Preparation of Compound 136

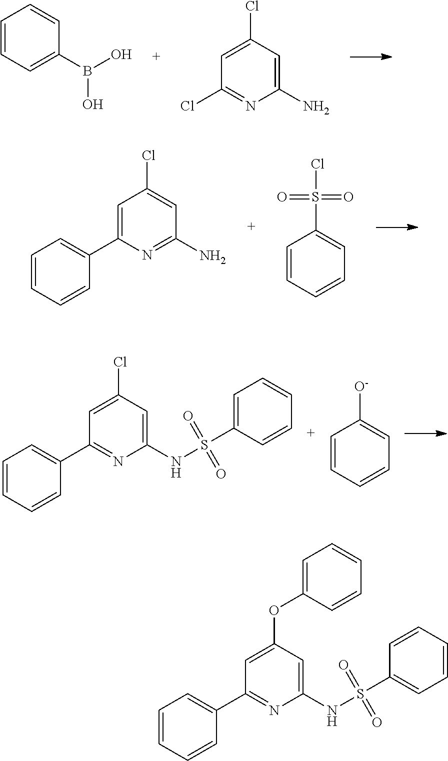

### Step 1: 4-Chloro-6-phenyl-pyridin-2-amine

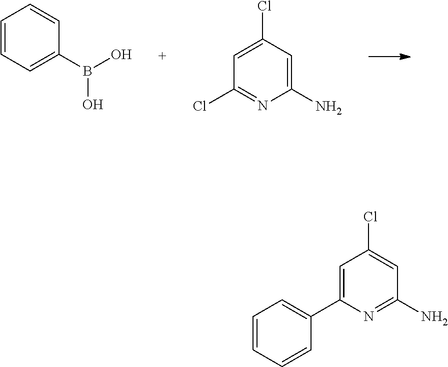 [SMILES: OB(O)c1ccccc1 ; Nc1cc(Cl)cc(Cl)n1 ; Nc1cc(Cl)cc(-c2ccccc2)n1]

A solution of phenylboronic acid (approximately 1.570 g, 12.88 mmol), 4,6-dichloropyridin-2-amine (approximately 2.000 g, 12.27 mmol), Cs₂CO₃ (approximately 9.996 g, 30.68 mmol), and Pd(dppf)Cl₂·DCM (approximately 496.1 mg, 0.6135 mmol) in DME (50 mL) and water (20 mL) was degassed by bubbling nitrogen through the reaction mixture for 10 minutes. The reaction mixture was then stirred at 80° C. for 16 hours. The reaction mixture was poured into water and extracted with EtOAc (×3). The organic extracts were combined, washed with brine, filtered through a short plug of silica gel, and evaporated to dryness. Purification by column chromatography (80 g silica; 0-30% EtOAc in hexanes) gave crude 4-chloro-6-phenyl-pyridin-2-amine (2.8 g, 84%). ESI-MS m/z calc. 204.04543, found 205.3 (M+1)⁺; Retention time: 0.36 minutes; LC method D.

### Step 2: N-(4-chloro-6-phenyl-2-pyridyl)benzenesulfonamide

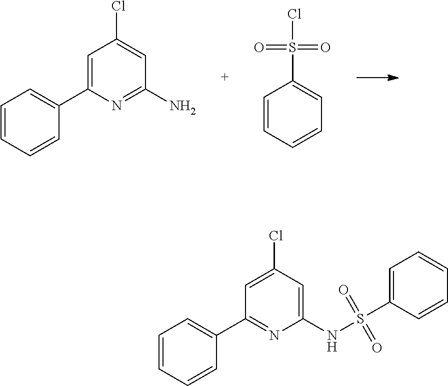 [SMILES: Nc1cc(Cl)cc(-c2ccccc2)n1 ; O=S(=O)(Cl)c1ccccc1 ; O=S(=O)(Nc1cc(Cl)cc(-c2ccccc2)n1)c1ccccc1]

To a solution of 4-chloro-6-phenyl-pyridin-2-amine (200 mg, 0.7329 mmol) in pyridine (2 mL) was added benzenesulfonyl chloride (95 μL, 0.7444 mmol), and the reaction was stirred at 200° C. for 35 minutes. EtOAc was added to the reaction and washed with water (×3). The organic layer was dried over Na₂SO₄, filtered, and concentrated to yield N-(4-chloro-6-phenyl-2-pyridyl)benzenesulfonamide (238 mg, 94%) as a brown viscous solid. The product was used in the next step without further purification. ESI-MS m/z calc. 344.03864, found 345.1 (M+1)⁺; Retention time: 0.7 minutes; LC method D.

### Step 3: N-(4-phenoxy-6-phenyl-2-pyridyl)benzenesulfonamide

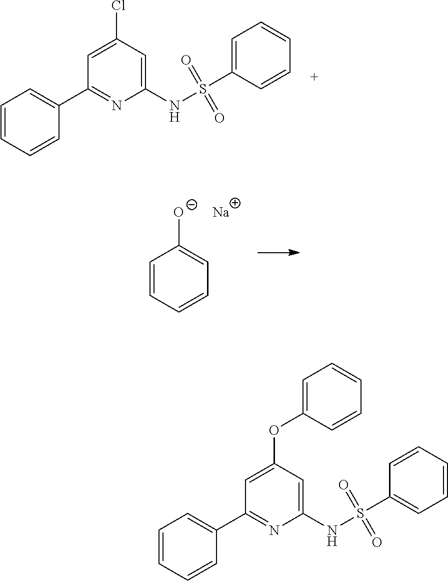 [SMILES: O=S(=O)(Nc1cc(Cl)cc(-c2ccccc2)n1)c1ccccc1 ; [O-]c1ccccc1 ; [Na+] ; O=S(=O)(Nc1cc(Oc2ccccc2)cc(-c2ccccc2)n1)c1ccccc1]

To N-(4-chloro-6-phenyl-2-pyridyl)benzenesulfonamide (106 mg, 0.3074 mmol), sodium phenoxide (73 mg, 0.6288 mmol) and DMF (1.3 mL) was added and the reaction was stirred at 200° C. for 4 hours. More sodium phenoxide (73 mg, 0.6288 mmol) was added to the reaction and it was heated at 200° C. for 16 hours. Water and EtOAc were added to the reaction, and the two layers were separated. The aqueous layer was extracted with EtOAc (×3). The combined organic layer was washed with water (×3), dried over Na₂SO₄, filtered and the solvent was evaporated under reduced pressure. The crude product was dissolved in DMSO, filtered, and purified using a reverse phase HPLC C₁₈ column and a dual gradient run from 1-99% mobile phase B over 30 minutes [(Mobile phase A=H₂O (5 mM HCl). Mobile phase B═CH₃CN)] to yield N-(4-phenoxy-6-phenyl-2-pyridyl)benzenesulfonamide (69.9 mg, 57%) as a cream solid. ¹H NMR (400 MHz, DMSO-d₆) δ 11.13 (s, 1H), 7.87 (s, 2H), 7.83-7.76 (m, 2H), 7.65-7.48 (m, 5H), 7.48-7.39 (m, 3H), 7.35 (t, J=7.4 Hz, 1H), 7.23-7.09 (m, 3H), 6.33 (d, J=2.0 Hz, 1H). ESI-MS m/z calc. 402.10382, found 403.2 (M+1)⁺; Retention time: 1.86 minutes, LC method A.

### Example 45: Preparation of Compound 137

### Step 1: N-[6-(2,3-Dimethylphenyl)-5-methyl-2-pyridyl]benzenesulfonamide

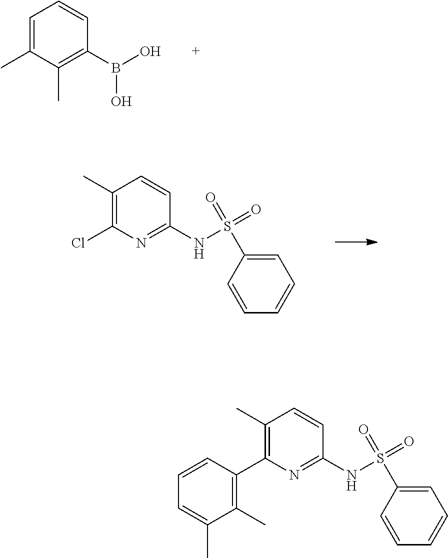 [SMILES: Cc1cccc(B(O)O)c1C ; "+" ; Cc1ccc(NS(=O)(=O)c2ccccc2)nc1Cl ; Cc1ccc(NS(=O)(=O)c2ccccc2)nc1-c1cccc(C)c1C]

The compound was prepared in a manner analogous to that described above using commercially available (2,3-dimethylphenyl)boronic acid to give N-[6-(2,3-dimethylphenyl)-5-methyl-2-pyridyl]benzenesulfonamide (10.9 mg, 42%). ESI-MS m/z calc. 352.12454, found 353.0 (M+1)⁺; Retention time: 1.67 minutes; LC method A. ¹H NMR (400 MHz, DMSO) δ 10.89 (s, 1H), 7.82 (d, J=7.1 Hz, 2H), 7.62 (dd, J=18.5, 7.8 Hz, 2H), 7.51 (t, J=7.6 Hz, 2H), 7.20 (d, J=7.6 Hz, 1H), 7.12 (dd, J=17.2, 10.2 Hz, 2H), 6.83 (s, 1H), 2.27 (s, 3H), 1.88 (s, 3H), 1.71 (s, 3H).

### Example 46: Preparation of Compound 138

### Step 1: N-[6-(2,5-Dimethylphenyl)-5-methyl-2-pyridyl]benzenesulfonamide

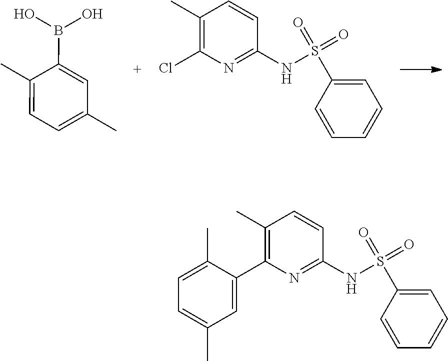

The compound was prepared in a manner analogous to that described above using commercially available (2,5-dimethylphenyl)boronic acid to give N-[6-(2,5-dimethylphenyl)-5-methyl-2-pyridyl]benzenesulfonamide (12.7 mg, 47%). ESI-MS m/z calc. 352.12454, found 353.0 (M+1)⁺; Retention time: 1.71 minutes; LC method A. ¹H NMR (400 MHz, DMSO) δ 10.87 (s, 1H), 7.81 (d, J=8.6 Hz, 2H), 7.62 (dd, J=19.8, 7.9 Hz, 2H), 7.51 (t, J=7.6 Hz, 2H), 7.19-7.02 (m, 3H), 6.81 (s, 1H), 2.27 (s, 3H), 1.90 (s, 3H), 1.78 (s, 3H).

### Example 47: Preparation of Compound 139

### Step 1: N-(6-Chloro-4,5-dimethyl-2-pyridyl)benzenesulfonamide

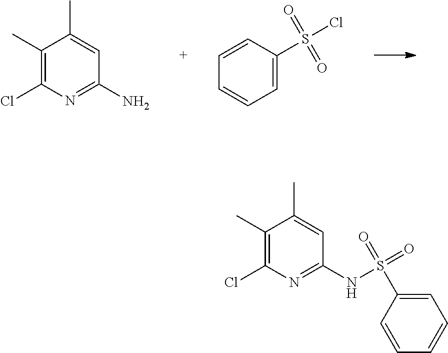

To a solution of 6-chloro-4,5-dimethyl-pyridin-2-amine (1.51 g, 9.642 mmol) in pyridine (15 mL) was added benzenesulfonyl chloride (1.4 mL, 10.97 mmol) and the reaction was stirred at room temperature overnight. The reaction mixture was diluted with ethyl acetate and extracted with 1 N HCl. The organic layer was washed with brine, dried over Na₂SO₄, and concentrated under reduced pressure. The crude was purified by silica using a gradient of hexanes/ethyl acetate. The product (beige solid) came out ˜30% ethyl acetate. N-(6-chloro-4,5-dimethyl-2-pyridyl)benzenesulfonamide (1.4692 g). ¹H NMR (400 MHz, DMSO) δ 11.15 (s, 1H), 7.92 (d, J=7.2 Hz, 2H), 7.61 (dt, J=24.6, 7.2 Hz, 3H), 6.88 (s, 1H), 2.23 (s, 3H), 2.14 (s, 3H). ESI-MS m/z calc. 296.03864, found 297.0 (M+1)⁺; Retention time: 1.53 minutes LC method A.

### Step 2: N-[6-(2,5-Dimethylphenyl)-4,5-dimethyl-2-pyridyl]benzenesulfonamide

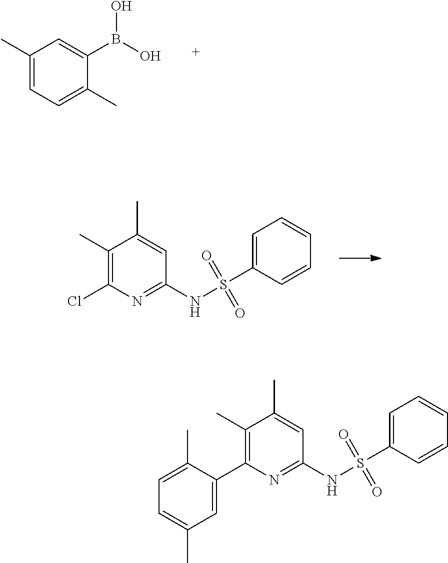 [SMILES: Cc1ccc(C)c(B(O)O)c1 ; "+" ; Cc1cc(NS(=O)(=O)c2ccccc2)nc(Cl)c1C ; Cc1ccc(C)c(-c2nc(NS(=O)(=O)c3ccccc3)cc(C)c2C)c1]

The compound was prepared in a manner analogous to that described above using commercially available (2,5-dimethylphenyl)boronic acid to give N-[6-(2,5-dimethylphenyl)-4,5-dimethyl-2-pyridyl]benzenesulfonamide (12.5 mg, 49%). ESI-MS m/z calc. 366.1402, found 367.0 (M+1)⁺; Retention time: 1.71 minutes; LC method A. ¹H NMR (400 MHz, DMSO) δ 7.82 (d, J=7.2 Hz, 2H), 7.59 (t, J=7.3 Hz, 1H), 7.50 (t, J=7.5 Hz, 2H), 7.15 (t, J=5.7 Hz, 2H), 7.01 (s, 1H), 6.80 (s, 1H), 2.26 (d, J=9.2 Hz, 6H), 1.80 (s, 6H).

### Example 48: Preparation of Compound 140

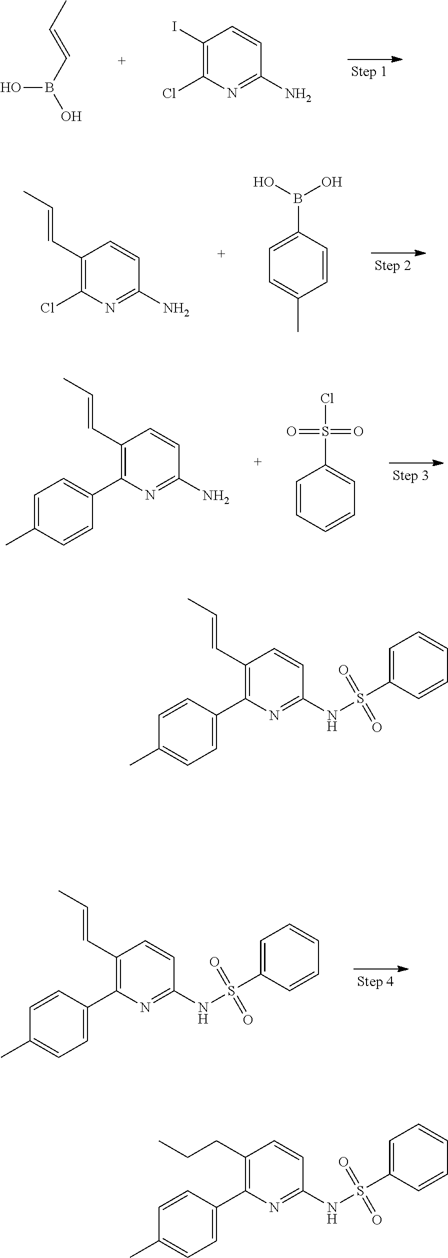

### Step 1: 6-Chloro-5-[(E)-prop-1-enyl]pyridin-2-amine

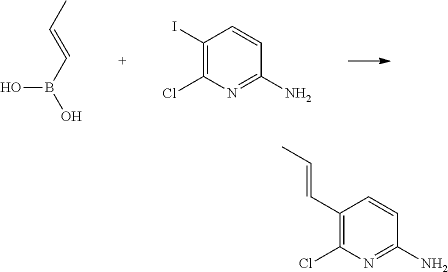

To 6-chloro-5-iodo-pyridin-2-amine (2 g, 7.860 mmol) was added [(E)-prop-1-enyl]boronic acid (1.4 g, 16.30 mmol), Fibre Cat 1032 (520 mg, 0.7951 mmol), N,N-dimethylformamide (58.00 mL) and sodium carbonate (8 mL of 2 M, 16.00 mmol). The reaction mixture was stirred at 110° C. for 18 hours. The reaction was filtered using ethyl acetate. Water was added to the reaction. The two layers were separated, and the aqueous layer was extracted with ethyl acetate (×3). The combined organic layer was dried over sodium sulfate, filtered and the solvent was removed under reduced pressure. The crude product was purified on 120 g of silica gel utilizing a gradient of 0-30% ethyl acetate in hexane to yield 6-chloro-5-[(E)-prop-1-enyl]pyridin-2-amine (667 mg, 50%) as a yellow solid. ESI-MS m/z calc. 168.04543, found 169.1 (M+1)⁺; Retention time: 1.14 minutes (LC method A).

### Step 2: 5-[(E)-prop-1-enyl]-6-(p-tolyl)pyridin-2-amine

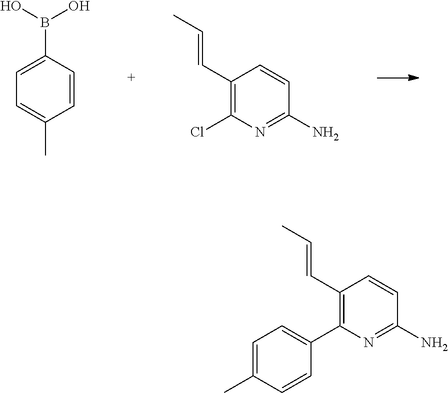

The mixture of 6-chloro-5-[(E)-prop-1-enyl]pyridin-2-amine (300.0 mg, 1.619 mmol), p-tolylboronic acid (242 mg, 1.780 mmol), Pd(dppf)Cl₂ (119 mg, 0.1626 mmol), and potassium carbonate (1.62 mL of 2 M, 3.240 mmol) in 1,2-dimethoxyethane (3.6 mL) was degassed by flow of nitrogen and stirred at 80° C. for 24 hours. EtOAc and water were added to the reaction and the two layers were separated. The organic layer was dried over Na₂SO₄, filtered through a plug of Celite, and concentrated. The crude product was purified on 80 g of silica gel utilizing a gradient of 0-30% ethyl acetate in hexane to yield 5-[(E)-prop-1-enyl]-6-(p-tolyl)pyridin-2-amine (321 mg, 88%) as a yellow viscous solid. ESI-MS m/z calc. 224.13135, found 225.2 (M+1)⁺; Retention time: 1.1 minutes, LC method A.

### Step 3: N-[5-[(E)-prop-1-enyl]-6-(p-tolyl)-2-pyridyl]benzenesulfonamide

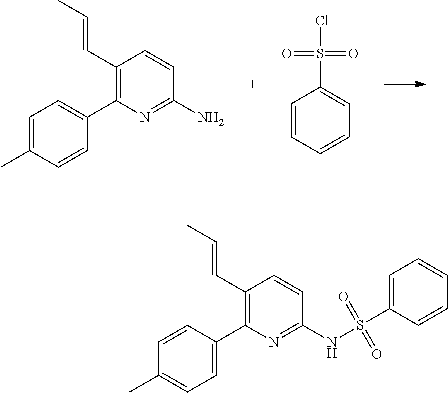

To a solution of 5-[(E)-prop-1-enyl]-6-(p-tolyl)pyridin-2-amine (50 mg, 0.2229 mmol) in pyridine (850 μL) was added benzenesulfonyl chloride (29 μL, 0.2272 mmol) and the reaction was stirred at 130° C. for 1 hour. EtOAc was added to the reaction and washed with water (×1). The organic layer was dried over Na₂SO₄, filtered, and concentrated. The crude product was dissolved in DMSO, filtered, and purified using a reverse phase HPLC C₁₈ column and a dual gradient run from 1-99% mobile phase B over 30 minutes [(Mobile phase A=H₂O (5 mM HCl). Mobile phase B═CH₃CN)] to yield N-[5-[(E)-prop-1-enyl]-6-(p-tolyl)-2-pyridyl]benzenesulfonamide (32.6 mg, 40%) as a yellow solid. ¹H NMR (400 MHz, DMSO-d₆) δ 11.10 (s, 1H), 7.93-7.79 (m, 3H), 7.69-7.59 (m, 1H), 7.58-7.51 (m, 2H), 7.26 (d, J=8.0 Hz, 2H), 7.20 (d, J=7.8 Hz, 2H), 6.97 (s, 1H), 6.33-6.01 (m, 2H), 2.37 (s, 3H), 1.75 (d, J=4.9 Hz, 3H). ESI-MS m/z calc. 364.12454, found 365.2 (M+1)⁺; Retention time: 1.88 minutes (LC method A).

### Step 4: N-[5-propyl-6-(p-tolyl)-2-pyridyl]benzenesulfonamide

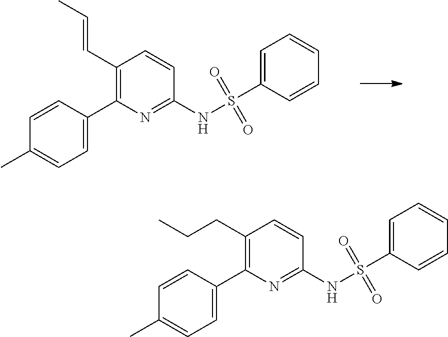 [SMILES: C/C=C/c1ccc(NS(=O)(=O)c2ccccc2)nc1-c1ccc(C)cc1 ; CCCc1ccc(NS(=O)(=O)c2ccccc2)nc1-c1ccc(C)cc1]

To a solution of N-[5-[(E)-prop-1-enyl]-6-(p-tolyl)-2-pyridyl]benzenesulfonamide (25 mg, 0.06859 mmol) in ethyl alcohol (1.5 mL) was added Pd/C (27 mg of 10% w/w, 0.02537 mmol) under N₂ atmosphere. The reaction was flushed with H₂, and the reaction was stirred under H₂ atmosphere for 1 hour. The crude product was filtered and purified using a reverse phase HPLC C₁₈ column and a dual gradient run from 1-99% mobile phase B over 30 minutes [(Mobile phase A=H₂O (5 mM HCl). Mobile phase B═CH₃CN)] to yield N-[5-propyl-6-(p-tolyl)-2-pyridyl]benzenesulfonamide (13.7 mg, 54%) as a colorless viscous solid. ¹H NMR (400 MHz, DMSO-d₆) δ 10.98 (s, 1H), 7.98-7.82 (m, 2H), 7.67-7.58 (m, 2H), 7.56-7.47 (m, 2H), 7.24 (d, J=7.9 Hz, 2H), 7.16 (d, J=8.0 Hz, 2H), 7.00 (d, J=8.5 Hz, 1H), 2.44 (t, J=7.8 Hz, 2H), 2.36 (s, 3H), 1.48-1.28 (m, 2H), 0.73 (t, J=7.3 Hz, 3H). ESI-MS m/z calc. 366.1402, found 367.2 (M+1)⁺; Retention time: 1.86 minutes (LC method A).

### Example 49: Preparation of Compound 141

### Step 1: N-(6-chloro-5-iodo-2-pyridyl)benzenesulfonamide

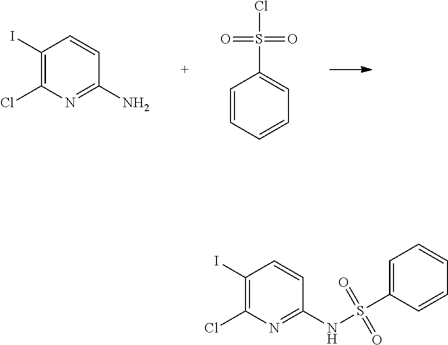 [SMILES: Nc1ccc(I)c(Cl)n1 ; "+" ; O=S(=O)(Cl)c1ccccc1 ; O=S(=O)(Nc1ccc(I)c(Cl)n1)c1ccccc1]

To a solution of 6-chloro-5-iodo-pyridin-2-amine (2 g, 7.860 mmol) in pyridine (30 mL) was added benzenesulfonyl chloride (1 mL, 7.836 mmol) and the reaction was stirred at room temperature for 63 hours. The reaction was heated at 60° C. for 4 hours. EtOAc was added to the reaction, and the organic phase was washed with water (×3). The organic layer was dried over Na₂SO₄, filtered, and concentrated. The crude product was purified on 120 g of silica gel utilizing a gradient of 0-15% ethyl acetate in dichloromethane to yield N-(6-chloro-5-iodo-2-pyridyl)benzenesulfonamide (1.93 g, 62%) as a light yellow solid. ¹H NMR (400 MHz, DMSO-d₆) δ 11.54 (s, 1H), 8.15 (d, J=8.4 Hz, 1H), 8.03-7.78 (m, 2H), 7.71-7.63 (m, 1H), 7.63-7.54 (m, 2H), 6.81 (d, J=8.4 Hz, 1H). ESI-MS m/z calc. 393.90396, found 394.9 (M+1)⁺; Retention time: 1.59 minutes (LC method A).

### Step 2: N-[6-chloro-5-[(E)-prop-1-enyl]-2-pyridyl]benzenesulfonamide

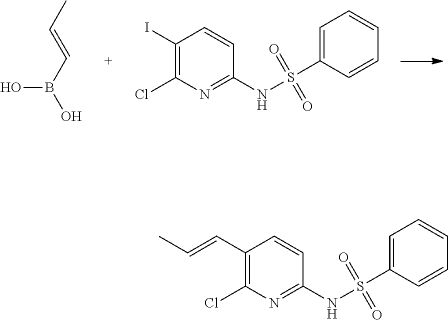

To N-(6-chloro-5-iodo-2-pyridyl)benzenesulfonamide (300 mg, 0.7602 mmol) was added [(E)-prop-1-enyl]boronic acid (approximately 135.0 mg, 1.572 mmol), Fibre Cat 1032 catalyst (50 mg, 0.07645 mmol), DMF (5.5 mL) and sodium carbonate (approximately 774.0 μL of 2 M, 1.548 mmol). The reaction mixture was stirred at 110° C. for 4 hours. The reaction was filtered. EtOAc and water were added to the filtrate. The two layers were separated, and the aqueous layer was extracted with EtOAc (×3). The combined organic layer was dried over Na₂SO₄ and filtered, and the solvent was removed under reduced pressure. The crude product was purified on 40 g of silica gel utilizing a gradient of 0-30% ethyl acetate in hexane to yield N-[6-chloro-5-[(E)-prop-1-enyl]-2-pyridyl]benzenesulfonamide (166 mg, 71%) as a white solid. ESI-MS m/z calc. 308.03864, found 309.1 (M+1)⁺; Retention time: 1.7 minutes (LC method A).

### Step 3: N-[6-phenoxy-5-[(E)-prop-1-enyl]-2-pyridyl]benzenesulfonamide

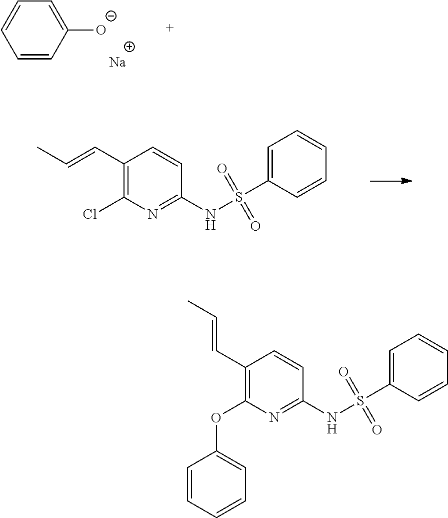 [SMILES: [O-]c1ccccc1 ; "+" ; [Na+] ; C/C=C/c1ccc(NS(=O)(=O)c2ccccc2)nc1Cl ; C/C=C/c1ccc(NS(=O)(=O)c2ccccc2)nc1Oc1ccccc1]

To N-[6-chloro-5-[(E)-prop-1-enyl]-2-pyridyl]benzenesulfonamide (50 mg, 0.1619 mmol), sodium phenoxide (19 mg, 0.1637 mmol) and DMF (1 mL) were added and the reaction was stirred at 110° C. for 5 hours. Sodium phenoxide (19 mg, 0.1637 mmol) was added, and the reaction was heated at 110° C. for 2 hours. The reaction was heated in a microwave oven at 200° C. for 6 hours and on a regular heat block at 200° C. for 5 hours. The crude product was filtered and purified using a reverse phase HPLC C₁₈ column and a dual gradient run from 1-99% mobile phase B over 30 minutes (Mobile phase A=H₂O (5 mM HCl). Mobile phase B═CH₃CN) to yield N-[6-phenoxy-5-[(E)-prop-1-enyl]-2-pyridyl]benzenesulfonamide (4.2 mg, 7%). ESI-MS m/z calc. 366.10382, found 367.1 (M+1)⁺; Retention time: 1.93 minutes (LC method A).

### Example 50: Preparation of Compound 142

### Step 1: 6-Phenoxy-4-phenyl-pyridin-2-amine

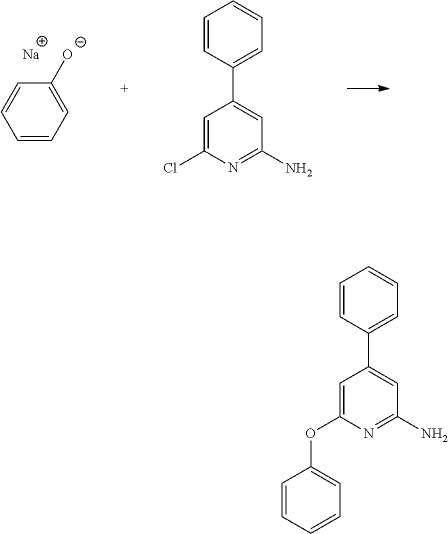 [SMILES: [O-]c1ccccc1 ; [Na+] ; "+" ; Nc1cc(-c2ccccc2)cc(Cl)n1 ; Nc1cc(-c2ccccc2)cc(Oc2ccccc2)n1]

To 6-chloro-4-phenyl-pyridin-2-amine (150 mg, 0.7329 mmol), sodium phenoxide (171 mg, 1.473 mmol) and DMF (3.000 mL) were added and the reaction was stirred at 200° C. for 5 hours in a pressure vessel. Water and EtOAc were added to the reaction and the two layers were separated. The aqueous layer was extracted with EtOAc (×3). The combined organic layer was washed with water (×3), dried over Na₂SO₄, filtered and the solvent was evaporated under reduced pressure. More sodium phenoxide (171 mg, 1.473 mmol) and DMF (3.000 mL) were added to the material, and the mixture was reacted at 200° C. for 3 hours. More sodium phenoxide (171 mg, 1.473 mmol) was added to the mixture was reacted at 200° C. for 17 hours. Water and EtOAc were added to the reaction, and the two layers were separated. The aqueous layer was extracted with EtOAc (×3). The combined organic layer was washed with water (×3), dried over Na₂SO₄, and filtered, and the solvent was evaporated under reduced pressure. The crude product was purified on 40 g of silica gel utilizing a gradient of 0-15% ethyl acetate in dichloromethane to yield 6-phenoxy-4-phenyl-pyridin-2-amine (80 mg, 42%). ESI-MS m/z calc. 262.11063, found 263.2 (M+1)⁺; Retention time: 1.41 minutes (LC method A).

### Step 2: N-(6-phenoxy-4-phenyl-2-pyridyl)benzenesulfonamide

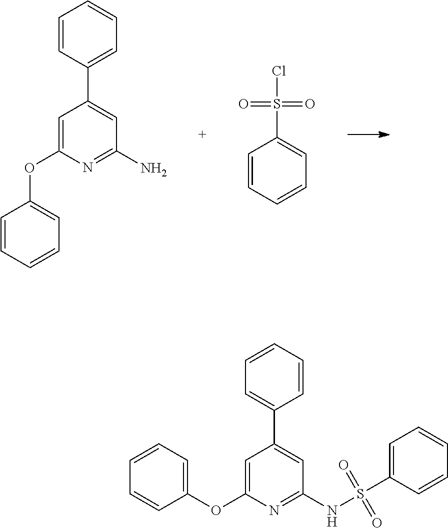 [SMILES: Nc1cc(-c2ccccc2)cc(Oc2ccccc2)n1 ; "+" ; O=S(=O)(Cl)c1ccccc1 ; O=S(=O)(Nc1cc(-c2ccccc2)cc(Oc2ccccc2)n1)c1ccccc1]

To a solution of 6-phenoxy-4-phenyl-pyridin-2-amine (80 mg, 0.3050 mmol) in pyridine (1 mL) was added benzenesulfonyl chloride (40 μL, 0.3134 mmol) and the reaction was stirred at room temperature for 2 hours. EtOAc was added to the reaction and washed with water (×1). The organic layer was dried over Na₂SO₄, filtered, and concentrated. The crude product was dissolved in DMSO, filtered, and purified using a reverse phase HPLC C₁₈ column and a dual gradient run from 1-99% mobile phase B over 15 minutes [(Mobile phase A=H₂O (5 mM HCl). Mobile phase B═CH₃CN)] to yield N-(6-phenoxy-4-phenyl-2-pyridyl)benzenesulfonamide (49 mg, 40%). ¹H NMR (400 MHz, DMSO-d₆) δ 11.15 (s, 1H), 7.68-7.62 (m, 2H), 7.60-7.44 (m, 8H), 7.44-7.37 (m, 2H), 7.34-7.23 (m, 1H), 7.16-7.05 (m, 2H), 6.90 (dd, J=11.2, 1.2 Hz, 2H). ESI-MS m/z calc. 402.10382, found 403.1 (M+1)⁺; Retention time: 1.97 minutes (LC method A).

### Example 51: Preparation of Compound 143

### Step 1: N-[4,6-bis(p-tolyl)-2-pyridyl]benzenesulfonamide

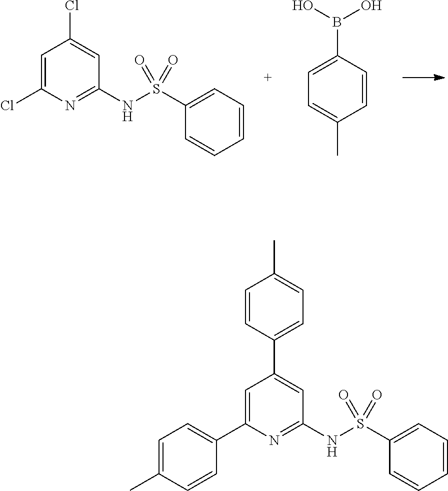

N-(4,6-dichloro-2-pyridyl)benzenesulfonamide (100 mg, 0.3299 mmol), p-tolylboronic acid (90 mg, 0.6620 mmol), potassium carbonate (approximately 660.0 μL of 2 M, 1.320 mmol), and 1,3-bis(2,6-diisopropylphenyl)-4,5-dihydroimidazole; 3-chloropyridine; dichloropalladium (approximately 24.00 mg, 0.03517 mmol) were combined in 2-propanol (2.600 mL) and the reaction was heated at 110° C. for 1 hour, 20 minutes. The reaction was filtered, and the solvent was evaporated under reduced pressure. The crude product was dissolved in DMSO, filtered, and purified using a reverse phase HPLC C₁₈ column and a dual gradient run from 1-99% mobile phase B over 30 minutes (Mobile phase A=H₂O (5 mM HCl). Mobile phase B═CH₃CN) to yield N-[4,6-bis(p-tolyl)-2-pyridyl]benzenesulfonamide (46.9 mg, 34%) as a yellow solid. ¹H NMR (400 MHz, DMSO-d₆) δ 11.18 (s, 1H), 8.05-7.95 (m, 2H), 7.89-7.80 (m, 2H), 7.73 (s, 1H), 7.67-7.63 (m, 2H), 7.63-7.53 (m, 3H), 7.38-7.29 (m, 2H), 7.26 (d, J=7.9 Hz, 2H), 7.12 (s, 1H), 2.37 (s, 3H), 2.35 (s, 3H). ESI-MS m/z calc. 414.1402, found 415.2 (M+1)⁺; Retention time: 2.13 minutes (LC method A).

### Example 52: Preparation of Compound 144

### Step 1: N-[5,6-bis(p-tolyl)-2-pyridyl]benzenesulfonamide

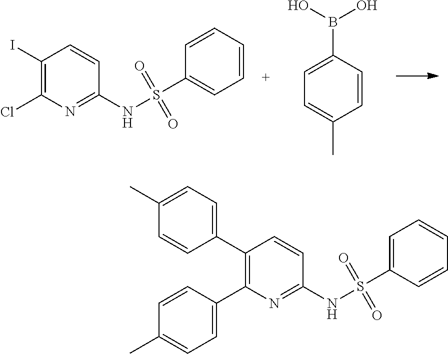

N-(6-chloro-5-iodo-2-pyridyl)benzenesulfonamide (100 mg, 0.2534 mmol), p-tolylboronic acid (69 mg, 0.5075 mmol), potassium carbonate (approximately 507.0 μL of 2 M, 1.014 mmol), and (1,3-Bis(2,6-diisopropylphenyl)imidazolidene) (3-chloropyridyl) palladium(II) dichloride (18 mg, 0.02637 mmol) were combined in 2-propanol (2 mL) and the reaction was heated at 80° C. for 19 hours. More p-tolylboronic acid (69 mg, 0.5075 mmol), potassium carbonate (approximately 507.0 μL of 2 M, 1.014 mmol), PEPPSI catalyst (74 mg) and 2-propanol (2 mL) were added and the reaction was heated at 180° C. for 4 hours. The reaction was filtered, and the solvent was evaporated under reduced pressure. The crude product was dissolved in DMSO, filtered, and purified using a reverse phase HPLC C₁₈ column and a dual gradient run from 1-99% mobile phase B over 30 minutes (Mobile phase A=H₂O [(5 mM HCl). Mobile phase B═CH₃CN)] to yield N-[5,6-bis(p-tolyl)-2-pyridyl]benzenesulfonamide (23.6 mg, 22%) as a yellow solid. ¹H NMR (400 MHz, DMSO-d₆) δ 11.20 (s, 1H), 8.00-7.91 (m, 2H), 7.67-7.61 (m, 2H), 7.59-7.53 (m, 2H), 7.08-7.01 (m, 5H), 7.00-6.93 (m, 4H), 2.26 (s, 3H), 2.26 (s, 3H). ESI-MS m/z calc. 414.1402, found 415.2 (M+1)⁺; Retention time: 2.03 minutes (LC method A).

### Example 53: Preparation of Compound 145

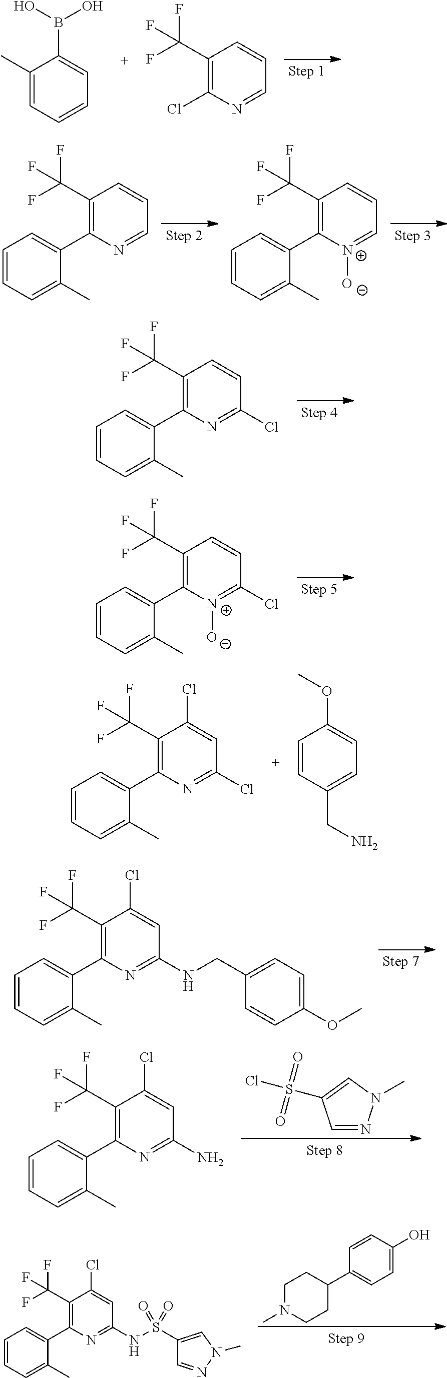

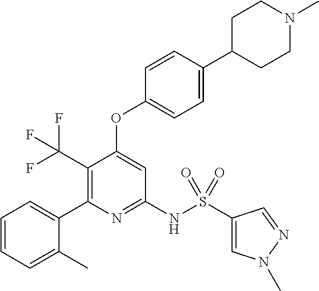 [SMILES: Cc1ccccc1-c1nc(NS(=O)(=O)c2cnn(C)c2)cc(Oc2ccc(C3CCN(C)CC3)cc2)c1C(F)(F)F]

### Step 1: 2-(o-Tolyl)-3-(trifluoromethyl)pyridine

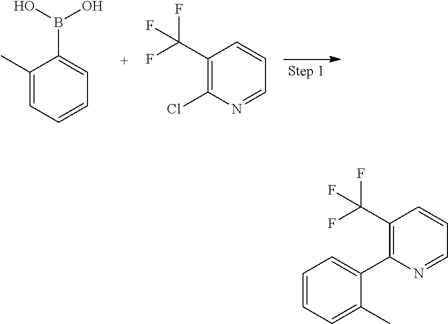

In a three-necked flask, nitrogen was bubbled through a mixture of toluene (240 mL) and water (20 mL) for 20 minutes, then potassium phosphate (29.3 g, 0.138 mol) was added. The mixture was stirred for 20 minutes under nitrogen bubbling, then 2-chloro-3-(trifluoromethyl)pyridine (10 g, 55.08 mmol), o-tolylboronic acid (10.1 g, 74.29 mmol) and PdCl₂(dppf)-DCM complex (1.32 g, 1.62 mmol) were successively added. The flask was then put in a pre-heated oil bath at 80° C. After being stirred at this temperature for 2 hours, the reaction mixture was cooled to room temperature then diluted with ethyl acetate (500 mL). The organic phase was washed with 5% aqueous NaHCO₃ (3×100 mL) and brine (2×100 mL), dried over Na₂SO₄, and filtered, and the solvent was removed under reduced pressure. The residue was purified by flash chromatography (Biotage SP1, dry-loaded, 120 g SiO₂) eluting with mixtures of 5-30% ethyl acetate in heptanes to afford 2-(o-tolyl)-3-(trifluoromethyl)pyridine (12.26 g, 91%) as an orange oil. ¹H NMR (300 MHz, CDCl₃) δ ppm 2.06 (s, 3H), 7.17 (d, J=7.5 Hz, 1H), 7.21-7.30 (m, 2H), 7.31-7.38 (m, 1H), 7.44 (dd, J=8.0, 5.0 Hz, 1H), 8.09 (dd, J=8.0, 1.3 Hz, 1H), 8.85 (d, J=5.0 Hz, 1H); ¹⁹F NMR (282 MHz, CDCl₃) δ ppm −59.8 (s, 3F); ESI-MS m/z calc. 237.0765, found 238.1 (M+1)⁺; Retention time: 2.22 minutes (LC method O).

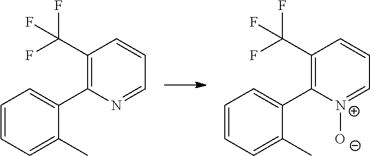 [SMILES: Cc1ccccc1-c1ncccc1C(F)(F)F ; Cc1ccccc1-c1c(C(F)(F)F)ccc[n+]1[O-]]

To a solution of 2-(o-tolyl)-3-(trifluoromethyl)pyridine (12.26 g, 51.68 mmol) in anhydrous dichloromethane (200 mL) at room temperature was added mCPBA (13.91 g, 62.07 mmol, 77% purity). After being stirred for 2 days at room temperature, the reaction mixture was diluted with ethyl acetate (500 mL) and the organic phase was washed with 5% aqueous NaHCO₃ (2×100 mL), 10% aqueous Na₂S₂O₃ (2×50 mL), 5% aqueous NaHCO₃ (2×100 mL) and brine (2×50 mL), dried over Na₂SO₄, filtered and the solvent was removed under reduced pressure. The residue was triturated in heptanes (1×40 mL), then in a mixture of MTBE (5 mL) and heptanes (40 mL), filtered and dried to afford 2-(o-tolyl)-1-oxido-3-(trifluoromethyl)pyridin-1-ium (11.06 g, 80%) as yellow solid. ¹H NMR (300 MHz, CDCl₃) δ ppm 2.12 (s, 3H), 7.15 (d, J=7.5 Hz, 1H), 7.27-7.36 (m, 2H), 7.37-7.46 (m, 2H), 7.63 (d, J=8.1 Hz, 1H), 8.49 (d, J=6.4 Hz, 1H); ¹⁹F NMR (282 MHz, CDCl₃): ppm −60.3 (s, 3F); ESI-MS m/z calc. 253.0714, found 254.1 (M+1)⁺; Retention time: 1.65 minutes (LC method C).

### Step 3: 6-Chloro-2-(o-tolyl)-3-(trifluoromethyl)pyridine

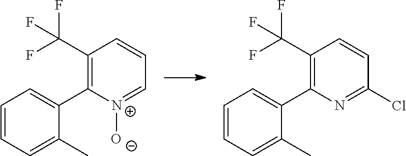 [SMILES: Cc1ccccc1-c1c(C(F)(F)F)ccc[n+]1[O-] ; Cc1ccccc1-c1nc(Cl)ccc1C(F)(F)F]

Phosphorus oxychloride (110 mL, 1.18 mol) was added to 2-(o-tolyl)-1-oxido-3-(trifluoromethyl)pyridin-1-ium (11.04 g, 41.42 mmol) at room temperature. The solution was heated to 105° C. (oil bath temperature) and was maintained at this temperature for 24 hours. After being cooled to room temperature, phosphorus oxychloride was removed under reduced pressure. The residue was taken up in MTBE (700 mL). The organic phase was treated with 5% aqueous NaHCO₃ until the pH of the aqueous phase had reached 7-8. The phases were separated, then the organic phase was washed with 5% aqueous NaHCO₃ (4×100 mL) and brine (2×100 mL), dried over Na₂SO₄, and filtered and the solvent was removed under reduced pressure. The residue was purified by flash chromatography (Biotage SP1, dry loaded, 120 g SiO₂) eluting with mixtures of 0-20% ethyl acetate in heptanes to afford 6-chloro-2-(o-tolyl)-3-(trifluoromethyl)pyridine (8.32 g, 70%) as a yellow oil. ¹H NMR (300 MHz, CDCl₃) δ ppm 2.09 (s, 3H), 7.16 (d, J=7.7 Hz, 1H), 7.19-7.29 (m, 2H), 7.30-7.38 (m, 1H), 7.47 (d, J=8.3 Hz, 1H), 8.04 (d, J=8.3 Hz, 1H); ¹⁹F NMR (282 MHz, CDCl₃) δ ppm −59.3 (s, 3F); ESI-MS m/z calc. 271.0376, found 272.1 (M+1)⁺; Retention time: 2.32 minutes (LC method N).

### Step 4: 6-Chloro-2-(o-tolyl)-1-oxido-3-(trifluoromethyl)pyridin-1-ium

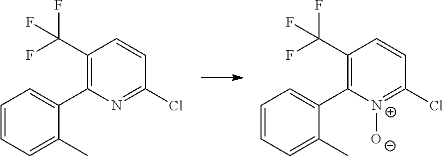 [SMILES: Cc1ccccc1-c1nc(Cl)ccc1C(F)(F)F ; Cc1ccccc1-c1c(C(F)(F)F)ccc(Cl)[n+]1[O-]]

To a solution of 6-chloro-2-(o-tolyl)-3-(trifluoromethyl)pyridine (8.3 g, 30.55 mmol) in anhydrous dichloromethane (205 mL) cooled to 0° C. was added urea hydrogen peroxide (5.87 g, 62.40 mmol), followed by the dropwise addition of trifluoroacetic anhydride (8.5 mL, 61.15 mmol). The reaction mixture was stirred for 40 minutes at 0° C. then the cooling bath was removed. After being stirred for 5 hours at room temperature, the reaction mixture was cooled to 0° C. and additional urea hydrogen peroxide (3.65 g, 38.80 mmol) was added followed by the dropwise addition of trifluoroacetic anhydride (5.30 mL, 38.13 mmol). The reaction mixture was stirred for 30 minutes at 0° C., then the cooling bath was removed. After being stirred at room temperature for 18 hours, the reaction mixture was diluted with ethyl acetate (700 mL). The organic phase was washed with 5% aqueous NaHCO₃ (3×150 mL), 10% aqueous Na₂S₂O₃ (2×100 mL), 5% aqueous NaHCO₃ (2×150 mL) and brine (2×100 mL), dried over Na₂SO₄, filtered and the solvent was removed under reduced pressure. The residue was triturated in water (1×75 mL) then filtered and dried. The residue was further purified by flash chromatography (Biotage SP1, dry loaded, 120 g SiO₂) eluting with mixture of 0-10% ethyl acetate in dichloromethane to afford 6-chloro-2-(o-tolyl)-1-oxido-3-(trifluoromethyl)pyridin-1-ium (7.37 g, 83%) as pale yellow solid. ¹H NMR (300 MHz, DMSO-d₆) δ ppm 1.99 (s, 3H), 7.21 (d, J=7.5 Hz, 1H), 7.26-7.37 (m, 2H), 7.37-7.45 (m, 1H), 7.80 (d, J=8.7 Hz, 1H), 8.11 (d, J=8.7 Hz, 1H); ¹⁹F NMR (282 MHz, DMSO-d₆) δ ppm −58.9 (s, 3F); ESI-MS m/z calc. 287.0325, found 288.1 (M+1)⁺; Retention time: 1.85 minutes (LC method N).

### Step 5: 4,6-Dichloro-2-(o-tolyl)-3-(trifluoromethyl)pyridine

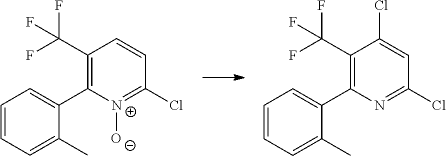 [SMILES: Cc1ccccc1-c1c(C(F)(F)F)ccc(Cl)[n+]1[O-] ; Cc1ccccc1-c1nc(Cl)cc(Cl)c1C(F)(F)F]

To 6-chloro-2-(o-tolyl)-1-oxido-3-(trifluoromethyl)pyridin-1-ium (7.25 g, 24.87 mmol) was added phosphorus oxychloride (80 mL, 0.858 mol) at room temperature. The mixture was then heated to 105° C. (oil bath temperature) and maintained at this temperature for 24 hours. After being cooled to room temperature, phosphorus oxychloride was removed under reduced pressure. More phosphorus oxychloride was removed by co-evaporating it with dichloromethane (2×200 mL). The residue was taken up in MTBE (900 mL) and dichloromethane (100 mL) and treated with 5% aqueous NaHCO₃ under stirring until pH 7-8 was obtained. The phases were separated, then the organic phase was washed with 5% aqueous NaHCO₃ (3×150 mL) and brine (2×150 mL), dried over Na₂SO₄, filtered and the solvent was removed under reduced pressure. The residue was purified by Isco Companion (dry loaded) (220 g SiO₂) eluting with mixtures of 0-10% ethyl acetate in heptanes to afford 4,6-dichloro-2-(o-tolyl)-3-(trifluoromethyl)pyridine (4.25 g, 38%, 67.5% purity) as a yellow oil and as a mixture with 6-chloro-2-(o-tolyl)-3-(trifluoromethyl)pyridine (10.8 mol % by 1H NMR) and 2,3-dichloro-6-(o-tolyl)-5-(trifluoromethyl)pyridine (21.6 mol % by 1H NMR). ESI-MS m/z calc. 304.9986, found 306.0 (M+1)⁺; Retention time: 2.62 minutes (LC method N).

### Step 6: 4-Chloro-N-[(4-methoxyphenyl)methyl]-6-(o-tolyl)-5-(trifluoromethyl)pyridin-2-amine

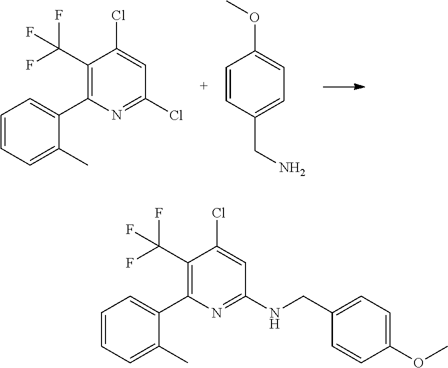

To a solution of 4,6-dichloro-2-(o-tolyl)-3-(trifluoromethyl)pyridine (4.2 g, 13.721 mmol) in anhydrous dioxane (84 mL) were added DIPEA (10 mL, 57.411 mmol) and (4-methoxyphenyl)methanamine (3.4 mL, 27.586 mmol). The mixture was heated to 70° C. and maintained at this temperature for 2 days. More (4-methoxyphenyl)methanamine (1.7 mL, 13.793 mmol) was added, and the mixture was stirred for 3 days at 70° C. After being cooled to room temperature, dioxane was removed under reduced pressure. The residue was taken up in ethyl acetate (500 mL), and the organic phase was washed with 5% aqueous NaHCO₃ (3×100 mL) and brine (2×100 mL), dried over Na₂SO₄, and filtered, and the solvent was removed under reduced pressure. The residue was purified by flash chromatography on silica gel (dry loaded) (120 g SiO₂) eluting with mixtures of 2-15% ethyl acetate in heptanes. 4-Chloro-N-[(4-methoxyphenyl)methyl]-6-(o-tolyl)-5-(trifluoromethyl)pyridin-2-amine eluted as a mixture with 6-chloro-N-[(4-methoxyphenyl)methyl]-2-(o-tolyl)-3-(trifluoromethyl)pyridin-4-amine. 3-Chloro-N-[(4-methoxyphenyl)methyl]-6-(o-tolyl)-5-(trifluoromethyl)pyridin-2-amine eluted as a mixture with 6-chloro-2-(o-tolyl)-3-(trifluoromethyl)pyridine. A second flash chromatography was done on the mixture of 4-Chloro-N-[(4-methoxyphenyl)methyl]-6-(o-tolyl)-5-(trifluoromethyl)pyridin-2-amine and 6-chloro-N-[(4-methoxyphenyl)methyl]-2-(o-tolyl)-3-(trifluoromethyl)pyridin-4-amine. The residues were purified by silica gel flash chromatography (dry loaded) (120 g SiO₂) eluting with mixtures of 5-15% ethyl acetate in heptanes to afford to afford 4-chloro-N-[(4-methoxyphenyl)methyl]-6-(o-tolyl)-5-(trifluoromethyl)pyridin-2-amine (2.28 g, 38%) as a colorless oil that solidified on standings. ¹H NMR (300 MHz, CDCl₃) δ ppm 2.14 (s, 3H), 3.80 (s, 3H), 4.32-4.47 (m, 2H), 5.36 (br s, 1H), 6.48 (s, 1H), 6.83-6.92 (m, 2H), 7.06-7.14 (m, 1H), 7.16-7.33 (m, 5H); ¹⁹F NMR (282 MHz, CDCl₃): ppm −53.8 (s, 3F); ESI-MS m/z calc. 406.106, found 407.2 (M+1)⁺; Retention time: 2.52 minutes (LC method N).

### Step 7: 4-Chloro-6-(o-tolyl)-5-(trifluoromethyl)pyridin-2-amine

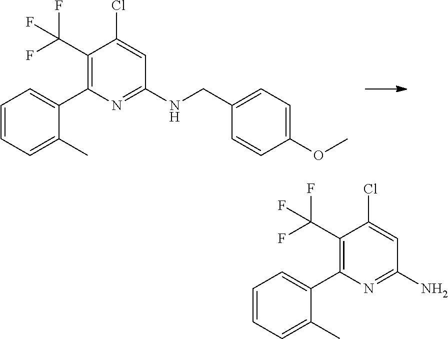

A mixture of 4-chloro-N-[(4-methoxyphenyl)methyl]-6-(o-tolyl)-5-(trifluoromethyl)pyridin-2-amine (2.28 g, 4.932 mmol) and trifluoroacetic acid (20 mL, 259.60 mmol) was heated to 50° C. and stirred at this temperature for 15 hours. After being cooled to room temperature, trifluoroacetic acid was removed under reduced pressure then co-evaporated with dichloromethane (4×40 mL). The residue was taken up in ethyl acetate (175 mL), and the organic phase was washed with 5% aqueous NaHCO₃ (4×50 mL) and brine (2×50 mL), dried over Na₂SO₄, filtered and the solvent was removed under reduced pressure. The residue was purified by Biotage SP1 (dry loaded) (120 g SiO₂) eluting with mixtures of 70-100% dichloromethane in heptanes. The fractions containing the desired compound were combined and concentrated under reduced pressure. The residue was purified a second time by silica gel flash chromatography (dry loaded) (80 g SiO₂) eluting with mixtures of 50-100% dichloromethane in heptanes to afford 4-chloro-6-(o-tolyl)-5-(trifluoromethyl)pyridin-2-amine (1.18 g, 83%) as an off-white solid. ¹H NMR (300 MHz, DMSO-d₆) δ ppm 2.04 (s, 3H), 6.66 (s 1H), 7.06 (d, J=7.1 Hz, 1H), 7.11 (br s, 2H), 7.15-7.30 (m, 3H); ¹⁹F NMR (282 MHz, DMSO-d₆) δ ppm −52.1 (s, 3F), ESI-MS m/z calc. 286.0485, found 287.1 (M+1)⁺; Retention time: 3.11 minutes (LC method N).

### Step 8: N-[4-chloro-6-(o-tolyl)-5-(trifluoromethyl)-2-pyridyl]-1-methyl-pyrazole-4-sulfonamide

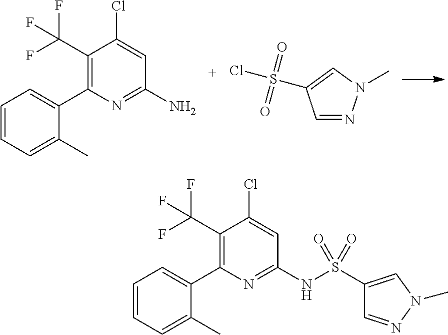

Solid 4-chloro-6-(o-tolyl)-5-(trifluoromethyl)pyridin-2-amine (167 mg, 0.5825 mmol) was added to a mixture NaH (55.6 mg of 60% w/w, 1.390 mmol) in DMF (1.5 mL) at 0° C. The reaction mixture was stirred for 20 minutes and then treated with solid 1-methylpyrazole-4-sulfonyl chloride (120 mg, 0.6644 mmol). HCl (200 μL of 1 M, 0.20 mmol) was added and the reaction mixture was filtered and purified on reverse phase HPLC (HCl modifier, 25-75% ACN-H₂O) to give N-[4-chloro-6-(o-tolyl)-5-(trifluoromethyl)-2-pyridyl]-1-methyl-pyrazole-4-sulfonamide (191.3 mg, 76%). ¹H NMR (400 MHz, DMSO-d₆) δ 11.87 (s, 1H), 8.11 (s, 1H), 7.64 (s, 1H), 7.36 (dt, J=13.3, 7.1 Hz, 2H), 7.28 (t, J=7.2 Hz, 1H), 7.19 (s, 1H), 7.10 (d, J=7.5 Hz, 1H), 3.76 (s, 3H), 1.98 (s, 3H). ESI-MS m/z calc. 430.04782, found 431.0 (M+1)⁺; Retention time: 1.72 minutes, LC method A.

### Step 9: 1-Methyl-N-[4-[4-(1-methyl-4-piperidyl)phenoxy]-6-(o-tolyl)-5-(trifluoromethyl)-2-pyridyl]pyrazole-4-sulfonamide

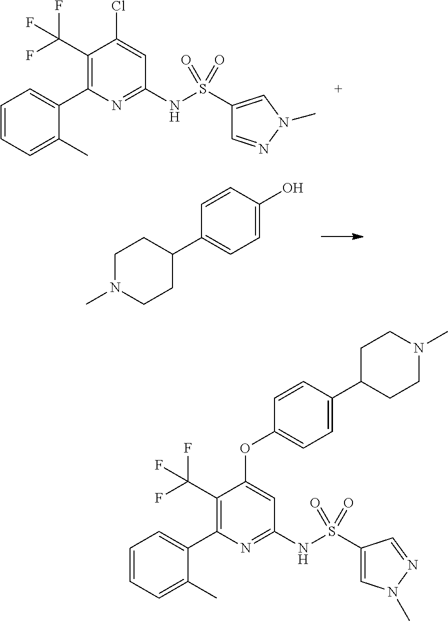

An NMP (1 mL) mixture of N-[4-chloro-6-(o-tolyl)-5-(trifluoromethyl)-2-pyridyl]-1-methyl-pyrazole-4-sulfonamide (100 mg, 0.2319 mmol), 4-(1-methyl-4-piperidyl)phenol (60 mg, 0.3137 mmol), and cesium carbonate (300 mg, 0.9208 mmol) was stirred at 100° C. for 150 minutes. The temperature of the reaction was increased to 110° C., and the mixture was stirred at this temperature for 24 hours. The reaction was stirred at 150° C. for 17 hours. The reaction mixture was cooled down to room temperature, filtered, and purified by reverse phase preparative chromatography using a C₁₈ column and a 15 minutes, gradient eluent of 25 to 75% acetonitrile in water containing 5 mM hydrochloric acid to give 1-methyl-N-[4-[4-(1-methyl-4-piperidyl)phenoxy]-6-(o-tolyl)-5-(trifluoromethyl)-2-pyridyl]pyrazole-4-sulfonamide (61.7 mg, 45%). ¹H NMR (400 MHz, DMSO-d₆) δ 10.71 (s, 2H), 7.92 (s, 1H), 7.45-7.40 (m, 3H), 7.38-7.33 (m, 2H), 7.27 (dd, J=17.6, 8.0 Hz, 4H), 7.13 (d, J=7.4 Hz, 1H), 6.37 (s, 1H), 3.78 (s, 3H), 3.08 (d, J=11.7 Hz, 3H), 2.89 (s, 2H), 2.77 (d, J=4.6 Hz, 3H), 2.18-2.03 (m, 9H). ESI-MS m/z calc. 585.20215, found 586.0 (M+1)⁺; Retention time: 1.33 minutes, LC method A.

### Example 54: Preparation of Compound 146

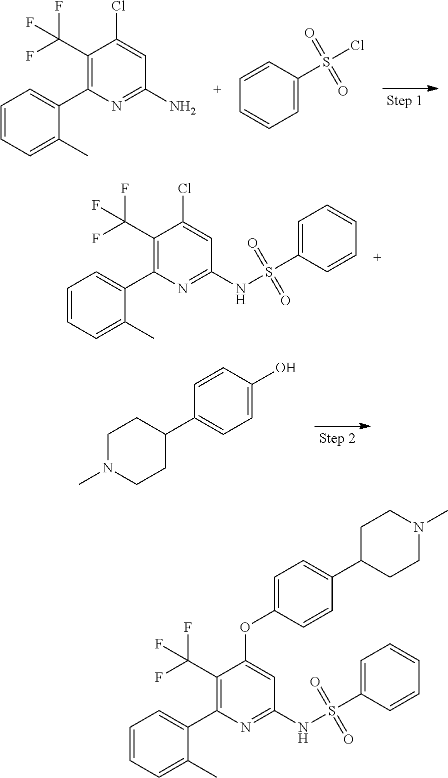 [SMILES: Cc1ccccc1-c1nc(N)cc(Cl)c1C(F)(F)F ; O=S(=O)(Cl)c1ccccc1 ; Cc1ccccc1-c1nc(NS(=O)(=O)c2ccccc2)cc(Cl)c1C(F)(F)F ; CN1CCC(c2ccc(O)cc2)CC1 ; Cc1ccccc1-c1nc(NS(=O)(=O)c2ccccc2)cc(Oc2ccc(C3CCN(C)CC3)cc2)c1C(F)(F)F]

### Step 1: N-[4-chloro-6-(o-tolyl)-5-(trifluoromethyl)-2-pyridyl]benzenesulfonamide

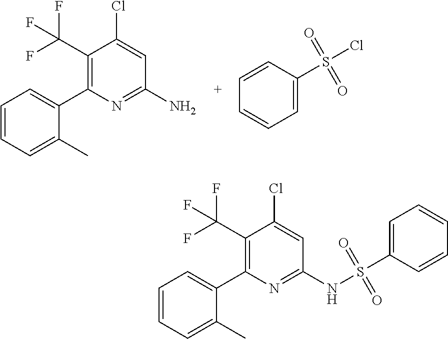 [SMILES: Cc1ccccc1-c1nc(N)cc(Cl)c1C(F)(F)F ; O=S(=O)(Cl)c1ccccc1 ; Cc1ccccc1-c1nc(NS(=O)(=O)c2ccccc2)cc(Cl)c1C(F)(F)F]

To a solution of 4-chloro-6-(o-tolyl)-5-(trifluoromethyl)pyridin-2-amine (200 mg, 0.6976 mmol) in DMF (3 mL) at 0° C. was slowly added NaH (116 mg of 60% w/w, 2.900 mmol) and the reaction was stirred at this temperature for 15 minutes. At this time, PhSO₂Cl (108 μL, 0.8463 mmol) was added, the cooling bath removed, and the reaction stirred at room temperature for 1 hour. The reaction mixture was poured into water and the pH brought to ˜4 with 1 N HCl. The precipitated product was filtered, washed with water, and desiccated to give N-[4-chloro-6-(o-tolyl)-5-(trifluoromethyl)-2-pyridyl]benzenesulfonamide (210 mg, 71%) as an off white solid. ESI-MS m/z calc. 426.04166, found 427.2 (M+1)⁺; Retention time: 0.76 minutes, LC method D.

### Step 2: N-[4-[4-(1-methyl-4-piperidyl)phenoxy]-6-(o-tolyl)-5-(trifluoromethyl)-2-pyridyl]benzenesulfonamide

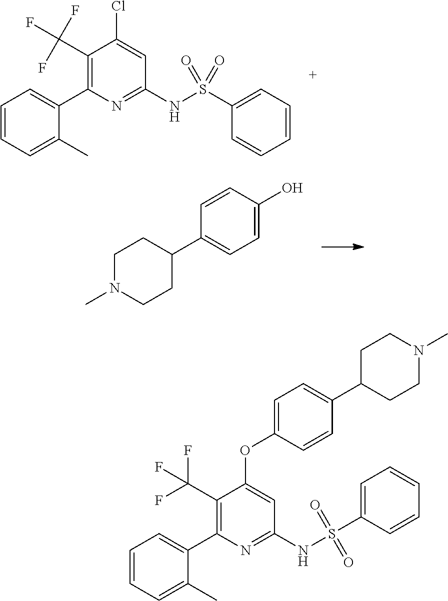

Solid N-[4-chloro-6-(o-tolyl)-5-(trifluoromethyl)-2-pyridyl]benzenesulfonamide (25 mg, 0.05857 mmol) and 4-(1-methyl-4-piperidyl)phenol (28 mg, 0.1464 mmol) was heated in a test tube with a heat gun for 60 seconds. This was repeated two more times. The residue was taken up in 1:1 DMSO:MeOH, filtered and purified by HPLC (1-99% ACN in water (HCl modifier)) to give N-[4-[4-(1-methyl-4-piperidyl)phenoxy]-6-(o-tolyl)-5-(trifluoromethyl)-2-pyridyl]benzenesulfonamide (hydrochloride salt) (4.2 mg, 11%). ESI-MS m/z calc. 581.196, found 582.4 (M+1)⁺; Retention time: 1.46 minutes, LC method A.

### Example 55: Preparation of Compound 147

### Step 1: N-[4-[4-(4-methylpiperazin-1-yl)phenoxy]-6-(o-tolyl)-5-(trifluoromethyl)-2-pyridyl]benzenesulfonamide

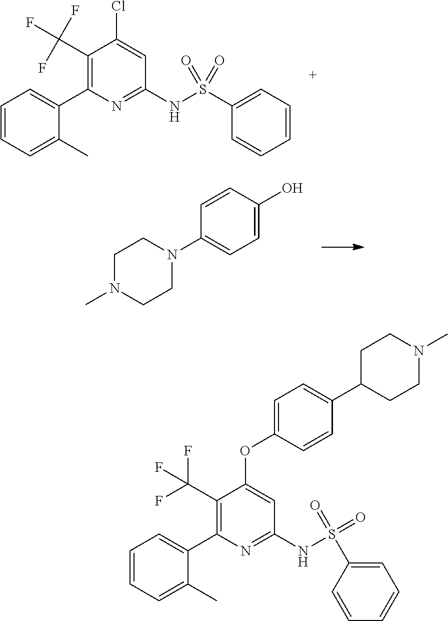

Solid N-[4-chloro-6-(o-tolyl)-5-(trifluoromethyl)-2-pyridyl]benzenesulfonamide (25 mg, 0.05857 mmol) and 4-(4-methylpiperazin-1-yl)phenol (33 mg, 0.1716 mmol) were heated with a heat gun for 60 seconds. This operation was repeated three times. The residue was taken up in DMSO and filtered. Purification by HPLC (1-99% ACN in water (HCl modifier)) gave N-[4-[4-(4-methylpiperazin-1-yl)phenoxy]-6-(o-tolyl)-5-(trifluoromethyl)-2-pyridyl]benzenesulfonamide (hydrochloride salt) (7.1 mg, 20%). ESI-MS m/z calc. 582.1912, found 583.4 (M+1)⁺; Retention time: 1.42 minutes, LC method A.

### Example 56: Characterization of Compounds 123 and 148-158

The compounds in the following tables were prepared in a manner analogous to those described above using commercially available reagents and intermediates described herein.

| Compound | | LCMS | Calc. | | LCMS |
|---|---|---|---|---|---|
| number | Structure | Rt (min) | mass | M + 1 | Method |
| 148 | 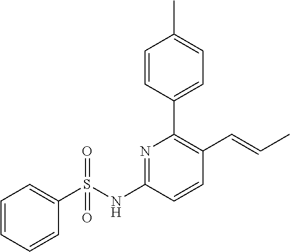 [SMILES: C/C=C/c1ccc(NS(=O)(=O)c2ccccc2)nc1-c1ccc(C)cc1] | 1.88 | 364.125 | 365.2 | A |
| 149 | 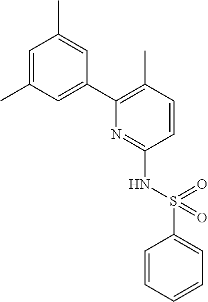 [SMILES: Cc1cc(C)cc(-c2nc(NS(=O)(=O)c3ccccc3)ccc2C)c1] | 1.77 | 352.125 | 353 | A |
| 150 | 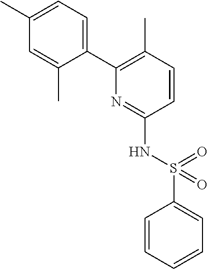 [SMILES: Cc1ccc(-c2nc(NS(=O)(=O)c3ccccc3)ccc2C)c(C)c1] | 1.7 | 352.125 | 353 | A |
| 151 | 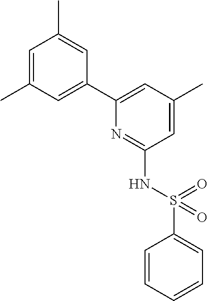 | 1.84 | 352.125 | 353 | A |
| 152 | 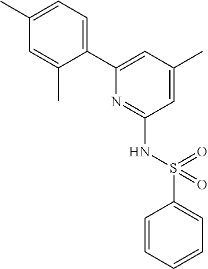 | 1.72 | 352.125 | 353 | A |
| 153 | 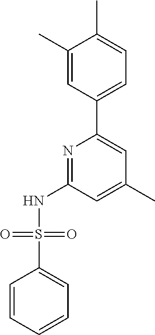 [SMILES: Cc1cc(NS(=O)(=O)c2ccccc2)nc(-c2ccc(C)c(C)c2)c1] | 1.81 | 352.125 | 353 | A |
| 154 | 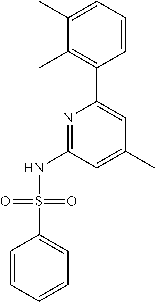 [SMILES: Cc1cc(NS(=O)(=O)c2ccccc2)nc(-c2cccc(C)c2C)c1] | 1.69 | 352.125 | 353 | A |
| 155 | 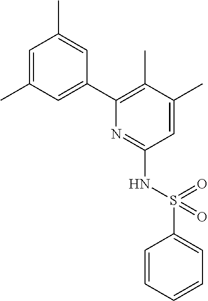 | 1.78 | 366.14 | 367 | A |
| 156 | 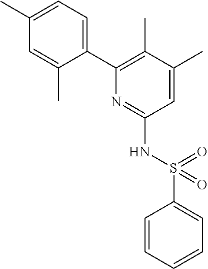 | 1.7 | 366.14 | 367 | A |
| 157 | 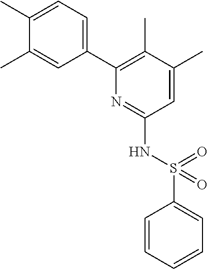 | 1.75 | 366.14 | 367 | A |
| 158 | 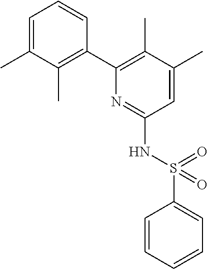 | 1.68 | 366.14 | 367 | A |
| 123 | 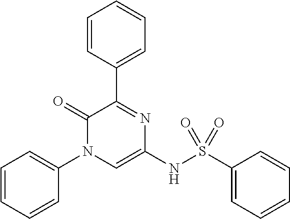 [SMILES: O=c1c(-c2ccccc2)nc(NS(=O)(=O)c2ccccc2)cn1-c1ccccc1] | 1.53 | 403.099 | 404.2 | |

| Com- | |
|---|---|
| pound | |
| number | NMR |
| 147 | ¹H NMR (400 MHz, DMSO-d₆) δ 11.10 (s, 1H), |
| | 7.93-7.79 (m, 3H), 7.69-7.59 (m, 1H), 7.58-7.51 |
| | (m, 2H), 7.26 (d, J = 8.0 Hz, 2H), 7.20 (d, J = 7.8 |
| | Hz, 2H), 6.97 (s, 1H), 6.33-6.01 (m, 2H), 2.37 (s, |
| | 3H), 1.75 (d, J = 4.9 Hz, 3H). |
| 149 | ¹H NMR (400 MHz, DMSO) δ 10.89 (s, 1H), |
| | 7.85-7.79 (m, 2H), 7.68-7.55 (m, 2H), 7.50 (t, J = |
| | 7.9 Hz, 2H), 7.14-6.99 (m, 3H), 6.92 (d, J = 6.0 |
| | Hz, 1H), 2.31 (s, 3H), 1.89 (s, 3H), 1.80 (s, 3H). |
| 150 | ¹H NMR (400 MHz, DMSO) δ 11.09 (s, 1H), |
| | 7.97 (d, J = 8.4 Hz, 2H), 7.65-7.55 (m, 3H), 7.40 |
| | (s, 2H), 7.34 (s, 1H), 7.01 (s, 1H), 6.76 (s, 1H), |
| | 2.30 (d, J = 5.1 Hz, 9H). |
| 151 | ¹H NMR (400 MHz, DMSO) δ 10.99 (s, 1H), |
| | 7.91-7.84 (m, 2H), 7.65-7.56 (m, 1H), 7.53 (t, J = |
| | 8.0 Hz, 2H), 6.99 (t, J = 37.9 Hz, 5H), 2.29 (d, J = |
| | 5.4 Hz, 6H), 2.08 (s, 3H). |
| 153 | ¹H NMR (400 MHz, DMSO) δ 10.91 (s, 1H), |
| | 7.87 (d, J = 7.1 Hz, 2H), 7.61 (t, J = 7.3 Hz, 1H), |
| | 7.53 (t, J = 8.0 Hz, 2H), 7.21 (d, J = 7.4 Hz, 1H), |
| | 7.12 (t, J = 7.5 Hz, 1H), 7.01-6.86 (m, 2H), 6.80 |
| | (s, 1H), 2.28 (d, J = 10.5 Hz, 6H), 1.96 (s, 3H). |
| 154 | ¹H NMR (400 MHz, DMSO) δ 10.86 (s, 1H), |
| | 7.87 (d, J = 7.5 Hz, 2H), 7.55 (dd, J = 20.8, 13.2 |
| | Hz, 3H), 7.02 (s, 1H), 6.82 (s, 3H), 2.30 (s, 6H), |
| | 2.23 (s, 3H), 2.03 (s, 3H). |
| 155 | ¹H NMR (400 MHz, DMSO) δ 10.70 (s, 1H), |
| | 7.86-7.79 (m, 2H), 7.57 (t, J = 7.3 Hz, 1H), 7.50 |
| | (t, J = 8.0 Hz, 2H), 7.14-6.98 (m, 3H), 6.92 (s, |
| | 1H), 2.32 (s, 3H), 2.25 (s, 3H), 1.82 (d, J = |
| | 21.7 Hz, 6H). |
| 156 | ¹H NMR (400 MHz, DMSO) δ 7.88 (d, J = 7.2 |
| | Hz, 2H), 7.62 (t, J = 7.1 Hz, 1H), 7.54 (t, J = 7.5 |
| | Hz, 2H), 7.18 (d, J = 7.6 Hz, 1H), 7.01 (d, J = 7.1 |
| | Hz, 2H), 6.91 (s, 1H), 3.17 (s, 1H), 2.29-2.21 |
| | (m, 9H), 2.03 (s, 3H). |
| 157 | ¹H NMR (400 MHz, DMSO) δ 7.82 (d, J = 8.6 Hz, |
| | 2H), 7.59 (t, J = 7.4 Hz, 1H), 7.51 (t, J = 7.5 Hz, |
| | 2H), 7.21 (d, J = 7.3 Hz, 1H), 7.13 (t, J = 7.5 Hz, |
| | 1H), 7.04 (s, 1H), 6.84 (d, J = 7.2 Hz, 1H), 2.26 (d, |
| | J = 5.7 Hz, 6H), 1.77 (d, J = 8.6 Hz, 6H). |

### V. Bioactivity Assays

A. 3t3 Assay

1. Membrane Potential Optical Methods for Assaying F508del Modulation Properties of Compounds

The assay utilizes fluorescent voltage sensing dyes to measure changes in membrane potential using a fluorescent plate reader (e.g., FLTPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.

2. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NUT 3T3 cells. The F508del NUT 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM ivacaftor.

3. Solutions

Bath Solution #1: (in mM) NaCl 160, KCl 4.5, CaCl₂ 2, MgCl₂ 1, HEPES 10, pH 7.4 with NaOH.

Chloride-free bath solution: Chloride salts in Bath Solution #1 (above) are substituted with gluconate salts.

4. Cell Culture

NIH3T3 mouse fibroblasts stably expressing F508del were used for optical measurements of membrane potential. The cells were maintained at 37° C. in 5% CO₂ and 90% humidity in Dulbecco's modified Eagle's medium supplemented with 2 mM glutamine, 10% fetal bovine serum, 1×NEAA, b-ME, 1×pen/strep, and 25 mM HEPES in 175 cm² culture flasks. For all optical assays, the cells were seeded at ˜20,000/well in 384-well Matrigel-coated plates. For the correction assays, the cells were cultured at 37° C. with and without compounds for 16-24 hours.

B. Enteroid Assay

1. Solutions

Base medium (ADF+++) consisted of Advanced DMEM/Ham's F12, 2 mM Glutamax, 10 mM HEPES, 1 μg/mL penicillin/streptomycin.

Intestinal enteroid maintenance medium (IEMM) consisted of ADF+++, 1×B27 supplement, 1×N₂ supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath1 Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45).

3. Enteroid Cell Harvesting and Seeding

Cells were recovered in cell recovery solution, collected by centrifugation at 650 rpm for 5 minutes at 4° C., resuspended in TrypLE, and incubated for 5 minutes at 37° C. Cells were then collected by centrifugation at 650 rpm for 5 minutes at 4° C. and resuspended in IEMM containing 10 μM ROCK inhibitor (RI). The cell suspension was passed through a 40 μm cell strainer and resuspended at 1×10⁶ cells/mL in IEMM containing 10 μM RI. Cells were seeded at 5000 cells/well into multi-well plates and incubated for overnight at 37° C., 95% humidity and 5% CO₂ prior to assay.

4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 minutes at room temperature. Following dye incubation, cells were washed 3 times in Chloride Free Dye Solution. Chloride transport was initiated by addition of Chloride Free Dye Stimulation Solution and the fluorescence signal was read for 15 minutes. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM (S)—N-((6-aminopyridin-2-yl)sulfonyl)-6-(3-fluoro-5-isobutoxyphenyl)-2-(2,2,4-trimethylpyrrolidin-1-yl)nicotinamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 minutes at room temperature. Following dye incubation, cells were washed 3 times in Chloride Free Dye Solution. Chloride transport was initiated by addition of Chloride Free Dye Stimulation Solution, and the fluorescence signal was read for 15 minutes. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

C. Biological Activity Data

The following table represent CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >60%; ++ is 30-60%; + is <30%).

| | | | 3T3 Max | | |
|---|---|---|---|---|---|
| Cmpd | | 3T3 EC50 | Activity | | |
| No. | Structure | (μM) | (%) | | |
| 1 | 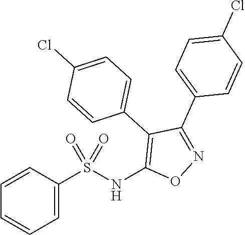 [SMILES: O=S(=O)(Nc1onc(-c2ccc(Cl)cc2)c1-c1ccc(Cl)cc1)c1ccccc1] | ++ | ++ | | |
| 2 | 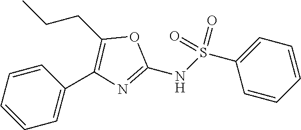 [SMILES: CCCc1oc(NS(=O)(=O)c2ccccc2)nc1-c1ccccc1] | + | ++ | | |
| 3 | 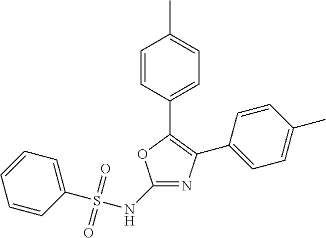 [SMILES: Cc1ccc(-c2nc(NS(=O)(=O)c3ccccc3)oc2-c2ccc(C)cc2)cc1] | ++ | +++ | | |
| 4 | 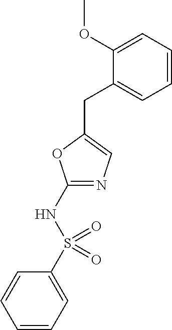 [SMILES: COc1ccccc1Cc1cnc(NS(=O)(=O)c2ccccc2)o1] | N.D. | + | | |
| 5 | 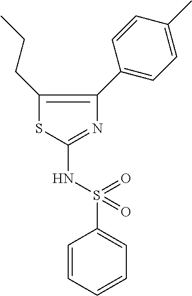 [SMILES: CCCc1sc(NS(=O)(=O)c2ccccc2)nc1-c1ccc(C)cc1] | +++ | +++ | | |
| 6 | 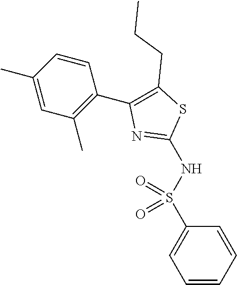 [SMILES: CCCc1sc(NS(=O)(=O)c2ccccc2)nc1-c1ccc(C)cc1C] | +++ | +++ | | |
| 7 | 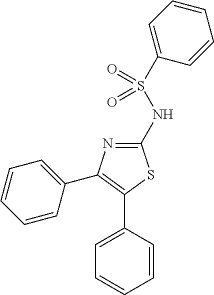 [SMILES: O=S(=O)(Nc1nc(-c2ccccc2)c(-c2ccccc2)s1)c1ccccc1] | ++ | +++ | | |
| 8 | 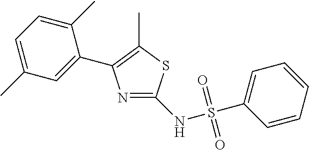 [SMILES: Cc1ccc(C)c(-c2nc(NS(=O)(=O)c3ccccc3)sc2C)c1] | +++ | +++ | | |
| 9 | 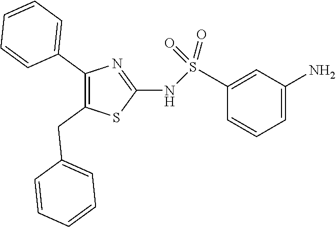 [SMILES: Nc1cccc(S(=O)(=O)Nc2nc(-c3ccccc3)c(Cc3ccccc3)s2)c1] | ++ | ++ | | |
| 10 | 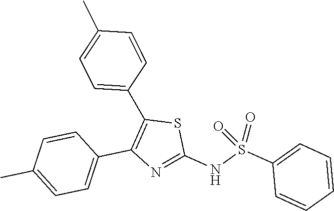 [SMILES: Cc1ccc(-c2nc(NS(=O)(=O)c3ccccc3)sc2-c2ccc(C)cc2)cc1] | +++ | ++ | | |
| 11 | 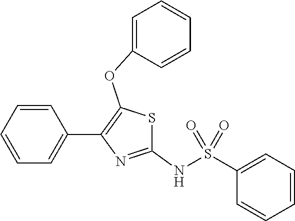 [SMILES: O=S(=O)(Nc1nc(-c2ccccc2)c(Oc2ccccc2)s1)c1ccccc1] | N.D. | + | | |
| 12 | 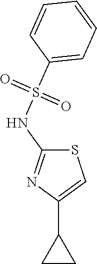 [SMILES: O=S(=O)(Nc1nc(C2CC2)cs1)c1ccccc1] | N.D. | + | | |
| 13 | 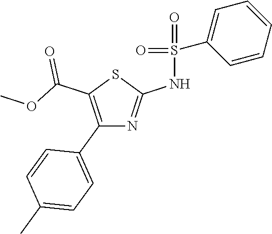 [SMILES: COC(=O)c1sc(NS(=O)(=O)c2ccccc2)nc1-c1ccc(C)cc1] | + | +++ | | |
| 14 | 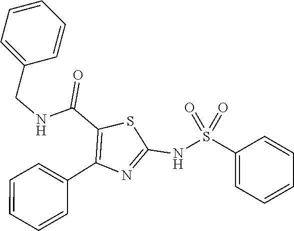 [SMILES: O=C(NCc1ccccc1)c1sc(NS(=O)(=O)c2ccccc2)nc1-c1ccccc1] | + | ++ | | |
| 15 | 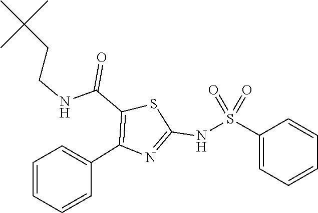 [SMILES: CC(C)(C)CCNC(=O)c1sc(NS(=O)(=O)c2ccccc2)nc1-c1ccccc1] | + | ++ | | |
| 16 | 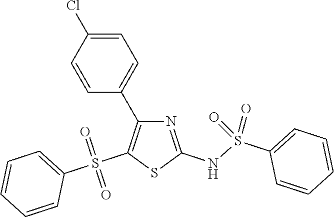 [SMILES: O=S(=O)(Nc1nc(-c2ccc(Cl)cc2)c(S(=O)(=O)c2ccccc2)s1)c1ccccc1] | ++ | ++ | | |
| 17 | 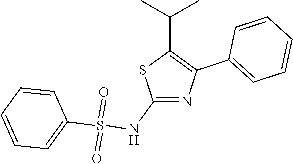 [SMILES: CC(C)c1sc(NS(=O)(=O)c2ccccc2)nc1-c1ccccc1] | + | +++ | | |
| 18 | 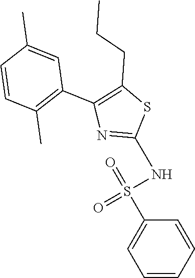 [SMILES: CCCc1sc(NS(=O)(=O)c2ccccc2)nc1-c1cc(C)ccc1C] | +++ | +++ | | |
| 19 | 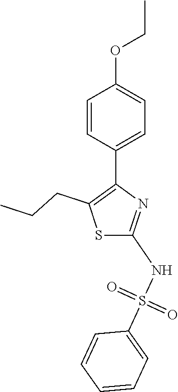 [SMILES: CCCc1sc(NS(=O)(=O)c2ccccc2)nc1-c1ccc(OCC)cc1] | ++ | ++ | | |
| 20 | 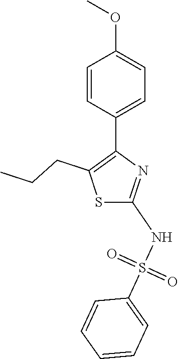 [SMILES: CCCc1sc(NS(=O)(=O)c2ccccc2)nc1-c1ccc(OC)cc1] | ++ | +++ | | |
| 21 | 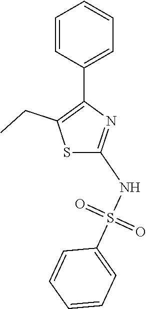 [SMILES: CCc1sc(NS(=O)(=O)c2ccccc2)nc1-c1ccccc1] | + | +++ | | |
| 22 | 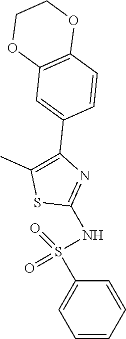 [SMILES: Cc1sc(NS(=O)(=O)c2ccccc2)nc1-c1ccc2c(c1)OCCO2] | + | ++ | | |
| 23 | 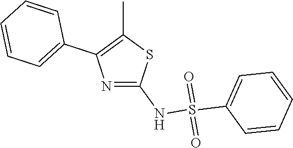 [SMILES: Cc1sc(NS(=O)(=O)c2ccccc2)nc1-c1ccccc1] | + | +++ | | |
| 24 | 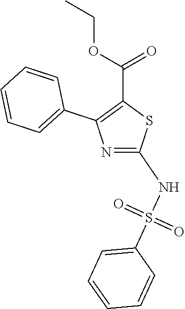 [SMILES: CCOC(=O)c1sc(NS(=O)(=O)c2ccccc2)nc1-c1ccccc1] | + | +++ | | |
| 25 | 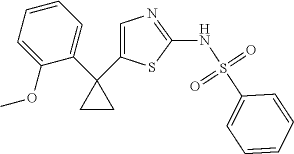 [SMILES: COc1ccccc1C1(c2cnc(NS(=O)(=O)c3ccccc3)s2)CC1] | + | +++ | | |
| 26 | 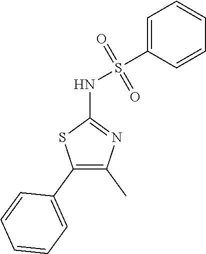 [SMILES: Cc1nc(NS(=O)(=O)c2ccccc2)sc1-c1ccccc1] | ++ | +++ | | |
| 27 | 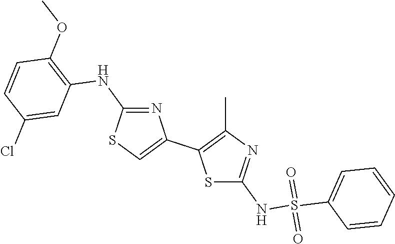 [SMILES: COc1ccc(Cl)cc1Nc1nc(-c2sc(NS(=O)(=O)c3ccccc3)nc2C)cs1] | ++ | ++ | | |
| 28 | 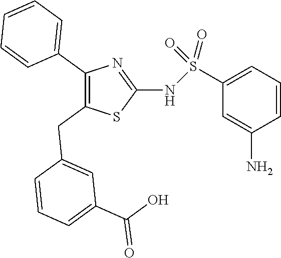 [SMILES: Nc1cccc(S(=O)(=O)Nc2nc(-c3ccccc3)c(Cc3cccc(C(=O)O)c3)s2)c1] | N.D. | + | | |
| 29 | 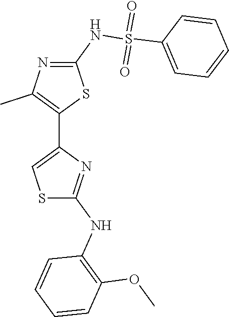 [SMILES: COc1ccccc1Nc1nc(-c2sc(NS(=O)(=O)c3ccccc3)nc2C)cs1] | + | ++ | | |
| 30 | 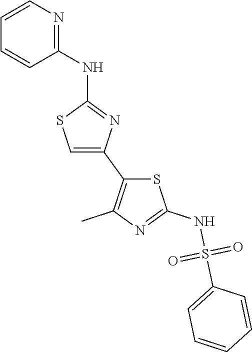 [SMILES: Cc1nc(NS(=O)(=O)c2ccccc2)sc1-c1csc(Nc2ccccn2)n1] | ++ | ++ | | |
| 31 | 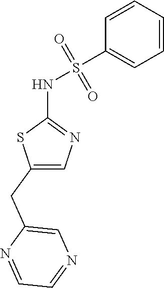 [SMILES: O=S(=O)(Nc1ncc(Cc2cnccn2)s1)c1ccccc1] | N.D. | + | | |
| 32 | 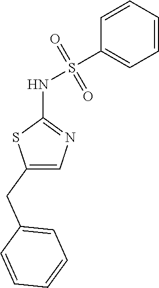 [SMILES: O=S(=O)(Nc1ncc(Cc2ccccc2)s1)c1ccccc1] | N.D. | ++ | | |
| 33 | 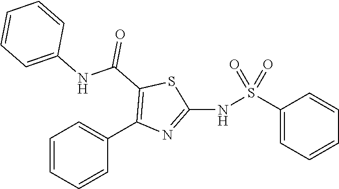 [SMILES: O=C(Nc1ccccc1)c1sc(NS(=O)(=O)c2ccccc2)nc1-c1ccccc1] | N.D. | + | | |
| 34 | 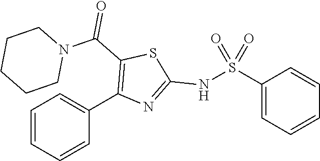 [SMILES: O=C(c1sc(NS(=O)(=O)c2ccccc2)nc1-c1ccccc1)N1CCCCC1] | N.D. | + | | |
| 35 | 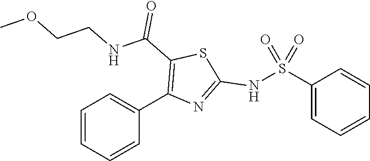 [SMILES: COCCNC(=O)c1sc(NS(=O)(=O)c2ccccc2)nc1-c1ccccc1] | N.D. | + | | |
| 36 | 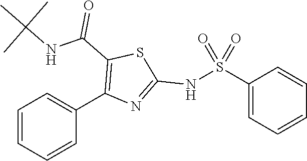 [SMILES: CC(C)(C)NC(=O)c1sc(NS(=O)(=O)c2ccccc2)nc1-c1ccccc1] | N.D. | + | | |
| 37 | 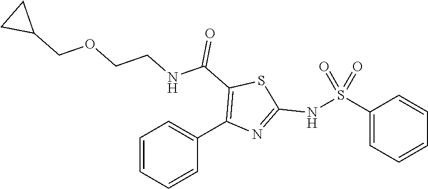 [SMILES: O=C(NCCOCC1CC1)c1sc(NS(=O)(=O)c2ccccc2)nc1-c1ccccc1] | N.D. | + | | |
| 38 | 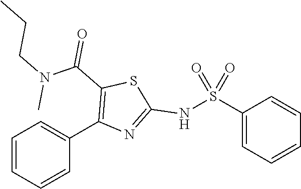 [SMILES: CCCN(C)C(=O)c1sc(NS(=O)(=O)c2ccccc2)nc1-c1ccccc1] | N.D. | + | | |
| 39 | 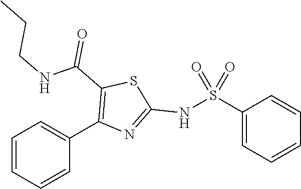 [SMILES: CCCNC(=O)c1sc(NS(=O)(=O)c2ccccc2)nc1-c1ccccc1] | N.D. | + | | |
| 40 | 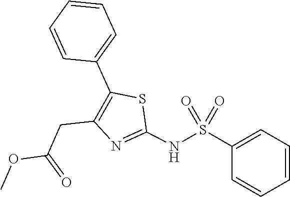 [SMILES: COC(=O)Cc1nc(NS(=O)(=O)c2ccccc2)sc1-c1ccccc1] | + | ++ | | |
| 41 | 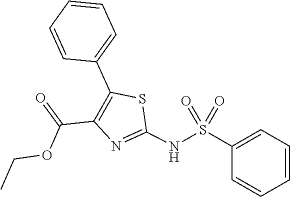 [SMILES: CCOC(=O)c1nc(NS(=O)(=O)c2ccccc2)sc1-c1ccccc1] | ++ | ++ | | |
| 42 | 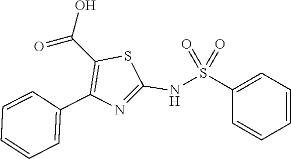 [SMILES: O=C(O)c1sc(NS(=O)(=O)c2ccccc2)nc1-c1ccccc1] | N.D. | ++ | | |
| 43 | 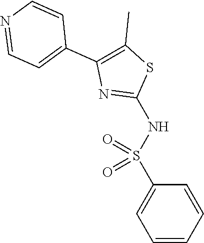 [SMILES: Cc1sc(NS(=O)(=O)c2ccccc2)nc1-c1ccncc1] | N.D. | + | | |
| 44 | 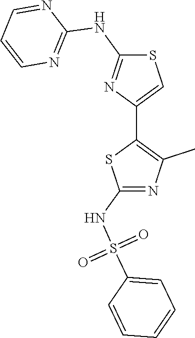 [SMILES: Cc1nc(NS(=O)(=O)c2ccccc2)sc1-c1csc(Nc2ncccn2)n1] | N.D. | + | | |
| 45 | 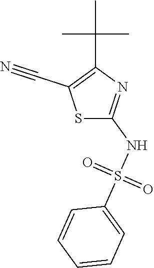 [SMILES: CC(C)(C)c1nc(NS(=O)(=O)c2ccccc2)sc1C#N] | N.D. | + | | |
| 46 | 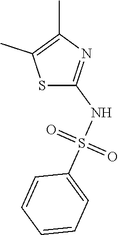 [SMILES: Cc1nc(NS(=O)(=O)c2ccccc2)sc1C] | N.D. | + | | |
| 47 | 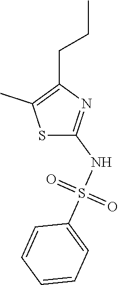 [SMILES: CCCc1nc(NS(=O)(=O)c2ccccc2)sc1C] | N.D. | + | | |
| 48 | 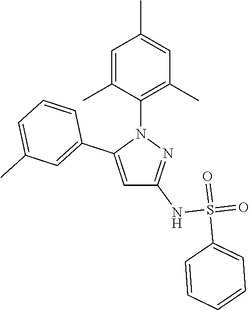 [SMILES: Cc1cccc(-c2cc(NS(=O)(=O)c3ccccc3)nn2-c2c(C)cc(C)cc2C)c1] | +++ | +++ | | |
| 49 | 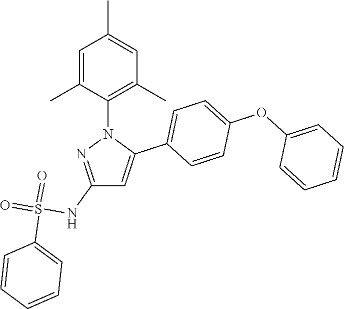 [SMILES: Cc1cc(C)c(-n2nc(NS(=O)(=O)c3ccccc3)cc2-c2ccc(Oc3ccccc3)cc2)c(C)c1] | +++ | ++ | | |
| 50 | 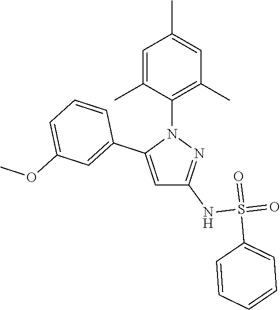 [SMILES: COc1cccc(-c2cc(NS(=O)(=O)c3ccccc3)nn2-c2c(C)cc(C)cc2C)c1] | ++ | +++ | | |
| 51 | 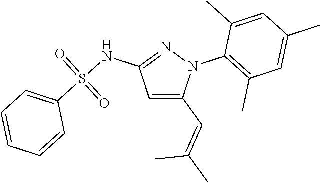 [SMILES: CC(C)=Cc1cc(NS(=O)(=O)c2ccccc2)nn1-c1c(C)cc(C)cc1C] | ++ | +++ | | |
| 52 | 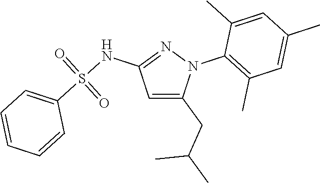 [SMILES: Cc1cc(C)c(-n2nc(NS(=O)(=O)c3ccccc3)cc2CC(C)C)c(C)c1] | ++ | +++ | | |
| 53 | 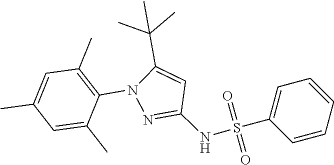 [SMILES: Cc1cc(C)c(-n2nc(NS(=O)(=O)c3ccccc3)cc2C(C)(C)C)c(C)c1] | N.D. | + | | |
| 54 | 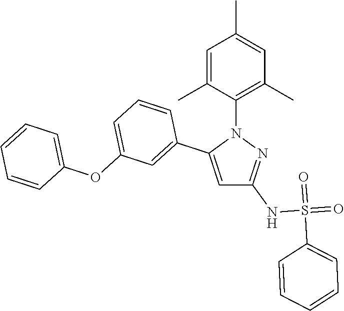 [SMILES: Cc1cc(C)c(-n2nc(NS(=O)(=O)c3ccccc3)cc2-c2cccc(Oc3ccccc3)c2)c(C)c1] | +++ | ++ | | |
| 55 | 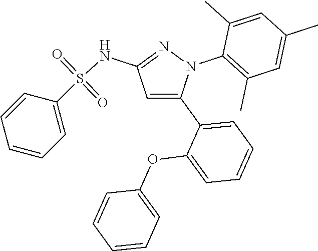 [SMILES: Cc1cc(C)c(-n2nc(NS(=O)(=O)c3ccccc3)cc2-c2ccccc2Oc2ccccc2)c(C)c1] | +++ | +++ | | |
| 56 | 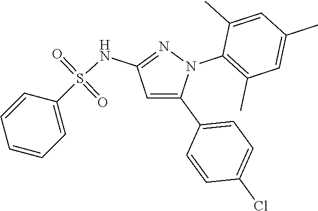 [SMILES: Cc1cc(C)c(-n2nc(NS(=O)(=O)c3ccccc3)cc2-c2ccc(Cl)cc2)c(C)c1] | +++ | ++ | | |
| 57 | 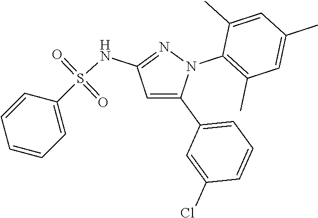 [SMILES: Cc1cc(C)c(-n2nc(NS(=O)(=O)c3ccccc3)cc2-c2cccc(Cl)c2)c(C)c1] | +++ | +++ | | |
| 58 | 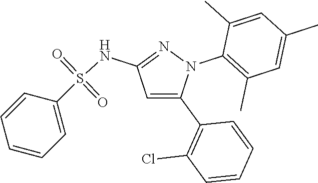 [SMILES: Cc1cc(C)c(-n2nc(NS(=O)(=O)c3ccccc3)cc2-c2ccccc2Cl)c(C)c1] | +++ | +++ | | |
| 59 | 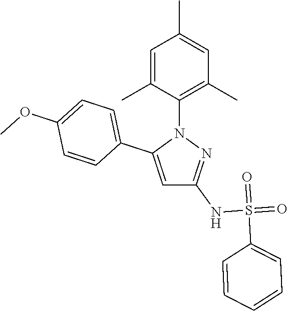 [SMILES: COc1ccc(-c2cc(NS(=O)(=O)c3ccccc3)nn2-c2c(C)cc(C)cc2C)cc1] | +++ | ++ | | |
| 60 | 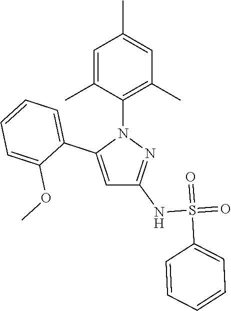 [SMILES: COc1ccccc1-c1cc(NS(=O)(=O)c2ccccc2)nn1-c1c(C)cc(C)cc1C] | ++ | +++ | | |
| 61 | 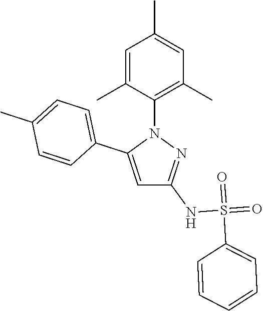 [SMILES: Cc1ccc(-c2cc(NS(=O)(=O)c3ccccc3)nn2-c2c(C)cc(C)cc2C)cc1] | +++ | ++ | | |
| 62 | 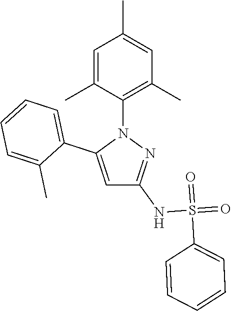 [SMILES: Cc1cc(C)c(-n2nc(NS(=O)(=O)c3ccccc3)cc2-c2ccccc2C)c(C)c1] | +++ | +++ | | |
| 63 | 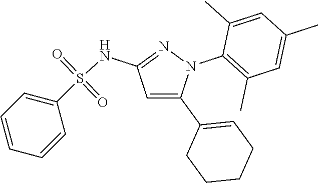 [SMILES: Cc1cc(C)c(-n2nc(NS(=O)(=O)c3ccccc3)cc2C2=CCCCC2)c(C)c1] | ++ | +++ | | |
| 64 | 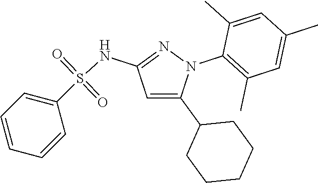 [SMILES: Cc1cc(C)c(-n2nc(NS(=O)(=O)c3ccccc3)cc2C2CCCCC2)c(C)c1] | +++ | +++ | | |
| 65 | 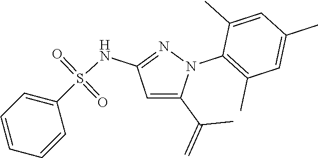 [SMILES: C=C(C)c1cc(NS(=O)(=O)c2ccccc2)nn1-c1c(C)cc(C)cc1C] | ++ | +++ | | |
| 66 | 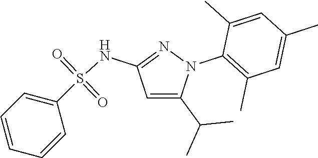 [SMILES: Cc1cc(C)c(-n2nc(NS(=O)(=O)c3ccccc3)cc2C(C)C)c(C)c1] | ++ | ++ | | |
| 67 | 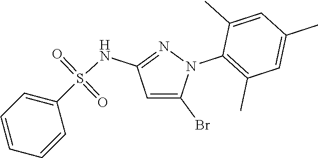 [SMILES: Cc1cc(C)c(-n2nc(NS(=O)(=O)c3ccccc3)cc2Br)c(C)c1] | ++ | +++ | | |
| 68 | 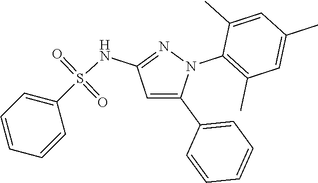 [SMILES: Cc1cc(C)c(-n2nc(NS(=O)(=O)c3ccccc3)cc2-c2ccccc2)c(C)c1] | ++ | +++ | | |
| 69 | 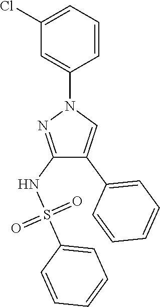 [SMILES: O=S(=O)(Nc1nn(-c2cccc(Cl)c2)cc1-c1ccccc1)c1ccccc1] | ++ | ++ | | |
| 70 | 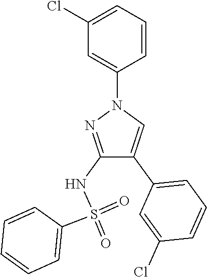 [SMILES: O=S(=O)(Nc1nn(-c2cccc(Cl)c2)cc1-c1cccc(Cl)c1)c1ccccc1] | +++ | ++ | | |
| 71 | 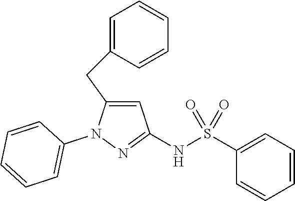 [SMILES: O=S(=O)(Nc1cc(Cc2ccccc2)n(-c2ccccc2)n1)c1ccccc1] | N.D. | + | | |
| 72 | 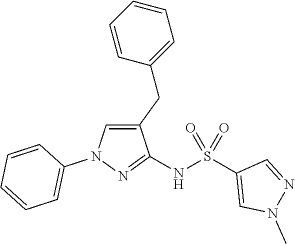 [SMILES: Cn1cc(S(=O)(=O)Nc2nn(-c3ccccc3)cc2Cc2ccccc2)cn1] | N.D. | + | | |
| 73 | 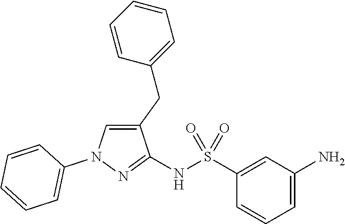 [SMILES: Nc1cccc(S(=O)(=O)Nc2nn(-c3ccccc3)cc2Cc2ccccc2)c1] | N.D. | ++ | | |
| 74 | 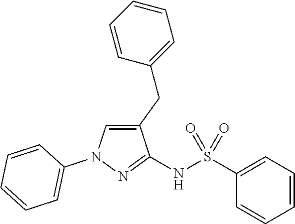 [SMILES: O=S(=O)(Nc1nn(-c2ccccc2)cc1Cc1ccccc1)c1ccccc1] | N.D. | + | | |
| 75 | 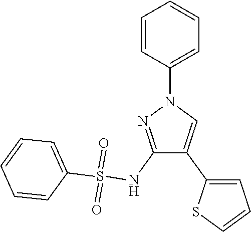 [SMILES: O=S(=O)(Nc1nn(-c2ccccc2)cc1-c1cccs1)c1ccccc1] | N.D. | + | | |
| 76 | 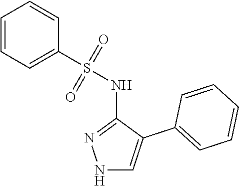 [SMILES: O=S(=O)(Nc1n[nH]cc1-c1ccccc1)c1ccccc1] | N.D. | + | | |
| 77 | 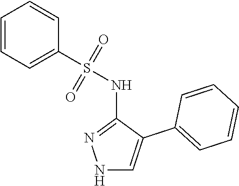 [SMILES: O=S(=O)(Nc1n[nH]cc1-c1ccccc1)c1ccccc1] | N.D. | + | | |
| 78 | 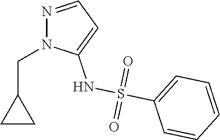 [SMILES: O=S(=O)(Nc1ccnn1CC1CC1)c1ccccc1] | N.D. | + | | |
| 79 | 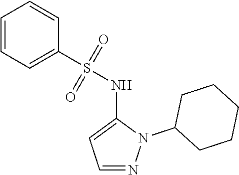 [SMILES: O=S(=O)(Nc1ccnn1C1CCCCC1)c1ccccc1] | N.D. | + | | |
| 80 | 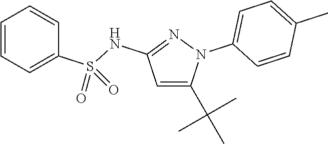 [SMILES: Cc1ccc(-n2nc(NS(=O)(=O)c3ccccc3)cc2C(C)(C)C)cc1] | N.D. | ++ | | |
| 81 | 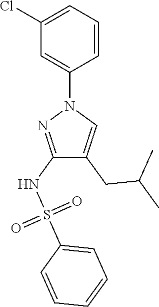 [SMILES: CC(C)Cc1cn(-c2cccc(Cl)c2)nc1NS(=O)(=O)c1ccccc1] | N.D. | + | | |
| 82 | 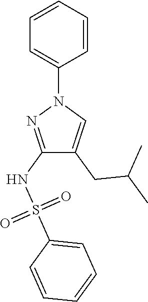 [SMILES: CC(C)Cc1cn(-c2ccccc2)nc1NS(=O)(=O)c1ccccc1] | N.D. | + | | |
| 83 | 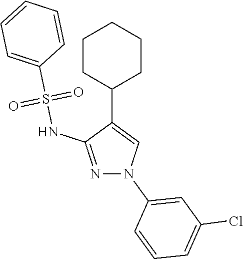 [SMILES: O=S(=O)(Nc1nn(-c2cccc(Cl)c2)cc1C1CCCCC1)c1ccccc1] | N.D. | + | | |
| 84 | 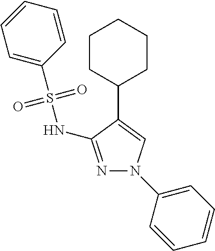 [SMILES: O=S(=O)(Nc1nn(-c2ccccc2)cc1C1CCCCC1)c1ccccc1] | N.D. | + | | |
| 85 | 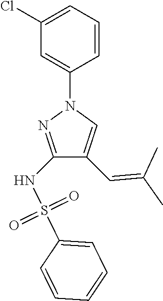 [SMILES: CC(C)=Cc1cn(-c2cccc(Cl)c2)nc1NS(=O)(=O)c1ccccc1] | + | ++ | | |
| 86 | 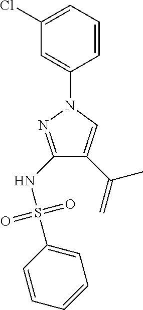 [SMILES: C=C(C)c1cn(-c2cccc(Cl)c2)nc1NS(=O)(=O)c1ccccc1] | + | ++ | | |
| 87 | 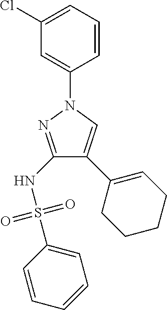 [SMILES: O=S(=O)(Nc1nn(-c2cccc(Cl)c2)cc1C1=CCCCC1)c1ccccc1] | ++ | ++ | | |
| 88 | 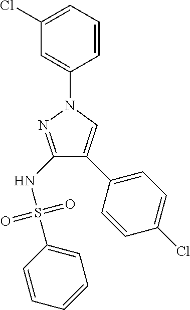 [SMILES: O=S(=O)(Nc1nn(-c2cccc(Cl)c2)cc1-c1ccc(Cl)cc1)c1ccccc1] | N.D. | + | | |
| 89 | 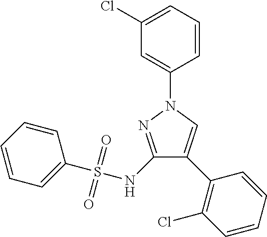 [SMILES: O=S(=O)(Nc1nn(-c2cccc(Cl)c2)cc1-c1ccccc1Cl)c1ccccc1] | N.D. | + | | |
| 90 | 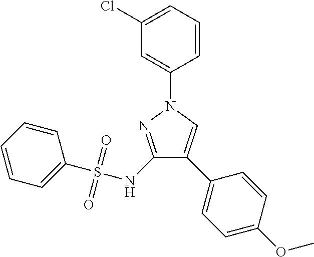 [SMILES: COc1ccc(-c2cn(-c3cccc(Cl)c3)nc2NS(=O)(=O)c2ccccc2)cc1] | +++ | + | | |
| 91 | 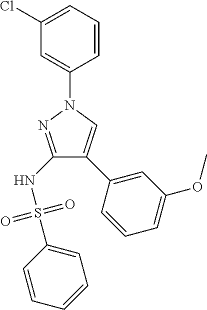 [SMILES: COc1cccc(-c2cn(-c3cccc(Cl)c3)nc2NS(=O)(=O)c2ccccc2)c1] | ++ | ++ | | |
| 92 | 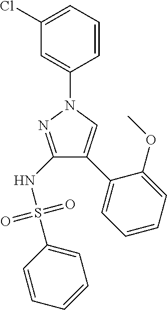 [SMILES: COc1ccccc1-c1cn(-c2cccc(Cl)c2)nc1NS(=O)(=O)c1ccccc1] | ++ | ++ | | |
| 93 | 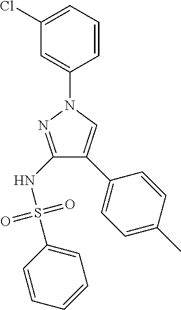 [SMILES: Cc1ccc(-c2cn(-c3cccc(Cl)c3)nc2NS(=O)(=O)c2ccccc2)cc1] | N.D. | + | | |
| 94 | 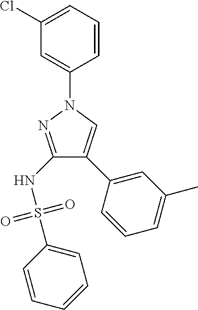 [SMILES: Cc1cccc(-c2cn(-c3cccc(Cl)c3)nc2NS(=O)(=O)c2ccccc2)c1] | ++ | ++ | | |
| 95 | 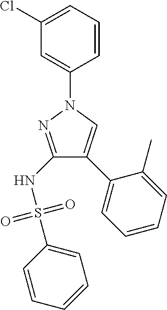 [SMILES: Cc1ccccc1-c1cn(-c2cccc(Cl)c2)nc1NS(=O)(=O)c1ccccc1] | N.D. | + | | |
| 96 | 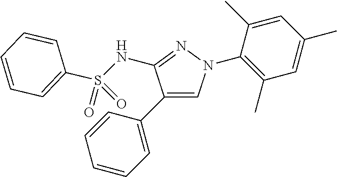 [SMILES: Cc1cc(C)c(-n2cc(-c3ccccc3)c(NS(=O)(=O)c3ccccc3)n2)c(C)c1] | +++ | ++ | | |
| 97 | 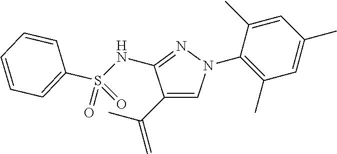 [SMILES: C=C(C)c1cn(-c2c(C)cc(C)cc2C)nc1NS(=O)(=O)c1ccccc1] | N.D. | + | | |
| 98 | 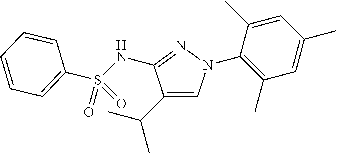 [SMILES: Cc1cc(C)c(-n2cc(C(C)C)c(NS(=O)(=O)c3ccccc3)n2)c(C)c1] | N.D. | + | | |
| 99 | 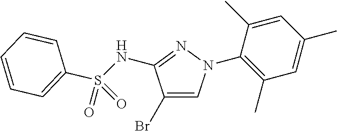 [SMILES: Cc1cc(C)c(-n2cc(Br)c(NS(=O)(=O)c3ccccc3)n2)c(C)c1] | N.D. | + | | |
| 100 | 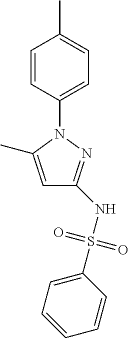 [SMILES: Cc1ccc(-n2nc(NS(=O)(=O)c3ccccc3)cc2C)cc1] | N.D. | + | | |
| 101 | 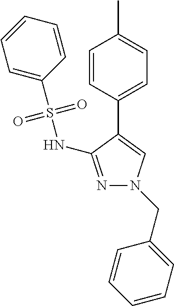 [SMILES: Cc1ccc(-c2cn(Cc3ccccc3)nc2NS(=O)(=O)c2ccccc2)cc1] | ++ | ++ | | |
| 102 | 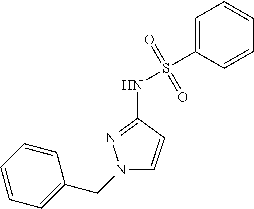 [SMILES: O=S(=O)(Nc1ccn(Cc2ccccc2)n1)c1ccccc1] | N.D. | + | | |
| 103 | 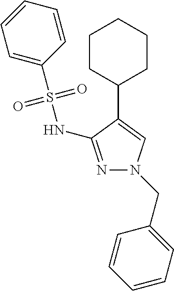 [SMILES: O=S(=O)(Nc1nn(Cc2ccccc2)cc1C1CCCCC1)c1ccccc1] | N.D. | + | | |
| 104 | 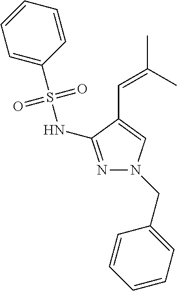 [SMILES: CC(C)=Cc1cn(Cc2ccccc2)nc1NS(=O)(=O)c1ccccc1] | N.D. | + | | |
| 105 | 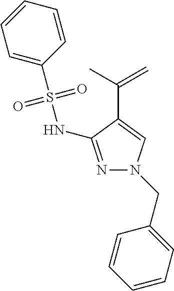 [SMILES: C=C(C)c1cn(Cc2ccccc2)nc1NS(=O)(=O)c1ccccc1] | N.D. | + | | |
| 106 | 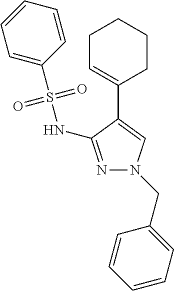 [SMILES: O=S(=O)(Nc1nn(Cc2ccccc2)cc1C1=CCCCC1)c1ccccc1] | N.D. | + | | |
| 107 | 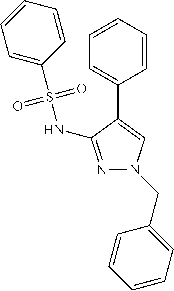 [SMILES: O=S(=O)(Nc1nn(Cc2ccccc2)cc1-c1ccccc1)c1ccccc1] | N.D. | + | | |
| 108 | 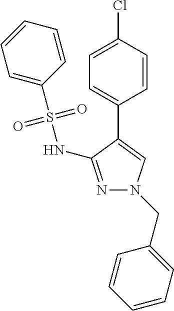 [SMILES: O=S(=O)(Nc1nn(Cc2ccccc2)cc1-c1ccc(Cl)cc1)c1ccccc1] | N.D. | ++ | | |
| 109 | 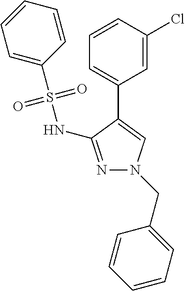 [SMILES: O=S(=O)(Nc1nn(Cc2ccccc2)cc1-c1cccc(Cl)c1)c1ccccc1] | N.D. | + | | |
| 110 | 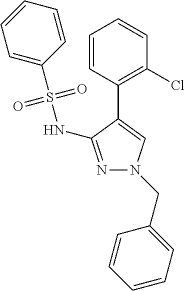 [SMILES: O=S(=O)(Nc1nn(Cc2ccccc2)cc1-c1ccccc1Cl)c1ccccc1] | N.D. | + | | |
| 111 | 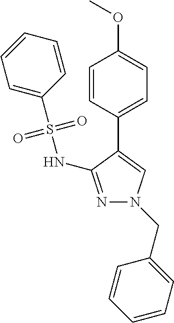 [SMILES: COc1ccc(-c2cn(Cc3ccccc3)nc2NS(=O)(=O)c2ccccc2)cc1] | N.D. | + | | |
| 112 | 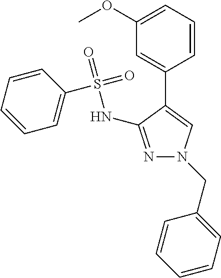 [SMILES: COc1cccc(-c2cn(Cc3ccccc3)nc2NS(=O)(=O)c2ccccc2)c1] | N.D. | + | | |
| 113 | 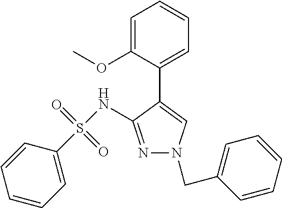 [SMILES: COc1ccccc1-c1cn(Cc2ccccc2)nc1NS(=O)(=O)c1ccccc1] | N.D. | + | | |
| 114 | 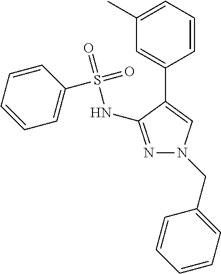 [SMILES: Cc1cccc(-c2cn(Cc3ccccc3)nc2NS(=O)(=O)c2ccccc2)c1] | N.D. | + | | |
| 115 | 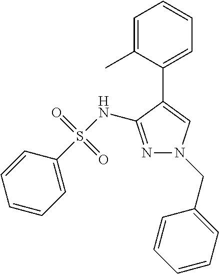 [SMILES: Cc1ccccc1-c1cn(Cc2ccccc2)nc1NS(=O)(=O)c1ccccc1] | N.D. | + | | |
| 116 | 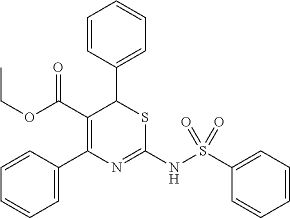 [SMILES: CCOC(=O)C1=C(c2ccccc2)N=C(NS(=O)(=O)c2ccccc2)SC1c1ccccc1] | +++ | +++ | | |
| 117 | 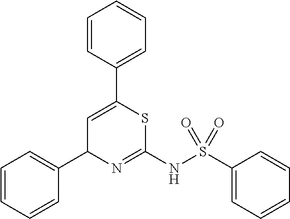 [SMILES: O=S(=O)(NC1=NC(c2ccccc2)C=C(c2ccccc2)S1)c1ccccc1] | ++ | ++ | | |
| 118 | 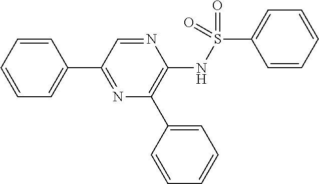 [SMILES: O=S(=O)(Nc1ncc(-c2ccccc2)nc1-c1ccccc1)c1ccccc1] | ++ | +++ | | |
| 119 | 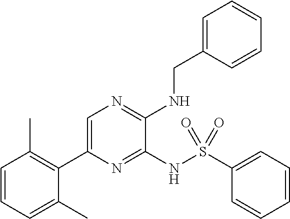 [SMILES: Cc1cccc(C)c1-c1cnc(NCc2ccccc2)c(NS(=O)(=O)c2ccccc2)n1] | ++ | ++ | | |
| 120 | 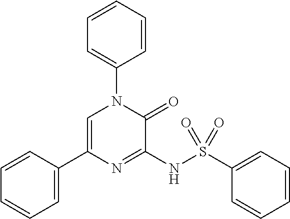 [SMILES: O=c1c(NS(=O)(=O)c2ccccc2)nc(-c2ccccc2)cn1-c1ccccc1] | ++ | ++ | | |
| 121 | 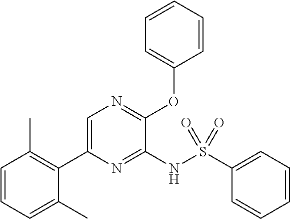 [SMILES: Cc1cccc(C)c1-c1cnc(Oc2ccccc2)c(NS(=O)(=O)c2ccccc2)n1] | N.D. | + | | |
| 122 | 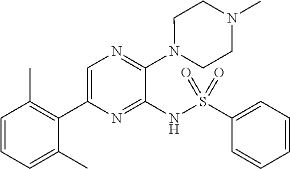 [SMILES: Cc1cccc(C)c1-c1cnc(N2CCN(C)CC2)c(NS(=O)(=O)c2ccccc2)n1] | N.D. | + | | |
| 123 | 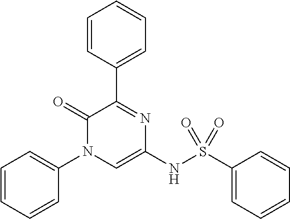 [SMILES: O=c1c(-c2ccccc2)nc(NS(=O)(=O)c2ccccc2)cn1-c1ccccc1] | N.D. | N.D. | | |
| 124 | 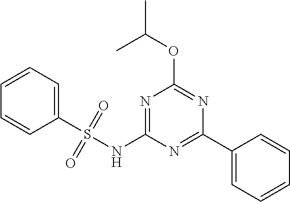 [SMILES: CC(C)Oc1nc(NS(=O)(=O)c2ccccc2)nc(-c2ccccc2)n1] | + | ++ | | |
| 125 | 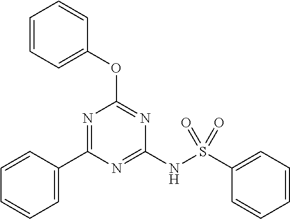 [SMILES: O=S(=O)(Nc1nc(Oc2ccccc2)nc(-c2ccccc2)n1)c1ccccc1] | +++ | ++ | | |
| 126 | 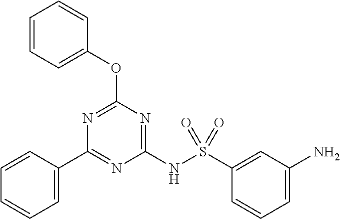 [SMILES: Nc1cccc(S(=O)(=O)Nc2nc(Oc3ccccc3)nc(-c3ccccc3)n2)c1] | + | ++ | | |
| 127 | 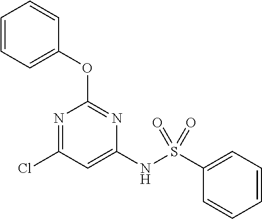 [SMILES: O=S(=O)(Nc1cc(Cl)nc(Oc2ccccc2)n1)c1ccccc1] | | | | |
| 128 | 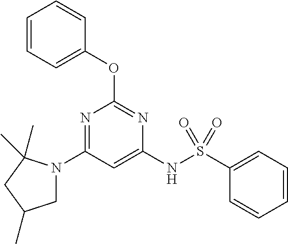 [SMILES: CC1CN(c2cc(NS(=O)(=O)c3ccccc3)nc(Oc3ccccc3)n2)C(C)(C)C1] | +++ | ++ | | |
| 129 | 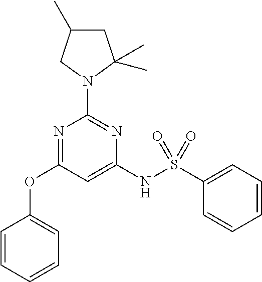 [SMILES: CC1CN(c2nc(NS(=O)(=O)c3ccccc3)cc(Oc3ccccc3)n2)C(C)(C)C1] | N.D. | + | | |
| 130 | 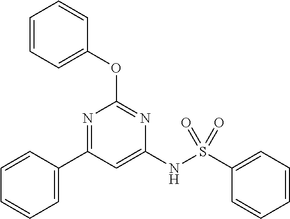 [SMILES: O=S(=O)(Nc1cc(-c2ccccc2)nc(Oc2ccccc2)n1)c1ccccc1] | ++ | ++ | | |
| 131 | 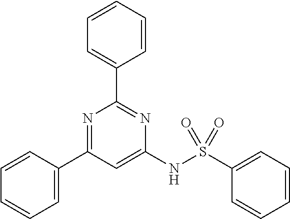 [SMILES: O=S(=O)(Nc1cc(-c2ccccc2)nc(-c2ccccc2)n1)c1ccccc1] | ++ | +++ | | |
| 132 | 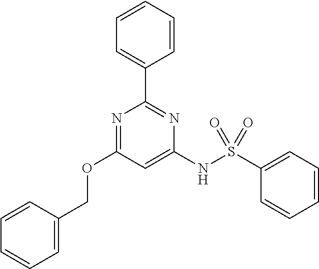 [SMILES: O=S(=O)(Nc1cc(OCc2ccccc2)nc(-c2ccccc2)n1)c1ccccc1] | ++ | ++ | | |
| 133 | 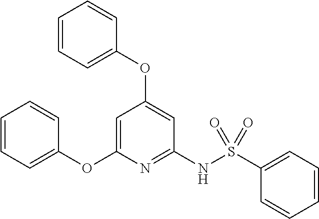 [SMILES: O=S(=O)(Nc1cc(Oc2ccccc2)cc(Oc2ccccc2)n1)c1ccccc1] | ++ | +++ | | |
| 134 | 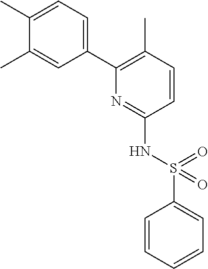 [SMILES: Cc1ccc(-c2nc(NS(=O)(=O)c3ccccc3)ccc2C)cc1C] | + | +++ | | |
| 135 | 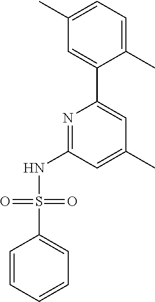 [SMILES: Cc1cc(NS(=O)(=O)c2ccccc2)nc(-c2cc(C)ccc2C)c1] | ++ | +++ | | |
| 136 | 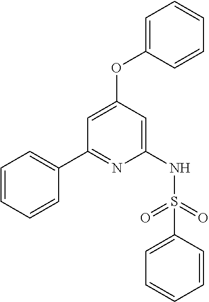 [SMILES: O=S(=O)(Nc1cc(Oc2ccccc2)cc(-c2ccccc2)n1)c1ccccc1] | ++ | +++ | | |
| 137 | 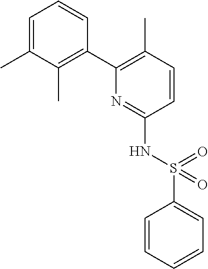 [SMILES: Cc1ccc(NS(=O)(=O)c2ccccc2)nc1-c1cccc(C)c1C] | ++ | +++ | | |
| 138 | 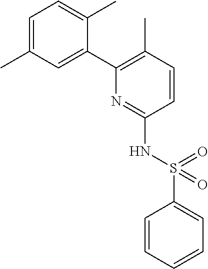 [SMILES: Cc1ccc(C)c(-c2nc(NS(=O)(=O)c3ccccc3)ccc2C)c1] | ++ | +++ | | |
| 139 | 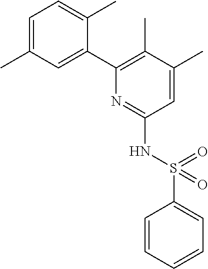 | ++ | +++ | | |
| 140 | 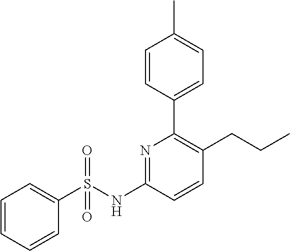 [SMILES: CCCc1ccc(NS(=O)(=O)c2ccccc2)nc1-c1ccc(C)cc1] | ++ | +++ | | |
| 141 | 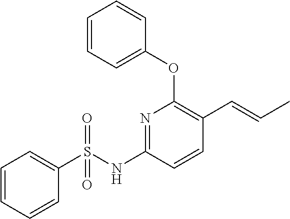 [SMILES: C/C=C/c1ccc(NS(=O)(=O)c2ccccc2)nc1Oc1ccccc1] | ++ | +++ | | |
| 142 | 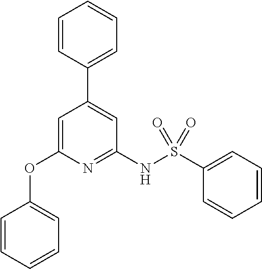 [SMILES: O=S(=O)(Nc1cc(-c2ccccc2)cc(Oc2ccccc2)n1)c1ccccc1] | +++ | +++ | | |
| 143 | 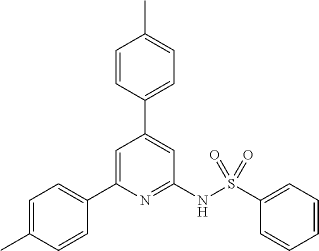 [SMILES: Cc1ccc(-c2cc(NS(=O)(=O)c3ccccc3)nc(-c3ccc(C)cc3)c2)cc1] | ++ | +++ | | |
| 144 | 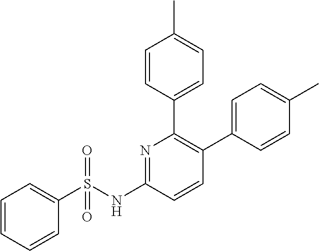 [SMILES: Cc1ccc(-c2ccc(NS(=O)(=O)c3ccccc3)nc2-c2ccc(C)cc2)cc1] | ++ | +++ | | |
| 145 | 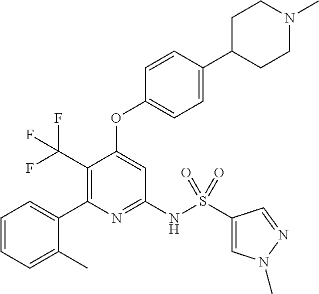 [SMILES: Cc1ccccc1-c1nc(NS(=O)(=O)c2cnn(C)c2)cc(Oc2ccc(C3CCN(C)CC3)cc2)c1C(F)(F)F] | | | N.D. | + |
| 146 | 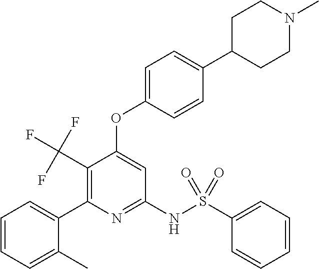 [SMILES: Cc1ccccc1-c1nc(NS(=O)(=O)c2ccccc2)cc(Oc2ccc(C3CCN(C)CC3)cc2)c1C(F)(F)F] | | | +++ | ++ |
| 147 | 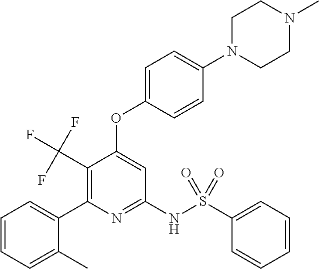 [SMILES: Cc1ccccc1-c1nc(NS(=O)(=O)c2ccccc2)cc(Oc2ccc(N3CCN(C)CC3)cc2)c1C(F)(F)F] | | | ++ | ++ |
| 148 | 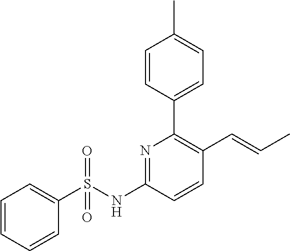 [SMILES: C/C=C/c1ccc(NS(=O)(=O)c2ccccc2)nc1-c1ccc(C)cc1] | ++ | +++ | | |
| 149 | 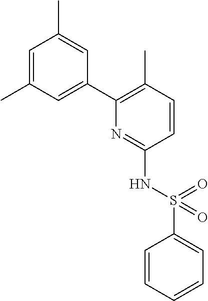 [SMILES: Cc1cc(C)cc(-c2nc(NS(=O)(=O)c3ccccc3)ccc2C)c1] | + | +++ | | |
| 150 | 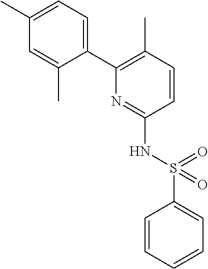 [SMILES: Cc1ccc(-c2nc(NS(=O)(=O)c3ccccc3)ccc2C)c(C)c1] | ++ | +++ | | |
| 151 | 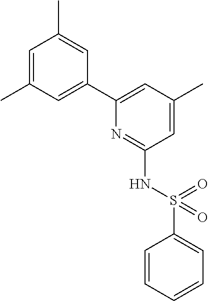 | + | ++ | | |
| 152 | 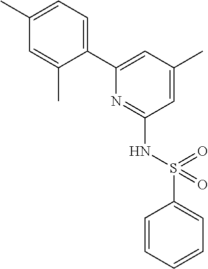 | + | +++ | | |
| 153 | 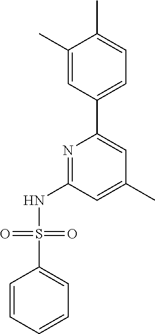 [SMILES: Cc1cc(NS(=O)(=O)c2ccccc2)nc(-c2ccc(C)c(C)c2)c1] | + | +++ | | |
| 154 | 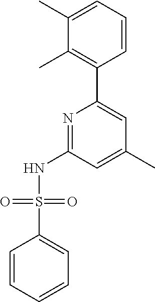 [SMILES: Cc1cc(NS(=O)(=O)c2ccccc2)nc(-c2cccc(C)c2C)c1] | + | +++ | | |
| 155 | 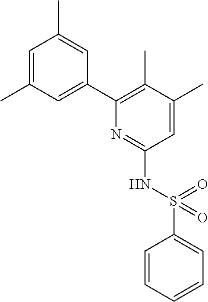 | ++ | +++ | | |
| 156 | 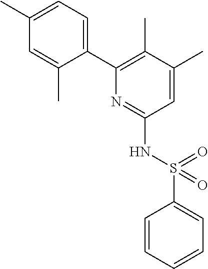 | + | +++ | | |
| 157 | 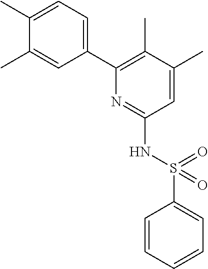 | ++ | +++ | | |
| 158 | 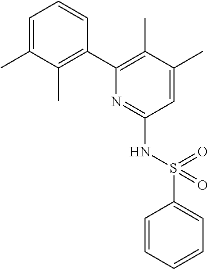 | + | +++ | | |

### VI. Synthesis of (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol

### A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 hours. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)+5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hours, then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. ¹⁹F NMR (282 MHz, CDCl₃) δ −63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 μM. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ −63.9 (s, 3F) ppm. ESI-MS m/z calc. 498.06134, Retention time: 2.34 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 hours, affording a yellow slurry. The mixture was cooled with an ice-bath and slowly acidified with HCl (1000 mL of 2 M, 2.000 mol), keeping the reaction temperature<15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et₂O (270 mL) at −78° C. was added bromo(but-3-enyl)magnesium in THE (190 mL of 0.817 M, 155.23 mmol) dropwise over a period of 1.5 hours (inner temperature −72° C. to −76° C.). The mixture was stirred at −78° C. for 20 minutes. The dry ice-acetone bath was removed. The mixture was slowly warm to 5° C. during 1 h, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃ (50 mL) and brine (20 mL), dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ −78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 minutes. Benzyl bromide (24.13 g, 141.08 mmol) and TBAI (8.68 g, 23.500 mmol) were added. The mixture was stirred at room temperature overnight. NH₄Cl (3 g, 0.6 eq) was added. The mixture was stirred for 10 min. 30 mL of EtOAc was added, then ice-water was added (400 g). The mixture was extracted with CH₂Cl₂ and the combined organic layers were concentrated. Purification by silica gel chromatography (0-20% CH₂Cl₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ −70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ −70.29 (s, 3F) ppm. ESI-MS m/z calc. 288.09732, found 287.1 (M−1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.10% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ −71.63 (br s, 3F) ppm. ESI-MS m/z calc. 288.0973, found 287.0 (M−1)⁻; Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over MgSO₄, filtered and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 μm particle) made by Waters (pn: 186002350) and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with IL of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T₃P (622 g of 50% w/w, 977.4 mmol) using an ice-water bath to keep the temperature<35° C. (temperature rose to 34° C.) and the reaction mixture was stirred at ambient temperature for 18 h. Added additional DIEA (100 mL, 574.1 mmol) and T₃P (95 g, 298.6 mmol) and stirred at ambient temperature for 2 days. Starting material was still observed and an additional T₃P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotovap bath until dissolved, then evaporated out 325 mL of solvent by rotary evaporation. The rotovap bath temp was allowed to drop to room temperature and product started crystallizing out during the evaporation. Then put the flask in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed water (3×3 L) and with brine (1×2.5 L). The organic phase was dried with MgSO₄, filtered and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotovap at 100 mbar pressure at room temperature for 1.5 h. The solid mass was stirred mechanically for 2 h at room temperature, resultant thick fine suspension was filtered, washed with dry ice cold heptane and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B ═CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z Mixture)

The following reaction was run, split equally between two, 12 L reaction flasks run in parallel. Mechanical stirring was employed, and reactions were subjected to a constant nitrogen gas purge using a course porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached ˜75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5]nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula I: 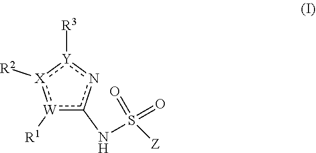
or a tautomer thereof, a deuterated derivative of the compound or tautomer, or a pharmaceutically acceptable salt of any of the foregoing, wherein:
W, X, and Y are each independently selected from C, S, O, and N;
wherein at least two of W, X, and Y are C;
Z is selected from phenyl (optionally substituted with NH₂) and pyrazole (optionally substituted with C₁₋₃ alkyl);
R¹ is absent or is selected from hydrogen, phenyl (optionally substituted with —C₁₋₃ alkyl, halogen, —C₁₋₄ alkoxy), —C₅₋₆ cycloalkyl, and —C₃₋₄ alkenyl;
R² is selected from hydrogen, phenyl (optionally substituted with halogen, —C₁₋₃ alkyl, —C₁₋₄ alkoxy, —O-phenyl), —C₁₋₆ alkyl (optionally substituted with 1-2 groups selected from ═O and —C₁₋₄ alkoxy), halogen, —C₃₋₆ cycloalkyl (optionally substituted with phenyl which is further optionally substituted with —C₁₋₄ alkoxy), —C₃₋₄ alkenyl, benzyl, —S(O)₂-phenyl, —C(O)NHC₁₋₆ alkyl, C(O)NHbenzyl, 5-6 membered heterocycle substituted with NH-phenyl (substituted with 1-2 groups selected from —C₁₋₄ alkoxy and halogen), 5-6 membered heteroaryl substituted with NH-phenyl (substituted with 1-2 groups selected from —C₁₋₄ alkoxy and halogen), heterocycle substituted with NH-heterocyclyl, and 5-6 membered heteroaryl substituted with NH-(5-6 membered heteroaryl),
wherein R² is not tert-butyl or C(O)NH-tert butyl; and
wherein at least one of R¹ and R² is absent or hydrogen; and
R³ is selected from phenyl (optionally substituted with 1-3 groups selected from halogen, C₁₋₄ alkyl, and C₁₋₄ alkoxy), −10 membered heteroaryl, and C₁₋₄ alkyl (optionally substituted with 1-2 groups selected from C₁₋₃ alkoxy, ═O, and phenyl).

2. The compound, tautomer, deuterated derivative, or pharmaceutically acceptable salt according to claim 1, selected from compounds of Formula Ia: 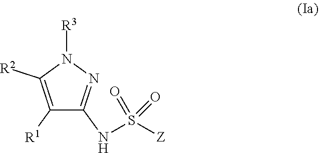
or a tautomer thereof, a deuterated derivative of the compound or tautomer, or a pharmaceutically acceptable salt of any of the foregoing, wherein variables R¹, R², R³, and Z are as defined in claim 1.

3. The compound, tautomer, deuterated derivative, or pharmaceutically acceptable salt according to claim 1, selected from compounds of Formula Ib: 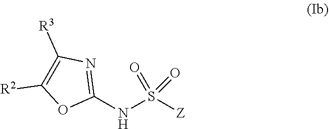
or a tautomer thereof, a deuterated derivative of the compound or tautomer, or a pharmaceutically acceptable salt of any of the foregoing, wherein variables R², R³, and Z are as defined in claim 1.

4. The compound, tautomer, deuterated derivative, or pharmaceutically acceptable salt according to claim 1, selected from compounds of Formula Ic: 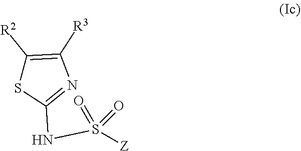
or a tautomer thereof, a deuterated derivative of the compound or tautomer, or a pharmaceutically acceptable salt of any of the foregoing, wherein variables R², R³, and Z are as defined in claim 1.

5. The compound, tautomer, deuterated derivative, or pharmaceutically acceptable salt according to any one of claims 1-4, wherein W is C and R¹ is selected from hydrogen and optionally substituted phenyl.

6. The compound, tautomer, deuterated derivative, or pharmaceutically acceptable salt according to any one of claims 1-5, wherein R² and R³ are optionally substituted phenyl.

7. A compound selected from:
N-[3,4-bis(4-chlorophenyl)isoxazol-5-yl]benzenesulfonamide (Compound 1);
N-(4-phenyl-5-propyl-oxazol-2-yl)benzenesulfonamide (Compound 2);
N-[4,5-bis(p-tolyl)oxazol-2-yl]benzenesulfonamide (Compound 3);
N-[5-[(2-methoxyphenyl)methyl]oxazol-2-yl]benzenesulfonamide (Compound 4);
N-[5-[(2-methoxyphenyl)methyl]oxazol-2-yl]benzenesulfonamide (Compound 5);
N-[4-(2,4-dimethylphenyl)-5-propyl-thiazol-2-yl]benzenesulfonamide (Compound 6);
N-(4,5-diphenylthiazol-2-yl)benzenesulfonamide (Compound 7);
N-[4-(2,5-dimethylphenyl)-5-methyl-thiazol-2-yl]benzenesulfonamide (Compound 8);
3-amino-N-(5-benzyl-4-phenyl-thiazol-2-yl)benzenesulfonamide (Compound 9);
N-[4,5-bis(p-tolyl)thiazol-2-yl]benzenesulfonamide (Compound 10);
N-(5-phenoxy-4-phenyl-thiazol-2-yl)benzenesulfonamide (Compound 11);
N-(4-cyclopropylthiazol-2-yl)benzenesulfonamide (Compound 12);
methyl 2-(benzenesulfonamido)-4-(p-tolyl)thiazole-5-carboxylate (Compound 13);
2-(benzenesulfonamido)-N-benzyl-4-phenyl-thiazole-5-carboxamide (Compound 14);
2-(benzenesulfonamido)-N-(3,3-dimethylbutyl)-4-phenyl-thiazole-5-carboxamide (Compound 15);
N-[5-(benzenesulfonyl)-4-(4-chlorophenyl)thiazol-2-yl]benzenesulfonamide (Compound 16);
N-(5-isopropyl-4-phenyl-thiazol-2-yl)benzenesulfonamide (Compound 17);
N-[4-(2,5-dimethylphenyl)-5-propyl-thiazol-2-yl]benzenesulfonamide (Compound 18);
N-[4-(4-ethoxyphenyl)-5-propyl-thiazol-2-yl]benzenesulfonamide (Compound 19);
N-[4-(4-methoxyphenyl)-5-propyl-thiazol-2-yl]benzenesulfonamide (Compound 20);
N-(5-ethyl-4-phenyl-thiazol-2-yl)benzenesulfonamide (Compound 21);
N-[4-(2,3-dihydro-1,4-benzodioxin-6-yl)-5-methyl-thiazol-2-yl]benzenesulfonamide (Compound 22);
N-(5-methyl-4-phenyl-thiazol-2-yl)benzenesulfonamide (Compound 23);
ethyl 2-(benzenesulfonamido)-4-phenyl-thiazole-5-carboxylate (Compound 24);
N-[5-[1-(2-methoxyphenyl)cyclopropyl]thiazol-2-yl]benzenesulfonamide (Compound 25);
N-(4-methyl-5-phenyl-thiazol-2-yl)benzenesulfonamide (Compound 26);
N-[5-[2-(5-chloro-2-methoxy-anilino)thiazol-4-yl]-4-methyl-thiazol-2-yl]benzenesulfonamide (Compound 27);
3-[[2-[(3-aminophenyl)sulfonylamino]-4-phenyl-thiazol-5-yl]methyl]benzoic acid (Compound 28);
N-[5-[2-(2-methoxyanilino)thiazol-4-yl]-4-methyl-thiazol-2-yl]benzenesulfonamide (Compound 29);
N-[4-methyl-5-[2-(2-pyridylamino)thiazol-4-yl]thiazol-2-yl]benzenesulfonamide (Compound 30);
N-[5-(pyrazin-2-ylmethyl)thiazol-2-yl]benzenesulfonamide (Compound 31);
N-[5-benzyl-thiazol-2-yl]benzenesulfonamide (Compound 32);
N,4-diphenyl-2-(phenylsulfonamido)thiazole-5-carboxamide (Compound 33);
N-[4-phenyl-5-(piperidine-1-carbonyl)thiazol-2-yl]benzenesulfonamide (Compound 34);
2-(benzenesulfonamido)-N-(2-methoxyethyl)-4-phenyl-thiazole-5-carboxamide (Compound 35);
2-(benzenesulfonamido)-N-tert-butyl-4-phenyl-thiazole-5-carboxamide (Compound 36);
2-(benzenesulfonamido)-N-[2-(cyclopropylmethoxy)ethyl]-4-phenyl-thiazole-5-carboxamide (Compound 37);
2-(benzenesulfonamido)-N-methyl-4-phenyl-N-propyl-thiazole-5-carboxamide (Compound 38);
2-(benzenesulfonamido)-4-phenyl-N-propyl-thiazole-5-carboxamide (Compound 39);
methyl 2-[2-(benzenesulfonamido)-5-phenyl-thiazol-4-yl]acetate (Compound 40);
ethyl 2-(benzenesulfonamido)-5-phenyl-thiazole-4-carboxylate (Compound 41);
4-phenyl-2-(phenylsulfonamido)thiazole-5-carboxylic acid (Compound 42);
N-[5-methyl-4-(4-pyridyl)thiazol-2-yl]benzenesulfonamide (Compound 43);
N-[4-methyl-5-[2-(pyrimidin-2-ylamino)thiazol-4-yl]thiazol-2-yl]benzenesulfonamide (Compound 44);
N-(4-tert-butyl-5-cyano-thiazol-2-yl)benzenesulfonamideN-(4-tert-butyl-5-cyano-thiazol-2-yl)benzenesulfonamide (Compound 45);
N-(4,5-dimethylthiazol-2-yl)benzenesulfonamide (Compound 46);
N-(5-methyl-4-propyl-thiazol-2-yl)benzenesulfonamide (Compound 47);
N-[5-(m-tolyl)-1-(2,4,6-trimethylphenyl)pyrazol-3-yl]benzenesulfonamide (Compound 48);
N-[5-(4-phenoxyphenyl)-1-(2,4,6-trimethylphenyl)pyrazol-3-yl]benzenesulfonamide (Compound 49);
N-[5-(3-methoxyphenyl)-1-(2,4,6-trimethylphenyl)pyrazol-3-yl]benzenesulfonamide (Compound 50);
N-[5-(2-methylprop-1-enyl)-1-(2,4,6-trimethylphenyl)pyrazol-3-yl]benzenesulfonamide (Compound 51);
N-[5-isobutyl-1-(2,4,6-trimethylphenyl)pyrazol-3-yl]benzenesulfonamide (Compound 52);
N-[5-tert-butyl-1-(2,4,6-trimethylphenyl)pyrazol-3-yl]benzenesulfonamide (Compound 53);
N-[5-(3-phenoxyphenyl)-1-(2,4,6-trimethylphenyl)pyrazol-3-yl]benzenesulfonamide (Compound 54);
N-[5-(2-phenoxyphenyl)-1-(2,4,6-trimethylphenyl)pyrazol-3-yl]benzenesulfonamide (Compound 55);
N-[5-(4-chlorophenyl)-1-(2,4,6-trimethylphenyl)pyrazol-3-yl]benzenesulfonamide (Compound 56);
N-[5-(3-chlorophenyl)-1-(2,4,6-trimethylphenyl)pyrazol-3-yl]benzenesulfonamide (Compound 57);
N-[5-(2-chlorophenyl)-1-(2,4,6-trimethylphenyl)pyrazol-3-yl]benzenesulfonamide (Compound 58);
N-[5-(4-methoxyphenyl)-1-(2,4,6-trimethylphenyl)pyrazol-3-yl]benzenesulfonamide (Compound 59);
N-[5-(2-methoxyphenyl)-1-(2,4,6-trimethylphenyl)pyrazol-3-yl]benzenesulfonamide (Compound 60);
N-[5-(p-tolyl)-1-(2,4,6-trimethylphenyl)pyrazol-3-yl]benzenesulfonamide (Compound 61);
N-[5-(p-tolyl)-1-(2,4,6-trimethylphenyl)pyrazol-3-yl]benzenesulfonamide (Compound 62);
N-[5-(cyclohexen-1-yl)-1-(2,4,6-trimethylphenyl)pyrazol-3-yl]benzenesulfonamide (Compound 63);
N-[5-cyclohexyl-1-(2,4,6-trimethylphenyl)pyrazol-3-yl]benzenesulfonamide (Compound 64);
N-[5-isopropenyl-1-(2,4,6-trimethylphenyl)pyrazol-3-yl]benzenesulfonamide (Compound 65);
N-[5-isopropyl-1-(2,4,6-trimethylphenyl)pyrazol-3-yl]benzenesulfonamide (Compound 66);
N-[5-bromo-1-(2,4,6-trimethylphenyl)pyrazol-3-yl]benzenesulfonamide (Compound 67);
N-[5-phenyl-1-(2,4,6-trimethylphenyl)pyrazol-3-yl]benzenesulfonamide (Compound 68);
N-[1-(3-chlorophenyl)-4-phenyl-pyrazol-3-yl]benzenesulfonamide (Compound 69);
N-[1,4-bis(3-chlorophenyl)pyrazol-3-yl]benzenesulfonamide (Compound 70);
N-(5-benzyl-1-phenyl-pyrazol-3-yl)benzenesulfonamide (Compound 71);
N-(4-benzyl-1-phenyl-pyrazol-3-yl)-1-methyl-pyrazole-4-sulfonamide (Compound 72);
3-amino-N-(4-benzyl-1-phenyl-pyrazol-3-yl)benzenesulfonamide (Compound 73);
N-(4-benzyl-1-phenyl-pyrazol-3-yl)benzenesulfonamide (Compound 74);
N-[1-phenyl-4-(2-thienyl)pyrazol-3-yl]benzenesulfonamide (Compound 75);
N-(2,5-diphenylpyrazol-3-yl)benzenesulfonamide (Compound 76);
N-(4-phenyl-1H-pyrazol-3-yl)benzenesulfonamide (Compound 77);
N-[2-(cyclopropylmethyl)pyrazol-3-yl]benzenesulfonamide (Compound 78);
N-(2-cyclohexylpyrazol-3-yl)benzenesulfonamide (Compound 79);
N-[5-tert-butyl-1-(p-tolyl)pyrazol-3-yl]benzenesulfonamide (Compound 80);
N-[1-(3-chlorophenyl)-4-isobutyl-pyrazol-3-yl]benzenesulfonamide (Compound 81);
N-(4-isobutyl-1-phenyl-pyrazol-3-yl)benzenesulfonamide (Compound 82);
N-[1-(3-chlorophenyl)-4-cyclohexyl-pyrazol-3-yl]benzenesulfonamide (Compound 83);
N-(4-cyclohexyl-1-phenyl-pyrazol-3-yl)benzenesulfonamide (Compound 84);
N-[1-(3-chlorophenyl)-4-(2-methylprop-1-enyl)pyrazol-3-yl]benzenesulfonamide (Compound 85);
N-[1-(3-chlorophenyl)-4-isopropenyl-pyrazol-3-yl]benzenesulfonamide (Compound 86);
N-[1-(3-chlorophenyl)-4-(cyclohexen-1-yl)pyrazol-3-yl]benzenesulfonamide (Compound 87);
N-[1-(3-chlorophenyl)-4-(4-chlorophenyl)pyrazol-3-yl]benzenesulfonamide (Compound 88);
N-[4-(2-chlorophenyl)-1-(3-chlorophenyl)pyrazol-3-yl]benzenesulfonamide (Compound 89);
N-[1-(3-chlorophenyl)-4-(4-methoxyphenyl)pyrazol-3-yl]benzenesulfonamide (Compound 90);
N-[1-(3-chlorophenyl)-4-(3-methoxyphenyl)pyrazol-3-yl]benzenesulfonamide (Compound 91);
N-[1-(3-chlorophenyl)-4-(2-methoxyphenyl)pyrazol-3-yl]benzenesulfonamide (Compound 92);
N-[1-(3-chlorophenyl)-4-(p-tolyl)pyrazol-3-yl]benzenesulfonamide (Compound 93);
N-[1-(3-chlorophenyl)-4-(m-tolyl)pyrazol-3-yl]benzenesulfonamide (Compound 94);
N-[1-(3-chlorophenyl)-4-(o-tolyl)pyrazol-3-yl]benzenesulfonamide (Compound 95);
N-[4-phenyl-1-(2,4,6-trimethylphenyl)pyrazol-3-yl]benzenesulfonamide (Compound 96);
N-[4-isopropenyl-1-(2,4,6-trimethylphenyl)pyrazol-3-yl]benzenesulfonamide (Compound 97);
N-[4-isopropenyl-1-(2,4,6-trimethylphenyl)pyrazol-3-yl]benzenesulfonamide (Compound 98);
N-[4-bromo-1-(2,4,6-trimethylphenyl)pyrazol-3-yl]benzenesulfonamide (Compound 99);
N-[5-methyl-1-(p-tolyl)pyrazol-3-yl]benzenesulfonamide (Compound 100);
N-[1-benzyl-4-(p-tolyl)pyrazol-3-yl]benzenesulfonamide (Compound 101);
N-(1-benzylpyrazol-3-yl)benzenesulfonamide (Compound 102);
N-(1-benzyl-4-cyclohexyl-pyrazol-3-yl)benzenesulfonamide (Compound 103);
N-[1-benzyl-4-(2-methylprop-1-enyl)pyrazol-3-yl]benzenesulfonamide (Compound 104);
N-(1-benzyl-4-isopropenyl-pyrazol-3-yl)benzenesulfonamide (Compound 105);
N-[1-benzyl-4-(cyclohexen-1-yl)pyrazol-3-yl]benzenesulfonamide (Compound 106);
N-(1-benzyl-4-phenyl-pyrazol-3-yl)benzenesulfonamide (Compound 107);
N-[1-benzyl-4-(4-chlorophenyl)pyrazol-3-yl]benzenesulfonamide (Compound 108);
N-[1-benzyl-4-(3-chlorophenyl)pyrazol-3-yl]benzenesulfonamide (Compound 109);
N-[1-benzyl-4-(2-chlorophenyl)pyrazol-3-yl]benzenesulfonamide (Compound 110);
N-[1-benzyl-4-(4-methoxyphenyl)pyrazol-3-yl]benzenesulfonamide (Compound 111);
N-[1-benzyl-4-(p-tolyl)pyrazol-3-yl]benzenesulfonamide (Compound 112);
N-[1-benzyl-4-(2-methoxyphenyl)pyrazol-3-yl]benzenesulfonamide (Compound 113);
N-[1-benzyl-4-(m-tolyl)pyrazol-3-yl]benzenesulfonamide (Compound 114); and
N-[5-cyclohexyl-1-(2,4,6-trimethylphenyl)pyrazol-3-yl]benzenesulfonamide (Compound 115),
or a tautomer thereof, a deuterated derivative of the compound or tautomer, or a pharmaceutically acceptable salt of any of the foregoing.

8. A compound selected from:
N-[3,4-bis(4-chlorophenyl)isoxazol-5-yl]benzenesulfonamide (Compound 1);
N-(4-phenyl-5-propyl-oxazol-2-yl)benzenesulfonamide (Compound 2);
N-[4,5-bis(p-tolyl)oxazol-2-yl]benzenesulfonamide (Compound 3);
N-[5-[(2-methoxyphenyl)methyl]oxazol-2-yl]benzenesulfonamide (Compound 5);
N-[4-(2,4-dimethylphenyl)-5-propyl-thiazol-2-yl]benzenesulfonamide (Compound 6);
N-(4,5-diphenylthiazol-2-yl)benzenesulfonamide (Compound 7);
N-[4-(2,5-dimethylphenyl)-5-methyl-thiazol-2-yl]benzenesulfonamide (Compound 8);
3-amino-N-(5-benzyl-4-phenyl-thiazol-2-yl)benzenesulfonamide (Compound 9);
N-[4,5-bis(p-tolyl)thiazol-2-yl]benzenesulfonamide (Compound 10);
methyl 2-(benzenesulfonamido)-4-(p-tolyl)thiazole-5-carboxylate (Compound 13);
2-(benzenesulfonamido)-N-benzyl-4-phenyl-thiazole-5-carboxamide (Compound 14);
2-(benzenesulfonamido)-N-(3,3-dimethylbutyl)-4-phenyl-thiazole-5-carboxamide (Compound 15);
N-[5-(benzenesulfonyl)-4-(4-chlorophenyl)thiazol-2-yl]benzenesulfonamide (Compound 16);
N-(5-isopropyl-4-phenyl-thiazol-2-yl)benzenesulfonamide (Compound 17);
N-[4-(2,5-dimethylphenyl)-5-propyl-thiazol-2-yl]benzenesulfonamide (Compound 18);
N-[4-(4-ethoxyphenyl)-5-propyl-thiazol-2-yl]benzenesulfonamide (Compound 19);
N-[4-(4-methoxyphenyl)-5-propyl-thiazol-2-yl]benzenesulfonamide (Compound 20);
N-(5-ethyl-4-phenyl-thiazol-2-yl)benzenesulfonamide (Compound 21);
N-[4-(2,3-dihydro-1,4-benzodioxin-6-yl)-5-methyl-thiazol-2-yl]benzenesulfonamide (Compound 22);
N-(5-methyl-4-phenyl-thiazol-2-yl)benzenesulfonamide (Compound 23);
ethyl 2-(benzenesulfonamido)-4-phenyl-thiazole-5-carboxylate (Compound 24);
N-[5-[1-(2-methoxyphenyl)cyclopropyl]thiazol-2-yl]benzenesulfonamide (Compound 25);
N-(4-methyl-5-phenyl-thiazol-2-yl)benzenesulfonamide (Compound 26);
N-[5-[2-(5-chloro-2-methoxy-anilino)thiazol-4-yl]-4-methyl-thiazol-2-yl]benzenesulfonamide (Compound 27);
N-[5-[2-(2-methoxyanilino)thiazol-4-yl]-4-methyl-thiazol-2-yl]benzenesulfonamide (Compound 29);
N-[4-methyl-5-[2-(2-pyridylamino)thiazol-4-yl]thiazol-2-yl]benzenesulfonamide (Compound 30);
methyl 2-[2-(benzenesulfonamido)-5-phenyl-thiazol-4-yl]acetate (Compound 40);
ethyl 2-(benzenesulfonamido)-5-phenyl-thiazole-4-carboxylate (Compound 41);
N-[5-(m-tolyl)-1-(2,4,6-trimethylphenyl)pyrazol-3-yl]benzenesulfonamide (Compound 48);
N-[5-(4-phenoxyphenyl)-1-(2,4,6-trimethylphenyl)pyrazol-3-yl]benzenesulfonamide (Compound 49);
N-[5-(3-methoxyphenyl)-1-(2,4,6-trimethylphenyl)pyrazol-3-yl]benzenesulfonamide (Compound 50);
N-[5-(2-methylprop-1-enyl)-1-(2,4,6-trimethylphenyl)pyrazol-3-yl]benzenesulfonamide (Compound 51);
N-[5-isobutyl-1-(2,4,6-trimethylphenyl)pyrazol-3-yl]benzenesulfonamide (Compound 52);
N-[5-(3-phenoxyphenyl)-1-(2,4,6-trimethylphenyl)pyrazol-3-yl]benzenesulfonamide (Compound 54);
N-[5-(2-phenoxyphenyl)-1-(2,4,6-trimethylphenyl)pyrazol-3-yl]benzenesulfonamide (Compound 55);
N-[5-(4-chlorophenyl)-1-(2,4,6-trimethylphenyl)pyrazol-3-yl]benzenesulfonamide (Compound 56);
N-[5-(3-chlorophenyl)-1-(2,4,6-trimethylphenyl)pyrazol-3-yl]benzenesulfonamide (Compound 57);
N-[5-(2-chlorophenyl)-1-(2,4,6-trimethylphenyl)pyrazol-3-yl]benzenesulfonamide (Compound 58);
N-[5-(4-methoxyphenyl)-1-(2,4,6-trimethylphenyl)pyrazol-3-yl]benzenesulfonamide (Compound 59);
N-[5-(2-methoxyphenyl)-1-(2,4,6-trimethylphenyl)pyrazol-3-yl]benzenesulfonamide (Compound 60);
N-[5-(p-tolyl)-1-(2,4,6-trimethylphenyl)pyrazol-3-yl]benzenesulfonamide (Compound 61);
N-[5-(p-tolyl)-1-(2,4,6-trimethylphenyl)pyrazol-3-yl]benzenesulfonamide (Compound 62);
N-[5-(cyclohexen-1-yl)-1-(2,4,6-trimethylphenyl)pyrazol-3-yl]benzenesulfonamide (Compound 63);
N-[5-cyclohexyl-1-(2,4,6-trimethylphenyl)pyrazol-3-yl]benzenesulfonamide (Compound 64);
N-[5-isopropenyl-1-(2,4,6-trimethylphenyl)pyrazol-3-yl]benzenesulfonamide (Compound 65);
N-[5-isopropyl-1-(2,4,6-trimethylphenyl)pyrazol-3-yl]benzenesulfonamide (Compound 66);
N-[5-bromo-1-(2,4,6-trimethylphenyl)pyrazol-3-yl]benzenesulfonamide (Compound 67);
N-[5-phenyl-1-(2,4,6-trimethylphenyl)pyrazol-3-yl]benzenesulfonamide (Compound 68);
N-[1-(3-chlorophenyl)-4-phenyl-pyrazol-3-yl]benzenesulfonamide (Compound 69);
N-[1,4-bis(3-chlorophenyl)pyrazol-3-yl]benzenesulfonamide (Compound 70);
N-[1-(3-chlorophenyl)-4-(2-methylprop-1-enyl)pyrazol-3-yl]benzenesulfonamide (Compound 85);
N-[1-(3-chlorophenyl)-4-isopropenyl-pyrazol-3-yl]benzenesulfonamide (Compound 86);
N-[1-(3-chlorophenyl)-4-(cyclohexen-1-yl)pyrazol-3-yl]benzenesulfonamide (Compound 87);
N-[1-(3-chlorophenyl)-4-(4-methoxyphenyl)pyrazol-3-yl]benzenesulfonamide (Compound 90);
N-[1-(3-chlorophenyl)-4-(3-methoxyphenyl)pyrazol-3-yl]benzenesulfonamide (Compound 91);
N-[1-(3-chlorophenyl)-4-(2-methoxyphenyl)pyrazol-3-yl]benzenesulfonamide (Compound 92);
N-[1-(3-chlorophenyl)-4-(m-tolyl)pyrazol-3-yl]benzenesulfonamide (Compound 94);
N-[4-phenyl-1-(2,4,6-trimethylphenyl)pyrazol-3-yl]benzenesulfonamide (Compound 96); and
N-[1-benzyl-4-(p-tolyl)pyrazol-3-yl]benzenesulfonamide (Compound 101),
or a tautomer thereof, a deuterated derivative of the compound or tautomer, or a pharmaceutically acceptable salt of any of the foregoing.

9. A compound of Formula II: 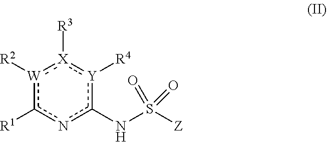
or a tautomer thereof, a deuterated derivative of the compound or tautomer, or a pharmaceutically acceptable salt of any of the foregoing, wherein:
W, X, and Y are independently selected from C, N, and S, wherein at least one of W, X, and Y is C, and wherein Y cannot be N unless W is also N;
Z is selected from phenyl (optionally substituted with NH₂) and pyrazole (optionally substituted with —C₁₋₃ alkyl);
R¹ is absent or is selected from hydrogen, -phenyl (optionally substituted with 1-3 groups independently selected from —C₁₋₃ alkyl), —O-phenyl, and —C₁₋₄ alkoxy;
R² is absent or is selected from hydrogen, —C₁₋₃ alkyl (optionally substituted with 1-3 halogen), —C₁₋₃ alkenyl, —C₁₋₄ alkoxy, —C(O)C₁₋₄ alkoxy, and -phenyl (optionally substituted with —C₁₋₃ alkyl);
R³ is absent or is selected from hydrogen, halogen, C₁₋₃ alkyl, -phenyl (optionally substituted with —C₁₋₃ alkyl), —C₁₋₄ alkoxy, —O-phenyl (optionally substituted with 5-6 membered heterocycle which is further optionally substituted with —C₁₋₃ alkyl), —O-benzyl, and 5-6 membered heterocycle (optionally substituted with 1-3 groups independently selected from —C₁₋₃ alkyl); and
R⁴ is absent or is selected from hydrogen, phenyl, NH-benzyl, and ═O.

10. The compound, tautomer, deuterated derivative, or pharmaceutically acceptable salt according to claim 9, selected from compounds of Formula IIa: 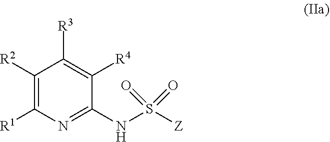
or a tautomer thereof, a deuterated derivative of the compound or tautomer, or a pharmaceutically acceptable salt of any of the foregoing, wherein variables R¹, R², R³, R⁴, and Z are as defined in claim 9.

11. The compound, tautomer, deuterated derivative, or pharmaceutically acceptable salt according to claim 9, selected from compounds of Formula IIb: 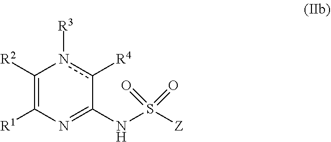
or a tautomer thereof, a deuterated derivative of the compound or tautomer, or a pharmaceutically acceptable salt of any of the foregoing, wherein variables R¹, R², R³, R⁴, and Z are as defined in claim 9.

12. The compound, tautomer, deuterated derivative, or pharmaceutically acceptable salt according to claim 9, selected from compounds of Formula IIc: 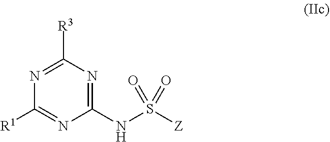
or a tautomer thereof, a deuterated derivative of the compound or tautomer, or a pharmaceutically acceptable salt of any of the foregoing, wherein variables R¹, R², and Z are as defined in claim 9.

13. The compound, tautomer, deuterated derivative, or pharmaceutically acceptable salt according to claim 9, selected from compounds of Formula IId: 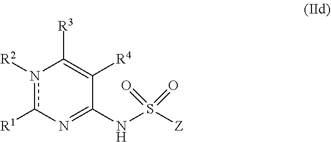
or a tautomer thereof, a deuterated derivative of the compound or tautomer, or a pharmaceutically acceptable salt of any of the foregoing, wherein variables R¹, R², R³, R⁴, and Z are as defined in claim 9.

14. The compound, tautomer, deuterated derivative, or pharmaceutically acceptable salt according to claim 9, selected from compounds of Formula IIe: 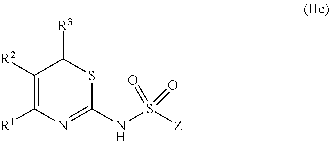
or a tautomer thereof, a deuterated derivative of the compound or tautomer, or a pharmaceutically acceptable salt of any of the foregoing, wherein variables R¹, R², R³, and Z are as defined in claim 9.

15. The compound, tautomer, deuterated derivative, or pharmaceutically acceptable salt according to any one of claims 9-14, wherein R¹ is selected from optionally substituted phenyl and O-phenyl.

16. The compound, tautomer, deuterated derivative, or pharmaceutically acceptable salt according to any one of claims 9-15, wherein R³ is selected from optionally substituted phenyl and optionally substituted O-phenyl.

17. A compound selected from:

| Cmpd No. | Structure |
|---|---|
| 116 | 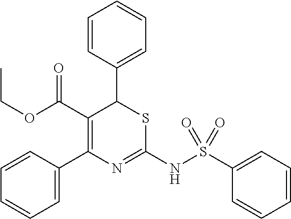 [SMILES: CCOC(=O)C1=C(c2ccccc2)N=C(NS(=O)(=O)c2ccccc2)SC1c1ccccc1] |
| 117 | 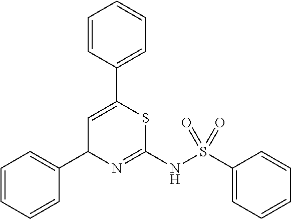 [SMILES: O=S(=O)(NC1=NC(c2ccccc2)C=C(c2ccccc2)S1)c1ccccc1] |
| 118 | 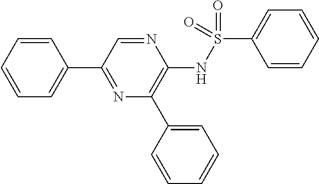 [SMILES: O=S(=O)(Nc1ncc(-c2ccccc2)nc1-c1ccccc1)c1ccccc1] |
| 119 | 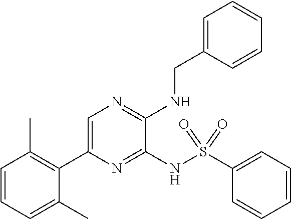 [SMILES: Cc1cccc(C)c1-c1cnc(NCc2ccccc2)c(NS(=O)(=O)c2ccccc2)n1] |
| 120 | 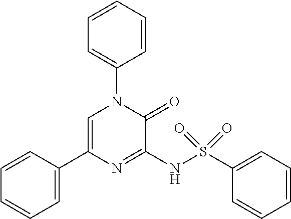 [SMILES: O=c1c(NS(=O)(=O)c2ccccc2)nc(-c2ccccc2)cn1-c1ccccc1] |
| 121 | 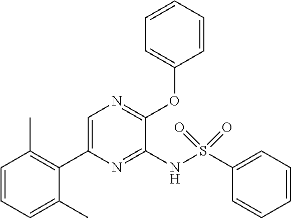 [SMILES: Cc1cccc(C)c1-c1cnc(Oc2ccccc2)c(NS(=O)(=O)c2ccccc2)n1] |
| 122 | 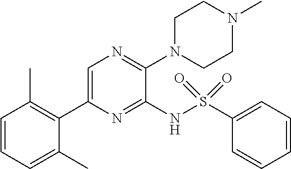 [SMILES: Cc1cccc(C)c1-c1cnc(N2CCN(C)CC2)c(NS(=O)(=O)c2ccccc2)n1] |
| 123 | 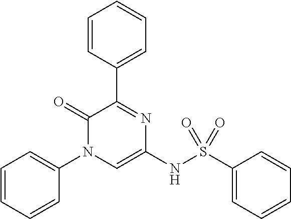 [SMILES: O=c1c(-c2ccccc2)nc(NS(=O)(=O)c2ccccc2)cn1-c1ccccc1] |
| 124 | 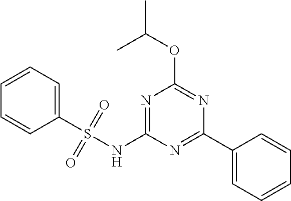 [SMILES: CC(C)Oc1nc(NS(=O)(=O)c2ccccc2)nc(-c2ccccc2)n1] |
| 125 | 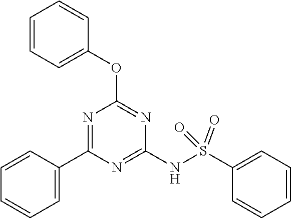 [SMILES: O=S(=O)(Nc1nc(Oc2ccccc2)nc(-c2ccccc2)n1)c1ccccc1] |
| 126 | 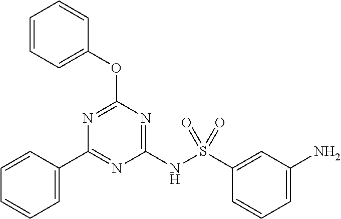 [SMILES: Nc1cccc(S(=O)(=O)Nc2nc(Oc3ccccc3)nc(-c3ccccc3)n2)c1] |
| 127 | 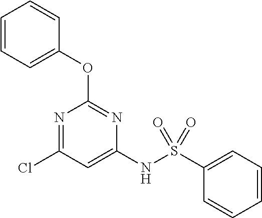 [SMILES: O=S(=O)(Nc1cc(Cl)nc(Oc2ccccc2)n1)c1ccccc1] |
| 128 | 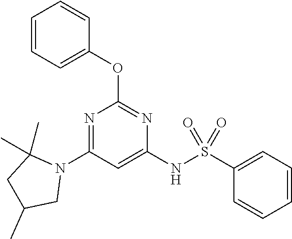 [SMILES: CC1CN(c2cc(NS(=O)(=O)c3ccccc3)nc(Oc3ccccc3)n2)C(C)(C)C1] |
| 129 | 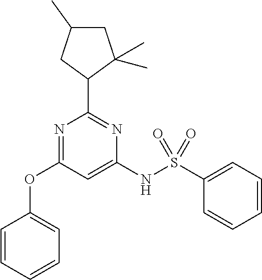 [SMILES: CC1CC(c2nc(NS(=O)(=O)c3ccccc3)cc(Oc3ccccc3)n2)C(C)(C)C1] |
| 130 | 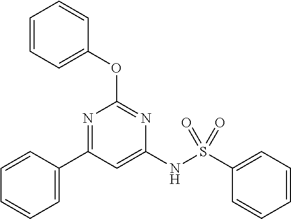 [SMILES: O=S(=O)(Nc1cc(-c2ccccc2)nc(Oc2ccccc2)n1)c1ccccc1] |
| 131 | 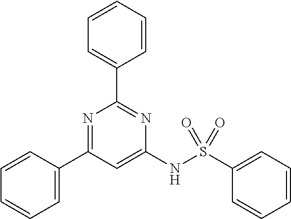 [SMILES: O=S(=O)(Nc1cc(-c2ccccc2)nc(-c2ccccc2)n1)c1ccccc1] |
| 132 | 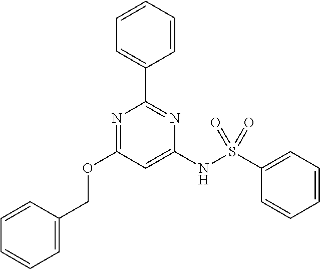 [SMILES: O=S(=O)(Nc1cc(OCc2ccccc2)nc(-c2ccccc2)n1)c1ccccc1] |
| 133 | 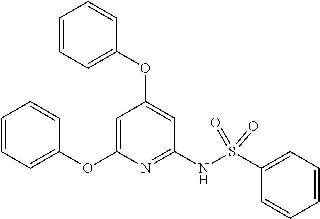 [SMILES: O=S(=O)(Nc1cc(Oc2ccccc2)cc(Oc2ccccc2)n1)c1ccccc1] |
| 134 | 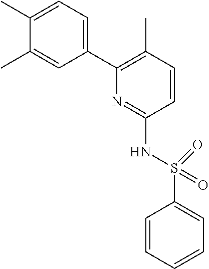 [SMILES: Cc1ccc(-c2nc(NS(=O)(=O)c3ccccc3)ccc2C)cc1C] |
| 135 | 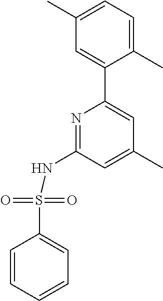 [SMILES: Cc1cc(NS(=O)(=O)c2ccccc2)nc(-c2cc(C)ccc2C)c1] |
| 136 | 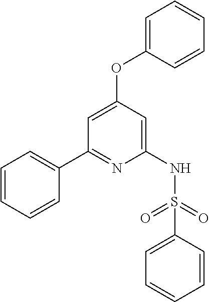 [SMILES: O=S(=O)(Nc1cc(Oc2ccccc2)cc(-c2ccccc2)n1)c1ccccc1] |
| 137 | 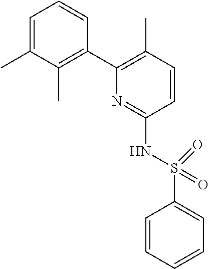 [SMILES: Cc1ccc(NS(=O)(=O)c2ccccc2)nc1-c1cccc(C)c1C] |
| 138 | 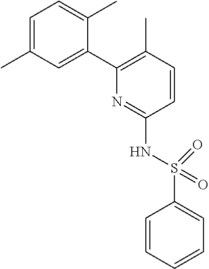 [SMILES: Cc1ccc(C)c(-c2nc(NS(=O)(=O)c3ccccc3)ccc2C)c1] |
| 139 | 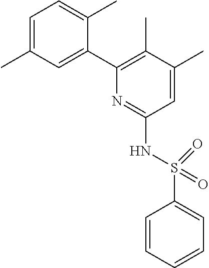 |
| 140 | 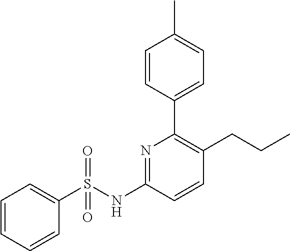 [SMILES: CCCc1ccc(NS(=O)(=O)c2ccccc2)nc1-c1ccc(C)cc1] |
| 141 | 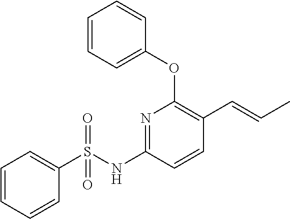 [SMILES: C/C=C/c1ccc(NS(=O)(=O)c2ccccc2)nc1Oc1ccccc1] |
| 142 | 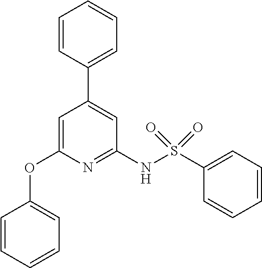 [SMILES: O=S(=O)(Nc1cc(-c2ccccc2)cc(Oc2ccccc2)n1)c1ccccc1] |
| 143 | 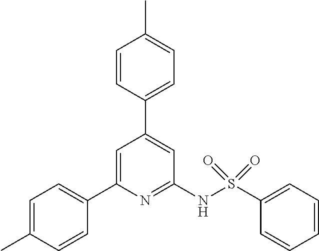 [SMILES: Cc1ccc(-c2cc(NS(=O)(=O)c3ccccc3)nc(-c3ccc(C)cc3)c2)cc1] |
| 144 | 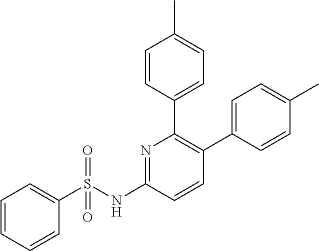 [SMILES: Cc1ccc(-c2ccc(NS(=O)(=O)c3ccccc3)nc2-c2ccc(C)cc2)cc1] |
| 145 | 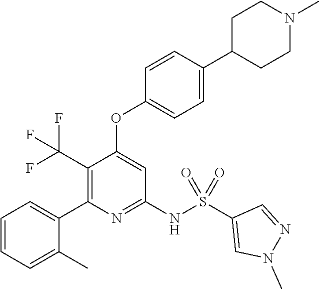 [SMILES: Cc1ccccc1-c1nc(NS(=O)(=O)c2cnn(C)c2)cc(Oc2ccc(C3CCN(C)CC3)cc2)c1C(F)(F)F] |
| 146 | 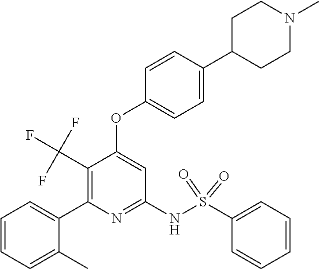 [SMILES: Cc1ccccc1-c1nc(NS(=O)(=O)c2ccccc2)cc(Oc2ccc(C3CCN(C)CC3)cc2)c1C(F)(F)F] |
| 147 | 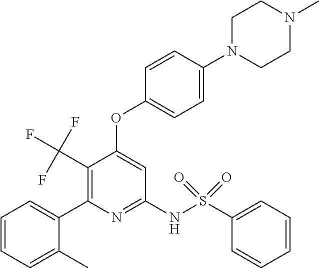 [SMILES: Cc1ccccc1-c1nc(NS(=O)(=O)c2ccccc2)cc(Oc2ccc(N3CCN(C)CC3)cc2)c1C(F)(F)F] |
| 148 | 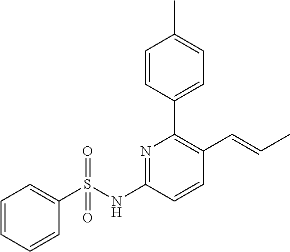 [SMILES: C/C=C/c1ccc(NS(=O)(=O)c2ccccc2)nc1-c1ccc(C)cc1] |
| 149 | 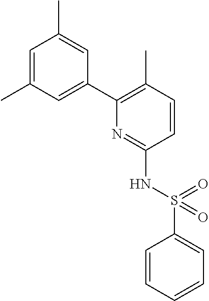 [SMILES: Cc1cc(C)cc(-c2nc(NS(=O)(=O)c3ccccc3)ccc2C)c1] |
| 150 | 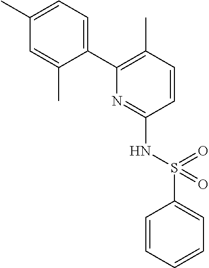 [SMILES: Cc1ccc(-c2nc(NS(=O)(=O)c3ccccc3)ccc2C)c(C)c1] |
| 151 | 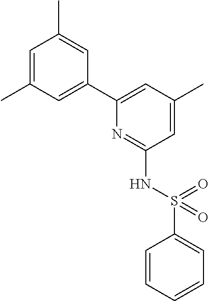 |
| 152 | 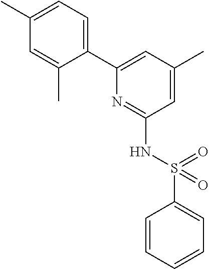 |
| 153 | 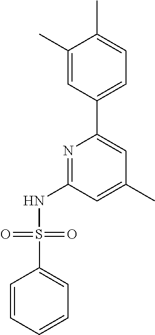 [SMILES: Cc1cc(NS(=O)(=O)c2ccccc2)nc(-c2ccc(C)c(C)c2)c1] |
| 154 | 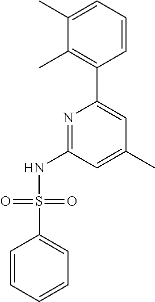 [SMILES: Cc1cc(NS(=O)(=O)c2ccccc2)nc(-c2cccc(C)c2C)c1] |
| 155 | 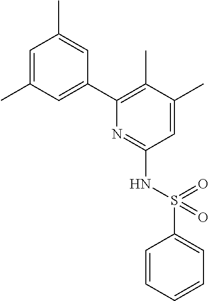 |
| 156 | 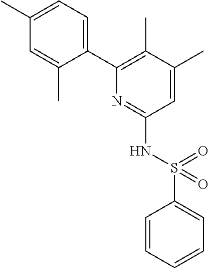 |
| 157 | 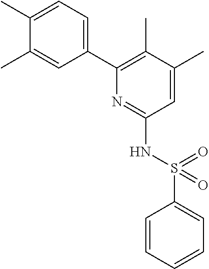 |
| 158 | 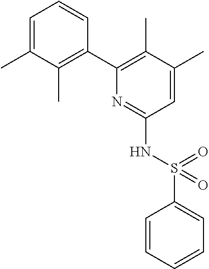 |

or a tautomer thereof, a deuterated derivative of the compound or tautomer, or a pharmaceutically acceptable salt of any of the foregoing.

18. A compound selected from:

| Cmpd No. | Structure |
|---|---|
| 116 | 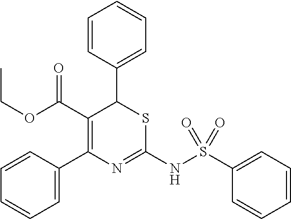 [SMILES: CCOC(=O)C1=C(c2ccccc2)N=C(NS(=O)(=O)c2ccccc2)SC1c1ccccc1] |
| 117 | 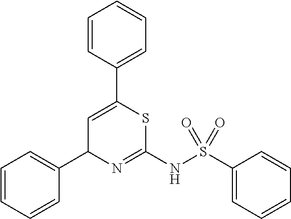 [SMILES: O=S(=O)(NC1=NC(c2ccccc2)C=C(c2ccccc2)S1)c1ccccc1] |
| 118 | 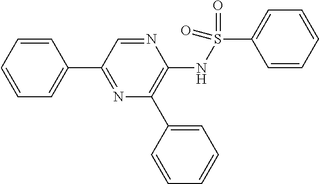 [SMILES: O=S(=O)(Nc1ncc(-c2ccccc2)nc1-c1ccccc1)c1ccccc1] |
| 119 | 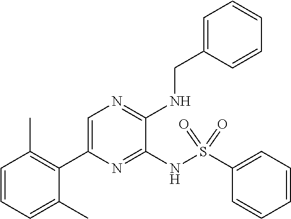 [SMILES: Cc1cccc(C)c1-c1cnc(NCc2ccccc2)c(NS(=O)(=O)c2ccccc2)n1] |
| 120 | 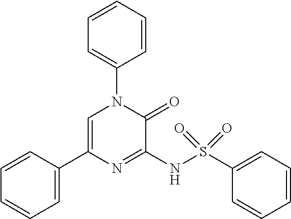 [SMILES: O=c1c(NS(=O)(=O)c2ccccc2)nc(-c2ccccc2)cn1-c1ccccc1] |
| 124 | 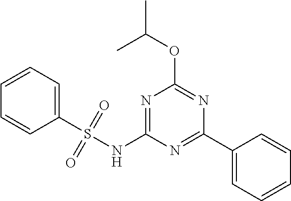 [SMILES: CC(C)Oc1nc(NS(=O)(=O)c2ccccc2)nc(-c2ccccc2)n1] |
| 125 | 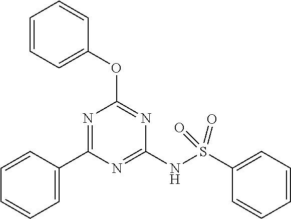 [SMILES: O=S(=O)(Nc1nc(Oc2ccccc2)nc(-c2ccccc2)n1)c1ccccc1] |
| 126 | 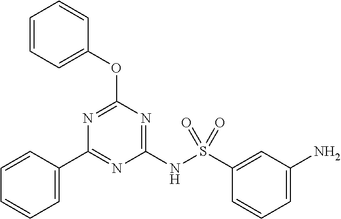 [SMILES: Nc1cccc(S(=O)(=O)Nc2nc(Oc3ccccc3)nc(-c3ccccc3)n2)c1] |
| 128 | 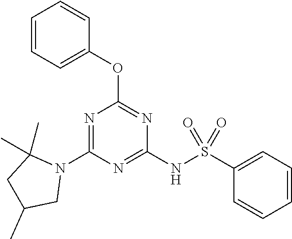 [SMILES: CC1CN(c2nc(NS(=O)(=O)c3ccccc3)nc(Oc3ccccc3)n2)C(C)(C)C1] |
| 130 | 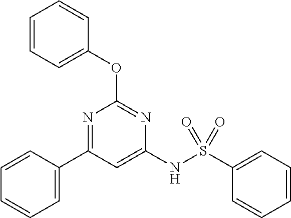 [SMILES: O=S(=O)(Nc1cc(-c2ccccc2)nc(Oc2ccccc2)n1)c1ccccc1] |
| 131 | 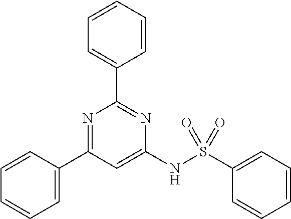 [SMILES: O=S(=O)(Nc1cc(-c2ccccc2)nc(-c2ccccc2)n1)c1ccccc1] |
| 132 | 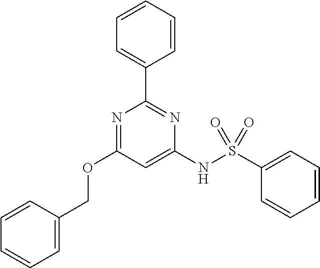 [SMILES: O=S(=O)(Nc1cc(OCc2ccccc2)nc(-c2ccccc2)n1)c1ccccc1] |
| 133 | 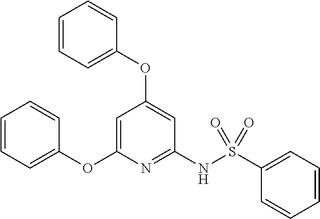 [SMILES: O=S(=O)(Nc1cc(Oc2ccccc2)cc(Oc2ccccc2)n1)c1ccccc1] |
| 134 | 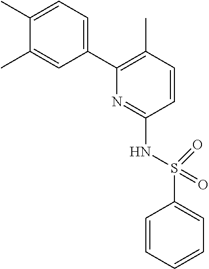 [SMILES: Cc1ccc(-c2nc(NS(=O)(=O)c3ccccc3)ccc2C)cc1C] |
| 135 | 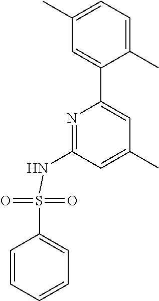 [SMILES: Cc1cc(NS(=O)(=O)c2ccccc2)nc(-c2cc(C)ccc2C)c1] |
| 136 | 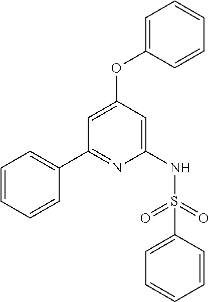 [SMILES: O=S(=O)(Nc1cc(Oc2ccccc2)cc(-c2ccccc2)n1)c1ccccc1] |
| 137 | 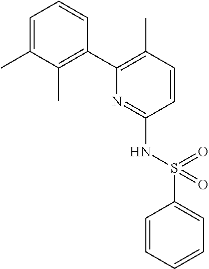 [SMILES: Cc1ccc(NS(=O)(=O)c2ccccc2)nc1-c1cccc(C)c1C] |
| 138 | 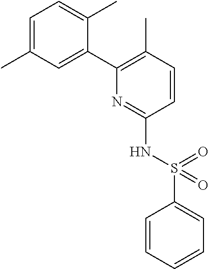 [SMILES: Cc1ccc(C)c(-c2nc(NS(=O)(=O)c3ccccc3)ccc2C)c1] |
| 139 | 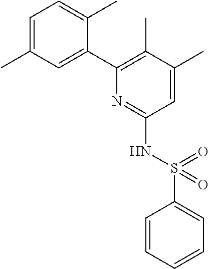 |
| 140 | 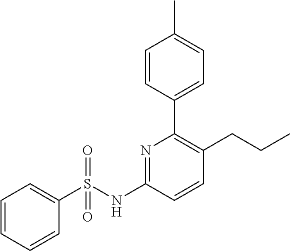 [SMILES: CCCc1ccc(NS(=O)(=O)c2ccccc2)nc1-c1ccc(C)cc1] |
| 141 | 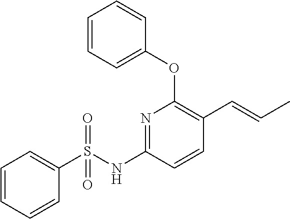 [SMILES: C/C=C/c1ccc(NS(=O)(=O)c2ccccc2)nc1Oc1ccccc1] |
| 142 | 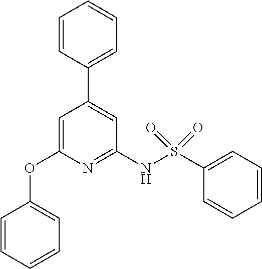 [SMILES: O=S(=O)(Nc1cc(-c2ccccc2)cc(Oc2ccccc2)n1)c1ccccc1] |
| 143 | 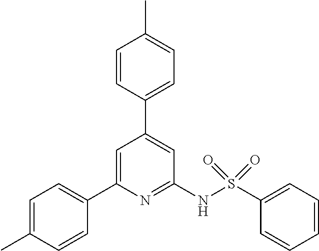 [SMILES: Cc1ccc(-c2cc(NS(=O)(=O)c3ccccc3)nc(-c3ccc(C)cc3)c2)cc1] |
| 144 | 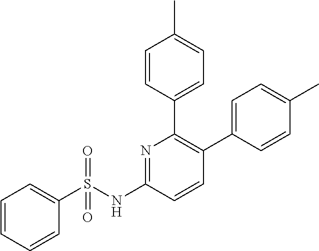 [SMILES: Cc1ccc(-c2ccc(NS(=O)(=O)c3ccccc3)nc2-c2ccc(C)cc2)cc1] |
| 146 | 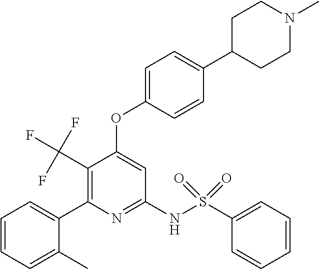 [SMILES: Cc1ccccc1-c1nc(NS(=O)(=O)c2ccccc2)cc(Oc2ccc(C3CCN(C)CC3)cc2)c1C(F)(F)F] |
| 147 | 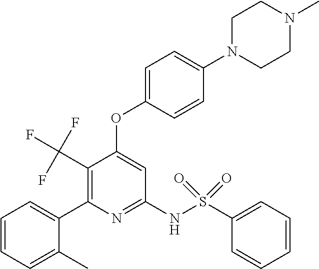 [SMILES: Cc1ccccc1-c1nc(NS(=O)(=O)c2ccccc2)cc(Oc2ccc(N3CCN(C)CC3)cc2)c1C(F)(F)F] |
| 148 | 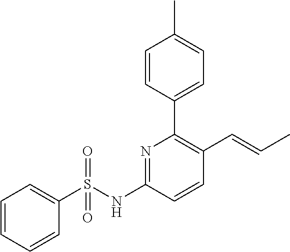 [SMILES: C/C=C/c1ccc(NS(=O)(=O)c2ccccc2)nc1-c1ccc(C)cc1] |
| 149 | 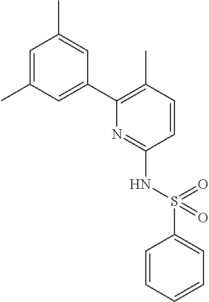 [SMILES: Cc1cc(C)cc(-c2nc(NS(=O)(=O)c3ccccc3)ccc2C)c1] |
| 150 | 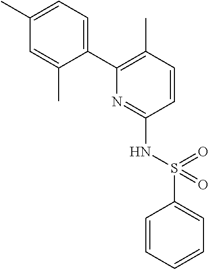 [SMILES: Cc1ccc(-c2nc(NS(=O)(=O)c3ccccc3)ccc2C)c(C)c1] |
| 151 | 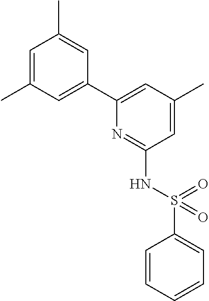 |
| 152 | 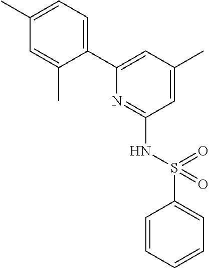 |
| 153 | 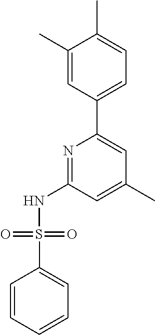 [SMILES: Cc1cc(NS(=O)(=O)c2ccccc2)nc(-c2ccc(C)c(C)c2)c1] |
| 154 | 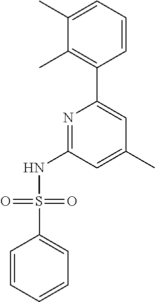 [SMILES: Cc1cc(NS(=O)(=O)c2ccccc2)nc(-c2cccc(C)c2C)c1] |
| 155 | 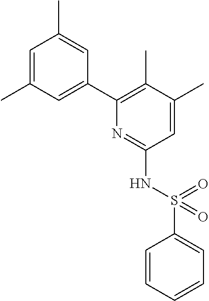 |
| 156 | 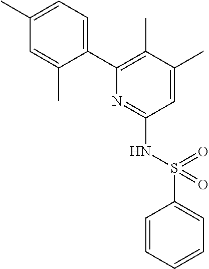 |
| 157 | 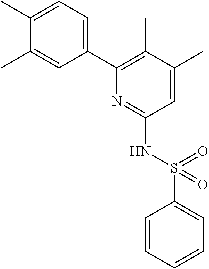 |
| 158 | 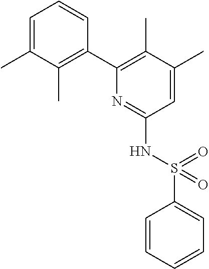 |

or a tautomer thereof, a deuterated derivative of the compound or tautomer, or a pharmaceutically acceptable salt of any of the foregoing.

19. A pharmaceutical composition comprising a compound, tautomer, deuterated derivative, or pharmaceutically acceptable salt of any one of claims 1-18 and a pharmaceutically acceptable carrier.

20. The pharmaceutical composition of claim 19, further comprising one or more additional therapeutic agent(s).

21. The pharmaceutical composition of claim 20, wherein the one or more additional therapeutic agent(s) comprise one or more CFTR modulator(s).

22. The pharmaceutical composition of claim 20, wherein the one or more additional therapeutic agent(s) comprise(s) one or more compound(s) selected from tezacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

23. A method of treating cystic fibrosis comprising administering to a patient in need thereof a compound, tautomer, deuterated derivative, or pharmaceutically acceptable salt of any one of claims 1-18 or a pharmaceutical composition according to any one of claims 19-22.

24. The compound, tautomer, deuterated derivative, or pharmaceutically acceptable salt of any one of claims 1-18 or the pharmaceutical composition according to any one of claims 19-22 for use in the treatment of cystic fibrosis.

25. The compound, tautomer, deuterated derivative, or pharmaceutically acceptable salt of any one of claims 1-18 or the pharmaceutical composition according to any one of claims 19-22 for use in the manufacture of a medicament for the treatment of cystic fibrosis.

26. A compound selected from Compounds 1-158, tautomers thereof, deuterated derivatives of those compounds and tautomers, and pharmaceutically acceptable salts of any of the foregoing.

27. A deuterated derivative of a compound selected from Compounds 1-158.

28. A pharmaceutically acceptable salt of a compound selected from Compounds 1-158.

29. A compound selected from Compounds 1-158.

30. A pharmaceutical composition comprising a compound selected from Compounds 1-158, tautomers thereof, deuterated derivatives of those compounds and tautomers, and pharmaceutically acceptable salts of any of the foregoing and a pharmaceutically acceptable carrier.

31. A pharmaceutical composition comprising a deuterated derivative of a compound selected from Compounds 1-158 and a pharmaceutically acceptable carrier.

32. A pharmaceutical composition comprising a pharmaceutically acceptable salt of a compound selected from Compounds 1-158 and a pharmaceutically acceptable carrier.

33. A pharmaceutical composition comprising a compound selected from Compounds 1-158 and a pharmaceutically acceptable carrier.

34. A pharmaceutical composition comprising (a) a compound selected from Compounds 1-158, tautomers thereof, deuterated derivatives of those compounds and tautomers, and pharmaceutically acceptable salts of any of the foregoing; (b) a CFTR potentiator; and (c) a pharmaceutically acceptable carrier.

35. A pharmaceutical composition composition comprising (a) a deuterated derivative of a compound selected from Compounds 1-158; (b) a CFTR potentiator; and (c) a pharmaceutically acceptable carrier.

36. A pharmaceutical comprising (a) a pharmaceutically acceptable salt of a compound selected from Compounds 1-158; (b) a CFTR potentiator; and (c) a pharmaceutically acceptable carrier.

37. A pharmaceutical composition comprising (a) a compound selected from Compounds 1-158; (b) a CFTR potentiator; and (c) a pharmaceutically acceptable carrier.

38. A pharmaceutical composition comprising (a) a compound selected from Compounds 1-158, tautomers thereof, deuterated derivatives of those compounds and tautomers, and pharmaceutically acceptable salts of any of the foregoing; (b) an additional CFTR corrector; and (c) a pharmaceutically acceptable carrier.

39. A pharmaceutical composition comprising (a) a deuterated derivative of a compound selected from Compounds 1-158; (b) an additional CFTR corrector; and (c) a pharmaceutically acceptable carrier.

40. A pharmaceutical composition comprising (a) a pharmaceutically acceptable salt of a compound selected from Compounds 1-158; (b) an additional CFTR corrector; and (c) a pharmaceutically acceptable carrier.

41. A pharmaceutical composition comprising (a) a compound selected from Compounds 1-158; (b) an additional CFTR corrector; and (c) a pharmaceutically acceptable carrier.

42. A pharmaceutical composition comprising (a) a compound selected from Compounds 1-158, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.

43. A pharmaceutical composition comprising (a) a deuterated derivative of a compound selected from Compounds 1-158; (b) an additional CFTR corrector; (c) a CFTR potentiator; and (d) a pharmaceutically acceptable carrier.

44. A pharmaceutical composition comprising (a) a pharmaceutically acceptable salt of a compound selected from Compounds 1-158; (b) an additional CFTR corrector; (c) a CFTR potentiator; and (d) a pharmaceutically acceptable carrier.

45. A pharmaceutical composition comprising (a) a compound selected from Compounds 1-158; (b) an additional CFTR corrector; (c) a CFTR potentiator; and (d) a pharmaceutically acceptable carrier.

46. A compound selected from Compounds 1-158, tautomers thereof, deuterated derivatives of those compounds and tautomers, and pharmaceutically acceptable salts of any of the foregoing for use in a method of treating cystic fibrosis.

47. A deuterated derivative of a compound selected from Compounds 1-158 for use in a method of treating cystic fibrosis.

48. A pharmaceutically acceptable salt of a compound selected from Compounds 1-158 for use in a method of treating cystic fibrosis.

49. A compound selected from Compounds 1-158 for use in a method of treating cystic fibrosis.

50. A pharmaceutical composition comprising a compound selected from Compounds 1-158, tautomers thereof, deuterated derivatives of those compounds and tautomers, and pharmaceutically acceptable salts of any of the foregoing and a pharmaceutically acceptable carrier for use in a method of treating cystic fibrosis.

51. A pharmaceutical composition comprising a deuterated derivative of a compound selected from Compounds 1-158 and a pharmaceutically acceptable carrier for use in a method of treating cystic fibrosis.

52. A pharmaceutical composition comprising a pharmaceutically acceptable salt of a compound selected from Compounds 1-158 and a pharmaceutically acceptable carrier for use in a method of treating cystic fibrosis.

53. A pharmaceutical composition comprising a compound selected from Compounds 1-158 and a pharmaceutically acceptable carrier for use in a method of treating cystic fibrosis.

54. A pharmaceutical composition comprising (a) a compound selected from Compounds 1-158, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.

55. A pharmaceutical comprising (a) a deuterated derivative of a compound selected from Compounds 1-158; (b) a CFTR potentiator; and (c) a pharmaceutically acceptable carrier for use in a method of treating cystic fibrosis.

56. A pharmaceutical composition comprising (a) a pharmaceutically acceptable salt of a compound selected from Compounds 1-158; (b) a CFTR potentiator; and (c) a pharmaceutically acceptable carrier for use in a method of treating cystic fibrosis.

57. A pharmaceutical composition comprising (a) a compound selected from Compounds 1-158; (b) a CFTR potentiator; and (c) a pharmaceutically acceptable carrier.

58. A pharmaceutical composition comprising (a) a compound selected from Compounds 1-158, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.

59. A pharmaceutical composition comprising (a) a deuterated derivative of a compound selected from Compounds 1-158; (b) an additional CFTR corrector; and (c) a pharmaceutically acceptable carrier for use in a method of treating cystic fibrosis.

60. A pharmaceutical composition comprising (a) a pharmaceutically acceptable salt of a compound selected from Compounds 1-158; (b) an additional CFTR corrector; and (c) a pharmaceutically acceptable carrier for use in a method of treating cystic fibrosis.

61. A pharmaceutical composition comprising (a) a compound selected from Compounds 1-158; (b) an additional CFTR corrector; and (c) a pharmaceutically acceptable carrier for use in a method of treating cystic fibrosis.

62. A pharmaceutical composition comprising (a) a compound selected from Compounds 1-158, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.

63. A pharmaceutical composition comprising (a) a deuterated derivative of a compound selected from Compounds 1-158; (b) an additional CFTR corrector; (c) a CFTR potentiator; and (d) a pharmaceutically acceptable carrier for use in a method of treating cystic fibrosis.

64. A pharmaceutical composition comprising (a) a pharmaceutically acceptable salt of a compound selected from Compounds 1-158; (b) an additional CFTR corrector; (c) a CFTR potentiator; and (d) a pharmaceutically acceptable carrier for use in a method of treating cystic fibrosis.

65. A pharmaceutical composition comprising (a) a compound selected from Compounds 1-158; (b) an additional CFTR corrector; (c) a CFTR potentiator; and (d) a pharmaceutically acceptable carrier for use in a method of treating cystic fibrosis.
